(19) United States
(12) Patent Application Publication
(10) Pub. No.: US 2025/0228132 A1
(43) Pub. Date: Jul. 10, 2025

(54) ORGANIC COMPOUNDS AND ORGANIC LIGHT EMITTING DIODE COMPRISING THE SAME

(71) Applicant: Material Science Co.,Ltd., Hwaseong-si (KR)

(72) Inventors: Seung Uk CHO, Hwaseong-si (KR); Da Hee PARK, Hwaseong-si (KR); Sang Mee KIM, Hwaseong-si (KR); Hani JEON, Hwaseong-si (KR); Tae Wan LEE, Hwaseong-si (KR); Sang Hoon HONG, Hwaseong-si (KR)

(21) Appl. No.: 19/009,130

(22) Filed: Jan. 3, 2025

(30) Foreign Application Priority Data
Jan. 4, 2024 (KR) 10-2024-0001318
Dec. 27, 2024 (KR) 10-2024-0197969

(51) Int. Cl. H10K 85/60; C07D 307/91; C07D 333/76; C07D 409/10; C07D 409/12; H10K 50/15

(52) U.S. Cl. CPC H10K 85/636; C07D 307/91; C07D 333/76; C07D 409/10; C07D 409/12; H10K 85/633; H10K 50/156; H10K 85/615; H10K 85/6574; H10K 85/6576

(57) ABSTRACT

Disclosed is an organic compound represented by a Chemical Formula A and an organic light emitting diode including the same, wherein an organic compound represented by a Chemical Formula A is contained in a hole transport layer or an hole transport auxiliary layer, thereby lowering an operation voltage of the diode, and improving efficiency, and lifetime characteristics of the organic light emitting diode.

### CROSS-REFERENCE TO RELATED APPLICATION

This application claims priority from Korean Patent Application No. 10-2024-0001318 filed on Jan. 4, 2024 and Korean Patent Application No. 10-2024-0197969 filed on Dec. 27, 2024 in the Korean Intellectual Property Office, and all the benefits accruing therefrom under 35 U.S.C. 119, the contents of which in its entirety are herein incorporated by reference.

### BACKGROUND

### Field

The present disclosure relates to an organic compound and an organic light emitting diode including the same.

### Description of Related Art

An organic light emitting diode (OLED) has a simpler structure compared to other flat panel display devices such as a liquid crystal display (LCD), a plasma display panel (PDP), and a field emission display (FED), and has various advantages in terms of a manufacturing process, and has excellent high luminance and wide viewing angle, fast response speed, and low operation voltage, and thus is being actively developed and commercialized as a flat display such as a wall-mounted TV, a backlight for a display, lighting, and billboards.

The organic light emitting diode includes two electrodes, and an organic material layer therebetween. Electrons and holes from two electrodes are injected into a light emitting layer in which excitons are generated via recombination of electrons and holes. When the generated excitons change from an excited state to a ground state, the light is generated.

The organic light emitting diode may include at least one light emitting layer. In general, the organic light emitting diode having a plurality of light emitting layers includes light emitting layers that emit light beams with different peak wavelengths. Thus, a specific color may be rendered via a combination of the light beams with the different peak wavelengths.

The organic light emitting diode may be classified into a top emission type light emitting diode and a bottom emission type light emitting diode. The top emission type light emitting diode emits light generated in the light emitting layer toward a translucent first electrode (anode) using a reflective second electrode (cathode). On the other hand, in the bottom emission type light emitting diode, light generated in the light emitting layer is reflected from a reflective first electrode (anode) to be directed toward a transparent second electrode (cathode), that is, toward a driving thin film transistor.

### SUMMARY

A purpose of the present disclosure is to provide a novel organic compound and an organic light emitting diode including the same.

Exemplary embodiments of the present invention may be used to achieve the above and other objects.

Purposes of the present disclosure are not limited to the above-mentioned purpose. Other purposes and advantages of the present disclosure that are not mentioned may be understood based on following descriptions, and may be more clearly understood based on embodiments of the present disclosure. Further, it will be easily understood that the purposes and advantages of the present disclosure may be realized using means shown in the claims and combinations thereof.

According to one embodiment of the present disclosure, an organic compound represented by a following Chemical Formula A is provided. The definition of the Chemical Formula A as set forth below is the same as described in the present specification and claims.

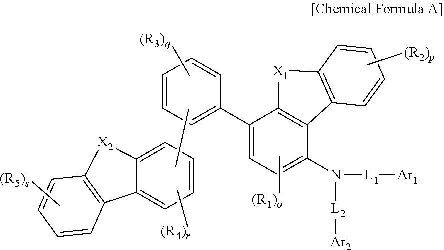

wherein in the Chemical Formula A,
o is an integer of 0 to 2, each of p, q, and s is an integer of 0 to 4, and r is an integer of 0 to 3,
X₁ and X₂ are identical with or different from each other, and each thereof is independently oxygen (O) or sulfur(S),
L₁ and L₂ are identical with or different from each other, and each thereof is independently selected from a single bond and a substituted or unsubstituted arylene group having 6 to 30 carbon atoms,
R₁ to R₅ are identical with or different from each other, and each thereof is independently selected from hydrogen and deuterium,
Ar₁ and Ar₂ are identical with or different from each other, and each thereof is independently selected from a substituted or unsubstituted aryl group having 6 to 30 carbon atoms and a substituted or unsubstituted heteroaryl group having 5 to 60 carbon atoms,
when L₁, L₂, Ar₁, and Ar₂ are respectively substituted with substituents, the substituents are identical with or different from each other, wherein each of substituents independently includes at least one selected from deuterium, a cyano group, a trifluoromethyl group, a nitro group, a halogen group, a hydroxy group, a trimethylsilyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms, wherein when each of substituents includes a plurality of substituents, the plurality of substituents are identical with or different from each other.

According to another embodiment of the present disclosure, there is provided an organic light emitting diode comprising: a first electrode; a second electrode facing the first electrode; and at least one organic layer disposed between the first electrode and the second electrode, wherein the organic layer includes at least one of a hole transport layer and an hole transport auxiliary layer, wherein at least one of the hole transport layer and the hole transport auxiliary layer includes the compound represented by the Chemical Formula A as described above.

The organic compound represented by the Chemical Formula A of the present disclosure may have excellent hole transport properties.

In addition, at least one of the hole transport layer and the hole transport auxiliary layer of the organic light emitting diode of the present disclosure contains the organic compound represented by the Chemical Formula A of the present disclosure, thereby lowering an operation voltage of the diode, and improving external quantum efficiency, and lifetime characteristics of the organic light emitting diode.

In addition, when the organic compound represented by the Chemical Formula A of the present disclosure is used as the hole transport auxiliary layer material, the compound may have a suitable energy level so as to act as the hole transport auxiliary layer serving to transfer holes from the hole transport layer to the light emitting layer and block electrons coming from the light emitting layer.

In addition, in the organic light emitting device of the present disclosure, even when the hole transport layer and/or the hole transport auxiliary layer including the organic compound represented by the Chemical Formula A of the present disclosure is combined with the light emitting layer emitting light of any color, the light emitting layer may emit light of a color having a target color coordinate excellently.

The effects of the present disclosure are not limited to the effects mentioned above, and other effects not mentioned will be clearly understood by those of ordinary skill in the art from the entire description of the present specification. The above effects and additional effects will be described in detail below.

### DETAILED DESCRIPTIONS

The above-mentioned purposes, features, and advantages are described in detail below, and accordingly, those skilled in the art in the technical field to which the present disclosure belongs will be able to easily implement the technical ideas of the present disclosure.

In describing the present disclosure, if it is determined that a detailed description of a related known technology may unnecessarily obscure the gist of the present disclosure, the detailed description is omitted.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, “including”, “contain”, “containing”, etc. when used in this specification, specify the presence of the stated features, integers, operations, elements, and/or components, but do not preclude the presence or addition of one or more other features, integers, operations, elements, components, and/or portions thereof.

In interpreting a numerical value, the value is interpreted as including an error range unless there is no separate explicit description thereof.

When an element such as a layer, film, region or substrate is referred to as being placed “above (or below)” or “on (or under)” another element, it can be directly placed on the other element, or intervening layer(s) may also be present.

As used herein, the term “halogen group” includes fluorine, chlorine, bromine and iodine.

As used herein, the term “alkyl group” refers to both straight-chain alkyl radicals and branched-chain alkyl radicals. Unless otherwise specified, an alkyl group contains 1 to 30 carbon atoms. In this case, the alkyl group may include methyl, ethyl, propyl, isopropyl, butyl, secondary butyl, isobutyl, tert-butyl, pentyl, isoamyl, hexyl, etc., but is not limited thereto. Additionally, the alkyl group may be optionally substituted.

As used herein, the term “cycloalkyl group” refers to a cyclic alkyl radical. Unless otherwise specified, a cycloalkyl group contains 3 to 20 carbon atoms. In this case, the cycloalkyl group may include cyclopropyl, cyclobutyl, cyclopentyl, cyclohexyl, norbornyl, adamantyl, etc., but is not limited thereto. Additionally, the cycloalkyl group may be optionally substituted.

As used herein, the term “alkenyl group” refers to both straight-chain alkenyl radicals and branched-chain alkenyl radicals having one or more carbon-carbon double bonds. Unless otherwise specified, an alkenyl group contains 2 to 30 carbon atoms. In this case, the alkenyl group may include vinyl, allyl, isopropenyl, 2-butenyl, etc., but is not limited thereto. Additionally, the alkenyl group may be optionally substituted.

As used herein, the term “cycloalkenyl group” refers to a cyclic alkenyl radical. Unless otherwise specified, a cycloalkenyl group contains 3 to 20 carbon atoms. Additionally, the cycloalkenyl group may be optionally substituted.

As used herein, the term “alkynyl group” refers to both straight-chain and branched-chain alkynyl radicals having one or more carbon-carbon triple bonds. Unless otherwise specified, an alkynyl group contains 2 to 30 carbon atoms. In this case, an alkynyl group may include, but is not limited to, ethynyl, 2-propynyl, etc. Additionally, the alkynyl group may be optionally substituted.

As used herein, the term “cycloalkynyl group” refers to a cyclic alkynyl radical. Unless otherwise specified, a cycloalkynyl group contains 3 to 20 carbon atoms. Additionally, cycloalkynyl groups may be optionally substituted.

The terms “aralkyl group” and “arylalkyl group” as used herein are used interchangeably with each other and refer to an alkyl group having an aromatic group as a substituent. Additionally, the aralkyl group (arylalkyl group) may be optionally substituted.

The terms “aryl group” and “aromatic group” as used herein are used as having the same meaning, and the aryl group includes both a monocyclic group and a polycyclic group. The polycyclic group may include a “fused ring” in which two or more rings are fused with each other such that two carbons are common to two adjacent rings. Moreover, in the polycyclic group, two or more rings may be simply attached or fused to each other. Unless otherwise specified, the aryl group contains 6 to 30 carbon atoms. In this case, the aryl group may include phenyl, naphthyl, phenanthryl, anthryl, fluorenyl, dimethylfluorenyl, diphenylfluorenyl, and spirobifluorenyl, etc. but is not limited thereto. Additionally, the aryl group may be optionally substituted.

The terms “heteroaryl group” and “heteroaromatic group” as used herein are used as having the same meaning, and the heteroaryl group includes both a monocyclic group and a polycyclic group. The polycyclic group may include a “fused ring” in which two or more rings are fused with each other such that two carbons or heteroatoms are common to two adjacent rings. Moreover, in the polycyclic group, two or more rings may be simply attached or fused to each other. Unless otherwise specified, the heteroaryl group contains 5 to 60 carbon atoms. In this regard, one or more carbons of a ring are replaced with heteroatoms such as oxygen (O), nitrogen (N), sulfur(S), or selenium (Se). In this case, the heteroaryl group may include a 6-membered monocyclic ring such as pyridyl, pyrazinyl, pyrimidinyl, pyridazinyl, and triazinyl, a polycyclic ring such as phenoxathiinyl, indolizinyl, indolyl, purinyl, quinolyl, isoquinolyl, benzooxyzolyl, benzothiazolyl, dibenzooxyzolyl, dibenzothiazolyl, benzoimidazolyl, benzofuranyl, dibenzofuranyl, benzothiophenyl, dibenzothiophenyl, carbazolyl, phenylcarbazolyl, 9-phenylcarbazolyl, etc. but is not limited thereto. Additionally, the heteroaryl group may be optionally substituted.

The term “heterocyclic group” as used herein means that at least one of the carbon atoms constituting an aryl group, a cycloalkyl group, a cycloalkenyl group, a cycloalkynyl group, an arylalkyl group, an arylamino group, etc. is substituted with a heteroatom such as oxygen (O), nitrogen (N), sulfur(S), etc. Referring to the above definition, the heterocyclic group may include a heteroaryl group, a heterocycloalkyl group, a heterocycloalkenyl group, a heterocycloalkynyl group, a heteroarylalkyl group, a heteroarylamino group, etc. Additionally, the heterocyclic group may be optionally substituted.

Unless otherwise specified, the term “carbon ring” as used herein may be used as including all of a “cycloalkyl group”, “cycloalkenyl group”, “cycloalkynyl group” as an alicyclic group and “aryl group (aromatic group”) as an aromatic ring group.

Each of the terms “heteroalkyl group”, “heteroalkenyl group”, “heteroalkynyl group”, and “heteroarylalkyl group” as used herein means that one or more of the carbon atoms constituting the group is substituted with a heteroatom such as oxygen (O), nitrogen (N), sulfur(S). Additionally, each of the heteroalkyl group, heteroalkenyl group, heteroalkynyl group, and heteroarylalkyl group may be optionally substituted.

As used herein, the terms “alkylamino group”, “arylalkylamino group”, “arylamino group”, and “heteroarylamino group” refer to groups in which the alkyl group, arylalkyl group, aryl group, or a heteroaryl group as a hetero-ring is substituted with an amine group. In this regard, the amino group may include all of the primary, secondary, and tertiary amines. Additionally, the alkylamino group, the arylalkylamino group, the arylamino group, and the heteroarylamino group may be optionally substituted

As used herein, the terms “alkylsilyl group”, “arylsilyl group”, “alkoxy group”, “aryloxy group”, “alkylthio group”, or “arylthio group” refer to groups in which each of the alkyl group and the aryl group is substituted with each of a silyl group, an oxy group, and a thio group. Additionally, the alkylsilyl group, the arylsilyl group, the alkoxy group, the aryloxy group, the alkylthio group, and the arylthio group may be optionally substituted.

The terms “arylene group”, “arylalkylene group”, “heteroarylene group”, or “heteroarylalkylene group” as used herein means a group having two-substitutions in which the aryl group, arylalkyl group, heteroaryl group, or heteroarylalkyl group further includes one substitution. Additionally, the arylene group, arylalkylene group, heteroarylene group, and heteroarylalkylene group may be optionally substituted.

As used herein, the term “substituted” means that a hydrogen atom (H) binding to a carbon atom or a nitrogen atom of the compound of the present disclosure is replaced with a substituent other than hydrogen. When there are a plurality of substituents, the substituents may be the same as or different from each other.

The substituent may independently include at least one selected from deuterium, a cyano group, a trifluoromethyl group, a nitro group, a halogen group, a hydroxy group, a trimethylsilyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms.

Unless otherwise specified, a position at which the substitution occurs is not particularly limited as long as a hydrogen atom can be substituted with a substituent at the position. When two or more substituents, that is, the plurality of substituents are present, the substituents may be identical to or different from each other.

Subjects and substituents as defined in the present disclosure may be the same as or different from each other unless otherwise specified.

As used herein, a unit is based on weight (wt), unless specifically stated. For example, when “%” is written, this is interpreted as weight % (wt %).

Hereinafter, an organic compound and an organic light emitting device including the same according to the present disclosure will be described in detail.

The organic compound in accordance with the present disclosure may be represented by a following Chemical Formula A:

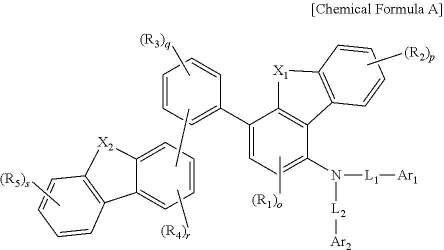

wherein in the Chemical Formula A, o may be an integer of 0 to 2, each of p, q, and s may be an integer of 0 to 4, and r may be an integer of 0 to 3,
X₁ and X₂ may be identical with or different from each other, and each thereof may be independently oxygen (O) or sulfur(S).

L₁ and L₂ may be identical with or different from each other, and each thereof may be independently selected from a single bond, a substituted or unsubstituted arylene group having 6 to 30 carbon atoms, a substituted or unsubstituted arylalkylene group having 7 to 30 carbon atoms, a substituted or unsubstituted heteroarylene group having 5 to 60 carbon atoms, and a substituted or unsubstituted heteroarylalkylene group having 6 to 60 carbon atoms. In one example, L₁ and L₂ may be identical with or different from each other, and each thereof may be independently selected from a single bond and a substituted or unsubstituted arylene group having 6 to 30 carbon atoms.

R₁ to R₅ may be identical with or different from each other, and each thereof may be independently selected from hydrogen, deuterium, a cyano group, a trifluoromethyl group, a nitro group, a halogen group, a hydroxy group, a trimethylsilyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, alkylamino group having 1 to 30 carbon atoms, arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms. In one example, R₁ to R₅ may be identical with or different from each other, and each thereof may be independently selected from hydrogen and deuterium.

Ar₁ and Ar₂ may be identical with or different from each other, and each thereof may be independently selected from a substituted or unsubstituted aryl group having 6 to 30 carbon atoms, a substituted or unsubstituted arylalkyl group having 7 to 30 carbon atoms, a substituted or unsubstituted heteroaryl group having 5 to 60 carbon atoms, a substituted or unsubstituted heteroarylalkyl group having 6 to 60 carbon atoms, a substituted or unsubstituted arylamino group having 6 to 30 carbon atoms, a substituted or unsubstituted arylalkylamino group having 6 to 30 carbon atoms, a substituted or unsubstituted heteroarylamino group having 5 to 60 carbon atoms, a substituted or unsubstituted arylsilyl group having 6 to 30 carbon atoms and a substituted or unsubstituted aryloxy group having 6 to 30 carbon atoms. In one example, Ar₁ and Ar₂ may be identical with or different from each other, and each thereof may be independently selected from a substituted or unsubstituted aryl group having 6 to 30 carbon atoms and a substituted or unsubstituted heteroaryl group having 5 to 60 carbon atoms. Optionally, adjacent groups may bind to each other to form a substituted or unsubstituted ring.

When L₁, L₂, Ar₁, and Ar₂ are respectively substituted with substituents, the substituents are identical with or different from each other, wherein each of substituents independently includes at least one selected from deuterium, a cyano group, a trifluoromethyl group, a nitro group, a halogen group, a hydroxy group, a trimethylsilyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms, wherein when each of substituents includes a plurality of substituents, the plurality of substituents are identical with or different from each other. Optionally, adjacent substituents among the substituents may bind to each other to form a ring.

According to one example of the present disclosure, the Chemical Formula A may be represented by any one of following Chemical Formulas 1 to 3:

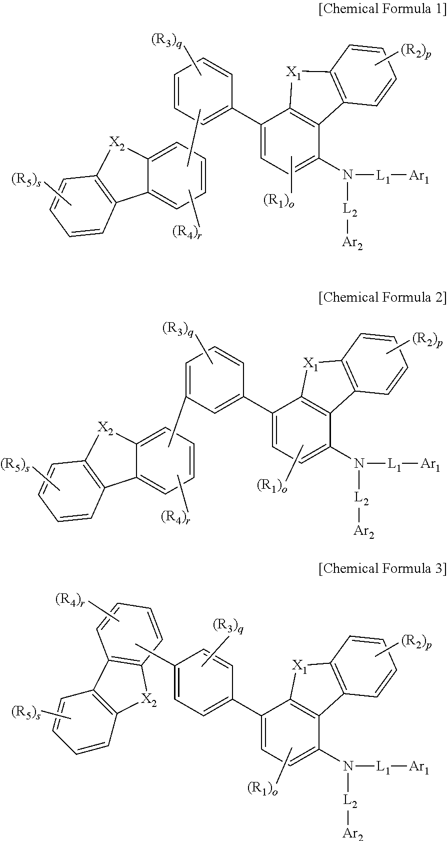

As may be identified from the structures of the Chemical Formulas 1 to 3, the Chemical Formula A may be represented by one of the Chemical Formulas 1 to 3 based on a position where the ring structure including X₂ is connected to the phenylene group serving as a linker between the ring structure including X₂ and the ring structure including X₁ in the Chemical Formula A. In the Chemical Formulas 1 to 3, each of o, p, q, r, s, X₁, X₂, L₁, L₂, R₁ to R₅, Ar₁ and Ar₂ may be the same as defined in the Chemical Formula A.

According to one example of the present disclosure, the Chemical Formula A may be preferably selected from the Chemical Formula 1 and the Chemical Formula 2.

According to one example of the present disclosure, the Chemical Formula 1 may be represented by any one of following Chemical Formulas 1-1 to 1-4:

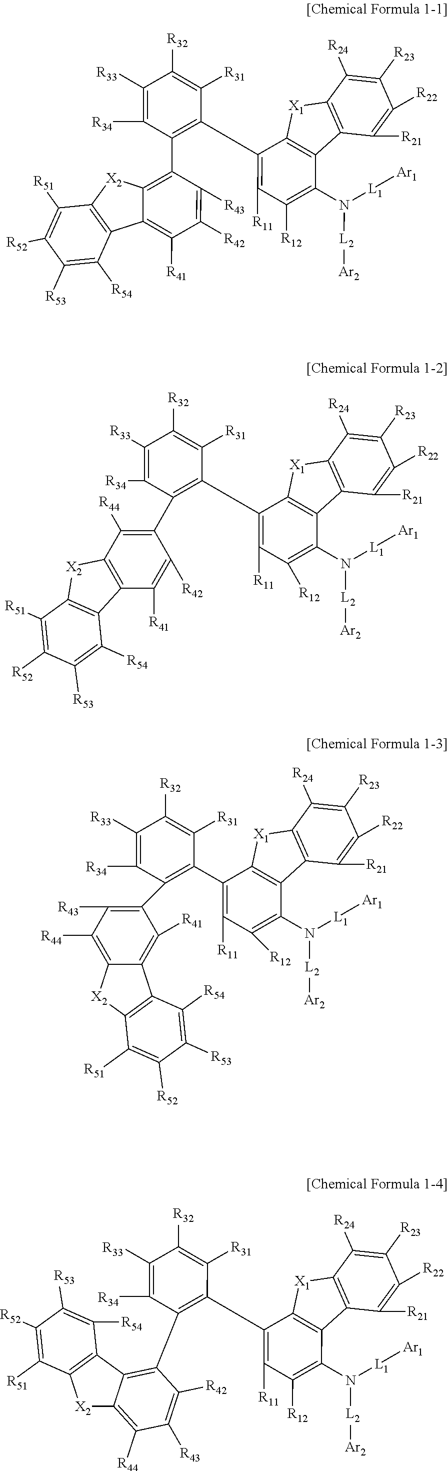

As may be identified from the structures of the Chemical Formulas 1-1 to 1-4, the Chemical Formula 1 may be represented by one of the Chemical Formulas 1-1 to 1-4, depending on a position at which the ring structure including X₂ is connected to the phenylene group as the linker in the Chemical Formula 1.

In the Chemical Formulas 1-1 to 1-4, R₁ of the Chemical Formula 1 is represented by R₁₁ to R₁₂, R₂ thereof is represented by R₂₁ to R₂₄, R₃ thereof is represented by R₃₁ to R₃₄, and R₄ thereof is represented by R₄₁ to R₄₄, and R₅ thereof is represented by R₅₁ to R₅₄, respectively. Thus, in the Chemical Formulas 1-1 to 1-4, each of X₁, X₂, L₁, L₂, Ar₁ and Ar₂ may be the same as defined in the Chemical Formula A, and each of R₁₁ to R₁₂ may be the same as defined in R₁, each of R₂₁ to R₂₄ may be the same as defined in R₂, and each of R₃₁ to R₃₄ may be the same as defined in R₃, each of R₄₁ to R₄₄ may be the same as defined in R₄, and each of R₅₁ to R₅₄ may be the same as defined in R₅.

According to one example of the present disclosure, the Chemical Formula 2 may be represented by any one of following Chemical Formulas 2-1 to 2-4:

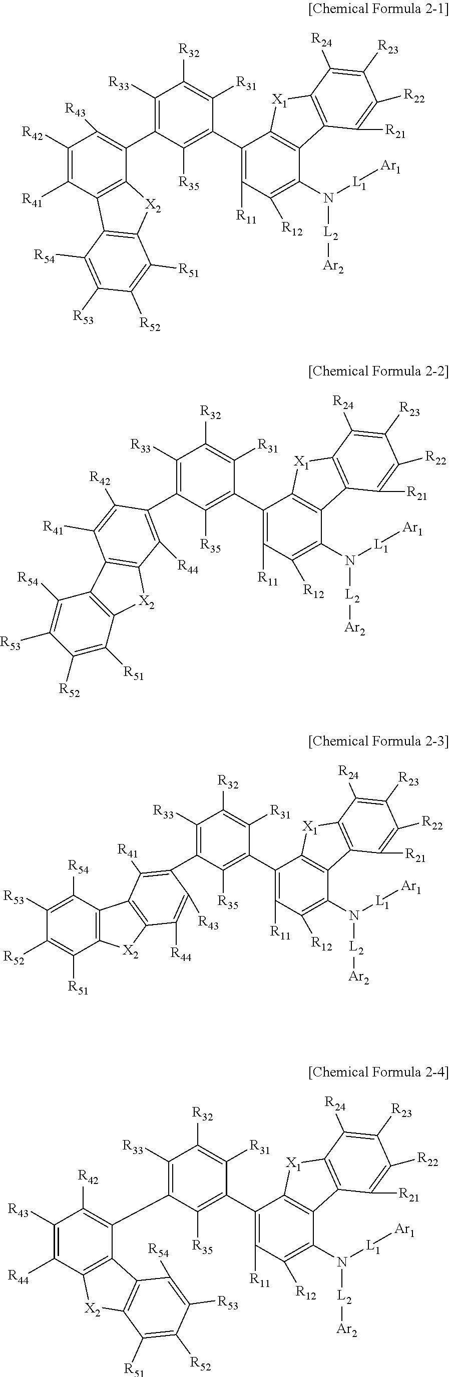

As may be identified from the structures of the Chemical Formulas 2-1 to 2-4, the Chemical Formula 2 may be represented by one of the Chemical Formulas 2-1 to 2-4, depending on a position at which the ring structure including X₂ is connected to the phenylene group as the linker in the Chemical Formula 2.

In the Chemical Formulas 2-1 to 2-4, R₁ of the Chemical Formula 2 is represented by R₁₁ to R₁₂, R₂ thereof is represented by R₂₁ to R₂₄, R₃ thereof is represented by R₃₁ to R₃₃, R₃₅, and R₄ thereof is represented by R₄₁ to R₄₄, and R₅ thereof is represented by R₅₁ to R₅₄, respectively. Thus, in the Chemical Formulas 2-1 to 2-4, each of X₁, X₂, L₁, L₂, Ar₁ and Ar₂ may be the same as defined in the Chemical Formula A, and each of R₁₁ to R₁₂ may be the same as defined in R₁, each of R₂₁ to R₂₄ may be the same as defined in R₂, and each of R₃₁ to R₃₃, R₃₅ may be the same as defined in R₃, each of R₄₁ to R₄₄ may be the same as defined in R₄, and each of R₅₁ to R₅₄ may be the same as defined in R₅.

According to one example of the present disclosure, the Chemical Formula 3 may be represented by any one of following Chemical Formulas 3-1 to 3-4:

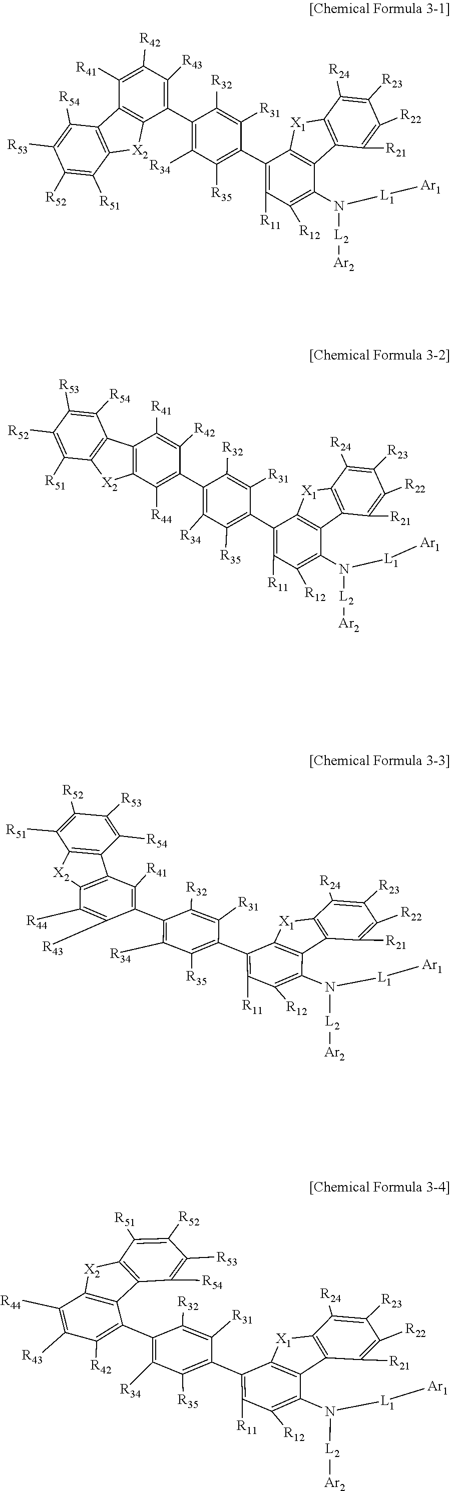

As may be identified from the structures of the Chemical Formulas 3-1 to 3-4, the Chemical Formula 3 may be represented by one of the Chemical Formulas 3-1 to 3-4, depending on a position at which the ring structure including X₂ is connected to the phenylene group as the linker in the Chemical Formula 3.

In the Chemical Formulas 3-1 to 3-4, R₁ of the Chemical Formula 3 is represented by R₁₁ to R₁₂, R₂ thereof is represented by R₂₁ to R₂₄, R₃ thereof is represented by R₃₁, R₃₂, R₃₄, R₃₅ and R₄ thereof is represented by R₄₁ to R₄₄, and R₅ thereof is represented by R₅₁ to R₅₄, respectively. Thus, in the Chemical Formulas 3-1 to 3-4, each of X₁, X₂, L₁, L₂, Ar₁ and Ar₂ may be the same as defined in the Chemical Formula A, and each of R₁₁ to R₁₂ may be the same as defined in R₁, each of R₂₁ to R₂₄ may be the same as defined in R₂, and each of R₃₁, R₃₂, R₃₄, R₃₅ may be the same as defined in R₃, each of R₄₁ to R₄₄ may be the same as defined in R₄, and each of R₅₁ to R₅₄ may be the same as defined in R₅.

According to one example of the present disclosure, L₁ and L₂ in each of Chemical Formulas 1-1 to 1-4, Chemical Formulas 2-1 to 2-4 and Chemical Formulas 3-1 to 3-4 may be identical with or different from each other, and each thereof may be independently selected from a single bond and a substituted or unsubstituted arylene group having 6 to 15 carbon atoms. For example, each of L₁ and L₂ may be selected from a single bond and a substituted or unsubstituted phenylene group.

According to one example of the present disclosure, Ar₁ and Ar₂ in each of Chemical Formulas 1-1 to 1-4, Chemical Formulas 2-1 to 2-4 and Chemical Formulas 3-1 to 3-4 may be identical with or different from each other, and each thereof may be independently a substituted or unsubstituted aryl group having 6 to 30 carbon atoms, or a substituted or unsubstituted heteroaryl group having 5 to 30 carbon atoms including at least one heteroatom selected from the group consisting of O, S, and N. For example, each of Ar₁ and Ar₂ may be selected from a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted dibenzofuranyl group, a substituted or unsubstituted dibenzothiophenyl group, a substituted or unsubstituted carbazolyl group, and a substituted or unsubstituted dimethylfluorenyl group.

According to one example of the present disclosure, when L₁, L₂, Ar₁, and Ar₂ are respectively substituted with substituents in each of Chemical Formulas 1-1 to 1-4, Chemical Formulas 2-1 to 2-4 and Chemical Formulas 3-1 to 3-4, the substituents may be identical with or different from each other, wherein each of substituents may independently include at least one selected from deuterium, a cyano group, a trifluoromethyl group, a nitro group, a halogen group, a hydroxy group, a trimethylsilyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms, wherein when each of substituents includes a plurality of substituents, the plurality of substituents may be identical with or different from each other. Optionally, adjacent substituents among the substituents may bind to each other to form a ring.

For example, the substituent may be one of deuterium, methyl, ethyl, propyl, butyl, pentyl, hexyl, cyclopropyl, cyclobutyl, cyclopentyl, cyclohexyl, adamantyl, phenyl, biphenyl, naphthyl, phenyl-naphthyl, anthracenyl, phenanthrenyl, dimethylfluorenyl, diphenylfluorenyl, spirofluorenyl, benzofuranyl, benzothiophenyl, dibenzofuranyl, dibenzothiophenyl, carbazolyl, phenylcarbazolyl and 9-phenylcarbazolyl.

According to one example of the present disclosure, in the Chemical Formula A, each of L₁ and L₂ may be selected from a single bond and following structures F1 and F2:

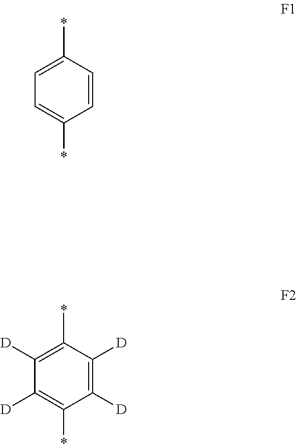 [SMILES: *c1ccc(*)cc1 ; *c1c([2H])c([2H])c(*)c([2H])c1[2H]]

In the above structures F1 and F2, “*” indicates a binding site.

According to one example of the present disclosure, in the Chemical Formula A, each of Ar₁ and Ar₂ may be selected from following structures M1 to M43. In the following structures M1 to M43, “*” indicates a binding site, and N in each of the following structures M28 and M29 means NH.

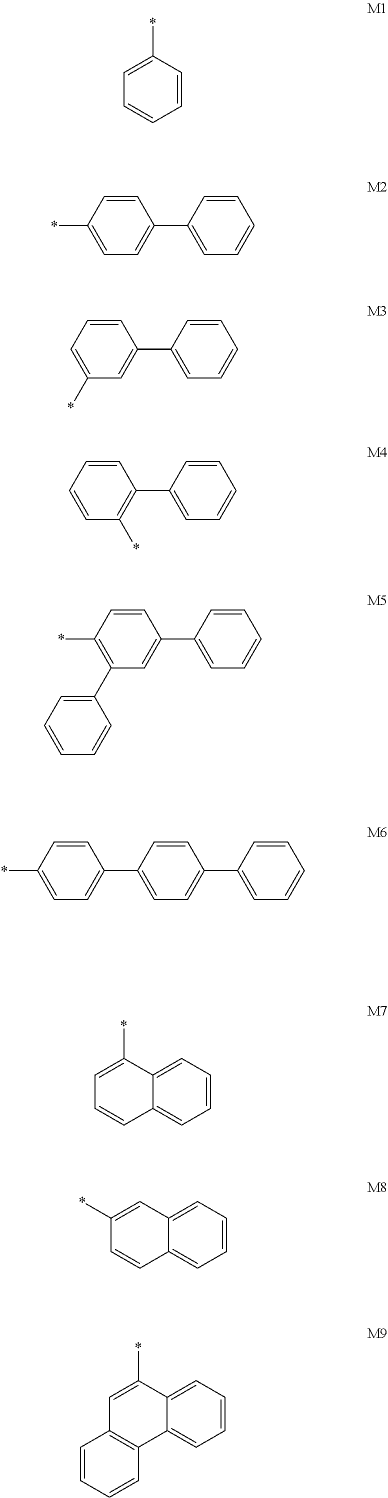

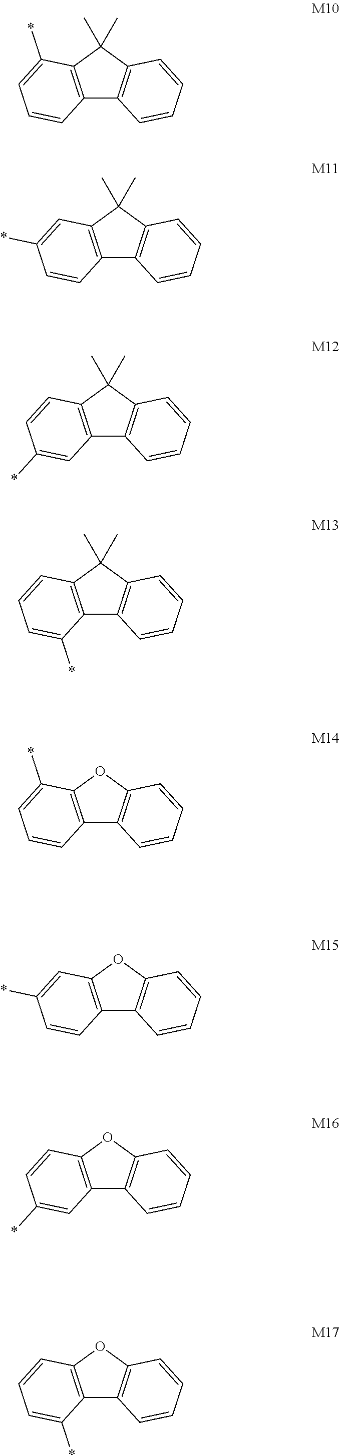

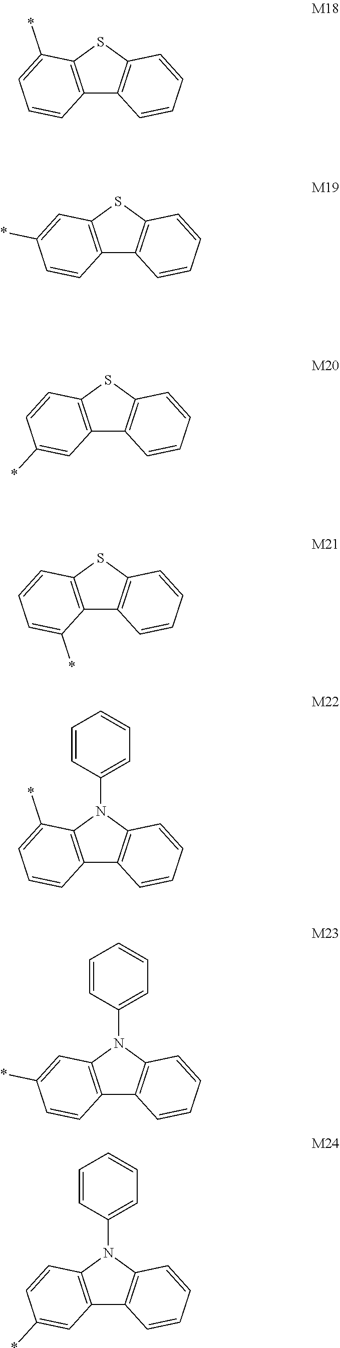

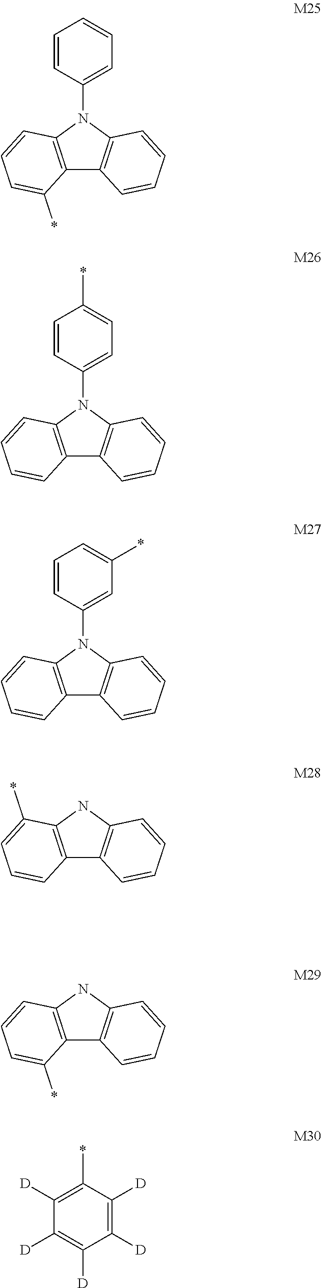 [SMILES: *c1cccc2c1c1ccccc1n2-c1ccccc1 ; *c1ccc(-n2c3ccccc3c3ccccc32)cc1 ; *c1cccc(-n2c3ccccc3c3ccccc32)c1 ; *c1cccc2c1[nH]c1ccccc12 ; *c1cccc2[nH]c3ccccc3c12 ; *c1c([2H])c([2H])c([2H])c([2H])c1[2H]]

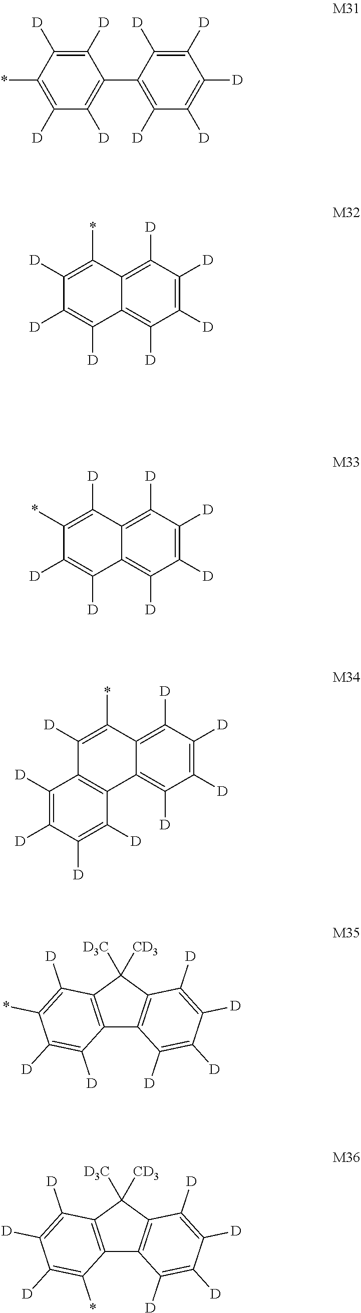 [SMILES: *c1c([2H])c([2H])c(-c2c([2H])c([2H])c([2H])c([2H])c2[2H])c([2H])c1[2H] ; *c1c([2H])c([2H])c([2H])c2c([2H])c([2H])c([2H])c([2H])c12 ; *c1c([2H])c([2H])c2c([2H])c([2H])c([2H])c([2H])c2c1[2H] ; *c1c([2H])c2c([2H])c([2H])c([2H])c([2H])c2c2c([2H])c([2H])c([2H])c([2H])c12 ; *c1c([2H])c([2H])c2c(c1[2H])C(C([2H])([2H])[2H])(C([2H])([2H])[2H])c1c([2H])c([2H])c([2H])c([2H])c1-2 ; *c1c([2H])c([2H])c([2H])c2c1-c1c([2H])c([2H])c([2H])c([2H])c1C2(C([2H])([2H])[2H])C([2H])([2H])[2H]]

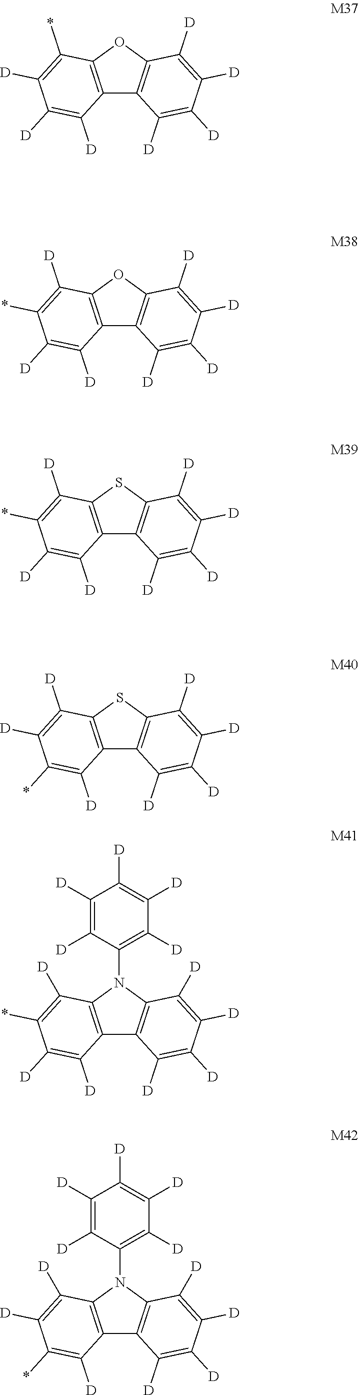 [SMILES: *c1c([2H])c([2H])c([2H])c2c1oc1c([2H])c([2H])c([2H])c([2H])c12 ; *c1c([2H])c([2H])c2c(oc3c([2H])c([2H])c([2H])c([2H])c32)c1[2H] ; *c1c([2H])c([2H])c2c(sc3c([2H])c([2H])c([2H])c([2H])c32)c1[2H] ; *c1c([2H])c([2H])c2sc3c([2H])c([2H])c([2H])c([2H])c3c2c1[2H] ; *c1c([2H])c([2H])c2c3c([2H])c([2H])c([2H])c([2H])c3n(-c3c([2H])c([2H])c([2H])c([2H])c3[2H])c2c1[2H] ; *c1c([2H])c([2H])c2c(c1[2H])c1c([2H])c([2H])c([2H])c([2H])c1n2-c1c([2H])c([2H])c([2H])c([2H])c1[2H]]

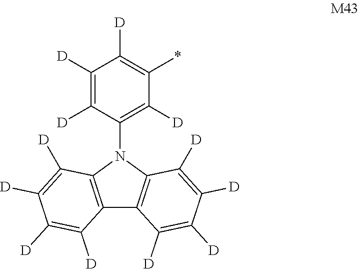 [SMILES: *c1c([2H])c([2H])c([2H])c(-n2c3c([2H])c([2H])c([2H])c([2H])c3c3c([2H])c([2H])c([2H])c([2H])c32)c1[2H]]

According to one example of the present disclosure, the compound represented by the Chemical Formula 1-1 may be selected from the group consisting of the compounds as shown in Tables 1 and 2 as set forth below. However, the present disclosure is not limited thereto. In the compounds as listed in Table 1 as set forth below, all of R₁₁ to R₁₂, R₂₁ to R₂₄, R₃₁ to R₃₄, R₄₁ to R₄₃, and R₅₁ to R₅₄ may be hydrogen. In the compounds as listed in Table 2 as set forth below, all of R₁₁ to R₁₂, R₂₁ to R₂₄, R₃₁ to R₃₄, R₄₁ to R₄₃, and R₅₁ to R₅₄ may be deuterium. In the following Tables, the mark “—” as each of X₁, X₂, L₁, L₂, Ar₁, and Ar₂ means a single bond.

TABLE 1

| Compound | | | | | | |
|---|---|---|---|---|---|---|
| No. | X₁ | X₂ | L₁ | Ar₁ | L₂ | Ar₂ |
| A1 | O | O | — | M1 | — | M1 |
| A2 | O | O | — | M7 | — | M1 |
| A3 | O | O | — | M8 | — | M1 |
| A4 | O | O | — | M10 | — | M1 |
| A5 | O | O | — | M2 | — | M1 |
| A6 | O | O | — | M4 | — | M1 |
| A7 | O | O | — | M5 | — | M1 |
| A8 | O | O | F1 | M7 | — | M1 |
| A9 | O | O | F1 | M10 | — | M1 |
| A10 | O | O | — | M9 | — | M1 |
| A11 | O | O | F1 | M9 | — | M1 |
| A12 | O | O | — | M14 | — | M1 |
| A13 | O | O | F1 | M14 | — | M1 |
| A14 | O | O | — | M18 | — | M1 |
| A15 | O | O | F1 | M18 | — | M1 |
| A16 | O | O | — | M24 | — | M1 |
| A17 | O | O | — | M26 | — | M1 |
| A18 | O | O | F1 | M22 | — | M1 |
| A19 | O | O | — | M8 | — | M7 |
| A20 | O | O | — | M6 | — | M7 |
| A21 | O | O | F1 | M7 | — | M7 |
| A22 | O | O | — | M7 | — | M7 |
| A23 | O | O | F1 | M11 | — | M7 |
| A24 | O | O | — | M9 | — | M7 |
| A25 | O | O | F1 | M9 | — | M7 |
| A26 | O | O | — | M15 | — | M7 |
| A27 | O | O | F1 | M14 | — | M7 |
| A28 | O | O | — | M19 | — | M7 |
| A29 | O | O | F1 | M20 | — | M7 |
| A30 | O | O | — | M24 | — | M7 |
| A31 | O | O | — | M26 | — | M7 |
| A32 | O | O | F1 | M23 | — | M7 |
| A33 | O | O | — | M11 | — | M8 |
| A34 | O | O | — | M3 | — | M8 |
| A35 | O | O | — | M5 | — | M8 |
| A36 | O | O | F1 | M16 | — | M8 |
| A37 | O | O | — | M11 | — | M10 |
| A38 | O | O | — | M2 | — | M10 |
| A39 | O | O | — | M3 | — | M10 |
| A40 | O | O | F1 | M8 | — | M10 |
| A41 | O | O | F1 | M10 | — | M10 |
| A42 | O | O | F1 | M9 | — | M10 |
| A43 | O | O | F1 | M14 | — | M10 |
| A44 | O | O | — | M19 | — | M10 |
| A45 | O | O | — | M24 | — | M10 |
| A46 | O | O | — | M2 | — | M11 |
| A47 | O | O | — | M5 | — | M11 |
| A48 | O | O | F1 | M7 | — | M11 |
| A49 | O | O | — | M9 | — | M11 |
| A50 | O | O | — | M15 | — | M11 |
| A51 | O | O | F1 | M14 | — | M11 |
| A52 | O | O | F1 | M20 | — | M11 |
| A53 | O | O | — | M26 | — | M11 |
| A54 | O | O | F1 | M24 | — | M11 |
| A55 | O | O | — | M4 | — | M12 |
| A56 | O | O | — | M5 | — | M12 |
| A57 | O | O | F1 | M17 | — | M12 |
| A58 | O | O | — | M3 | — | M13 |
| A59 | O | O | F1 | M16 | — | M13 |
| A60 | O | O | — | M2 | — | M2 |
| A61 | O | O | — | M4 | — | M2 |
| A62 | O | O | F1 | M11 | — | M2 |
| A63 | O | O | — | M11 | — | M2 |
| A64 | O | O | F1 | M12 | — | M2 |
| A65 | O | O | — | M9 | — | M2 |
| A66 | O | O | F1 | M16 | — | M2 |
| A67 | O | O | — | M18 | — | M2 |
| A68 | O | O | — | M23 | — | M2 |
| A69 | O | O | — | M6 | — | M4 |
| A70 | O | O | F1 | M8 | — | M4 |
| A71 | O | O | F1 | M10 | — | M4 |
| A72 | O | O | F1 | M15 | — | M4 |
| A73 | O | O | F1 | M21 | — | M4 |
| A74 | O | O | — | M24 | — | M4 |
| A75 | O | O | F1 | M24 | — | M4 |
| A76 | O | O | — | M2 | — | M3 |
| A77 | O | O | — | M3 | — | M3 |
| A78 | O | O | — | M5 | — | M3 |
| A79 | O | O | — | M6 | — | M3 |
| A80 | O | O | F1 | M12 | — | M3 |
| A81 | O | O | F1 | M9 | — | M3 |
| A82 | O | O | — | M14 | — | M3 |
| A83 | O | O | F1 | M17 | — | M3 |
| A84 | O | O | — | M19 | — | M3 |
| A85 | O | O | F1 | M20 | — | M3 |
| A86 | O | O | — | M5 | — | M6 |
| A87 | O | O | F1 | M7 | — | M6 |
| A88 | O | O | F1 | M10 | — | M6 |
| A89 | O | O | — | M14 | — | M6 |
| A90 | O | O | F1 | M14 | — | M6 |
| A91 | O | O | F1 | M20 | — | M6 |
| A92 | O | O | — | M26 | — | M6 |
| A93 | O | O | F1 | M8 | — | M5 |
| A94 | O | O | F1 | M11 | — | M5 |
| A95 | O | O | F1 | M9 | — | M5 |
| A96 | O | O | — | M15 | — | M5 |
| A97 | O | O | F1 | M16 | — | M5 |
| A98 | O | O | — | M19 | — | M5 |
| A99 | O | O | F1 | M19 | — | M5 |
| A100 | O | O | — | M24 | — | M5 |
| A101 | O | O | F1 | M23 | — | M5 |
| A102 | O | O | F1 | M8 | F1 | M7 |
| A103 | O | O | — | M9 | F1 | M7 |
| A104 | O | O | F1 | M15 | F1 | M7 |
| A105 | O | O | F1 | M18 | F1 | M7 |
| A106 | O | O | — | M24 | F1 | M7 |
| A107 | O | O | F1 | M23 | F1 | M8 |
| A108 | O | O | — | M14 | F1 | M10 |
| A109 | O | O | F1 | M14 | F1 | M10 |
| A110 | O | O | F1 | M18 | F1 | M10 |
| A111 | O | O | — | M9 | F1 | M11 |
| A112 | O | O | F1 | M16 | F1 | M11 |
| A113 | O | O | — | M19 | F1 | M11 |
| A114 | O | O | — | M23 | F1 | M11 |
| A115 | O | O | — | M26 | F1 | M11 |
| A116 | O | O | F1 | M24 | F1 | M12 |
| A117 | O | O | — | M15 | F1 | M13 |
| A118 | O | O | F1 | M16 | F1 | M13 |
| A119 | O | O | F1 | M9 | — | M9 |
| A120 | O | O | — | M15 | — | M9 |
| A121 | O | O | F1 | M14 | — | M9 |
| A122 | O | O | — | M19 | — | M9 |
| A123 | O | O | F1 | M18 | — | M9 |
| A124 | O | O | — | M24 | — | M9 |
| A125 | O | O | — | M26 | — | M9 |
| A126 | O | O | — | M14 | F1 | M9 |
| A127 | O | O | — | M15 | — | M14 |
| A128 | O | O | — | M19 | — | M14 |
| A129 | O | O | — | M24 | — | M14 |
| A130 | O | O | F1 | M24 | — | M14 |
| A131 | O | O | F1 | M15 | — | M15 |
| A132 | O | O | F1 | M20 | — | M15 |
| A133 | O | O | — | M26 | — | M15 |
| A134 | O | O | F1 | M17 | F1 | M14 |
| A135 | O | O | — | M19 | F1 | M14 |
| A136 | O | O | F1 | M19 | F1 | M14 |
| A137 | O | O | — | M23 | F1 | M14 |
| A138 | O | O | F1 | M23 | F1 | M15 |
| A139 | O | O | — | M18 | F1 | M16 |
| A140 | O | O | F1 | M24 | F1 | M16 |
| A141 | O | O | F1 | M19 | — | M18 |
| A142 | O | O | — | M24 | — | M19 |
| A143 | O | O | F1 | M24 | — | M19 |
| A144 | O | O | — | M23 | F1 | M19 |
| A145 | O | O | — | M26 | F1 | M19 |
| A146 | O | O | — | M26 | — | M24 |
| A147 | O | O | — | M23 | — | M24 |
| A148 | O | O | F1 | M23 | — | M24 |
| A149 | O | O | F1 | M24 | — | M26 |
| A150 | O | O | F1 | M23 | F1 | M24 |
| A151 | O | O | — | M30 | — | M31 |
| A152 | O | O | — | M30 | F2 | M32 |
| A153 | O | S | — | M1 | — | M1 |
| A154 | O | S | — | M7 | — | M1 |
| A155 | O | S | — | M10 | — | M1 |
| A156 | O | S | — | M2 | — | M1 |
| A157 | O | S | — | M5 | — | M1 |
| A158 | O | S | F1 | M7 | — | M1 |
| A159 | O | S | F1 | M10 | — | M1 |
| A160 | O | S | — | M9 | — | M1 |
| A161 | O | S | — | M14 | — | M1 |
| A162 | O | S | — | M15 | — | M1 |
| A163 | O | S | F1 | M14 | — | M1 |
| A164 | O | S | — | M18 | — | M1 |
| A165 | O | S | F1 | M18 | — | M1 |
| A166 | O | S | — | M24 | — | M1 |
| A167 | O | S | — | M6 | — | M7 |
| A168 | O | S | F1 | M7 | — | M7 |
| A169 | O | S | F1 | M9 | — | M7 |
| A170 | O | S | — | M15 | — | M7 |
| A171 | O | S | F1 | M14 | — | M7 |
| A172 | O | S | — | M19 | — | M7 |
| A173 | O | S | F1 | M20 | — | M7 |
| A174 | O | S | — | M26 | — | M7 |
| A175 | O | S | — | M3 | — | M8 |
| A176 | O | S | F1 | M16 | — | M8 |
| A177 | O | S | F1 | M10 | — | M10 |
| A178 | O | S | F1 | M9 | — | M10 |
| A179 | O | S | F1 | M14 | — | M10 |
| A180 | O | S | — | M19 | — | M10 |
| A181 | O | S | — | M24 | — | M10 |
| A182 | O | S | — | M2 | — | M11 |
| A183 | O | S | — | M5 | — | M11 |
| A184 | O | S | F1 | M7 | — | M11 |
| A185 | O | S | — | M9 | — | M11 |
| A186 | O | S | — | M15 | — | M11 |
| A187 | O | S | F1 | M14 | — | M11 |
| A188 | O | S | F1 | M20 | — | M11 |
| A189 | O | S | — | M26 | — | M11 |
| A190 | O | S | F1 | M17 | — | M12 |
| A191 | O | S | F1 | M16 | — | M13 |
| A192 | O | S | — | M2 | — | M2 |
| A193 | O | S | — | M9 | — | M2 |
| A194 | O | S | F1 | M16 | — | M2 |
| A195 | O | S | — | M18 | — | M2 |
| A196 | O | S | — | M23 | — | M2 |
| A197 | O | S | F1 | M8 | — | M4 |
| A198 | O | S | F1 | M15 | — | M4 |
| A199 | O | S | F1 | M21 | — | M4 |
| A200 | O | S | — | M6 | — | M3 |
| A201 | O | S | — | M5 | — | M3 |
| A202 | O | S | F1 | M12 | — | M3 |
| A203 | O | S | F1 | M9 | — | M3 |
| A204 | O | S | F1 | M17 | — | M3 |
| A205 | O | S | — | M19 | — | M3 |
| A206 | O | S | F1 | M20 | — | M3 |
| A207 | O | S | — | M5 | — | M6 |
| A208 | O | S | F1 | M7 | — | M6 |
| A209 | O | S | F1 | M10 | — | M6 |
| A210 | O | S | — | M14 | — | M6 |
| A211 | O | S | F1 | M14 | — | M6 |
| A212 | O | S | F1 | M20 | — | M6 |
| A213 | O | S | — | M26 | — | M6 |
| A214 | O | S | F1 | M11 | — | M5 |
| A215 | O | S | F1 | M9 | — | M5 |
| A216 | O | S | — | M15 | — | M5 |
| A217 | O | S | F1 | M16 | — | M5 |
| A218 | O | S | — | M19 | — | M5 |
| A219 | O | S | F1 | M19 | — | M5 |
| A220 | O | S | — | M24 | — | M5 |
| A221 | O | S | — | M9 | F1 | M7 |
| A222 | O | S | F1 | M15 | F1 | M7 |
| A223 | O | S | F1 | M18 | F1 | M7 |
| A224 | O | S | — | M14 | F1 | M10 |
| A225 | O | S | F1 | M14 | F1 | M10 |
| A226 | O | S | F1 | M18 | F1 | M10 |
| A227 | O | S | — | M9 | F1 | M11 |
| A228 | O | S | — | M19 | F1 | M11 |
| A229 | O | S | — | M23 | F1 | M11 |
| A230 | O | S | — | M15 | — | M9 |
| A231 | O | S | F1 | M14 | — | M9 |
| A232 | O | S | — | M19 | — | M9 |
| A233 | O | S | F1 | M18 | — | M9 |
| A234 | O | S | — | M24 | — | M9 |
| A235 | O | S | — | M15 | — | M14 |
| A236 | O | S | — | M19 | — | M14 |
| A237 | O | S | F1 | M15 | — | M15 |
| A238 | O | S | F1 | M20 | — | M15 |
| A239 | O | S | — | M26 | — | M15 |
| A240 | O | S | F1 | M17 | F1 | M14 |
| A241 | O | S | — | M19 | F1 | M14 |
| A242 | O | S | F1 | M19 | F1 | M14 |
| A243 | O | S | F1 | M23 | F1 | M15 |
| A244 | O | S | — | M18 | F1 | M16 |
| A245 | O | S | F1 | M19 | — | M18 |
| A246 | O | S | F1 | M24 | — | M19 |
| A247 | O | S | — | M23 | F1 | M19 |
| A248 | S | O | — | M1 | F1 | M14 |
| A249 | S | O | — | M1 | — | M15 |
| A250 | S | O | — | M2 | — | M2 |
| A251 | S | S | — | M1 | — | M1 |
| A252 | S | S | — | M2 | — | M2 |

TABLE 2

| Compound | | | | | | |
|---|---|---|---|---|---|---|
| No. | X₁ | X₂ | L₁ | Ar₁ | L₂ | Ar₂ |
| A253 | O | O | — | M1 | — | M2 |
| A254 | O | O | — | M30 | — | M31 |
| A255 | O | O | — | M31 | — | M31 |
| A256 | O | O | — | M1 | F1 | M7 |
| A257 | O | O | — | M30 | F2 | M32 |
| A258 | O | O | — | M31 | — | M35 |
| A259 | O | S | — | M31 | — | M31 |
| A260 | O | S | — | M30 | — | M38 |
| A261 | O | S | — | M30 | F2 | M37 |
| A262 | S | O | — | M31 | — | M31 |
| A263 | S | O | — | M30 | F2 | M37 |
| A264 | S | S | — | M30 | — | M30 |

According to one example of the present disclosure, the compound represented by the Chemical Formula 1-2 may be selected from the group consisting of the compounds as shown in Tables 3 and 4 as set forth below. However, the present disclosure is not limited thereto. In the compounds as listed in Table 3 as set forth below, all of R₁₁ to R₁₂, R₂₁ to R₂₄, R₃₁ to R₃₄, R₄₁ to R₄₃, and R₅₁ to R₅₄ may be hydrogen. In the compounds as listed in Table 4 as set forth below, all of R₁₁ to R₁₂, R₂₁ to R₂₄, R₃₁ to R₃₄, R₄₁ to R₄₃, and R₅₁ to R₅₄ may be deuterium. In the following Tables, the mark “—” as each of X₁, X₂, L₁, L₂, Ar₁, and Ar₂ means a single bond.

TABLE 3

| Compound | | | | | | |
|---|---|---|---|---|---|---|
| No. | X₁ | X₂ | L₁ | Ar₁ | L₂ | Ar₂ |
| A265 | O | O | — | M1 | — | M1 |
| A266 | O | O | — | M2 | — | M1 |
| A267 | O | O | — | M7 | — | M1 |
| A268 | O | O | — | M8 | — | M1 |
| A269 | O | O | — | M10 | — | M1 |
| A270 | O | O | — | M4 | — | M1 |
| A271 | O | O | — | M5 | — | M1 |
| A272 | O | O | F1 | M7 | — | M1 |
| A273 | O | O | F1 | M10 | — | M1 |
| A274 | O | O | — | M9 | — | M1 |
| A275 | O | O | — | M14 | — | M1 |
| A276 | O | O | F1 | M14 | — | M1 |
| A277 | O | O | — | M18 | — | M1 |
| A278 | O | O | F1 | M18 | — | M1 |
| A279 | O | O | — | M24 | — | M1 |
| A280 | O | O | — | M26 | — | M1 |
| A281 | O | O | F1 | M22 | — | M1 |
| A282 | O | O | — | M8 | — | M7 |
| A283 | O | O | — | M6 | — | M7 |
| A284 | O | O | — | M7 | — | M7 |
| A285 | O | O | F1 | M7 | — | M7 |
| A286 | O | O | F1 | M11 | — | M7 |
| A287 | O | O | F1 | M9 | — | M7 |
| A288 | O | O | — | M15 | — | M7 |
| A289 | O | O | F1 | M14 | — | M7 |
| A290 | O | O | — | M19 | — | M7 |
| A291 | O | O | F1 | M20 | — | M7 |
| A292 | O | O | — | M24 | — | M7 |
| A293 | O | O | — | M26 | — | M7 |
| A294 | O | O | F1 | M23 | — | M7 |
| A295 | O | O | — | M11 | — | M8 |
| A296 | O | O | — | M3 | — | M8 |
| A297 | O | O | — | M11 | — | M10 |
| A298 | O | O | — | M2 | — | M10 |
| A299 | O | O | F1 | M8 | — | M10 |
| A300 | O | O | F1 | M10 | — | M10 |
| A301 | O | O | F1 | M9 | — | M10 |
| A302 | O | O | — | M19 | — | M10 |
| A303 | O | O | — | M24 | — | M10 |
| A304 | O | O | — | M2 | — | M11 |
| A305 | O | O | — | M5 | — | M11 |
| A306 | O | O | F1 | M7 | — | M11 |
| A307 | O | O | — | M9 | — | M11 |
| A308 | O | O | — | M15 | — | M11 |
| A309 | O | O | F1 | M14 | — | M11 |
| A310 | O | O | F1 | M20 | — | M11 |
| A311 | O | O | — | M26 | — | M11 |
| A312 | O | O | F1 | M24 | — | M11 |
| A313 | O | O | — | M2 | — | M2 |
| A314 | O | O | — | M3 | — | M2 |
| A315 | O | O | — | M4 | — | M2 |
| A316 | O | O | — | M9 | — | M2 |
| A317 | O | O | — | M10 | — | M2 |
| A318 | O | O | F1 | M12 | — | M2 |
| A319 | O | O | — | M18 | — | M2 |
| A320 | O | O | — | M23 | — | M2 |
| A321 | O | O | — | M6 | — | M4 |
| A322 | O | O | F1 | M8 | — | M4 |
| A323 | O | O | F1 | M10 | — | M4 |
| A324 | O | O | F1 | M15 | — | M4 |
| A325 | O | O | F1 | M16 | — | M4 |
| A326 | O | O | — | M24 | — | M4 |
| A327 | O | O | F1 | M24 | — | M4 |
| A328 | O | O | — | M3 | — | M3 |
| A329 | O | O | — | M5 | — | M3 |
| A330 | O | O | — | M6 | — | M3 |
| A331 | O | O | F1 | M9 | — | M3 |
| A332 | O | O | — | M10 | — | M3 |
| A333 | O | O | — | M14 | — | M3 |
| A334 | O | O | F1 | M20 | — | M3 |
| A335 | O | O | — | M5 | — | M6 |
| A336 | O | O | F1 | M7 | — | M6 |
| A337 | O | O | F1 | M10 | — | M6 |
| A338 | O | O | — | M14 | — | M6 |
| A339 | O | O | F1 | M14 | — | M6 |
| A340 | O | O | F1 | M20 | — | M6 |
| A341 | O | O | — | M26 | — | M6 |
| A342 | O | O | F1 | M8 | — | M5 |
| A343 | O | O | F1 | M9 | — | M5 |
| A344 | O | O | — | M15 | — | M5 |
| A345 | O | O | F1 | M16 | — | M5 |
| A346 | O | O | — | M19 | — | M5 |
| A347 | O | O | F1 | M19 | — | M5 |
| A348 | O | O | — | M24 | — | M5 |
| A349 | O | O | F1 | M23 | — | M5 |
| A350 | O | O | F1 | M8 | F1 | M7 |
| A351 | O | O | — | M9 | F1 | M7 |
| A352 | O | O | F1 | M15 | F1 | M7 |
| A353 | O | O | F1 | M18 | F1 | M7 |
| A354 | O | O | — | M24 | F1 | M7 |
| A355 | O | O | F1 | M23 | F1 | M8 |
| A356 | O | O | F1 | M14 | F1 | M10 |
| A357 | O | O | F1 | M18 | F1 | M10 |
| A358 | O | O | — | M9 | F1 | M11 |
| A359 | O | O | — | M19 | F1 | M11 |
| A360 | O | O | — | M23 | F1 | M11 |
| A361 | O | O | — | M26 | F1 | M11 |
| A362 | O | O | — | M15 | F1 | M12 |
| A363 | O | O | F1 | M24 | F1 | M12 |
| A364 | O | O | F1 | M9 | — | M9 |
| A365 | O | O | — | M15 | — | M9 |
| A366 | O | O | F1 | M14 | — | M9 |
| A367 | O | O | — | M19 | — | M9 |
| A368 | O | O | F1 | M18 | — | M9 |
| A369 | O | O | — | M24 | — | M9 |
| A370 | O | O | — | M26 | — | M9 |
| A371 | O | O | — | M14 | F1 | M9 |
| A372 | O | O | — | M19 | — | M14 |
| A373 | O | O | — | M24 | — | M14 |
| A374 | O | O | F1 | M24 | — | M14 |
| A375 | O | O | F1 | M15 | — | M15 |
| A376 | O | O | F1 | M20 | — | M15 |
| A377 | O | O | — | M26 | — | M15 |
| A378 | O | O | — | M19 | F1 | M14 |
| A379 | O | O | F1 | M19 | F1 | M14 |
| A380 | O | O | — | M23 | F1 | M14 |
| A381 | O | O | F1 | M23 | F1 | M15 |
| A382 | O | O | F1 | M24 | F1 | M16 |
| A383 | O | O | F1 | M19 | — | M18 |
| A384 | O | O | — | M24 | — | M19 |
| A385 | O | O | F1 | M24 | — | M19 |
| A386 | O | O | — | M23 | F1 | M19 |
| A387 | O | O | — | M26 | F1 | M19 |
| A388 | O | O | — | M23 | — | M24 |
| A389 | O | O | — | M26 | — | M24 |
| A390 | O | O | F1 | M23 | — | M24 |
| A391 | O | O | F1 | M24 | — | M26 |
| A392 | O | O | F1 | M23 | F1 | M24 |
| A393 | O | S | — | M7 | — | M1 |
| A394 | O | S | — | M10 | — | M1 |
| A395 | O | S | — | M2 | — | M1 |
| A396 | O | S | — | M5 | — | M1 |
| A397 | O | S | F1 | M7 | — | M1 |
| A398 | O | S | F1 | M10 | — | M1 |
| A399 | O | S | — | M9 | — | M1 |
| A400 | O | S | — | M14 | — | M1 |
| A401 | O | S | F1 | M14 | — | M1 |
| A402 | O | S | — | M18 | — | M1 |
| A403 | O | S | — | M20 | — | M1 |
| A404 | O | S | F1 | M18 | — | M1 |
| A405 | O | S | F1 | M19 | — | M1 |
| A406 | O | S | — | M24 | — | M1 |
| A407 | O | S | — | M6 | — | M7 |
| A408 | O | S | F1 | M7 | — | M7 |
| A409 | O | S | F1 | M9 | — | M7 |
| A410 | O | S | — | M15 | — | M7 |
| A411 | O | S | F1 | M14 | — | M7 |
| A412 | O | S | — | M19 | — | M7 |
| A413 | O | S | F1 | M20 | — | M7 |
| A414 | O | S | — | M26 | — | M7 |
| A415 | O | S | — | M3 | — | M8 |
| A416 | O | S | F1 | M16 | — | M8 |
| A417 | O | S | F1 | M10 | — | M10 |
| A418 | O | S | F1 | M9 | — | M10 |
| A419 | O | S | F1 | M14 | — | M10 |
| A420 | O | S | — | M19 | — | M10 |
| A421 | O | S | — | M24 | — | M10 |
| A422 | O | S | — | M2 | — | M11 |
| A423 | O | S | — | M5 | — | M11 |
| A424 | O | S | F1 | M7 | — | M11 |
| A425 | O | S | — | M9 | — | M11 |
| A426 | O | S | — | M15 | — | M11 |
| A427 | O | S | F1 | M14 | — | M11 |
| A428 | O | S | F1 | M20 | — | M11 |
| A429 | O | S | — | M26 | — | M11 |
| A430 | O | S | F1 | M17 | — | M12 |
| A431 | O | S | F1 | M16 | — | M13 |
| A432 | O | S | — | M2 | — | M2 |
| A433 | O | S | — | M9 | — | M2 |
| A434 | O | S | F1 | M16 | — | M2 |
| A435 | O | S | — | M18 | — | M2 |
| A436 | O | S | — | M23 | — | M2 |
| A437 | O | S | F1 | M8 | — | M4 |
| A438 | O | S | — | M6 | — | M3 |
| A439 | O | S | — | M5 | — | M3 |
| A440 | O | S | F1 | M12 | — | M3 |
| A441 | O | S | F1 | M9 | — | M3 |
| A442 | O | S | — | M19 | — | M3 |
| A443 | O | S | F1 | M20 | — | M3 |
| A444 | O | S | — | M5 | — | M6 |
| A445 | O | S | F1 | M7 | — | M6 |
| A446 | O | S | F1 | M10 | — | M6 |
| A447 | O | S | — | M14 | — | M6 |
| A448 | O | S | F1 | M14 | — | M6 |
| A449 | O | S | — | M26 | — | M6 |
| A450 | O | S | F1 | M11 | — | M5 |
| A451 | O | S | F1 | M16 | — | M5 |
| A452 | O | S | — | M19 | — | M5 |
| A453 | O | S | F1 | M19 | — | M5 |
| A454 | O | S | — | M24 | — | M5 |
| A455 | O | S | — | M9 | F1 | M7 |
| A456 | O | S | F1 | M15 | F1 | M7 |
| A457 | O | S | — | M14 | F1 | M10 |
| A458 | O | S | F1 | M14 | F1 | M10 |
| A459 | O | S | F1 | M18 | F1 | M10 |
| A460 | O | S | — | M9 | F1 | M11 |
| A461 | O | S | — | M19 | F1 | M11 |
| A462 | O | S | — | M23 | F1 | M11 |
| A463 | O | S | — | M15 | — | M9 |
| A464 | O | S | F1 | M14 | — | M9 |
| A465 | O | S | — | M19 | — | M9 |
| A466 | O | S | F1 | M18 | — | M9 |
| A467 | O | S | — | M24 | — | M9 |
| A468 | O | S | — | M15 | — | M14 |
| A469 | O | S | F1 | M15 | — | M15 |
| A470 | O | S | F1 | M17 | F1 | M14 |
| A471 | O | S | — | M19 | F1 | M14 |
| A472 | O | S | F1 | M19 | F1 | M14 |
| A473 | O | S | F1 | M23 | F1 | M15 |
| A474 | O | S | — | M18 | F1 | M16 |
| A475 | O | S | — | M23 | F1 | M19 |
| A476 | S | O | — | M1 | F1 | M19 |
| A477 | S | O | — | M1 | — | M20 |
| A478 | S | O | — | M20 | — | M1 |
| A479 | S | S | F1 | M7 | — | M1 |

TABLE 4

| Compound No. | X₁ | X₂ | L₁ | Ar₁ | L₂ | Ar₂ |
|---|---|---|---|---|---|---|
| A480 | O | O | — | M31 | — | M31 |
| A481 | O | O | — | M30 | — | M33 |
| A482 | O | O | — | M31 | — | M35 |
| A483 | O | S | — | M31 | — | M31 |
| A484 | S | O | — | M30 | — | M40 |
| A485 | S | S | — | M30 | F2 | M32 |

According to one example of the present disclosure, the compound represented by the Chemical Formula 1-3 may be selected from the group consisting of the compounds as shown in Tables 5 and 6 as set forth below. However, the present disclosure is not limited thereto. In the compounds as listed in Table 5 as set forth below, all of R₁₁ to R₁₂, R₂₁ to R₂₄, R₃₁ to R₃₄, R₄₁ to R₄₃, and R₅₁ to R₅₄ may be hydrogen. In the compounds as listed in Table 6 as set forth below, all of R₁₁ to R₁₂, R₂₁ to R₂₄, R₃₁ to R₃₄, R₄₁ to R₄₃, and R₅₁ to R₅₄ may be deuterium. In the following Tables, the mark “—” as each of X₁, X₂, L₁, L₂, Ar₁, and Ar₂ means a single bond.

TABLE 5

| Compound No. | X₁ | X₂ | L₁ | Ar₁ | L₂ | Ar₂ |
|---|---|---|---|---|---|---|
| A486 | O | O | — | M2 | — | M1 |
| A487 | O | O | — | M7 | — | M1 |
| A488 | O | O | — | M11 | — | M1 |
| A489 | O | O | — | M4 | — | M1 |
| A490 | O | O | — | M5 | — | M1 |
| A491 | O | O | F1 | M7 | — | M1 |
| A492 | O | O | F1 | M10 | — | M1 |
| A493 | O | O | — | M9 | — | M1 |
| A494 | O | O | — | M14 | — | M1 |
| A495 | O | O | F1 | M14 | — | M1 |
| A496 | O | O | — | M18 | — | M1 |
| A497 | O | O | F1 | M18 | — | M1 |
| A498 | O | O | — | M24 | — | M1 |
| A499 | O | O | — | M8 | — | M7 |
| A500 | O | O | — | M6 | — | M7 |
| A501 | O | O | F1 | M7 | — | M7 |
| A502 | O | O | F1 | M11 | — | M7 |
| A503 | O | O | F1 | M9 | — | M7 |
| A504 | O | O | — | M15 | — | M7 |
| A505 | O | O | F1 | M14 | — | M7 |
| A506 | O | O | — | M19 | — | M7 |
| A507 | O | O | F1 | M20 | — | M7 |
| A508 | O | O | F1 | M23 | — | M7 |
| A509 | O | O | — | M11 | — | M8 |
| A510 | O | O | — | M3 | — | M8 |
| A511 | O | O | — | M11 | — | M10 |
| A512 | O | O | F1 | M10 | — | M10 |
| A513 | O | O | — | M20 | — | M10 |
| A514 | O | O | — | M2 | — | M11 |
| A515 | O | O | — | M5 | — | M11 |
| A516 | O | O | F1 | M7 | — | M11 |
| A517 | O | O | — | M9 | — | M11 |
| A518 | O | O | — | M15 | — | M11 |
| A519 | O | O | F1 | M14 | — | M11 |
| A520 | O | O | F1 | M20 | — | M11 |
| A521 | O | O | — | M2 | — | M2 |
| A522 | O | O | — | M9 | — | M2 |
| A523 | O | O | — | M18 | — | M2 |
| A524 | O | O | — | M23 | — | M2 |
| A525 | O | O | — | M6 | — | M4 |
| A526 | O | O | F1 | M10 | — | M4 |
| A527 | O | O | F1 | M20 | — | M3 |
| A528 | O | O | — | M5 | — | M6 |
| A529 | O | O | F1 | M7 | — | M6 |
| A530 | O | O | F1 | M10 | — | M6 |
| A531 | O | O | — | M14 | — | M6 |
| A532 | O | O | F1 | M14 | — | M6 |
| A533 | O | O | F1 | M20 | — | M6 |
| A534 | O | O | — | M26 | — | M6 |
| A535 | O | O | F1 | M8 | — | M5 |
| A536 | O | O | F1 | M9 | — | M5 |
| A537 | O | O | — | M15 | — | M5 |
| A538 | O | O | F1 | M16 | — | M5 |
| A539 | O | O | — | M19 | — | M5 |
| A540 | O | O | F1 | M19 | — | M5 |
| A541 | O | O | F1 | M23 | — | M5 |
| A542 | O | O | F1 | M8 | F1 | M7 |
| A543 | O | O | — | M9 | F1 | M7 |
| A544 | O | O | F1 | M15 | F1 | M7 |
| A545 | O | O | F1 | M18 | F1 | M7 |
| A546 | O | O | F1 | M14 | F1 | M10 |
| A547 | O | O | F1 | M8 | F1 | M10 |
| A548 | O | O | — | M9 | F1 | M11 |
| A549 | O | O | — | M19 | F1 | M11 |
| A550 | O | O | — | M23 | F1 | M11 |
| A551 | O | O | — | M15 | F1 | M12 |
| A552 | O | O | F1 | M9 | — | M9 |
| A553 | O | O | — | M15 | — | M9 |
| A554 | O | O | F1 | M14 | — | M9 |
| A555 | O | O | — | M19 | — | M9 |
| A556 | O | O | F1 | M18 | — | M9 |
| A557 | O | O | — | M24 | — | M9 |
| A558 | O | O | — | M26 | — | M9 |
| A559 | O | O | — | M19 | — | M14 |
| A560 | O | O | — | M24 | — | M14 |
| A561 | O | O | F1 | M15 | — | M15 |
| A562 | O | O | F1 | M20 | — | M15 |
| A563 | O | O | — | M26 | — | M15 |
| A564 | O | O | F1 | M19 | F1 | M14 |
| A565 | O | O | F1 | M23 | F1 | M15 |
| A566 | O | O | F1 | M19 | — | M18 |
| A567 | O | O | — | M24 | — | M19 |
| A568 | O | O | — | M23 | F1 | M19 |
| A569 | O | O | — | M23 | — | M24 |
| A570 | O | O | — | M26 | — | M24 |
| A571 | O | S | — | M7 | — | M1 |
| A572 | O | S | — | M10 | — | M1 |
| A573 | O | S | — | M2 | — | M1 |
| A574 | O | S | — | M5 | — | M1 |
| A575 | O | S | F1 | M10 | — | M1 |
| A576 | O | S | — | M9 | — | M1 |
| A577 | O | S | — | M14 | — | M1 |
| A578 | O | S | F1 | M14 | — | M1 |
| A579 | O | S | — | M18 | — | M1 |
| A580 | O | S | F1 | M19 | — | M1 |
| A581 | O | S | — | M24 | — | M1 |
| A582 | O | S | — | M28 | — | M1 |
| A583 | O | S | — | M6 | — | M7 |
| A584 | O | S | F1 | M7 | — | M7 |
| A585 | O | S | F1 | M9 | — | M7 |
| A586 | O | S | — | M15 | — | M7 |
| A587 | O | S | F1 | M14 | — | M7 |
| A588 | O | S | — | M19 | — | M7 |
| A589 | O | S | F1 | M20 | — | M7 |
| A590 | O | S | — | M3 | — | M8 |
| A591 | O | S | F1 | M16 | — | M8 |
| A592 | O | S | F1 | M10 | — | M10 |
| A593 | O | S | F1 | M9 | — | M10 |
| A594 | O | S | F1 | M14 | — | M10 |
| A595 | O | S | — | M19 | — | M10 |
| A596 | O | S | — | M24 | — | M10 |
| A597 | O | S | — | M2 | — | M11 |
| A598 | O | S | — | M5 | — | M11 |
| A599 | O | S | F1 | M7 | — | M11 |
| A600 | O | S | — | M9 | — | M11 |
| A601 | O | S | — | M15 | — | M11 |
| A602 | O | S | F1 | M14 | — | M11 |
| A603 | O | S | F1 | M20 | — | M11 |
| A604 | O | S | — | M26 | — | M11 |
| A605 | O | S | F1 | M17 | — | M12 |
| A606 | O | S | — | M2 | — | M2 |
| A607 | O | S | — | M6 | — | M2 |
| A608 | O | S | — | M9 | — | M2 |
| A609 | O | S | F1 | M16 | — | M2 |
| A610 | O | S | F1 | M18 | — | M2 |
| A611 | O | S | — | M23 | — | M2 |
| A612 | O | S | F1 | M8 | — | M4 |
| A613 | O | S | F1 | M12 | — | M3 |
| A614 | O | S | F1 | M9 | — | M3 |
| A615 | O | S | — | M19 | — | M3 |
| A616 | O | S | F1 | M20 | — | M3 |
| A617 | O | S | F1 | M10 | — | M6 |
| A618 | O | S | — | M14 | — | M6 |
| A619 | O | S | F1 | M14 | — | M6 |
| A620 | O | S | — | M26 | — | M6 |
| A621 | O | S | F1 | M11 | — | M5 |
| A622 | O | S | F1 | M16 | — | M5 |
| A623 | O | S | F1 | M19 | — | M5 |
| A624 | O | S | — | M24 | — | M5 |
| A625 | O | S | — | M9 | F1 | M7 |
| A626 | O | S | — | M14 | F1 | M10 |
| A627 | O | S | F1 | M14 | F1 | M10 |
| A628 | O | S | F1 | M18 | F1 | M10 |
| A629 | O | S | — | M9 | F1 | M11 |
| A630 | O | S | — | M19 | F1 | M11 |
| A631 | O | S | — | M15 | — | M9 |
| A632 | O | S | F1 | M14 | — | M9 |
| A633 | O | S | — | M19 | — | M9 |
| A634 | O | S | F1 | M18 | — | M9 |
| A635 | O | S | — | M24 | — | M9 |
| A636 | O | S | — | M15 | — | M14 |
| A637 | O | S | F1 | M15 | — | M15 |
| A638 | O | S | — | M19 | F1 | M14 |
| A639 | O | S | F1 | M19 | F1 | M14 |
| A640 | O | S | F1 | M23 | F1 | M15 |
| A641 | O | S | — | M23 | F1 | M19 |
| A642 | S | O | — | M3 | — | M8 |
| A643 | S | O | — | M1 | F1 | M29 |
| A644 | S | S | — | M1 | F1 | M13 |

TABLE 6

| Compound No. | X₁ | X₂ | L₁ | Ar₁ | L₂ | Ar₂ |
|---|---|---|---|---|---|---|
| A645 | O | O | — | M31 | — | M31 |
| A646 | O | O | — | M30 | — | M35 |
| A647 | O | O | — | M31 | — | M35 |
| A648 | O | S | — | M30 | — | M41 |
| A650 | S | O | — | M30 | — | M41 |
| A651 | S | S | — | M30 | F2 | M36 |

According to one example of the present disclosure, the compound represented by the Chemical Formula 1-4 may be selected from the group consisting of the compounds as shown in Tables 7 and 8 as set forth below. However, the present disclosure is not limited thereto. In the compounds as listed in Table 7 as set forth below, all of R₁₁ to R₁₂, R₂₁ to R₂₄, R₃₁ to R₃₄, R₄₁ to R₄₃, and R₅₁ to R₅₄ may be hydrogen. In the compounds as listed in Table 8 as set forth below, all of R₁₁ to R₁₂, R₂₁ to R₂₄, R₃₁ to R₃₄, R₄₁ to R₄₃, and R₅₁ to R₅₄ may be deuterium. In the following Tables, the mark “—” as each of X₁, X₂, L₁, L₂, Ar₁, and Ar₂ means a single bond.

TABLE 7

| Compound No. | X₁ | X₂ | L₁ | Ar₁ | L₂ | Ar₂ |
|---|---|---|---|---|---|---|
| A652 | O | O | — | M2 | — | M1 |
| A653 | O | O | — | M7 | — | M1 |
| A654 | O | O | — | M11 | — | M1 |
| A655 | O | O | — | M2 | — | M1 |
| A656 | O | O | — | M4 | — | M1 |
| A657 | O | O | — | M5 | — | M1 |
| A658 | O | O | F1 | M7 | — | M1 |
| A659 | O | O | F1 | M10 | — | M1 |
| A660 | O | O | — | M9 | — | M1 |
| A661 | O | O | — | M14 | — | M1 |
| A662 | O | O | F1 | M14 | — | M1 |
| A663 | O | O | — | M18 | — | M1 |
| A664 | O | O | F1 | M18 | — | M1 |
| A665 | O | O | — | M24 | — | M1 |
| A666 | O | O | — | M8 | — | M7 |
| A667 | O | O | — | M6 | — | M7 |
| A668 | O | O | F1 | M7 | — | M7 |
| A669 | O | O | F1 | M11 | — | M7 |
| A670 | O | O | F1 | M9 | — | M7 |
| A671 | O | O | — | M15 | — | M7 |
| A672 | O | O | F1 | M14 | — | M7 |
| A673 | O | O | — | M19 | — | M7 |
| A674 | O | O | F1 | M20 | — | M7 |
| A675 | O | O | F1 | M23 | — | M7 |
| A676 | O | O | — | M11 | — | M8 |
| A677 | O | O | — | M3 | — | M8 |
| A678 | O | O | — | M11 | — | M10 |
| A679 | O | O | F1 | M10 | — | M10 |
| A680 | O | O | — | M20 | — | M10 |
| A681 | O | O | — | M2 | — | M11 |
| A682 | O | O | — | M5 | — | M11 |
| A683 | O | O | F1 | M7 | — | M11 |
| A684 | O | O | — | M9 | — | M11 |
| A685 | O | O | — | M15 | — | M11 |
| A686 | O | O | F1 | M14 | — | M11 |
| A687 | O | O | F1 | M20 | — | M11 |
| A688 | O | O | — | M2 | — | M2 |
| A689 | O | O | — | M9 | — | M2 |
| A690 | O | O | — | M18 | — | M2 |
| A691 | O | O | — | M23 | — | M2 |
| A692 | O | O | — | M6 | — | M4 |
| A693 | O | O | F1 | M10 | — | M4 |
| A694 | O | O | F1 | M20 | — | M3 |
| A695 | O | O | — | M5 | — | M6 |
| A696 | O | O | F1 | M7 | — | M6 |
| A697 | O | O | F1 | M10 | — | M6 |
| A698 | O | O | — | M14 | — | M6 |
| A699 | O | O | F1 | M14 | — | M6 |
| A700 | O | O | F1 | M20 | — | M6 |
| A701 | O | O | — | M26 | — | M6 |
| A702 | O | O | F1 | M8 | — | M5 |
| A703 | O | O | F1 | M9 | — | M5 |
| A704 | O | O | — | M15 | — | M5 |
| A705 | O | O | — | M19 | — | M5 |
| A706 | O | O | F1 | M19 | — | M5 |
| A707 | O | O | F1 | M8 | F1 | M7 |
| A708 | O | O | — | M9 | F1 | M7 |
| A709 | O | O | F1 | M18 | F1 | M7 |
| A710 | O | O | F1 | M14 | F1 | M10 |
| A711 | O | O | F1 | M18 | F1 | M10 |
| A712 | O | O | — | M9 | F1 | M11 |
| A713 | O | O | — | M19 | F1 | M11 |
| A714 | O | O | — | M23 | F1 | M11 |
| A715 | O | O | — | M15 | F1 | M12 |
| A716 | O | O | F1 | M9 | — | M9 |
| A717 | O | O | — | M15 | — | M9 |
| A718 | O | O | F1 | M14 | — | M9 |
| A719 | O | O | — | M19 | — | M9 |
| A720 | O | O | F1 | M18 | — | M9 |
| A721 | O | O | — | M24 | — | M9 |
| A722 | O | O | — | M19 | — | M14 |
| A723 | O | O | F1 | M15 | — | M15 |
| A724 | O | O | F1 | M20 | — | M15 |
| A725 | O | O | — | M26 | — | M15 |
| A726 | O | O | F1 | M19 | F1 | M14 |
| A727 | O | O | F1 | M23 | F1 | M14 |
| A728 | O | O | F1 | M19 | — | M18 |
| A729 | O | O | — | M24 | — | M19 |
| A730 | O | O | — | M23 | F1 | M19 |
| A731 | O | O | — | M26 | — | M24 |
| A732 | O | S | — | M7 | — | M1 |
| A733 | O | S | — | M10 | — | M1 |
| A734 | O | S | — | M2 | — | M1 |
| A735 | O | S | — | M5 | — | M1 |
| A736 | O | S | F1 | M10 | — | M1 |
| A737 | O | S | — | M9 | — | M1 |
| A738 | O | S | — | M14 | — | M1 |
| A739 | O | S | F1 | M14 | — | M1 |
| A740 | O | S | — | M18 | — | M1 |
| A741 | O | S | F1 | M19 | — | M1 |
| A742 | O | S | — | M23 | — | M1 |
| A743 | O | S | F1 | M27 | — | M1 |
| A744 | O | S | — | M6 | — | M7 |
| A745 | O | S | F1 | M16 | — | M8 |
| A746 | O | S | — | M19 | — | M10 |
| A747 | O | S | — | M24 | — | M10 |
| A748 | O | S | — | M2 | — | M11 |
| A749 | O | S | — | M5 | — | M11 |
| A750 | O | S | — | M9 | — | M11 |
| A751 | O | S | — | M15 | — | M11 |
| A752 | O | S | F1 | M14 | — | M11 |
| A753 | O | S | — | M26 | — | M11 |
| A754 | O | S | F1 | M17 | — | M12 |
| A755 | O | S | — | M2 | — | M2 |
| A756 | O | S | — | M9 | — | M2 |
| A757 | O | S | — | M10 | — | M2 |
| A758 | O | S | F1 | M16 | — | M2 |
| A759 | O | S | — | M23 | — | M2 |
| A760 | O | S | F1 | M8 | — | M4 |
| A761 | O | S | F1 | M12 | — | M3 |
| A762 | O | S | F1 | M9 | — | M3 |
| A763 | O | S | — | M19 | — | M3 |
| A764 | O | S | F1 | M20 | — | M3 |
| A765 | O | S | — | M5 | — | M6 |
| A766 | O | S | F1 | M7 | — | M6 |
| A767 | O | S | F1 | M10 | — | M6 |
| A768 | O | S | — | M24 | — | M5 |
| A769 | O | S | — | M9 | — | M7 |
| A770 | O | S | — | M14 | F1 | M10 |
| A771 | O | S | F1 | M14 | F1 | M10 |
| A772 | O | S | F1 | M18 | F1 | M10 |
| A773 | O | S | — | M9 | F1 | M11 |
| A774 | O | S | — | M15 | — | M9 |
| A775 | O | S | F1 | M14 | — | M9 |
| A776 | O | S | F1 | M18 | — | M9 |
| A777 | O | S | — | M24 | — | M9 |
| A778 | O | S | F1 | M15 | — | M15 |
| A779 | O | S | F1 | M28 | — | M15 |
| A780 | O | S | F1 | M23 | F1 | M15 |
| A781 | O | S | F1 | M28 | — | M24 |
| A782 | S | O | F1 | M27 | — | M1 |
| A783 | S | O | — | M30 | F2 | M43 |
| A784 | S | S | F1 | M9 | — | M1 |

TABLE 8

| Compound No. | X₁ | X₂ | L₁ | Ar₁ | L₂ | Ar₂ |
|---|---|---|---|---|---|---|
| A785 | O | O | — | M30 | — | M31 |
| A786 | O | O | — | M31 | — | M31 |
| A787 | O | O | — | M30 | — | M34 |
| A788 | O | O | — | M31 | — | M35 |
| A789 | O | S | — | M30 | — | M41 |
| A790 | S | O | — | M30 | F2 | M43 |
| A791 | S | S | — | M30 | F2 | M34 |

Further, the compounds corresponding to the Chemical Formulas 1-1 to 1-4 may be selected from the group consisting of the Compounds represented by Table 9 as set forth below (all of R₁₁, R₁₂, R₂₁ to R₂₄, R₃₁ to R₃₄, R₄₁ to R₄₄, and R₅₁ to R₅₄ of Table 9 are hydrogen).

TABLE 9

| Compound | Chemical | | | | | | |
|---|---|---|---|---|---|---|---|
| NO. | Formula | X₁ | X₂ | L₁ | Ar₁ | L₂ | Ar₂ |
| A792 | 1-1 | O | O | — | M7 | — | M7 |
| A793 | 1-1 | O | O | F1 | M7 | F1 | M7 |
| A794 | 1-1 | O | O | F1 | M10 | F1 | M10 |
| A795 | 1-1 | O | O | — | M15 | — | M15 |
| A796 | 1-1 | O | O | F1 | M14 | F1 | M14 |
| A797 | 1-1 | O | O | — | M19 | — | M19 |
| A798 | 1-1 | O | O | F1 | M18 | F1 | M18 |
| A799 | 1-1 | O | O | — | M24 | — | M24 |
| A800 | 1-1 | O | O | — | M26 | — | M26 |
| A801 | 1-1 | O | S | — | M11 | — | M11 |
| A802 | 1-1 | O | S | — | M3 | — | M3 |
| A803 | 1-1 | O | S | F1 | M10 | F1 | M10 |
| A804 | 1-1 | O | S | — | M15 | — | M15 |
| A805 | 1-1 | O | S | F1 | M14 | F1 | M14 |
| A806 | 1-1 | O | S | — | M19 | — | M19 |
| A807 | 1-1 | O | S | F1 | M18 | F1 | M18 |
| A808 | 1-1 | O | S | — | M26 | — | M26 |
| A809 | 1-2 | O | O | F1 | M10 | F1 | M10 |
| A810 | 1-2 | O | O | — | M15 | — | M15 |
| A811 | 1-2 | O | O | F1 | M14 | F1 | M14 |
| A812 | 1-2 | O | O | — | M19 | — | M19 |
| A813 | 1-2 | O | O | F1 | M18 | F1 | M18 |
| A814 | 1-2 | O | O | — | M24 | — | M24 |
| A815 | 1-2 | O | O | — | M26 | — | M26 |
| A816 | 1-2 | O | S | — | M1 | — | M1 |
| A817 | 1-2 | O | S | — | M11 | — | M11 |
| A818 | 1-2 | O | S | — | M3 | — | M3 |
| A819 | 1-2 | O | S | F1 | M10 | F1 | M10 |
| A820 | 1-2 | O | S | — | M15 | — | M15 |
| A821 | 1-2 | O | S | F1 | M14 | F1 | M14 |
| A822 | 1-2 | O | S | — | M19 | — | M19 |
| A823 | 1-2 | O | S | F1 | M18 | F1 | M18 |
| A824 | 1-2 | O | S | — | M26 | — | M26 |
| A825 | 1-3 | O | O | — | M1 | — | M1 |
| A826 | 1-3 | O | O | — | M3 | — | M3 |
| A827 | 1-3 | O | O | F1 | M10 | F1 | M10 |
| A828 | 1-3 | O | O | — | M15 | — | M15 |
| A829 | 1-3 | O | O | F1 | M14 | F1 | M14 |
| A830 | 1-3 | O | O | — | M19 | — | M19 |
| A831 | 1-3 | O | O | F1 | M18 | F1 | M18 |
| A832 | 1-3 | O | O | — | M24 | — | M24 |
| A833 | 1-3 | O | O | — | M26 | — | M26 |
| A834 | 1-3 | O | S | — | M1 | — | M1 |
| A835 | 1-3 | O | S | — | M11 | — | M11 |
| A836 | 1-3 | O | S | — | M3 | — | M3 |
| A837 | 1-3 | O | S | F1 | M10 | F1 | M10 |
| A838 | 1-3 | O | S | — | M15 | — | M15 |
| A839 | 1-3 | O | S | F1 | M14 | F1 | M14 |
| A840 | 1-3 | O | S | F1 | M18 | F1 | M18 |
| A841 | 1-3 | O | S | — | M26 | — | M26 |
| A842 | 1-4 | O | O | — | M1 | — | M1 |
| A843 | 1-4 | O | O | — | M15 | — | M15 |
| A844 | 1-4 | O | O | — | M19 | — | M19 |
| A845 | 1-4 | O | O | F1 | M18 | F1 | M18 |
| A846 | 1-4 | O | S | — | M1 | — | M1 |
| A847 | 1-4 | O | S | — | M11 | — | M1 |
| A848 | 1-4 | O | S | — | M3 | — | M3 |
| A849 | 1-4 | O | S | F1 | M10 | F1 | M10 |
| A850 | 1-4 | O | S | F1 | M14 | F1 | M14 |
| A851 | 1-4 | O | S | — | M26 | — | M26 |

According to one example of the present disclosure, the compound represented by the Chemical Formula 2-1 may be selected from the group consisting of the compounds as shown in Tables 10 and 11 as set forth below. However, the present disclosure is not limited thereto. In the compounds as listed in Table 10 as set forth below, all of R₁₁ to R₁₂, R₂₁ to R₂₄, R₃₁ to R₃₃, R₃₅, R₄₁ to R₄₃, and R₅₁ to R₅₄ may be hydrogen. In the compounds as listed in Table 11 as set forth below, all of R₁₁ to R₁₂, R₂₁ to R₂₄, R₃₁ to R₃₃, R₃₅, R₄₁ to R₄₃, and R₅₁ to R₅₄ may be deuterium. In the following Tables, the mark “—” as each of X₁, X₂, L₁, L₂, Ar₁, and Ar₂ means a single bond.

TABLE 10

| Compound No. | X₁ | X₂ | L₁ | Ar₁ | L₂ | Ar₂ |
|---|---|---|---|---|---|---|
| B1 | O | O | — | M1 | — | M1 |
| B2 | O | O | — | M8 | — | M1 |
| B3 | O | O | — | M10 | — | M1 |
| B4 | O | O | — | M2 | — | M1 |
| B5 | O | O | — | M5 | — | M1 |
| B6 | O | O | F1 | M7 | — | M1 |
| B7 | O | O | F1 | M10 | — | M1 |
| B8 | O | O | — | M10 | — | M1 |
| B9 | O | O | — | M9 | — | M1 |
| B10 | O | O | — | M14 | — | M1 |
| B11 | O | O | F1 | M14 | — | M1 |
| B12 | O | O | — | M18 | — | M1 |
| B13 | O | O | F1 | M18 | — | M1 |
| B14 | O | O | — | M24 | — | M1 |
| B15 | O | O | — | M26 | — | M1 |
| B16 | O | O | — | M8 | — | M7 |
| B17 | O | O | — | M6 | — | M7 |
| B18 | O | O | F1 | M7 | — | M7 |
| B19 | O | O | F1 | M11 | — | M7 |
| B20 | O | O | — | M9 | — | M7 |
| B21 | O | O | F1 | M9 | — | M7 |
| B22 | O | O | — | M15 | — | M7 |
| B23 | O | O | F1 | M14 | — | M7 |
| B24 | O | O | — | M19 | — | M7 |
| B25 | O | O | F1 | M20 | — | M7 |
| B26 | O | O | — | M24 | — | M7 |
| B27 | O | O | — | M11 | — | M8 |
| B28 | O | O | — | M3 | — | M8 |
| B29 | O | O | F1 | M16 | — | M8 |
| B30 | O | O | F1 | M10 | — | M10 |
| B31 | O | O | F1 | M9 | — | M10 |
| B32 | O | O | F1 | M14 | — | M10 |
| B33 | O | O | — | M19 | — | M10 |
| B34 | O | O | — | M24 | — | M10 |
| B35 | O | O | — | M2 | — | M11 |
| B36 | O | O | — | M5 | — | M11 |
| B37 | O | O | F1 | M7 | — | M11 |
| B38 | O | O | — | M9 | — | M11 |
| B39 | O | O | — | M15 | — | M11 |
| B40 | O | O | F1 | M14 | — | M11 |
| B41 | O | O | F1 | M20 | — | M11 |
| B42 | O | O | — | M26 | — | M11 |
| B43 | O | O | F1 | M17 | — | M12 |
| B44 | O | O | — | M3 | — | M13 |
| B45 | O | O | F1 | M16 | — | M13 |
| B46 | O | O | — | M4 | — | M2 |
| B47 | O | O | — | M9 | — | M2 |
| B48 | O | O | F1 | M16 | — | M2 |
| B49 | O | O | — | M18 | — | M2 |
| B50 | O | O | — | M23 | — | M2 |
| B51 | O | O | F1 | M10 | — | M4 |
| B52 | O | O | F1 | M15 | — | M4 |
| B53 | O | O | F1 | M21 | — | M4 |
| B54 | O | O | — | M24 | — | M4 |
| B55 | O | O | F1 | M24 | — | M4 |
| B56 | O | O | — | M6 | — | M3 |
| B57 | O | O | F1 | M9 | — | M3 |
| B58 | O | O | F1 | M17 | — | M3 |
| B59 | O | O | — | M19 | — | M3 |
| B60 | O | O | F1 | M20 | — | M3 |
| B61 | O | O | — | M5 | — | M6 |
| B62 | O | O | F1 | M7 | — | M6 |
| B63 | O | O | F1 | M10 | — | M6 |
| B64 | O | O | — | M14 | — | M6 |
| B65 | O | O | F1 | M14 | — | M6 |
| B66 | O | O | F1 | M20 | — | M6 |
| B67 | O | O | — | M26 | — | M6 |
| B68 | O | O | F1 | M8 | — | M5 |
| B69 | O | O | F1 | M11 | — | M5 |
| B70 | O | O | F1 | M9 | — | M5 |
| B71 | O | O | — | M15 | — | M5 |
| B72 | O | O | F1 | M16 | — | M5 |
| B73 | O | O | — | M19 | — | M5 |
| B74 | O | O | F1 | M19 | — | M5 |
| B75 | O | O | — | M24 | — | M5 |
| B76 | O | O | F1 | M23 | — | M5 |
| B77 | O | O | F1 | M8 | F1 | M7 |
| B78 | O | O | — | M9 | F1 | M7 |
| B79 | O | O | F1 | M15 | F1 | M7 |
| B80 | O | O | F1 | M18 | F1 | M7 |
| B81 | O | O | — | M24 | F1 | M7 |
| B82 | O | O | F1 | M23 | F1 | M8 |
| B83 | O | O | — | M14 | F1 | M10 |
| B84 | O | O | F1 | M14 | F1 | M10 |
| B85 | O | O | F1 | M18 | F1 | M10 |
| B86 | O | O | — | M9 | F1 | M11 |
| B87 | O | O | F1 | M16 | F1 | M11 |
| B88 | O | O | — | M23 | F1 | M11 |
| B89 | O | O | — | M15 | F1 | M13 |
| B90 | O | O | F1 | M16 | F1 | M13 |
| B91 | O | O | F1 | M9 | — | M9 |
| B92 | O | O | — | M15 | — | M9 |
| B93 | O | O | F1 | M14 | — | M9 |
| B94 | O | O | — | M19 | — | M9 |
| B95 | O | O | F1 | M18 | — | M9 |
| B96 | O | O | — | M24 | — | M9 |
| B97 | O | O | — | M26 | — | M9 |
| B98 | O | O | — | M14 | F1 | M9 |
| B99 | O | O | — | M15 | — | M14 |
| B100 | O | O | — | M24 | — | M14 |
| B101 | O | O | F1 | M15 | — | M15 |
| B102 | O | O | F1 | M20 | — | M15 |
| B103 | O | O | — | M26 | — | M15 |
| B104 | O | O | F1 | M17 | F1 | M14 |
| B105 | O | O | — | M19 | F1 | M14 |
| B106 | O | O | F1 | M19 | F1 | M14 |
| B107 | O | O | — | M23 | F1 | M14 |
| B108 | O | O | — | M18 | F1 | M16 |
| B109 | O | O | F1 | M19 | — | M18 |
| B110 | O | O | — | M24 | — | M19 |
| B111 | O | O | — | M26 | F1 | M19 |
| B112 | O | O | — | M23 | — | M24 |
| B113 | O | S | — | M7 | — | M1 |
| B114 | O | S | — | M10 | — | M1 |
| B115 | O | S | — | M2 | — | M1 |
| B116 | O | S | — | M5 | — | M1 |
| B117 | O | S | F1 | M7 | — | M1 |
| B118 | O | S | F1 | M10 | — | M1 |
| B119 | O | S | — | M9 | — | M1 |
| B120 | O | S | — | M15 | — | M1 |
| B121 | O | S | — | M14 | — | M1 |
| B122 | O | S | F1 | M14 | — | M1 |
| B123 | O | S | — | M18 | — | M1 |
| B124 | O | S | F1 | M18 | — | M1 |
| B125 | O | S | — | M24 | — | M1 |
| B126 | O | S | — | M6 | — | M7 |
| B127 | O | S | F1 | M9 | — | M7 |
| B128 | O | S | — | M15 | — | M7 |
| B129 | O | S | — | M19 | — | M7 |
| B130 | O | S | F1 | M20 | — | M7 |
| B131 | O | S | — | M26 | — | M7 |
| B132 | O | S | — | M3 | — | M8 |
| B133 | O | S | F1 | M16 | — | M8 |
| B134 | O | S | F1 | M10 | — | M10 |
| B135 | O | S | F1 | M9 | — | M10 |
| B136 | O | S | — | M19 | — | M10 |
| B137 | O | S | — | M24 | — | M10 |
| B138 | O | S | — | M2 | — | M11 |
| B139 | O | S | — | M5 | — | M11 |
| B140 | O | S | F1 | M7 | — | M11 |
| B141 | O | S | — | M9 | — | M11 |
| B142 | O | S | — | M15 | — | M11 |
| B143 | O | S | F1 | M14 | — | M11 |
| B144 | O | S | F1 | M20 | — | M11 |
| B145 | O | S | — | M26 | — | M11 |
| B146 | O | S | F1 | M17 | — | M12 |
| B147 | O | S | F1 | M16 | — | M13 |
| B148 | O | S | — | M2 | — | M2 |
| B149 | O | S | — | M9 | — | M2 |
| B150 | O | S | F1 | M16 | — | M2 |
| B151 | O | S | — | M18 | — | M2 |
| B152 | O | S | — | M23 | — | M2 |
| B153 | O | S | F1 | M8 | — | M4 |
| B154 | O | S | F1 | M21 | — | M4 |
| B155 | O | S | — | M6 | — | M3 |
| B156 | O | S | — | M5 | — | M3 |
| B157 | O | S | F1 | M12 | — | M3 |
| B158 | O | S | F1 | M9 | — | M3 |
| B159 | O | S | F1 | M17 | — | M3 |
| B160 | O | S | — | M19 | — | M3 |
| B161 | O | S | F1 | M20 | — | M3 |
| B162 | O | S | — | M5 | — | M6 |
| B163 | O | S | F1 | M7 | — | M6 |
| B164 | O | S | F1 | M10 | — | M6 |
| B165 | O | S | — | M14 | — | M6 |
| B166 | O | S | F1 | M14 | — | M6 |
| B167 | O | S | F1 | M20 | — | M6 |
| B168 | O | S | — | M26 | — | M6 |
| B169 | O | S | F1 | M11 | — | M5 |
| B170 | O | S | F1 | M16 | — | M5 |
| B171 | O | S | — | M19 | — | M5 |
| B172 | O | S | F1 | M19 | — | M5 |
| B173 | O | S | — | M24 | — | M5 |
| B174 | O | S | — | M9 | F1 | M7 |
| B175 | O | S | F1 | M15 | F1 | M7 |
| B176 | O | S | F1 | M18 | F1 | M7 |
| B177 | O | S | — | M14 | F1 | M10 |
| B178 | O | S | F1 | M14 | F1 | M10 |
| B179 | O | S | F1 | M18 | F1 | M10 |
| B180 | O | S | — | M9 | F1 | M11 |
| B181 | O | S | — | M19 | F1 | M11 |
| B182 | O | S | — | M23 | F1 | M11 |
| B183 | O | S | — | M15 | — | M9 |
| B184 | O | S | F1 | M14 | — | M9 |
| B185 | O | S | — | M19 | — | M9 |
| B186 | O | S | F1 | M18 | — | M9 |
| B187 | O | S | — | M24 | — | M9 |
| B188 | O | S | — | M15 | — | M14 |
| B189 | O | S | — | M19 | — | M14 |
| B190 | O | S | F1 | M15 | — | M15 |
| B191 | O | S | F1 | M20 | — | M15 |
| B192 | O | S | — | M26 | — | M15 |
| B193 | O | S | F1 | M17 | F1 | M14 |
| B194 | O | S | — | M19 | F1 | M14 |
| B195 | O | S | F1 | M19 | F1 | M14 |
| B196 | O | S | F1 | M23 | F1 | M15 |
| B197 | O | S | — | M18 | F1 | M16 |
| B198 | O | S | F1 | M19 | — | M18 |
| B199 | S | O | F1 | M14 | — | M1 |
| B200 | S | O | — | M15 | — | M1 |
| B201 | S | S | — | M1 | — | M1 |
| B202 | S | S | — | M30 | — | M30 |

TABLE 11

| Compound No. | X₁ | X₂ | L₁ | Ar₁ | L₂ | Ar₂ |
|---|---|---|---|---|---|---|
| B203 | O | O | — | M30 | — | M31 |
| B204 | O | S | — | M30 | — | M38 |
| B205 | O | S | F2 | M37 | — | M30 |
| B206 | S | O | — | M30 | F2 | M39 |
| B207 | S | S | — | M30 | — | M30 |

According to one example of the present disclosure, the compound represented by the Chemical Formula 2-2 may be selected from the group consisting of the compounds as shown in Tables 12 and 13 as set forth below. However, the present disclosure is not limited thereto. In the compounds as listed in Table 12 as set forth below, all of R₁₁ to R₁₂, R₂₁ to R₂₄, R₃₁ to R₃₃, R₃₅, R₄₁ to R₄₃, and R₅₁ to R₅₄ may be hydrogen. In the compounds as listed in Table 13 as set forth below, all of R₁₁ to R₁₂, R₂₁ to R₂₄, R₃₁ to R₃₃, R₃₅, R₄₁ to R₄₃, and R₅₁ to R₅₄ may be deuterium. In the following Tables, the mark “—” as each of X₁, X₂, L₁, L₂, Ar₁, and Ar₂ means a single bond.

TABLE 12

| Compound No. | X₁ | X₂ | L₁ | Ar₁ | L₂ | Ar₂ |
|---|---|---|---|---|---|---|
| B208 | O | O | — | M1 | — | M1 |
| B209 | O | O | — | M8 | — | M1 |
| B210 | O | O | — | M10 | — | M1 |
| B211 | O | O | — | M2 | — | M1 |
| B212 | O | O | — | M5 | — | M1 |
| B213 | O | O | F1 | M7 | — | M1 |
| B214 | O | O | F1 | M10 | — | M1 |
| B215 | O | O | — | M9 | — | M1 |
| B216 | O | O | — | M14 | — | M1 |
| B217 | O | O | F1 | M14 | — | M1 |
| B218 | O | O | — | M18 | — | M1 |
| B219 | O | O | — | M20 | — | M1 |
| B220 | O | O | F1 | M18 | — | M1 |
| B221 | O | O | — | M24 | — | M1 |
| B222 | O | O | — | M8 | — | M7 |
| B223 | O | O | — | M6 | — | M7 |
| B224 | O | O | F1 | M7 | — | M7 |
| B225 | O | O | F1 | M11 | — | M7 |
| B226 | O | O | — | M9 | — | M7 |
| B227 | O | O | F1 | M9 | — | M7 |
| B228 | O | O | — | M15 | — | M7 |
| B229 | O | O | F1 | M14 | — | M7 |
| B230 | O | O | — | M19 | — | M7 |
| B231 | O | O | F1 | M20 | — | M7 |
| B232 | O | O | — | M11 | — | M8 |
| B233 | O | O | — | M3 | — | M8 |
| B234 | O | O | F1 | M16 | — | M8 |
| B235 | O | O | F1 | M10 | — | M10 |
| B236 | O | O | F1 | M9 | — | M10 |
| B237 | O | O | F1 | M14 | — | M10 |
| B238 | O | O | — | M19 | — | M10 |
| B239 | O | O | — | M2 | — | M11 |
| B240 | O | O | — | M5 | — | M11 |
| B241 | O | O | F1 | M7 | — | M11 |
| B242 | O | O | — | M9 | — | M11 |
| B243 | O | O | — | M15 | — | M11 |
| B244 | O | O | F1 | M14 | — | M11 |
| B245 | O | O | F1 | M20 | — | M11 |
| B246 | O | O | — | M24 | — | M11 |
| B247 | O | O | F1 | M17 | — | M12 |
| B248 | O | O | — | M3 | — | M13 |
| B249 | O | O | F1 | M16 | — | M13 |
| B250 | O | O | — | M4 | — | M2 |
| B251 | O | O | — | M9 | — | M2 |
| B252 | O | O | F1 | M16 | — | M2 |
| B253 | O | O | — | M18 | — | M2 |
| B254 | O | O | — | M23 | — | M2 |
| B255 | O | O | F1 | M10 | — | M4 |
| B256 | O | O | F1 | M14 | — | M4 |
| B257 | O | O | F1 | M15 | — | M4 |
| B258 | O | O | F1 | M21 | — | M4 |
| B259 | O | O | F1 | M24 | — | M4 |
| B260 | O | O | — | M6 | — | M3 |
| B261 | O | O | F1 | M9 | — | M3 |
| B262 | O | O | F1 | M17 | — | M3 |
| B263 | O | O | — | M19 | — | M3 |
| B264 | O | O | F1 | M20 | — | M3 |
| B265 | O | O | — | M5 | — | M6 |
| B266 | O | O | F1 | M7 | — | M6 |
| B267 | O | O | F1 | M10 | — | M6 |
| B268 | O | O | — | M14 | — | M6 |
| B269 | O | O | F1 | M14 | — | M6 |
| B270 | O | O | F1 | M20 | — | M6 |
| B271 | O | O | F1 | M8 | — | M5 |
| B272 | O | O | F1 | M11 | — | M5 |
| B273 | O | O | F1 | M9 | — | M5 |
| B274 | O | O | — | M15 | — | M5 |
| B275 | O | O | F1 | M16 | — | M5 |
| B276 | O | O | — | M19 | — | M5 |
| B277 | O | O | F1 | M19 | — | M5 |
| B278 | O | O | F1 | M8 | F1 | M7 |
| B279 | O | O | — | M9 | F1 | M7 |
| B280 | O | O | F1 | M15 | F1 | M7 |
| B281 | O | O | F1 | M18 | F1 | M7 |
| B282 | O | O | — | M24 | F1 | M7 |
| B283 | O | O | — | M14 | F1 | M10 |
| B284 | O | O | F1 | M14 | F1 | M10 |
| B285 | O | O | F1 | M18 | F1 | M10 |
| B286 | O | O | — | M9 | F1 | M11 |
| B287 | O | O | F1 | M16 | F1 | M11 |
| B288 | O | O | — | M23 | F1 | M11 |
| B289 | O | O | — | M15 | F1 | M13 |
| B290 | O | O | F1 | M16 | F1 | M13 |
| B291 | O | O | F1 | M9 | — | M9 |
| B292 | O | O | — | M15 | — | M9 |
| B293 | O | O | F1 | M14 | — | M9 |
| B294 | O | O | — | M19 | — | M9 |
| B295 | O | O | F1 | M18 | — | M9 |
| B296 | O | O | — | M26 | — | M9 |
| B297 | O | O | — | M14 | — | M9 |
| B298 | O | O | — | M15 | — | M14 |
| B299 | O | O | F1 | M15 | — | M15 |
| B300 | O | O | F1 | M20 | — | M15 |
| B301 | O | O | — | M24 | — | M15 |
| B302 | O | O | F1 | M17 | F1 | M14 |
| B303 | O | O | — | M19 | F1 | M14 |
| B304 | O | O | F1 | M19 | F1 | M14 |
| B305 | O | O | — | M18 | F1 | M16 |
| B306 | O | O | F1 | M19 | — | M18 |
| B307 | O | O | — | M24 | — | M19 |
| B308 | O | O | — | M23 | — | M24 |
| B309 | O | S | — | M7 | — | M1 |
| B310 | O | S | — | M10 | — | M1 |
| B311 | O | S | — | M2 | — | M1 |
| B312 | O | S | — | M5 | — | M1 |
| B313 | O | S | F1 | M7 | — | M1 |
| B314 | O | S | F1 | M10 | — | M1 |
| B315 | O | S | — | M9 | — | M1 |
| B316 | O | S | — | M14 | — | M1 |
| B317 | O | S | F1 | M14 | — | M1 |
| B318 | O | S | — | M18 | — | M1 |
| B319 | O | S | — | M20 | — | M1 |
| B320 | O | S | F1 | M19 | — | M1 |
| B321 | O | S | — | M24 | — | M1 |
| B322 | O | S | — | M6 | — | M7 |
| B323 | O | S | F1 | M9 | — | M7 |
| B324 | O | S | — | M15 | — | M7 |
| B325 | O | S | — | M19 | — | M7 |
| B326 | O | S | F1 | M20 | — | M7 |
| B327 | O | S | — | M26 | — | M7 |
| B328 | O | S | — | M3 | — | M8 |
| B329 | O | S | F1 | M16 | — | M8 |
| B330 | O | S | F1 | M10 | — | M10 |
| B331 | O | S | F1 | M9 | — | M10 |
| B332 | O | S | — | M19 | — | M10 |
| B333 | O | S | — | M24 | — | M10 |
| B334 | O | S | — | M2 | — | M11 |
| B335 | O | S | — | M5 | — | M11 |
| B336 | O | S | F1 | M7 | — | M11 |
| B337 | O | S | — | M9 | — | M11 |
| B338 | O | S | — | M15 | — | M11 |
| B339 | O | S | F1 | M14 | — | M11 |
| B340 | O | S | F1 | M20 | — | M11 |
| B341 | O | S | — | M26 | — | M11 |
| B342 | O | S | F1 | M17 | — | M12 |
| B343 | O | S | F1 | M16 | — | M13 |
| B344 | O | S | — | M2 | — | M2 |
| B345 | O | S | — | M9 | — | M2 |
| B346 | O | S | F1 | M16 | — | M2 |
| B347 | O | S | — | M18 | — | M2 |
| B348 | O | S | — | M23 | — | M2 |
| B349 | O | S | F1 | M8 | — | M4 |
| B350 | O | S | — | M3 | — | M3 |
| B351 | O | S | — | M6 | — | M3 |
| B352 | O | S | — | M5 | — | M3 |
| B353 | O | S | F1 | M12 | — | M3 |
| B354 | O | S | F1 | M9 | — | M3 |
| B355 | O | S | F1 | M20 | — | M3 |
| B356 | O | S | — | M5 | — | M6 |
| B357 | O | S | F1 | M7 | — | M6 |
| B358 | O | S | F1 | M10 | — | M6 |
| B359 | O | S | — | M14 | — | M6 |
| B360 | O | S | F1 | M14 | — | M6 |
| B361 | O | S | — | M26 | — | M6 |
| B362 | O | S | F1 | M11 | — | M5 |
| B363 | O | S | F1 | M16 | — | M5 |
| B364 | O | S | — | M19 | — | M5 |
| B365 | O | S | F1 | M19 | — | M5 |
| B366 | O | S | — | M24 | — | M5 |
| B367 | O | S | — | M9 | F1 | M7 |
| B368 | O | S | F1 | M15 | F1 | M7 |
| B369 | O | S | — | M14 | F1 | M10 |
| B370 | O | S | F1 | M14 | F1 | M10 |
| B371 | O | S | F1 | M18 | F1 | M10 |
| B372 | O | S | — | M9 | F1 | M11 |
| B373 | O | S | — | M19 | F1 | M11 |
| B374 | O | S | — | M23 | F1 | M11 |
| B375 | O | S | — | M15 | — | M9 |
| B376 | O | S | F1 | M14 | — | M9 |
| B377 | O | S | — | M19 | — | M9 |
| B378 | O | S | F1 | M18 | — | M9 |
| B379 | O | S | — | M24 | — | M9 |
| B380 | O | S | — | M15 | — | M14 |
| B381 | O | S | F1 | M15 | — | M15 |
| B382 | O | S | F1 | M17 | F1 | M14 |
| B383 | O | S | — | M19 | F1 | M14 |
| B384 | O | S | F1 | M19 | F1 | M14 |
| B385 | O | S | F1 | M23 | F1 | M15 |
| B386 | O | S | — | M18 | F1 | M16 |
| B387 | O | S | F1 | M19 | — | M18 |
| B388 | S | O | F1 | M19 | — | M1 |
| B389 | S | O | — | M20 | — | M1 |
| B390 | S | S | F1 | M7 | — | M1 |

TABLE 13

| Compound No. | X₁ | X₂ | L₁ | Ar₁ | L₂ | Ar₂ |
|---|---|---|---|---|---|---|
| B391 | O | O | — | M30 | — | M33 |
| B392 | O | S | — | M30 | — | M39 |
| B393 | O | S | — | M30 | — | M40 |
| B394 | O | S | — | M30 | F2 | M32 |
| B395 | S | O | — | M30 | F2 | M39 |
| B396 | S | S | — | M30 | F2 | M32 |

According to one example of the present disclosure, the compound represented by the Chemical Formula 2-3 may be selected from the group consisting of the compounds as shown in Tables 14 and 15 as set forth below. However, the present disclosure is not limited thereto. In the compounds as listed in Table 14 as set forth below, all of R₁₁ to R₁₂, R₂₁ to R₂₄, R₃₁ to R₃₃, R₃₅, R₄₁ to R₄₃, and R₅₁ to R₅₄ may be hydrogen. In the compounds as listed in Table 15 as set forth below, all of R₁₁ to R₁₂, R₂₁ to R₂₄, R₃₁ to R₃₃, R₃₅, R₄₁ to R₄₃, and R₅₁ to R₅₄ may be deuterium. In the following Tables, the mark “—” as each of X₁, X₂, L₁, L₂, Ar₁, and Ar₂ means a single bond.

TABLE 14

| Compound No. | X₁ | X₂ | L₁ | Ar₁ | L₂ | Ar₂ |
|---|---|---|---|---|---|---|
| B397 | O | O | — | M8 | — | M1 |
| B398 | O | O | — | M11 | — | M1 |
| B399 | O | O | — | M2 | — | M1 |
| B400 | O | O | — | M5 | — | M1 |
| B401 | O | O | F1 | M7 | — | M1 |
| B402 | O | O | F1 | M10 | — | M1 |
| B403 | O | O | — | M9 | — | M1 |
| B404 | O | O | — | M14 | — | M1 |
| B405 | O | O | F1 | M14 | — | M1 |
| B406 | O | O | — | M18 | — | M1 |
| B407 | O | O | F1 | M18 | — | M1 |
| B408 | O | O | — | M24 | — | M1 |
| B409 | O | O | — | M8 | — | M7 |
| B410 | O | O | — | M6 | — | M7 |
| B411 | O | O | F1 | M7 | — | M7 |
| B412 | O | O | F1 | M11 | — | M7 |
| B413 | O | O | F1 | M9 | — | M7 |
| B414 | O | O | — | M15 | — | M7 |
| B415 | O | O | — | M19 | — | M7 |
| B416 | O | O | — | M11 | — | M8 |
| B417 | O | O | — | M3 | — | M8 |
| B418 | O | O | F1 | M16 | — | M8 |
| B419 | O | O | F1 | M10 | — | M10 |
| B420 | O | O | — | M2 | — | M11 |
| B421 | O | O | — | M5 | — | M11 |
| B422 | O | O | F1 | M7 | — | M11 |
| B423 | O | O | — | M9 | — | M11 |
| B424 | O | O | — | M15 | — | M11 |
| B425 | O | O | F1 | M15 | — | M11 |
| B426 | O | O | — | M19 | — | M11 |
| B427 | O | O | F1 | M20 | — | M11 |
| B428 | O | O | — | M24 | — | M11 |
| B429 | O | O | F1 | M17 | — | M12 |
| B430 | O | O | — | M3 | — | M13 |
| B431 | O | O | — | M4 | — | M2 |
| B432 | O | O | — | M9 | — | M2 |
| B433 | O | O | F1 | M16 | — | M2 |
| B434 | O | O | — | M18 | — | M2 |
| B435 | O | O | — | M23 | — | M2 |
| B436 | O | O | F1 | M10 | — | M4 |
| B437 | O | O | F1 | M15 | — | M4 |
| B438 | O | O | — | M6 | — | M3 |
| B439 | O | O | F1 | M9 | — | M3 |
| B440 | O | O | F1 | M17 | — | M3 |
| B441 | O | O | — | M19 | — | M3 |
| B442 | O | O | F1 | M20 | — | M3 |
| B443 | O | O | — | M5 | — | M6 |
| B444 | O | O | F1 | M7 | — | M6 |
| B445 | O | O | F1 | M10 | — | M6 |
| B446 | O | O | — | M14 | — | M6 |
| B447 | O | O | F1 | M14 | — | M6 |
| B448 | O | O | F1 | M8 | — | M5 |
| B449 | O | O | F1 | M11 | — | M5 |
| B450 | O | O | — | M15 | — | M5 |
| B451 | O | O | F1 | M8 | F1 | M7 |
| B452 | O | O | — | M9 | F1 | M7 |
| B453 | O | O | F1 | M15 | F1 | M7 |
| B454 | O | O | — | M14 | F1 | M10 |
| B455 | O | O | F1 | M18 | F1 | M10 |
| B456 | O | O | — | M9 | F1 | M11 |
| B457 | O | O | F1 | M16 | F1 | M11 |
| B458 | O | O | — | M23 | F1 | M11 |
| B459 | O | O | F1 | M9 | — | M9 |
| B460 | O | O | — | M15 | — | M9 |
| B461 | O | O | F1 | M14 | — | M9 |
| B462 | O | O | — | M19 | — | M9 |
| B463 | O | O | F1 | M18 | — | M9 |
| B464 | O | O | — | M26 | — | M9 |
| B465 | O | O | — | M14 | F1 | M9 |
| B466 | O | O | — | M15 | — | M14 |
| B467 | O | O | F1 | M15 | — | M15 |
| B468 | O | O | F1 | M18 | — | M15 |
| B469 | O | O | — | M24 | — | M15 |
| B470 | O | O | F1 | M17 | F1 | M14 |
| B471 | O | O | F1 | M19 | F1 | M14 |
| B472 | O | O | — | M18 | F1 | M16 |
| B473 | O | O | — | M23 | | M24 |
| B474 | O | S | — | M7 | — | M1 |
| B475 | O | S | — | M10 | — | M1 |
| B476 | O | S | — | M2 | — | M1 |
| B477 | O | S | — | M5 | — | M1 |
| B478 | O | S | F1 | M10 | — | M1 |
| B479 | O | S | — | M9 | — | M1 |
| B480 | O | S | — | M14 | — | M1 |
| B481 | O | S | F1 | M14 | — | M1 |
| B482 | O | S | — | M18 | — | M1 |
| B483 | O | S | F1 | M19 | — | M1 |
| B484 | O | S | F1 | M21 | — | M1 |
| B485 | O | S | — | M24 | — | M1 |
| B486 | O | S | — | M28 | — | M1 |
| B487 | O | S | — | M6 | — | M7 |
| B488 | O | S | F1 | M7 | — | M7 |
| B489 | O | S | F1 | M9 | — | M7 |
| B490 | O | S | — | M15 | — | M7 |
| B491 | O | S | — | M19 | — | M7 |
| B492 | O | S | F1 | M20 | — | M7 |
| B493 | O | S | — | M3 | — | M8 |
| B494 | O | S | F1 | M16 | — | M8 |
| B495 | O | S | F1 | M10 | — | M10 |
| B496 | O | S | F1 | M9 | — | M10 |
| B497 | O | S | — | M19 | — | M10 |
| B498 | O | S | — | M24 | — | M10 |
| B499 | O | S | — | M2 | — | M11 |
| B500 | O | S | — | M5 | — | M11 |
| B501 | O | S | F1 | M7 | — | M11 |
| B502 | O | S | — | M9 | — | M11 |
| B503 | O | S | — | M15 | — | M11 |
| B504 | O | S | F1 | M14 | — | M11 |
| B505 | O | S | F1 | M20 | — | M11 |
| B506 | O | S | F1 | M17 | — | M12 |
| B507 | O | S | — | M2 | — | M2 |
| B508 | O | S | — | M9 | — | M2 |
| B509 | O | S | — | M18 | — | M2 |
| B510 | O | S | — | M23 | — | M2 |
| B511 | O | S | F1 | M8 | — | M4 |
| B512 | O | S | F1 | M12 | — | M3 |
| B513 | O | S | F1 | M9 | — | M3 |
| B514 | O | S | — | M19 | — | M3 |
| B515 | O | S | F1 | M20 | — | M3 |
| B516 | O | S | F1 | M10 | — | M6 |
| B517 | O | S | — | M14 | — | M6 |
| B518 | O | S | F1 | M14 | — | M6 |
| B519 | O | S | F1 | M11 | — | M5 |
| B520 | O | S | F1 | M16 | — | M5 |
| B521 | O | S | F1 | M19 | — | M5 |
| B522 | O | S | — | M9 | F1 | M7 |
| B523 | O | S | — | M14 | F1 | M10 |
| B524 | O | S | F1 | M14 | F1 | M10 |
| B525 | O | S | F1 | M18 | F1 | M10 |
| B526 | O | S | — | M9 | F1 | M11 |
| B527 | O | S | — | M19 | F1 | M11 |
| B528 | O | S | — | M15 | — | M9 |
| B529 | O | S | F1 | M14 | — | M9 |
| B530 | O | S | — | M19 | — | M9 |
| B531 | O | S | F1 | M18 | — | M9 |
| B532 | O | S | — | M24 | — | M9 |
| B533 | O | S | — | M15 | — | M14 |
| B534 | O | S | F1 | M15 | — | M15 |
| B535 | O | S | — | M19 | F1 | M14 |
| B536 | O | S | F1 | M19 | F1 | M14 |
| B537 | O | S | F1 | M23 | F1 | M15 |
| B538 | S | O | — | M1 | F1 | M29 |
| B539 | S | S | — | M1 | F1 | M13 |

TABLE 15

| Compound No. | X₁ | X₂ | L₁ | Ar₁ | L₂ | Ar₂ |
|---|---|---|---|---|---|---|
| B540 | O | O | — | M30 | — | M35 |
| B541 | O | S | — | M31 | — | M31 |
| B542 | S | O | — | M1 | F1 | M29 |
| B543 | S | S | — | M30 | F2 | M36 |

According to one example of the present disclosure, the compound represented by the Chemical Formula 2-4 may be selected from the group consisting of the compounds as shown in Tables 16 and 17 as set forth below. However, the present disclosure is not limited thereto. In the compounds as listed in Table 16 as set forth below, all of R₁ to R₁₂, R₂₁ to R₂₄, R₃₁ to R₃₃, R₃₅, R₄₁ to R₄₃, and R₅₁ to R₅₄ may be hydrogen. In the compounds as listed in Table 17 as set forth below, all of R₁₁ to R₁₂, R₂₁ to R₂₄, R₃₁ to R₃₃, R₃₅, R₄₁ to R₄₃, and R₅₁ to R₅₄ may be deuterium. In the following Tables, the mark “—” as each of X₁, X₂, L₁, L₂, Ar₁, and Ar₂ means a single bond.

TABLE 16

| Compound No. | X₁ | X₂ | L₁ | Ar₁ | L₂ | Ar₂ |
|---|---|---|---|---|---|---|
| B544 | O | O | — | M1 | — | M1 |
| B545 | O | O | — | M11 | — | M1 |
| B546 | O | O | — | M2 | — | M1 |
| B547 | O | O | — | M5 | — | M1 |
| B548 | O | O | F1 | M7 | — | M1 |
| B549 | O | O | F1 | M10 | — | M1 |
| B550 | O | O | — | M9 | — | M1 |
| B551 | O | O | — | M13 | — | M1 |
| B552 | O | O | — | M14 | — | M1 |
| B553 | O | O | F1 | M14 | — | M1 |
| B554 | O | O | — | M18 | — | M1 |
| B555 | O | O | F1 | M18 | — | M1 |
| B556 | O | O | — | M24 | — | M1 |
| B557 | O | O | — | M8 | — | M7 |
| B558 | O | O | — | M6 | — | M7 |
| B559 | O | O | F1 | M7 | — | M7 |
| B560 | O | O | F1 | M11 | — | M7 |
| B561 | O | O | F1 | M9 | — | M7 |
| B562 | O | O | — | M15 | — | M7 |
| B563 | O | O | — | M19 | — | M7 |
| B564 | O | O | — | M3 | — | M8 |
| B565 | O | O | F1 | M16 | — | M8 |
| B566 | O | O | — | M2 | — | M11 |
| B567 | O | O | — | M5 | — | M11 |
| B568 | O | O | — | M9 | — | M11 |
| B569 | O | O | — | M15 | — | M11 |
| B570 | O | O | F1 | M15 | — | M11 |
| B571 | O | O | F1 | M19 | — | M11 |
| B572 | O | O | — | M19 | — | M11 |
| B573 | O | O | — | M24 | — | M11 |
| B574 | O | O | F1 | M17 | — | M12 |
| B575 | O | O | — | M3 | — | M13 |
| B576 | O | O | — | M9 | — | M2 |
| B577 | O | O | F1 | M16 | — | M2 |
| B578 | O | O | — | M18 | — | M2 |
| B579 | O | O | — | M23 | — | M2 |
| B580 | O | O | F1 | M15 | — | M4 |
| B581 | O | O | — | M6 | — | M3 |
| B582 | O | O | F1 | M9 | — | M3 |
| B583 | O | O | F1 | M17 | — | M3 |
| B584 | O | O | — | M19 | — | M3 |
| B585 | O | O | F1 | M20 | — | M3 |
| B586 | O | O | — | M5 | — | M6 |
| B587 | O | O | F1 | M10 | — | M6 |
| B588 | O | O | — | M14 | — | M6 |
| B589 | O | O | F1 | M14 | — | M6 |
| B590 | O | O | F1 | M20 | — | M6 |
| B591 | O | O | F1 | M8 | — | M5 |
| B592 | O | O | F1 | M11 | — | M5 |
| B593 | O | O | — | M9 | F1 | M7 |
| B594 | O | O | F1 | M15 | F1 | M7 |
| B595 | O | O | — | M9 | F1 | M11 |
| B596 | O | O | F1 | M16 | F1 | M11 |
| B597 | O | O | — | M23 | F1 | M11 |
| B598 | O | O | F1 | M9 | — | M9 |
| B599 | O | O | — | M15 | — | M9 |
| B600 | O | O | F1 | M14 | — | M9 |
| B601 | O | O | — | M19 | — | M9 |
| B602 | O | O | F1 | M18 | — | M9 |
| B603 | O | O | — | M26 | — | M9 |
| B604 | O | O | — | M14 | F1 | M9 |
| B605 | O | O | — | M15 | — | M14 |
| B606 | O | O | F1 | M15 | — | M15 |
| B607 | O | O | F1 | M18 | — | M15 |
| B608 | O | O | — | M24 | — | M15 |
| B609 | O | O | — | M19 | F1 | M15 |
| B610 | O | S | — | M7 | — | M1 |
| B611 | O | S | — | M10 | — | M1 |
| B612 | O | S | — | M2 | — | M1 |
| B613 | O | S | — | M5 | — | M1 |
| B614 | O | S | F1 | M10 | — | M1 |
| B615 | O | S | — | M9 | — | M1 |
| B616 | O | S | — | M14 | — | M |
| B617 | O | S | F1 | M14 | — | M1 |
| B618 | O | S | — | M18 | — | M1 |
| B619 | O | S | F1 | M19 | — | M1 |
| B620 | O | S | — | M23 | — | M1 |
| B621 | O | S | — | M24 | — | M1 |
| B622 | O | S | F1 | M27 | — | M1 |
| B623 | O | S | — | M6 | — | M7 |
| B624 | O | S | F1 | M16 | — | M8 |
| B625 | O | S | — | M19 | — | M10 |
| B626 | O | S | — | M24 | — | M10 |
| B627 | O | S | — | M2 | — | M11 |
| B628 | O | S | — | M5 | — | M11 |
| B629 | O | S | — | M9 | — | M11 |
| B630 | O | S | — | M15 | — | M11 |
| B631 | O | S | F1 | M14 | — | M11 |
| B632 | O | S | — | M26 | — | M11 |
| B633 | O | S | F1 | M17 | — | M12 |
| B634 | O | S | — | M2 | — | M2 |
| B635 | O | S | — | M9 | — | M2 |
| B636 | O | S | — | M23 | — | M2 |
| B637 | O | S | F1 | M8 | — | M4 |
| B638 | O | S | F1 | M12 | — | M3 |
| B639 | O | S | F1 | M9 | — | M3 |
| B640 | O | S | — | M19 | — | M3 |
| B641 | O | S | F1 | M20 | — | M3 |
| B642 | O | S | F1 | M10 | — | M6 |
| B643 | O | S | — | M14 | — | M6 |
| B644 | O | S | — | M9 | F1 | M7 |
| B645 | O | S | — | M14 | F1 | M10 |
| B646 | O | S | F1 | M14 | F1 | M10 |
| B647 | O | S | F1 | M18 | F1 | M10 |
| B648 | O | S | — | M9 | F1 | M11 |
| B649 | O | S | — | M15 | — | M9 |
| B650 | O | S | F1 | M14 | — | M9 |
| B651 | O | S | F1 | M18 | — | M9 |
| B652 | O | S | — | M24 | — | M9 |
| B653 | O | S | F1 | M15 | — | M15 |
| B654 | O | S | F1 | M28 | — | M15 |
| B655 | O | S | F1 | M23 | F1 | M15 |
| B656 | S | O | F1 | M27 | — | M1 |
| B657 | S | S | F1 | M9 | — | M1 |

TABLE 17

| Compound No. | X₁ | X₂ | L₁ | Ar₁ | L₂ | Ar₂ |
|---|---|---|---|---|---|---|
| B658 | O | O | — | M30 | — | M34 |
| B659 | O | S | — | M30 | — | M42 |
| B660 | S | O | — | M30 | F2 | M43 |
| B661 | S | S | — | M30 | F2 | M34 |

Further, the compounds corresponding to Chemical Formulas 2-1 to 2-4 may be selected from the group consisting of Compounds represented by Table 18 as set forth below (all of R₁₁, R₁₂, R₂₁ to R₂₄, R₃₁ to R₃₃, R₃₅, R₄₁ to R₄₄, R₅₁ to R₅₄ of Table 18 are hydrogen).

TABLE 18

| Compound | Chemical | | | | | | |
|---|---|---|---|---|---|---|---|
| No. | Formula | X₁ | X₂ | L₁ | Ar₁ | L₂ | Ar₂ |
| B662 | 2-1 | O | O | F1 | M10 | F1 | M10 |
| B663 | 2-1 | O | O | — | M15 | — | M15 |
| B664 | 2-1 | O | O | F1 | M14 | F1 | M14 |
| B665 | 2-1 | O | O | — | M19 | — | M19 |
| B666 | 2-1 | O | O | F1 | M18 | F1 | M18 |
| B667 | 2-1 | O | O | — | M23 | — | M23 |
| B668 | 2-1 | O | O | — | M26 | — | M26 |
| B669 | 2-1 | O | S | — | M1 | — | M1 |
| B670 | 2-1 | O | S | — | M11 | — | M11 |
| B671 | 2-1 | O | S | — | M3 | — | M3 |
| B672 | 2-1 | O | S | F1 | M10 | F1 | M10 |
| B673 | 2-1 | O | S | — | M15 | — | M15 |
| B674 | 2-1 | O | S | F1 | M14 | F1 | M14 |
| B675 | 2-1 | O | S | — | M26 | — | M26 |
| B676 | 2-2 | O | O | F1 | M10 | F1 | M10 |
| B677 | 2-2 | O | O | — | M15 | — | M15 |
| B678 | 2-2 | O | O | F1 | M14 | F1 | M14 |
| B679 | 2-2 | O | O | — | M19 | — | M19 |
| B680 | 2-2 | O | O | — | M26 | — | M26 |
| B681 | 2-2 | O | S | — | M1 | — | M1 |
| B682 | 2-2 | O | S | — | M11 | — | M11 |
| B683 | 2-2 | O | S | F1 | M10 | F1 | M10 |
| B684 | 2-2 | O | S | — | M15 | — | M15 |
| B685 | 2-2 | O | S | F1 | M14 | F1 | M14 |
| B686 | 2-2 | O | S | — | M26 | — | M26 |
| B687 | 2-3 | O | O | — | M1 | — | M1 |
| B688 | 2-3 | O | O | F1 | M10 | F1 | M10 |
| B689 | 2-3 | O | O | — | M15 | — | M15 |
| B690 | 2-3 | O | O | — | M19 | — | M19 |
| B691 | 2-3 | O | S | — | M1 | — | M1 |
| B692 | 2-3 | O | S | — | M11 | — | M11 |
| B693 | 2-3 | O | S | — | M3 | — | M3 |
| B694 | 2-3 | O | S | F1 | M10 | F1 | M10 |
| B695 | 2-3 | O | S | — | M15 | — | M15 |
| B696 | 2-3 | O | S | — | M26 | — | M26 |
| B697 | 2-4 | O | O | F1 | M10 | F1 | M10 |
| B698 | 2-4 | O | O | — | M15 | — | M15 |
| B699 | 2-4 | O | O | F1 | M14 | F1 | M14 |
| B700 | 2-4 | O | S | — | M1 | — | M1 |
| B701 | 2-4 | O | S | — | M11 | — | M11 |
| B702 | 2-4 | O | S | — | M3 | — | M3 |
| B703 | 2-4 | O | S | F1 | M10 | F1 | M10 |
| B704 | 2-4 | O | S | F1 | M14 | F1 | M14 |
| B705 | 2-4 | O | S | — | M26 | — | M26 |

According to one example of the present disclosure, some structures of the Compounds displayed in the Tables 1 to 18 as set forth above may be represented as follows.

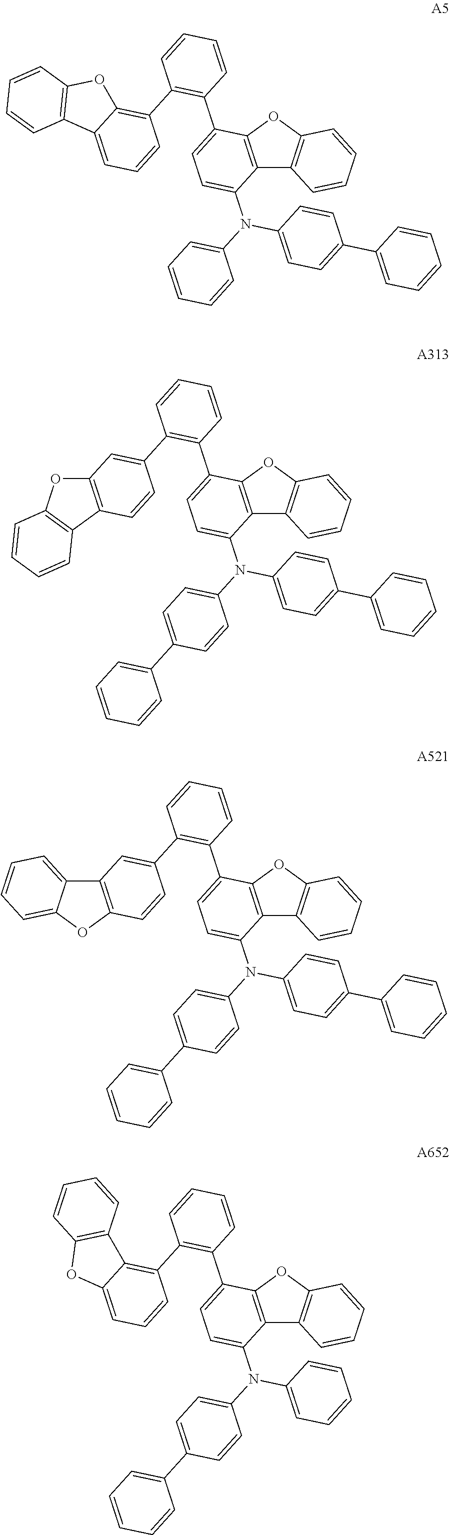 [SMILES: c1ccc(-c2ccc(N(c3ccccc3)c3ccc(-c4ccccc4-c4cccc5c4oc4ccccc45)c4oc5ccccc5c34)cc2)cc1 ; c1ccc(-c2ccc(N(c3ccc(-c4ccccc4)cc3)c3ccc(-c4ccccc4-c4ccc5c(c4)oc4ccccc45)c4oc5ccccc5c34)cc2)cc1 ; c1ccc(-c2ccc(N(c3ccc(-c4ccccc4)cc3)c3ccc(-c4ccccc4-c4ccc5oc6ccccc6c5c4)c4oc5ccccc5c34)cc2)cc1 ; c1ccc(-c2ccc(N(c3ccccc3)c3ccc(-c4ccccc4-c4cccc5oc6ccccc6c45)c4oc5ccccc5c34)cc2)cc1]

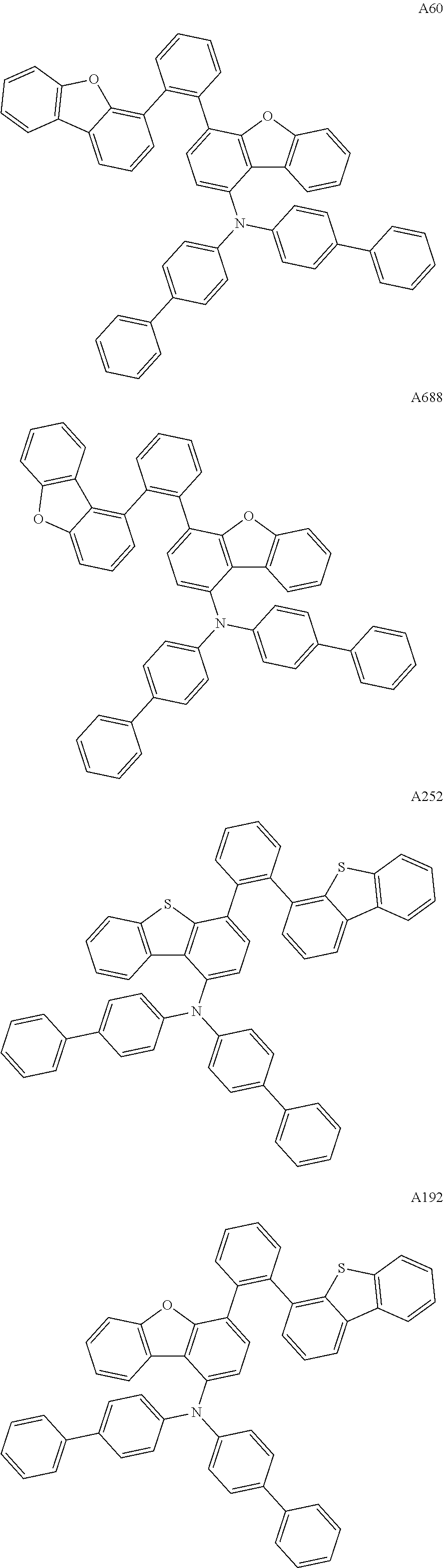 [SMILES: c1ccc(-c2ccc(N(c3ccc(-c4ccccc4)cc3)c3ccc(-c4ccccc4-c4cccc5c4oc4ccccc45)c4oc5ccccc5c34)cc2)cc1 ; c1ccc(-c2ccc(N(c3ccc(-c4ccccc4)cc3)c3ccc(-c4ccccc4-c4cccc5oc6ccccc6c45)c4oc5ccccc5c34)cc2)cc1 ; c1ccc(-c2ccc(N(c3ccc(-c4ccccc4)cc3)c3ccc(-c4ccccc4-c4cccc5c4sc4ccccc45)c4sc5ccccc5c34)cc2)cc1 ; c1ccc(-c2ccc(N(c3ccc(-c4ccccc4)cc3)c3ccc(-c4ccccc4-c4cccc5c4sc4ccccc45)c4oc5ccccc5c34)cc2)cc1]

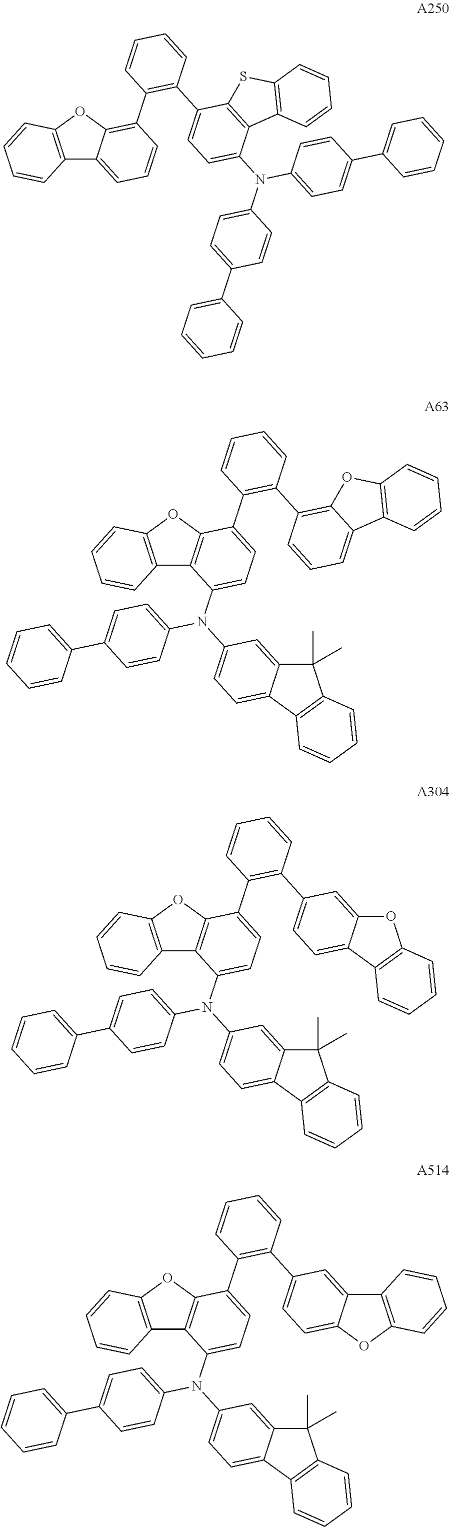 [SMILES: c1ccc(-c2ccc(N(c3ccc(-c4ccccc4)cc3)c3ccc(-c4ccccc4-c4cccc5c4oc4ccccc45)c4sc5ccccc5c34)cc2)cc1 ; CC1(C)c2ccccc2-c2ccc(N(c3ccc(-c4ccccc4)cc3)c3ccc(-c4ccccc4-c4cccc5c4oc4ccccc45)c4oc5ccccc5c34)cc21 ; CC1(C)c2ccccc2-c2ccc(N(c3ccc(-c4ccccc4)cc3)c3ccc(-c4ccccc4-c4ccc5c(c4)oc4ccccc45)c4oc5ccccc5c34)cc21 ; CC1(C)c2ccccc2-c2ccc(N(c3ccc(-c4ccccc4)cc3)c3ccc(-c4ccccc4-c4ccc5oc6ccccc6c5c4)c4oc5ccccc5c34)cc21]

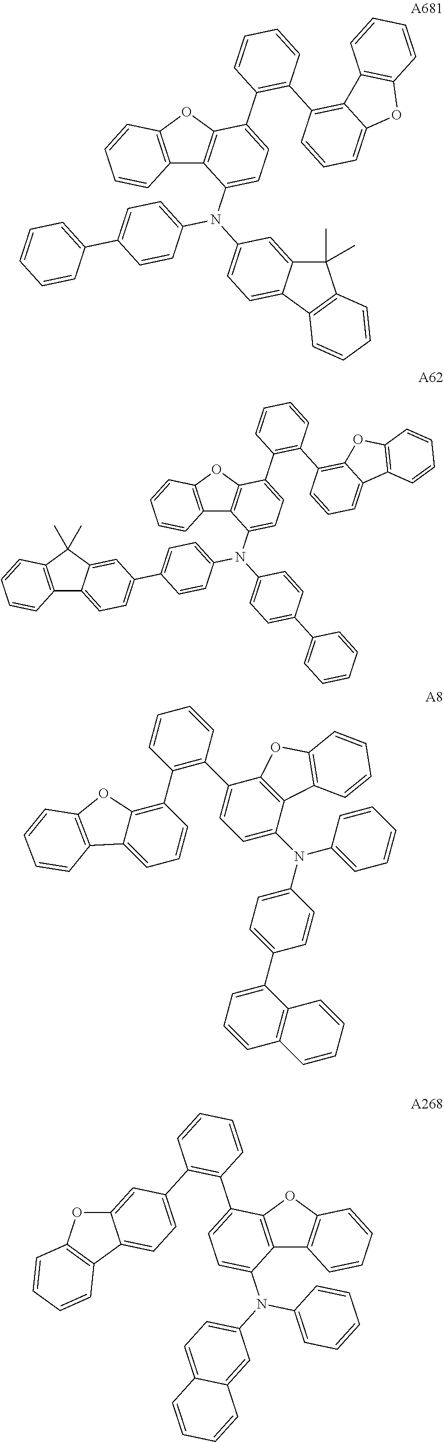 [SMILES: CC1(C)c2ccccc2-c2ccc(N(c3ccc(-c4ccccc4)cc3)c3ccc(-c4ccccc4-c4cccc5oc6ccccc6c45)c4oc5ccccc5c34)cc21 ; CC1(C)c2ccccc2-c2ccc(-c3ccc(N(c4ccc(-c5ccccc5)cc4)c4ccc(-c5ccccc5-c5cccc6c5oc5ccccc56)c5oc6ccccc6c45)cc3)cc21 ; c1ccc(N(c2ccc(-c3cccc4ccccc34)cc2)c2ccc(-c3ccccc3-c3cccc4c3oc3ccccc34)c3oc4ccccc4c23)cc1 ; c1ccc(N(c2ccc3ccccc3c2)c2ccc(-c3ccccc3-c3ccc4c(c3)oc3ccccc34)c3oc4ccccc4c23)cc1]

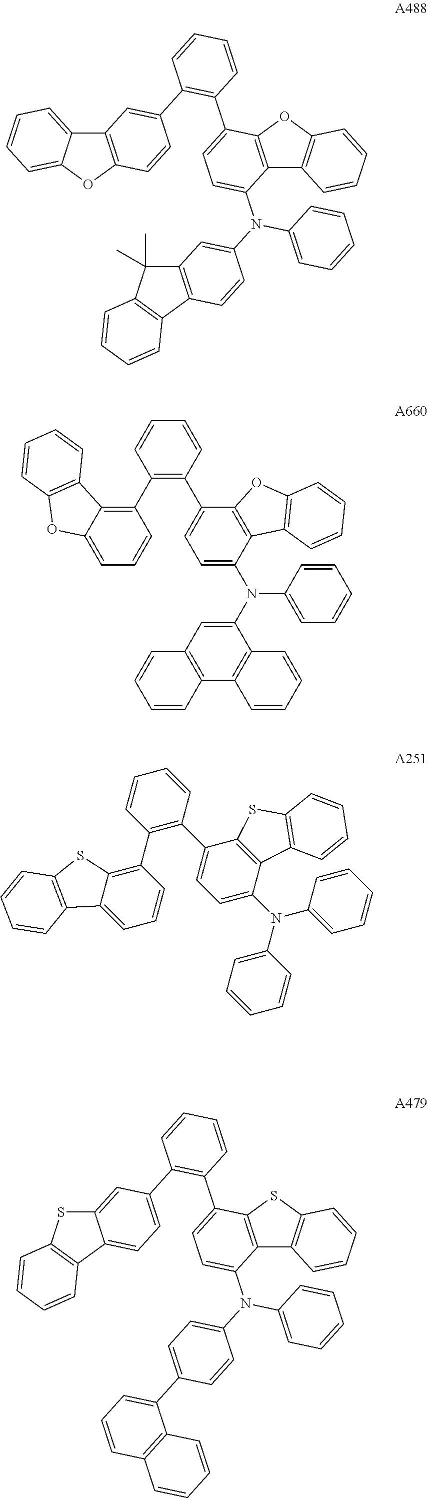 [SMILES: CC1(C)c2ccccc2-c2ccc(N(c3ccccc3)c3ccc(-c4ccccc4-c4ccc5oc6ccccc6c5c4)c4oc5ccccc5c34)cc21 ; c1ccc(N(c2cc3ccccc3c3ccccc23)c2ccc(-c3ccccc3-c3cccc4oc5ccccc5c34)c3oc4ccccc4c23)cc1 ; c1ccc(N(c2ccccc2)c2ccc(-c3ccccc3-c3cccc4c3sc3ccccc34)c3sc4ccccc4c23)cc1 ; c1ccc(N(c2ccc(-c3cccc4ccccc34)cc2)c2ccc(-c3ccccc3-c3ccc4c(c3)sc3ccccc34)c3sc4ccccc4c23)cc1]

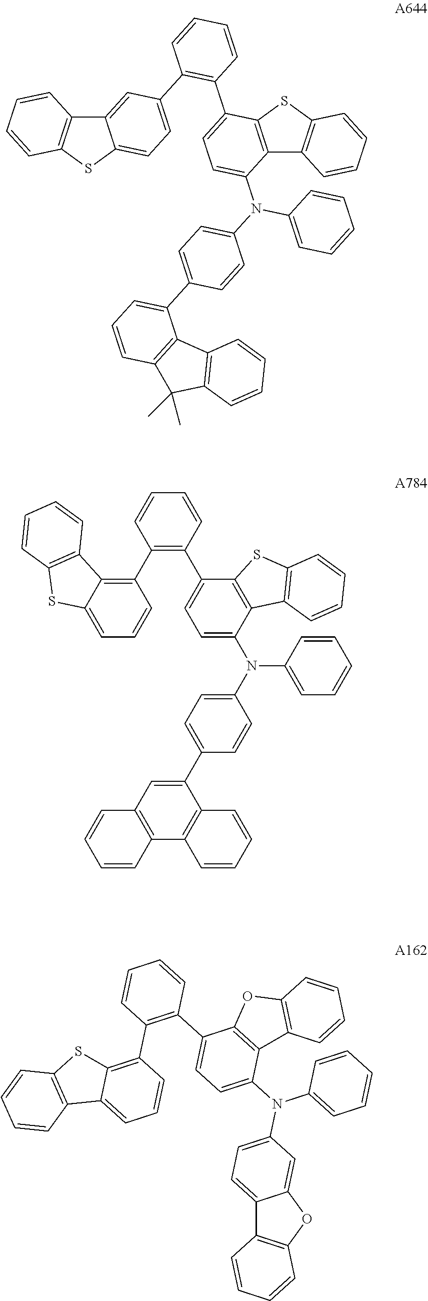 [SMILES: CC1(C)c2ccccc2-c2c(-c3ccc(N(c4ccccc4)c4ccc(-c5ccccc5-c5ccc6sc7ccccc7c6c5)c5sc6ccccc6c45)cc3)cccc21 ; c1ccc(N(c2ccc(-c3cc4ccccc4c4ccccc34)cc2)c2ccc(-c3ccccc3-c3cccc4sc5ccccc5c34)c3sc4ccccc4c23)cc1 ; c1ccc(N(c2ccc3c(c2)oc2ccccc23)c2ccc(-c3ccccc3-c3cccc4c3sc3ccccc34)c3oc4ccccc4c23)cc1]

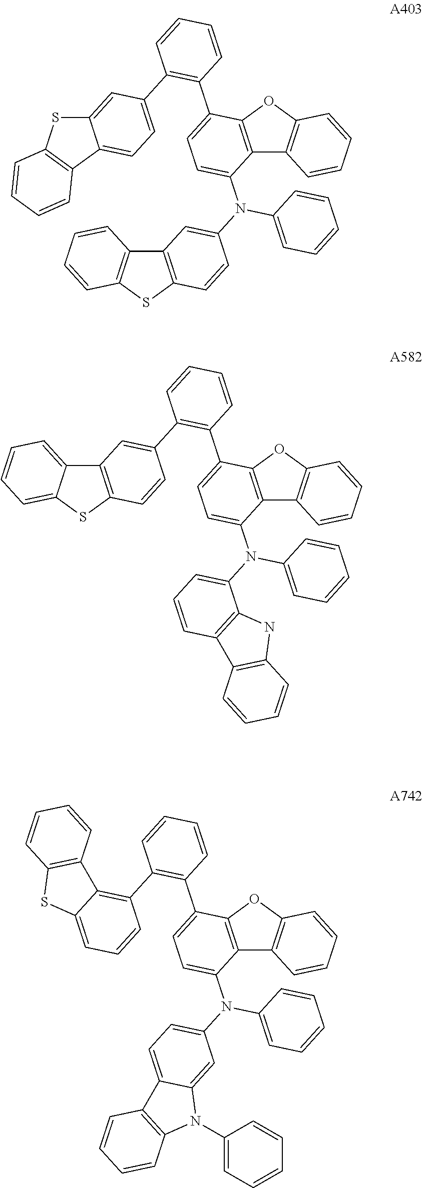 [SMILES: c1ccc(N(c2ccc3sc4ccccc4c3c2)c2ccc(-c3ccccc3-c3ccc4c(c3)sc3ccccc34)c3oc4ccccc4c23)cc1 ; c1ccc(N(c2cccc3c2[nH]c2ccccc23)c2ccc(-c3ccccc3-c3ccc4sc5ccccc5c4c3)c3oc4ccccc4c23)cc1 ; c1ccc(N(c2ccc3c4ccccc4n(-c4ccccc4)c3c2)c2ccc(-c3ccccc3-c3cccc4sc5ccccc5c34)c3oc4ccccc4c23)cc1]

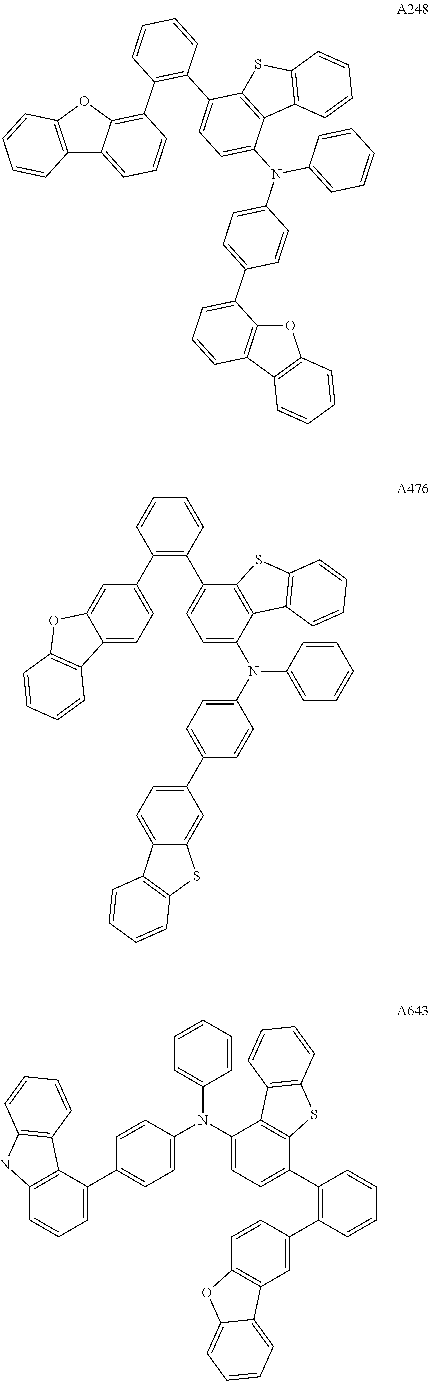 [SMILES: c1ccc(N(c2ccc(-c3cccc4c3oc3ccccc34)cc2)c2ccc(-c3ccccc3-c3cccc4c3oc3ccccc34)c3sc4ccccc4c23)cc1 ; c1ccc(N(c2ccc(-c3ccc4c(c3)sc3ccccc34)cc2)c2ccc(-c3ccccc3-c3ccc4c(c3)oc3ccccc34)c3sc4ccccc4c23)cc1 ; c1ccc(N(c2ccc(-c3cccc4[nH]c5ccccc5c34)cc2)c2ccc(-c3ccccc3-c3ccc4oc5ccccc5c4c3)c3sc4ccccc4c23)cc1]

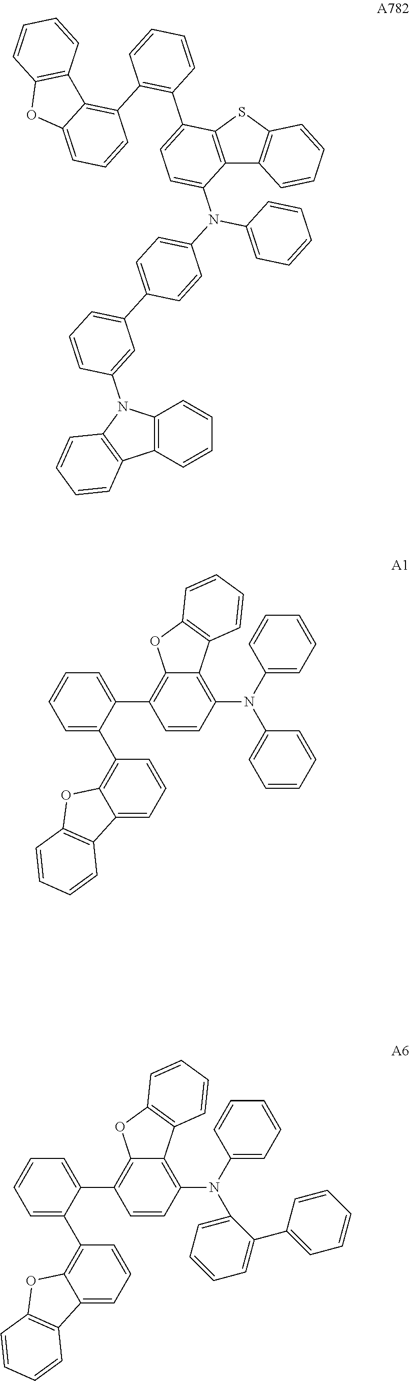 [SMILES: c1ccc(N(c2ccc(-c3cccc(-n4c5ccccc5c5ccccc54)c3)cc2)c2ccc(-c3ccccc3-c3cccc4oc5ccccc5c34)c3sc4ccccc4c23)cc1 ; c1ccc(N(c2ccccc2)c2ccc(-c3ccccc3-c3cccc4c3oc3ccccc34)c3oc4ccccc4c23)cc1 ; c1ccc(-c2ccccc2N(c2ccccc2)c2ccc(-c3ccccc3-c3cccc4c3oc3ccccc34)c3oc4ccccc4c23)cc1]

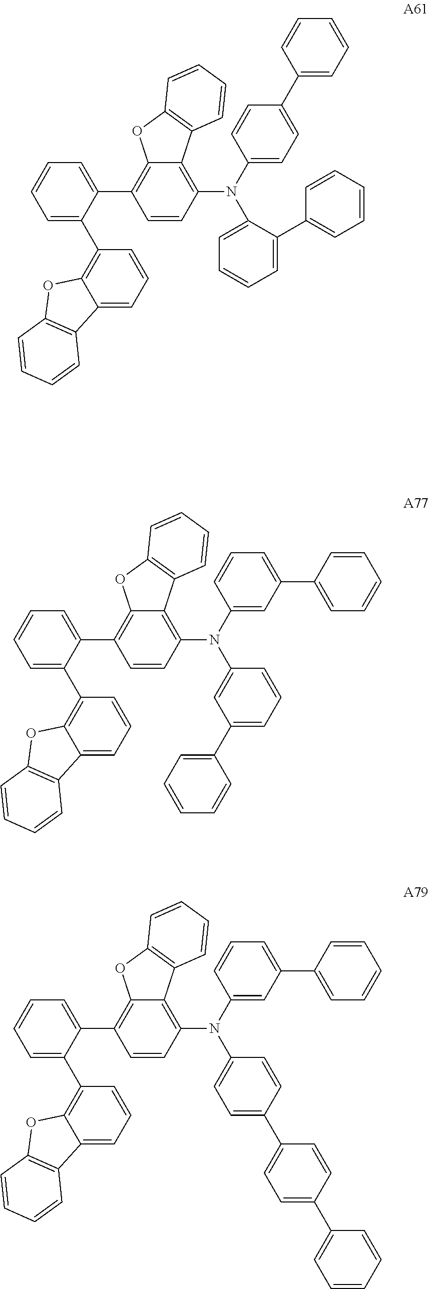 [SMILES: c1ccc(-c2ccc(N(c3ccccc3-c3ccccc3)c3ccc(-c4ccccc4-c4cccc5c4oc4ccccc45)c4oc5ccccc5c34)cc2)cc1 ; c1ccc(-c2cccc(N(c3cccc(-c4ccccc4)c3)c3ccc(-c4ccccc4-c4cccc5c4oc4ccccc45)c4oc5ccccc5c34)c2)cc1 ; c1ccc(-c2ccc(-c3ccc(N(c4cccc(-c5ccccc5)c4)c4ccc(-c5ccccc5-c5cccc6c5oc5ccccc56)c5oc6ccccc6c45)cc3)cc2)cc1]

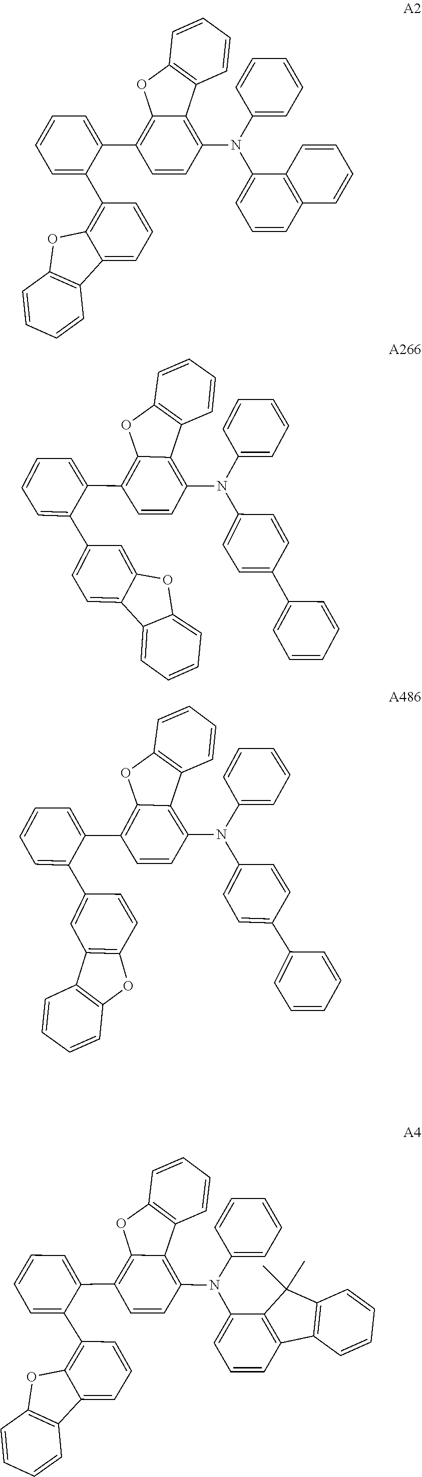

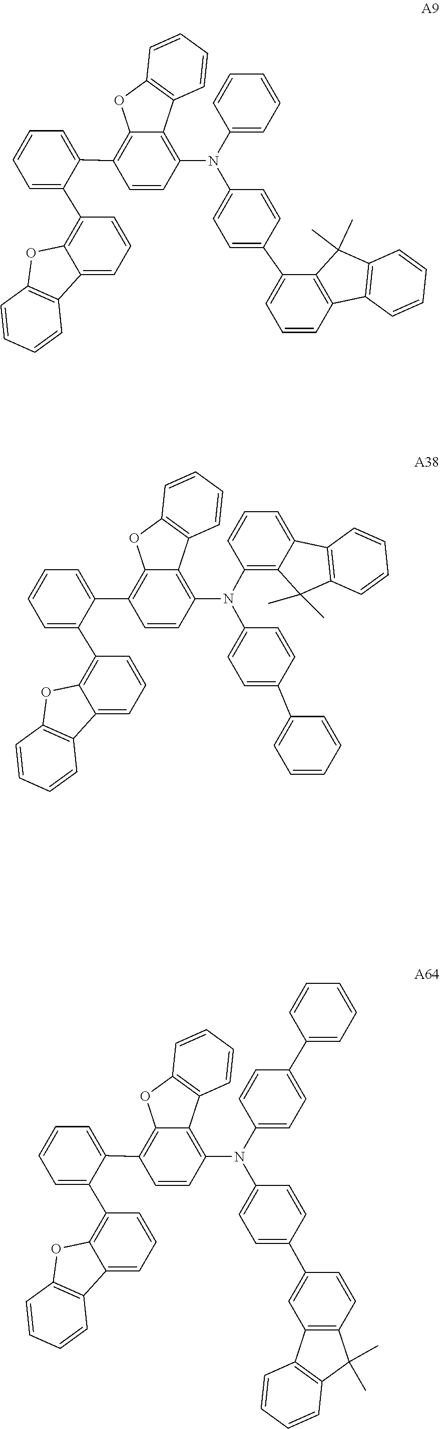 [SMILES: CC1(C)c2ccccc2-c2cccc(-c3ccc(N(c4ccccc4)c4ccc(-c5ccccc5-c5cccc6c5oc5ccccc56)c5oc6ccccc6c45)cc3)c21 ; CC1(C)c2ccccc2-c2cccc(N(c3ccc(-c4ccccc4)cc3)c3ccc(-c4ccccc4-c4cccc5c4oc4ccccc45)c4oc5ccccc5c34)c21 ; CC1(C)c2ccccc2-c2cc(-c3ccc(N(c4ccc(-c5ccccc5)cc4)c4ccc(-c5ccccc5-c5cccc6c5oc5ccccc56)c5oc6ccccc6c45)cc3)ccc21]

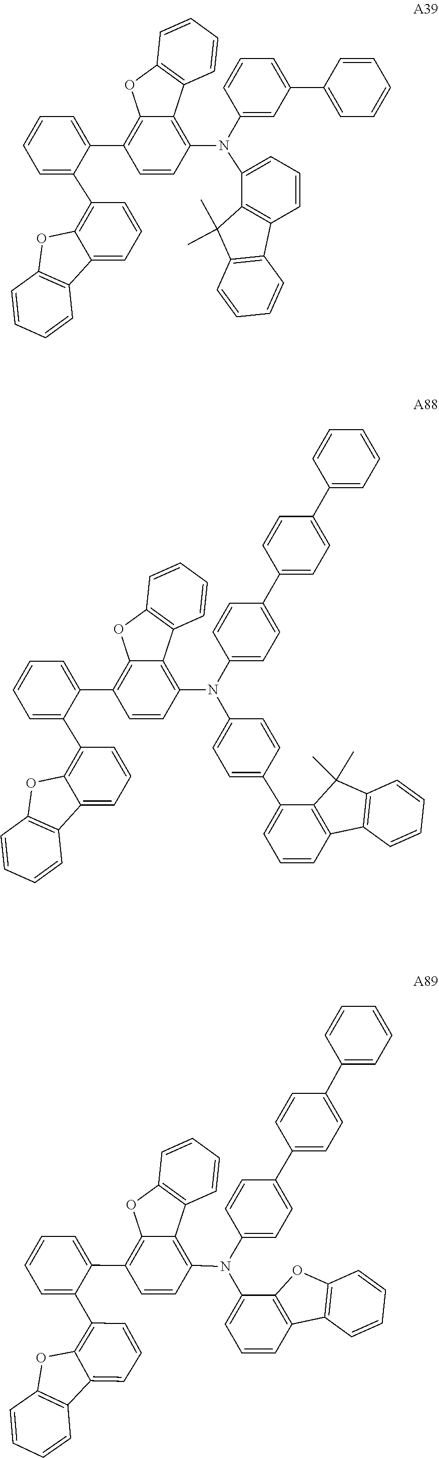 [SMILES: CC1(C)c2ccccc2-c2cccc(N(c3cccc(-c4ccccc4)c3)c3ccc(-c4ccccc4-c4cccc5c4oc4ccccc45)c4oc5ccccc5c34)c21 ; CC1(C)c2ccccc2-c2cccc(-c3ccc(N(c4ccc(-c5ccc(-c6ccccc6)cc5)cc4)c4ccc(-c5ccccc5-c5cccc6c5oc5ccccc56)c5oc6ccccc6c45)cc3)c21 ; c1ccc(-c2ccc(-c3ccc(N(c4cccc5c4oc4ccccc45)c4ccc(-c5ccccc5-c5cccc6c5oc5ccccc56)c5oc6ccccc6c45)cc3)cc2)cc1]

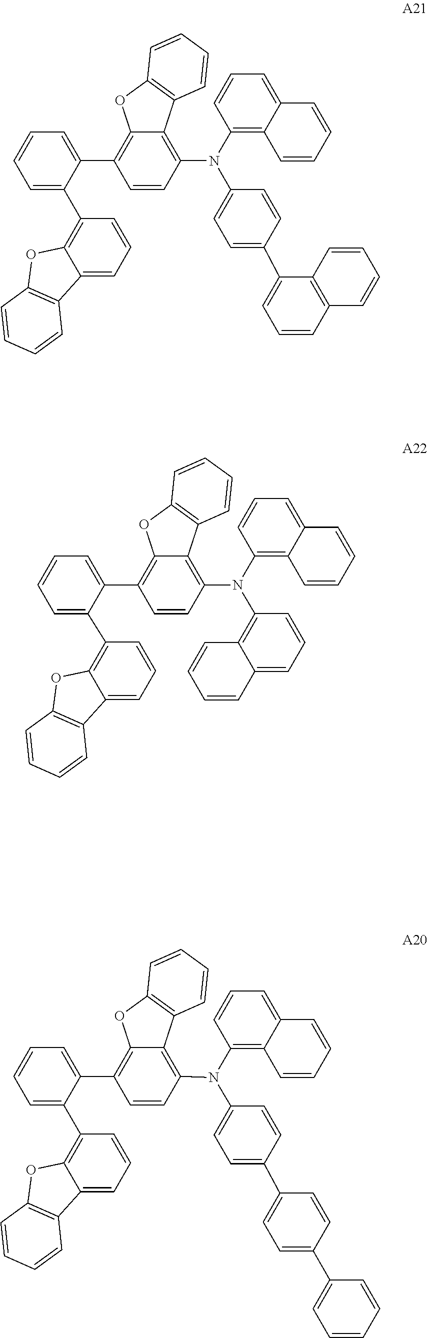 [SMILES: c1ccc(-c2ccc(N(c3ccc(-c4cccc5ccccc45)cc3)c3cccc4ccccc34)c3c2oc2ccccc23)c(-c2cccc3c2oc2ccccc23)c1 ; c1ccc(-c2ccc(N(c3cccc4ccccc34)c3cccc4ccccc34)c3c2oc2ccccc23)c(-c2cccc3c2oc2ccccc23)c1 ; c1ccc(-c2ccc(-c3ccc(N(c4cccc5ccccc45)c4ccc(-c5ccccc5-c5cccc6c5oc5ccccc56)c5oc6ccccc6c45)cc3)cc2)cc1]

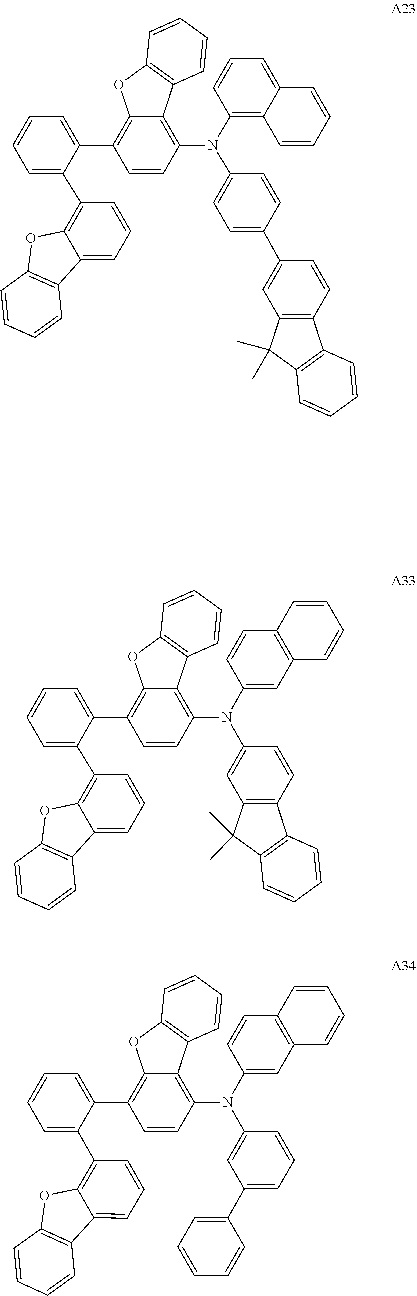 [SMILES: CC1(C)c2ccccc2-c2ccc(-c3ccc(N(c4cccc5ccccc45)c4ccc(-c5ccccc5-c5cccc6c5oc5ccccc56)c5oc6ccccc6c45)cc3)cc21 ; CC1(C)c2ccccc2-c2ccc(N(c3ccc4ccccc4c3)c3ccc(-c4ccccc4-c4cccc5c4oc4ccccc45)c4oc5ccccc5c34)cc21 ; c1ccc(-c2cccc(N(c3ccc4ccccc4c3)c3ccc(-c4ccccc4-c4cccc5c4oc4ccccc45)c4oc5ccccc5c34)c2)cc1]

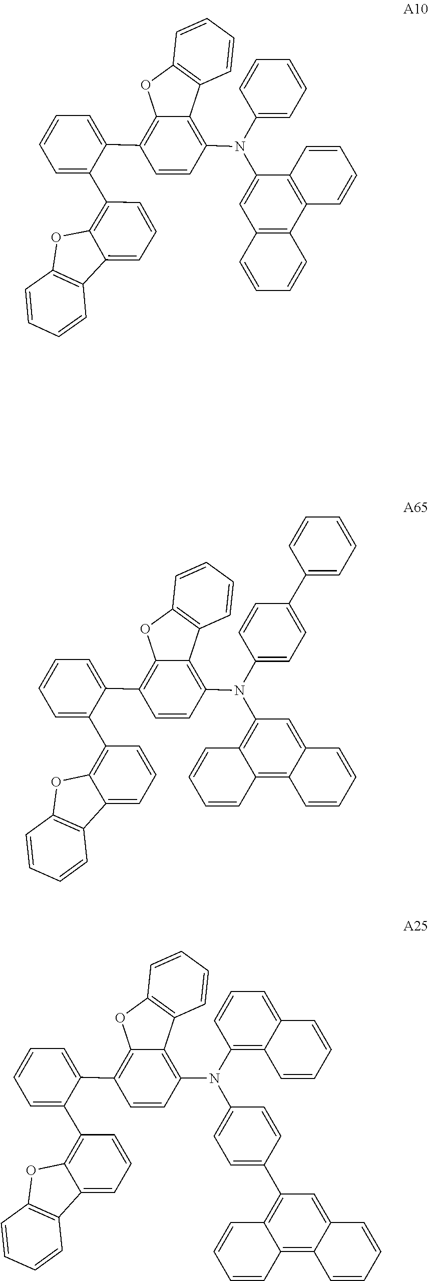 [SMILES: c1ccc(N(c2cc3ccccc3c3ccccc23)c2ccc(-c3ccccc3-c3cccc4c3oc3ccccc34)c3oc4ccccc4c23)cc1 ; c1ccc(-c2ccc(N(c3cc4ccccc4c4ccccc34)c3ccc(-c4ccccc4-c4cccc5c4oc4ccccc45)c4oc5ccccc5c34)cc2)cc1 ; c1ccc(-c2ccc(N(c3ccc(-c4cc5ccccc5c5ccccc45)cc3)c3cccc4ccccc34)c3c2oc2ccccc23)c(-c2cccc3c2oc2ccccc23)c1]

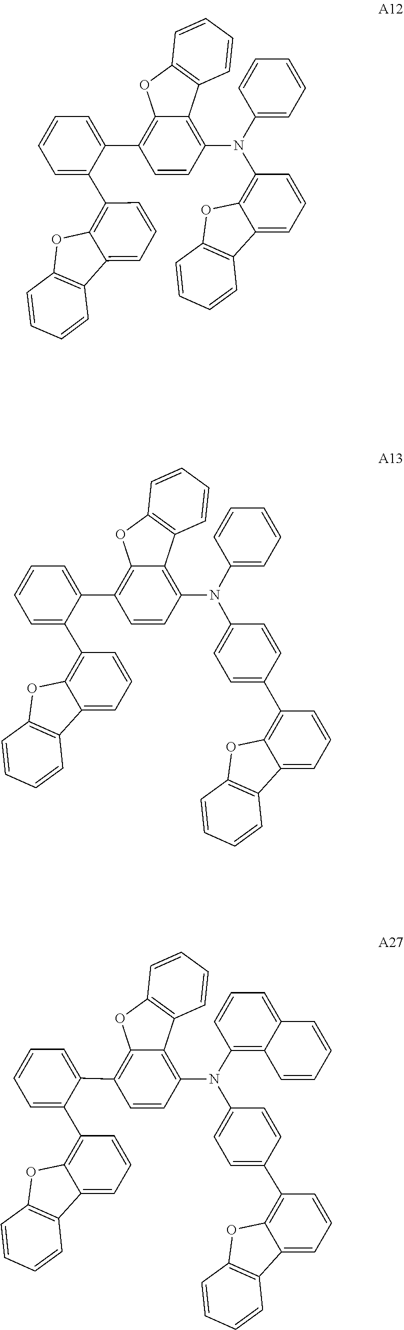 [SMILES: c1ccc(N(c2cccc3c2oc2ccccc23)c2ccc(-c3ccccc3-c3cccc4c3oc3ccccc34)c3oc4ccccc4c23)cc1 ; c1ccc(N(c2ccc(-c3cccc4c3oc3ccccc34)cc2)c2ccc(-c3ccccc3-c3cccc4c3oc3ccccc34)c3oc4ccccc4c23)cc1 ; c1ccc(-c2ccc(N(c3ccc(-c4cccc5c4oc4ccccc45)cc3)c3cccc4ccccc34)c3c2oc2ccccc23)c(-c2cccc3c2oc2ccccc23)c1]

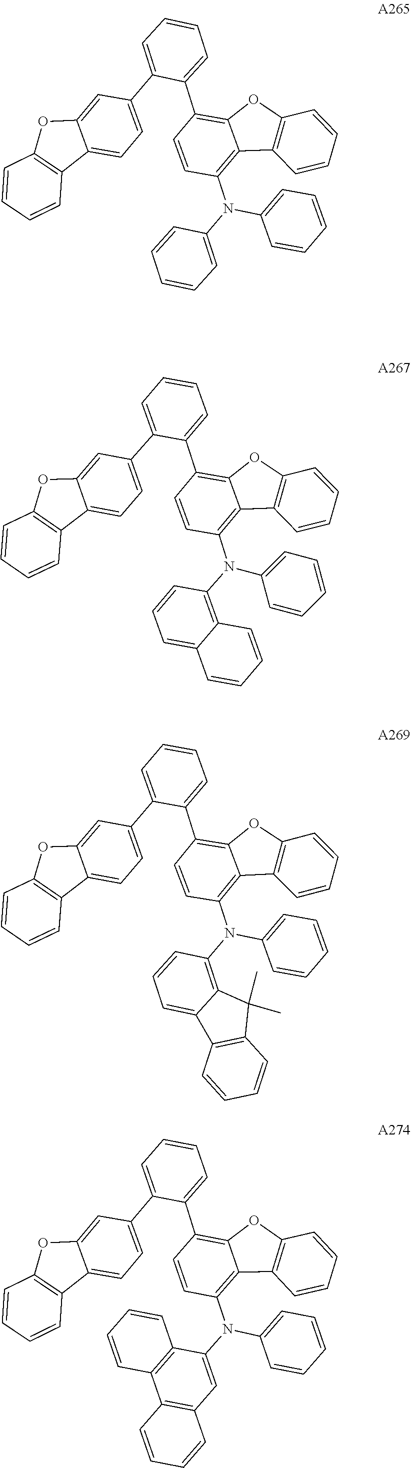 [SMILES: c1ccc(N(c2ccccc2)c2ccc(-c3ccccc3-c3ccc4c(c3)oc3ccccc34)c3oc4ccccc4c23)cc1 ; c1ccc(N(c2cccc3ccccc23)c2ccc(-c3ccccc3-c3ccc4c(c3)oc3ccccc34)c3oc4ccccc4c23)cc1 ; CC1(C)c2ccccc2-c2cccc(N(c3ccccc3)c3ccc(-c4ccccc4-c4ccc5c(c4)oc4ccccc45)c4oc5ccccc5c34)c21 ; c1ccc(N(c2cc3ccccc3c3ccccc23)c2ccc(-c3ccccc3-c3ccc4c(c3)oc3ccccc34)c3oc4ccccc4c23)cc1]

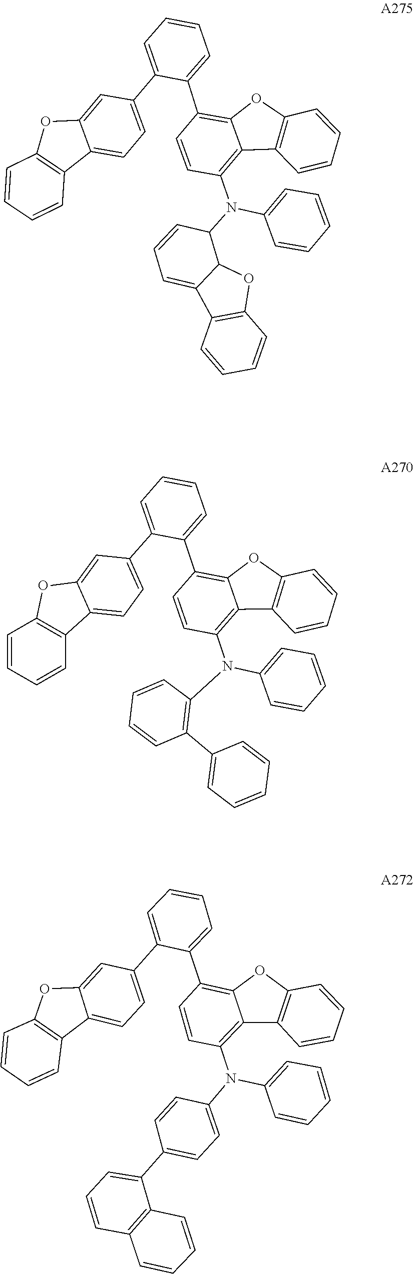 [SMILES: C1=CC(N(c2ccccc2)c2ccc(-c3ccccc3-c3ccc4c(c3)oc3ccccc34)c3oc4ccccc4c23)C2Oc3ccccc3C2=C1 ; c1ccc(-c2ccccc2N(c2ccccc2)c2ccc(-c3ccccc3-c3ccc4c(c3)oc3ccccc34)c3oc4ccccc4c23)cc1 ; c1ccc(N(c2ccc(-c3cccc4ccccc34)cc2)c2ccc(-c3ccccc3-c3ccc4c(c3)oc3ccccc34)c3oc4ccccc4c23)cc1]

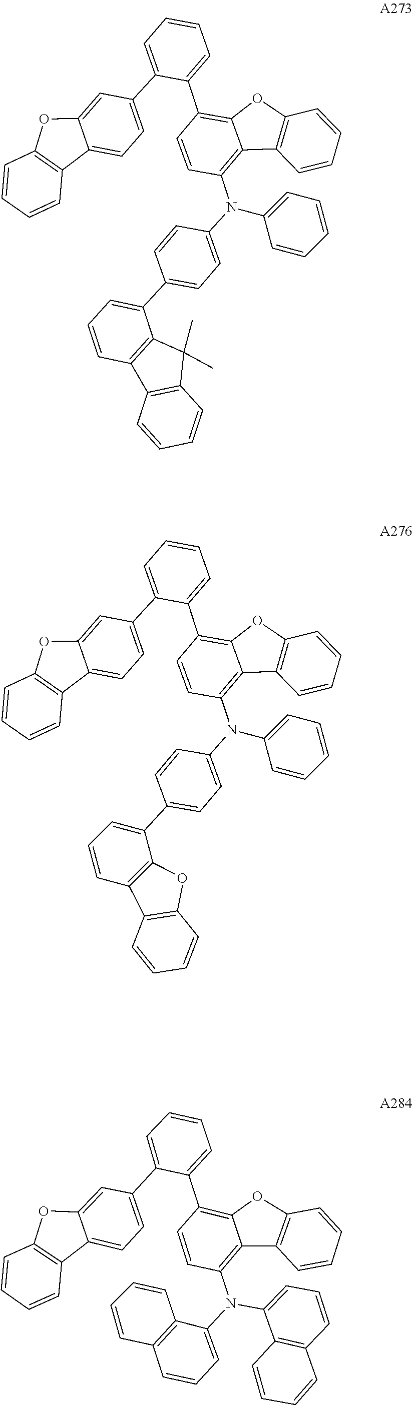 [SMILES: CC1(C)c2ccccc2-c2cccc(-c3ccc(N(c4ccccc4)c4ccc(-c5ccccc5-c5ccc6c(c5)oc5ccccc56)c5oc6ccccc6c45)cc3)c21 ; c1ccc(N(c2ccc(-c3cccc4c3oc3ccccc34)cc2)c2ccc(-c3ccccc3-c3ccc4c(c3)oc3ccccc34)c3oc4ccccc4c23)cc1 ; c1ccc(-c2ccc(N(c3cccc4ccccc34)c3cccc4ccccc34)c3c2oc2ccccc23)c(-c2ccc3c(c2)oc2ccccc23)c1]

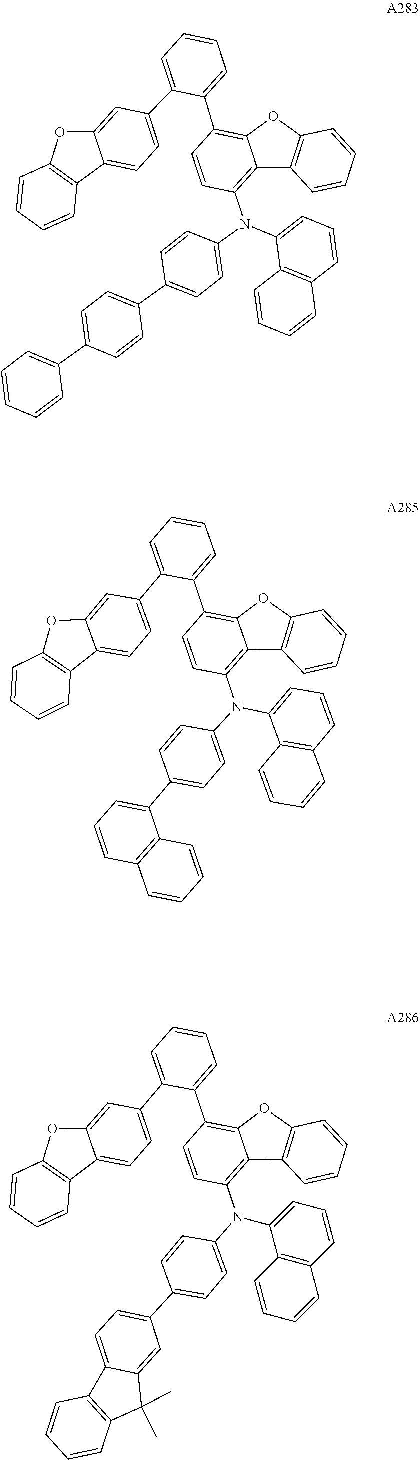 [SMILES: c1ccc(-c2ccc(-c3ccc(N(c4cccc5ccccc45)c4ccc(-c5ccccc5-c5ccc6c(c5)oc5ccccc56)c5oc6ccccc6c45)cc3)cc2)cc1 ; c1ccc(-c2ccc(N(c3ccc(-c4cccc5ccccc45)cc3)c3cccc4ccccc34)c3c2oc2ccccc23)c(-c2ccc3c(c2)oc2ccccc23)c1 ; CC1(C)c2ccccc2-c2ccc(-c3ccc(N(c4cccc5ccccc45)c4ccc(-c5ccccc5-c5ccc6c(c5)oc5ccccc56)c5oc6ccccc6c45)cc3)cc21]

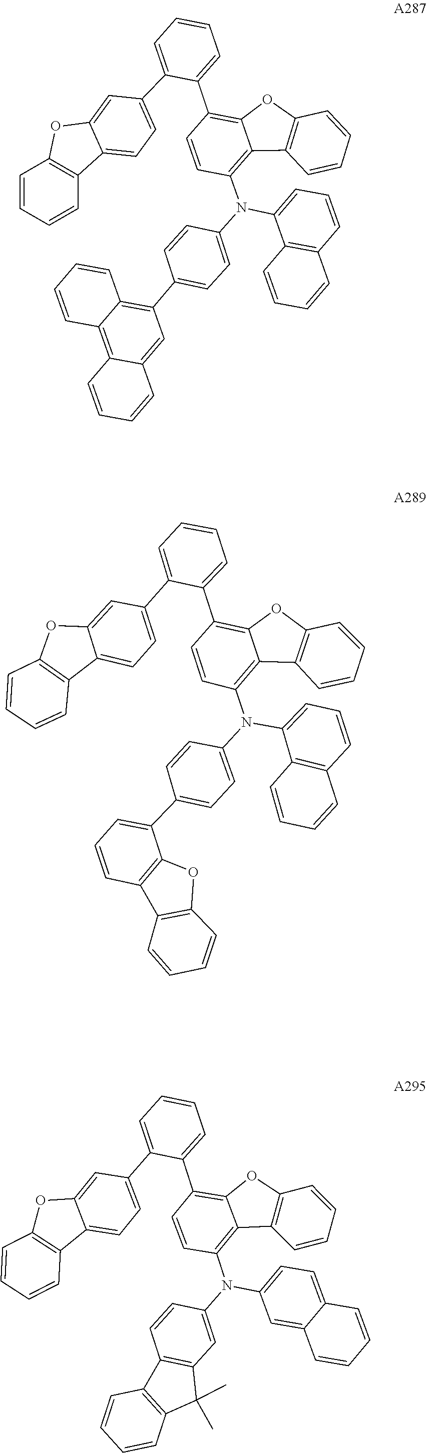 [SMILES: c1ccc(-c2ccc(N(c3ccc(-c4cc5ccccc5c5ccccc45)cc3)c3cccc4ccccc34)c3c2oc2ccccc23)c(-c2ccc3c(c2)oc2ccccc23)c1 ; c1ccc(-c2ccc(N(c3ccc(-c4cccc5c4oc4ccccc45)cc3)c3cccc4ccccc34)c3c2oc2ccccc23)c(-c2ccc3c(c2)oc2ccccc23)c1 ; CC1(C)c2ccccc2-c2ccc(N(c3ccc4ccccc4c3)c3ccc(-c4ccccc4-c4ccc5c(c4)oc4ccccc45)c4oc5ccccc5c34)cc21]

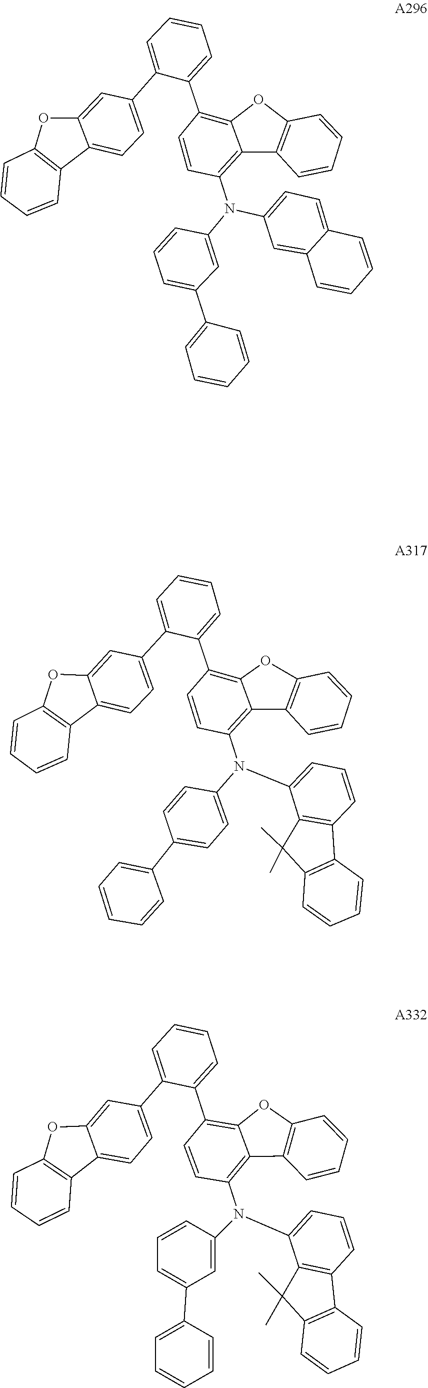 [SMILES: c1ccc(-c2cccc(N(c3ccc4ccccc4c3)c3ccc(-c4ccccc4-c4ccc5c(c4)oc4ccccc45)c4oc5ccccc5c34)c2)cc1 ; CC1(C)c2ccccc2-c2cccc(N(c3ccc(-c4ccccc4)cc3)c3ccc(-c4ccccc4-c4ccc5c(c4)oc4ccccc45)c4oc5ccccc5c34)c21 ; CC1(C)c2ccccc2-c2cccc(N(c3cccc(-c4ccccc4)c3)c3ccc(-c4ccccc4-c4ccc5c(c4)oc4ccccc45)c4oc5ccccc5c34)c21]

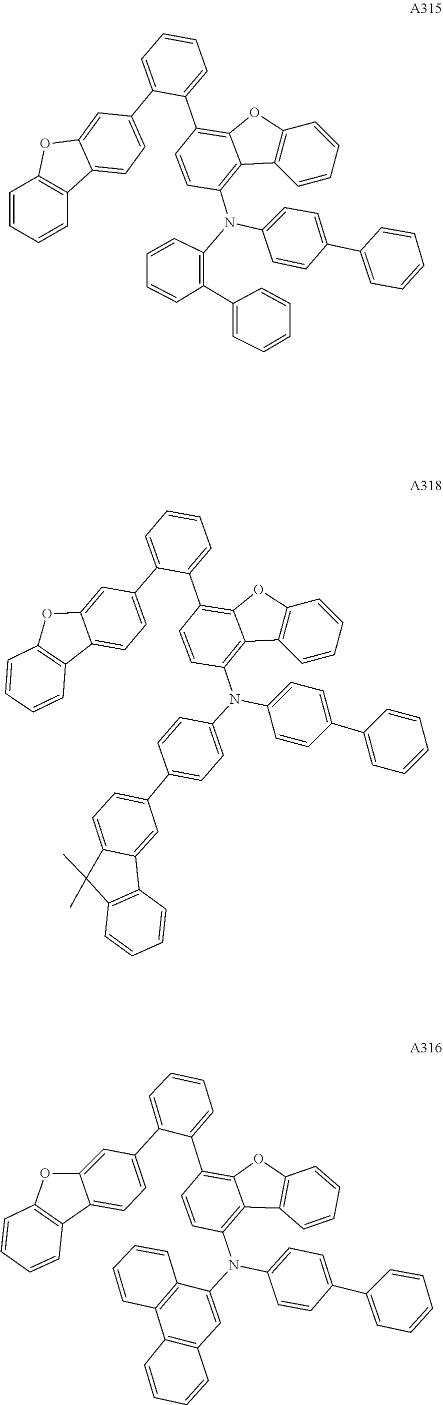 [SMILES: c1ccc(-c2ccc(N(c3ccccc3-c3ccccc3)c3ccc(-c4ccccc4-c4ccc5c(c4)oc4ccccc45)c4oc5ccccc5c34)cc2)cc1 ; CC1(C)c2ccccc2-c2cc(-c3ccc(N(c4ccc(-c5ccccc5)cc4)c4ccc(-c5ccccc5-c5ccc6c(c5)oc5ccccc56)c5oc6ccccc6c45)cc3)ccc21 ; c1ccc(-c2ccc(N(c3cc4ccccc4c4ccccc34)c3ccc(-c4ccccc4-c4ccc5c(c4)oc4ccccc45)c4oc5ccccc5c34)cc2)cc1]

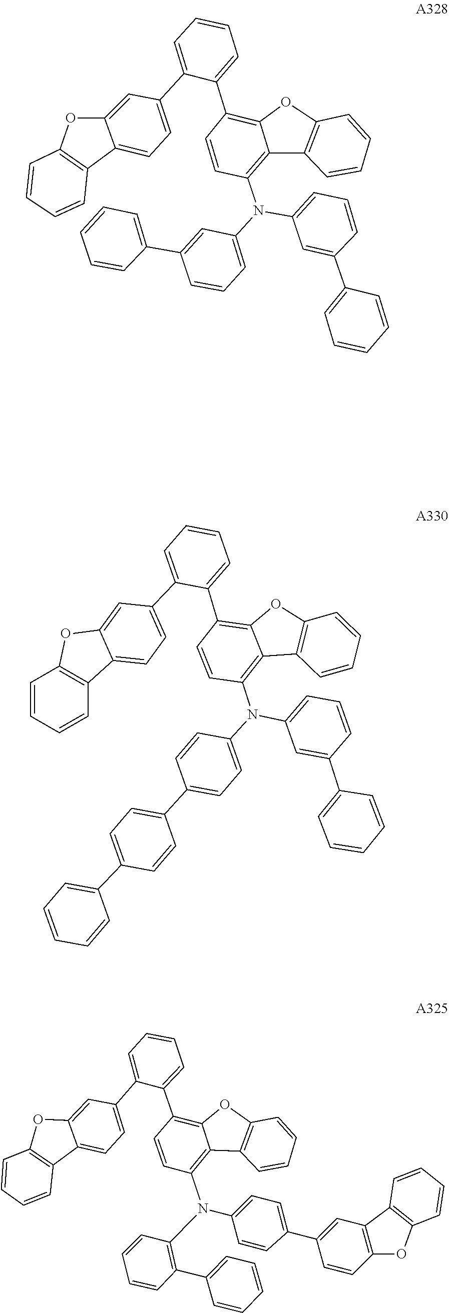 [SMILES: c1ccc(-c2cccc(N(c3cccc(-c4ccccc4)c3)c3ccc(-c4ccccc4-c4ccc5c(c4)oc4ccccc45)c4oc5ccccc5c34)c2)cc1 ; c1ccc(-c2ccc(-c3ccc(N(c4cccc(-c5ccccc5)c4)c4ccc(-c5ccccc5-c5ccc6c(c5)oc5ccccc56)c5oc6ccccc6c45)cc3)cc2)cc1 ; c1ccc(-c2ccccc2N(c2ccc(-c3ccc4oc5ccccc5c4c3)cc2)c2ccc(-c3ccccc3-c3ccc4c(c3)oc3ccccc34)c3oc4ccccc4c23)cc1]

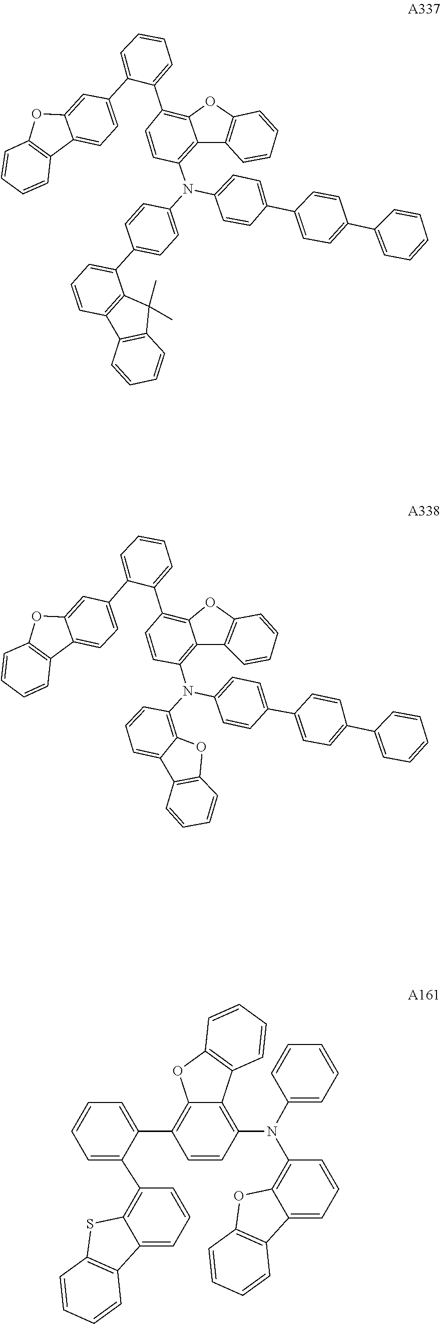

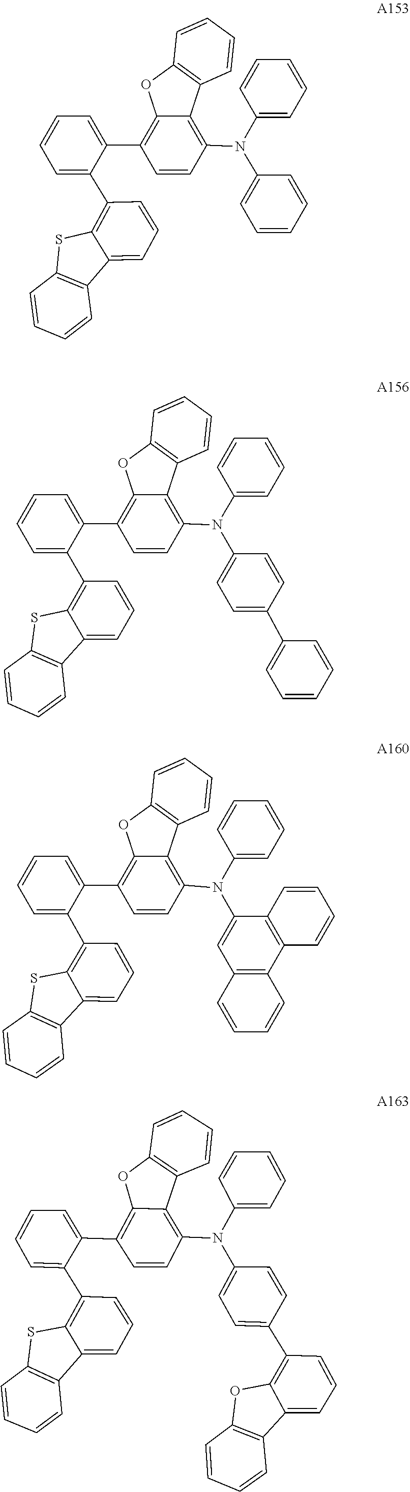 [SMILES: c1ccc(N(c2ccccc2)c2ccc(-c3ccccc3-c3cccc4c3sc3ccccc34)c3oc4ccccc4c23)cc1 ; c1ccc(-c2ccc(N(c3ccccc3)c3ccc(-c4ccccc4-c4cccc5c4sc4ccccc45)c4oc5ccccc5c34)cc2)cc1 ; c1ccc(N(c2cc3ccccc3c3ccccc23)c2ccc(-c3ccccc3-c3cccc4c3sc3ccccc34)c3oc4ccccc4c23)cc1 ; c1ccc(N(c2ccc(-c3cccc4c3oc3ccccc34)cc2)c2ccc(-c3ccccc3-c3cccc4c3sc3ccccc34)c3oc4ccccc4c23)cc1]

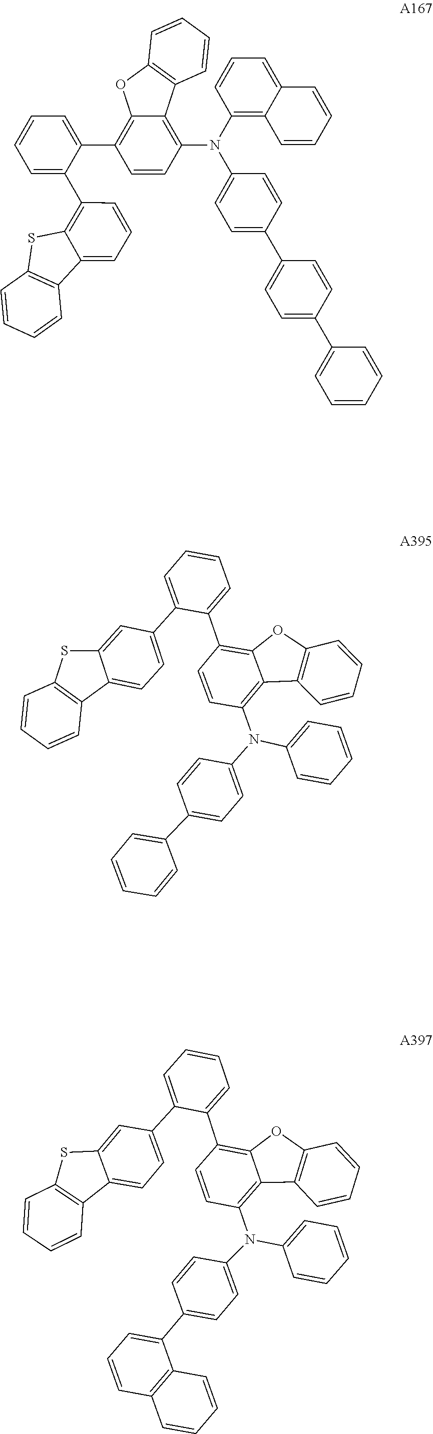 [SMILES: c1ccc(-c2ccc(-c3ccc(N(c4cccc5ccccc45)c4ccc(-c5ccccc5-c5cccc6c5sc5ccccc56)c5oc6ccccc6c45)cc3)cc2)cc1 ; c1ccc(-c2ccc(N(c3ccccc3)c3ccc(-c4ccccc4-c4ccc5c(c4)sc4ccccc45)c4oc5ccccc5c34)cc2)cc1 ; c1ccc(N(c2ccc(-c3cccc4ccccc34)cc2)c2ccc(-c3ccccc3-c3ccc4c(c3)sc3ccccc34)c3oc4ccccc4c23)cc1]

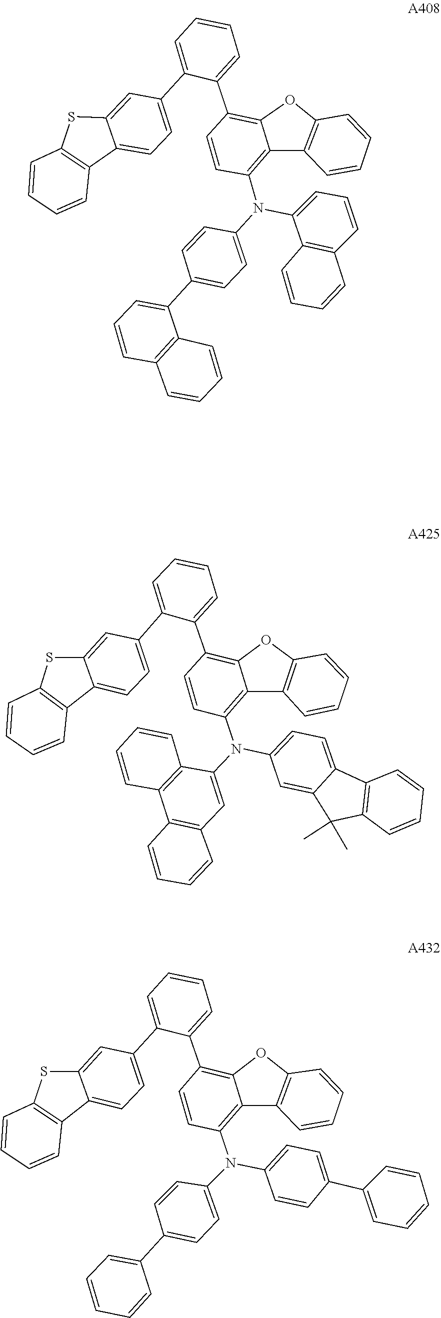 [SMILES: c1ccc(-c2ccc(N(c3ccc(-c4cccc5ccccc45)cc3)c3cccc4ccccc34)c3c2oc2ccccc23)c(-c2ccc3c(c2)sc2ccccc23)c1 ; CC1(C)c2ccccc2-c2ccc(N(c3cc4ccccc4c4ccccc34)c3ccc(-c4ccccc4-c4ccc5c(c4)sc4ccccc45)c4oc5ccccc5c34)cc21 ; c1ccc(-c2ccc(N(c3ccc(-c4ccccc4)cc3)c3ccc(-c4ccccc4-c4ccc5c(c4)sc4ccccc45)c4oc5ccccc5c34)cc2)cc1]

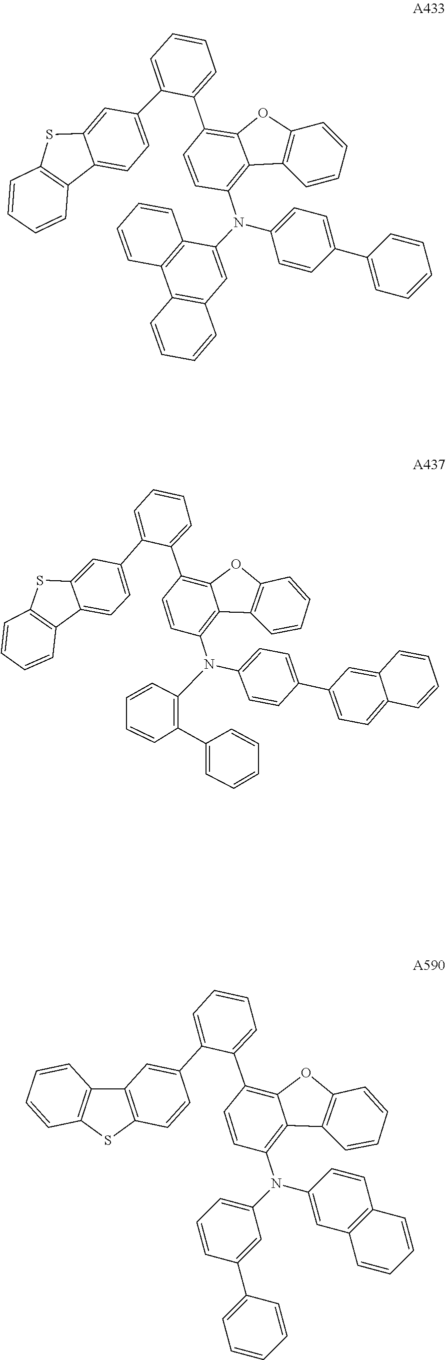 [SMILES: c1ccc(-c2ccc(N(c3cc4ccccc4c4ccccc34)c3ccc(-c4ccccc4-c4ccc5c(c4)sc4ccccc45)c4oc5ccccc5c34)cc2)cc1 ; c1ccc(-c2ccccc2N(c2ccc(-c3ccc4ccccc4c3)cc2)c2ccc(-c3ccccc3-c3ccc4c(c3)sc3ccccc34)c3oc4ccccc4c23)cc1 ; c1ccc(-c2cccc(N(c3ccc4ccccc4c3)c3ccc(-c4ccccc4-c4ccc5sc6ccccc6c5c4)c4oc5ccccc5c34)c2)cc1]

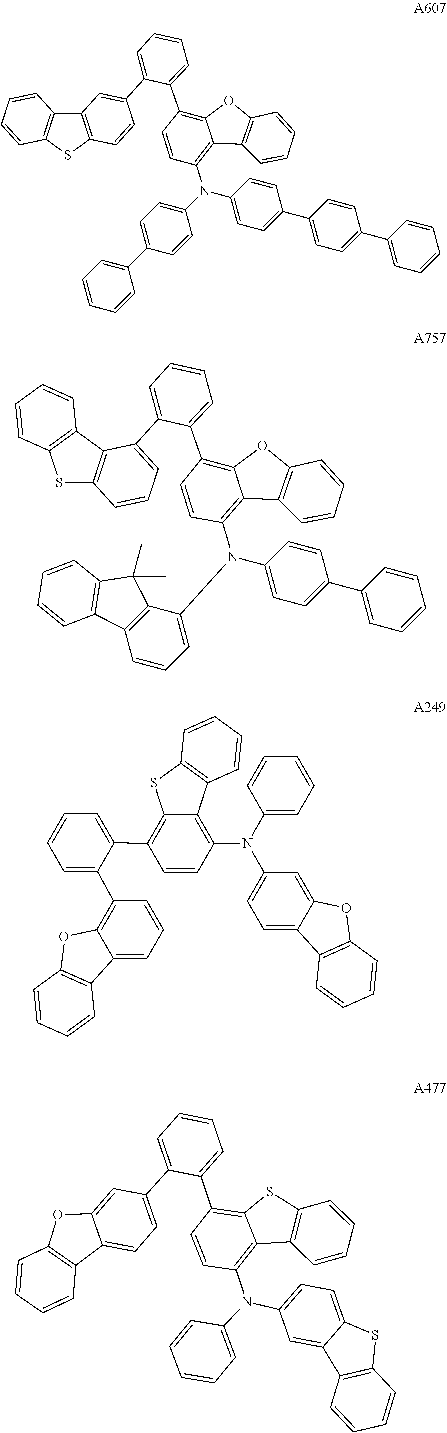 [SMILES: c1ccc(-c2ccc(-c3ccc(N(c4ccc(-c5ccccc5)cc4)c4ccc(-c5ccccc5-c5ccc6sc7ccccc7c6c5)c5oc6ccccc6c45)cc3)cc2)cc1 ; CC1(C)c2ccccc2-c2cccc(N(c3ccc(-c4ccccc4)cc3)c3ccc(-c4ccccc4-c4cccc5sc6ccccc6c45)c4oc5ccccc5c34)c21 ; c1ccc(N(c2ccc3c(c2)oc2ccccc23)c2ccc(-c3ccccc3-c3cccc4c3oc3ccccc34)c3sc4ccccc4c23)cc1 ; c1ccc(N(c2ccc3sc4ccccc4c3c2)c2ccc(-c3ccccc3-c3ccc4c(c3)oc3ccccc34)c3sc4ccccc4c23)cc1]

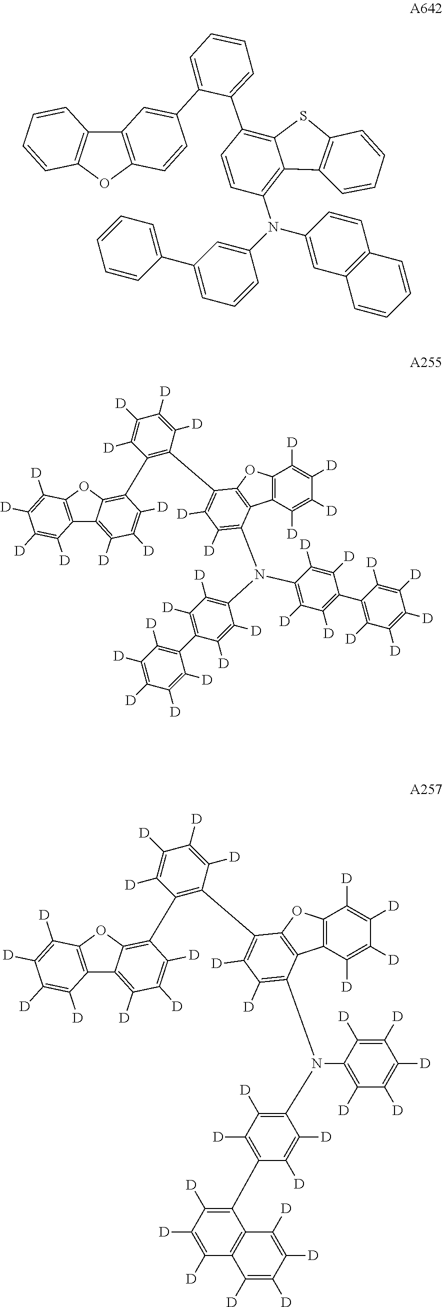

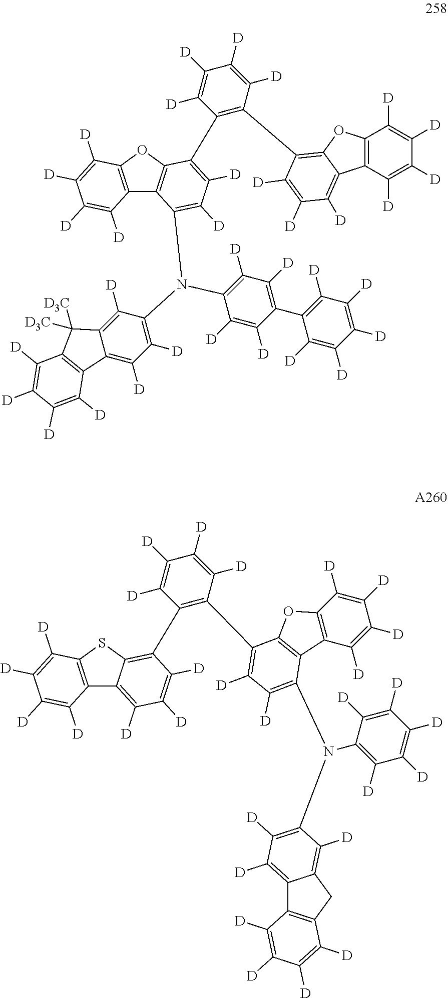

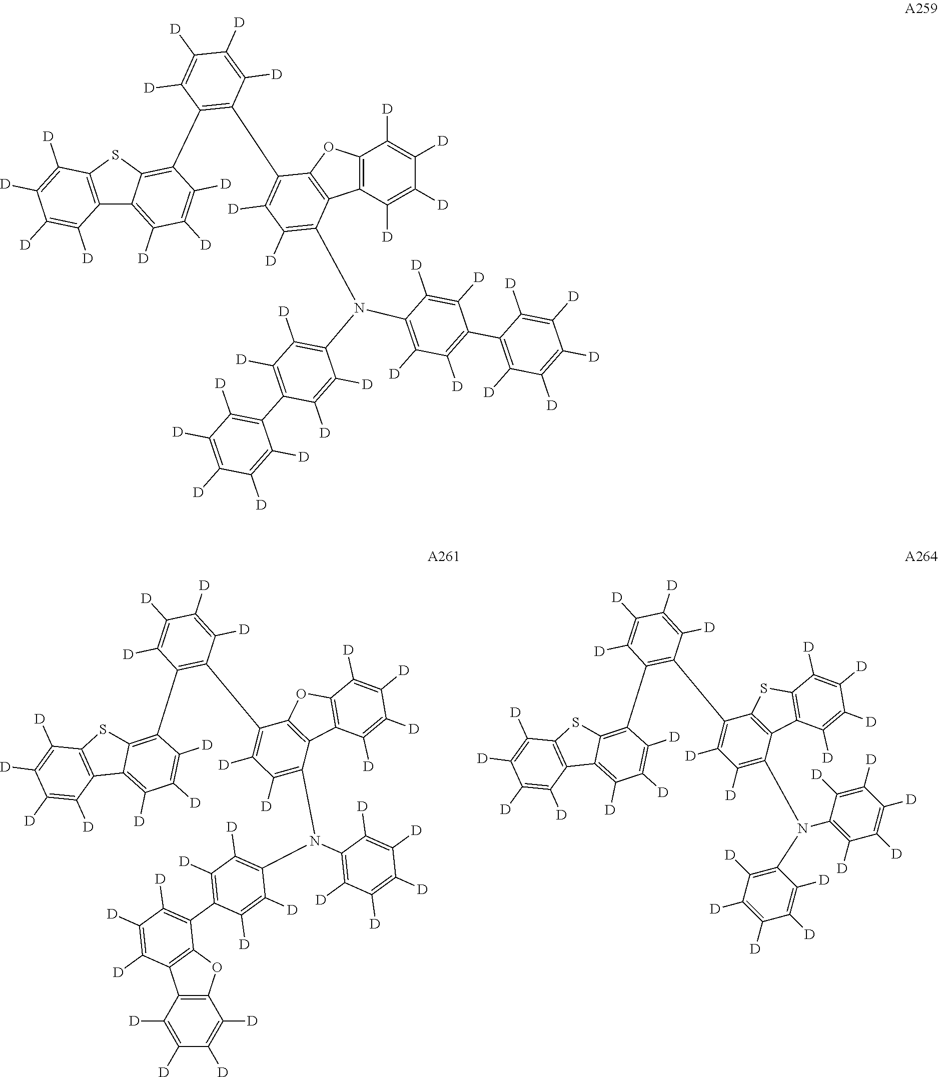

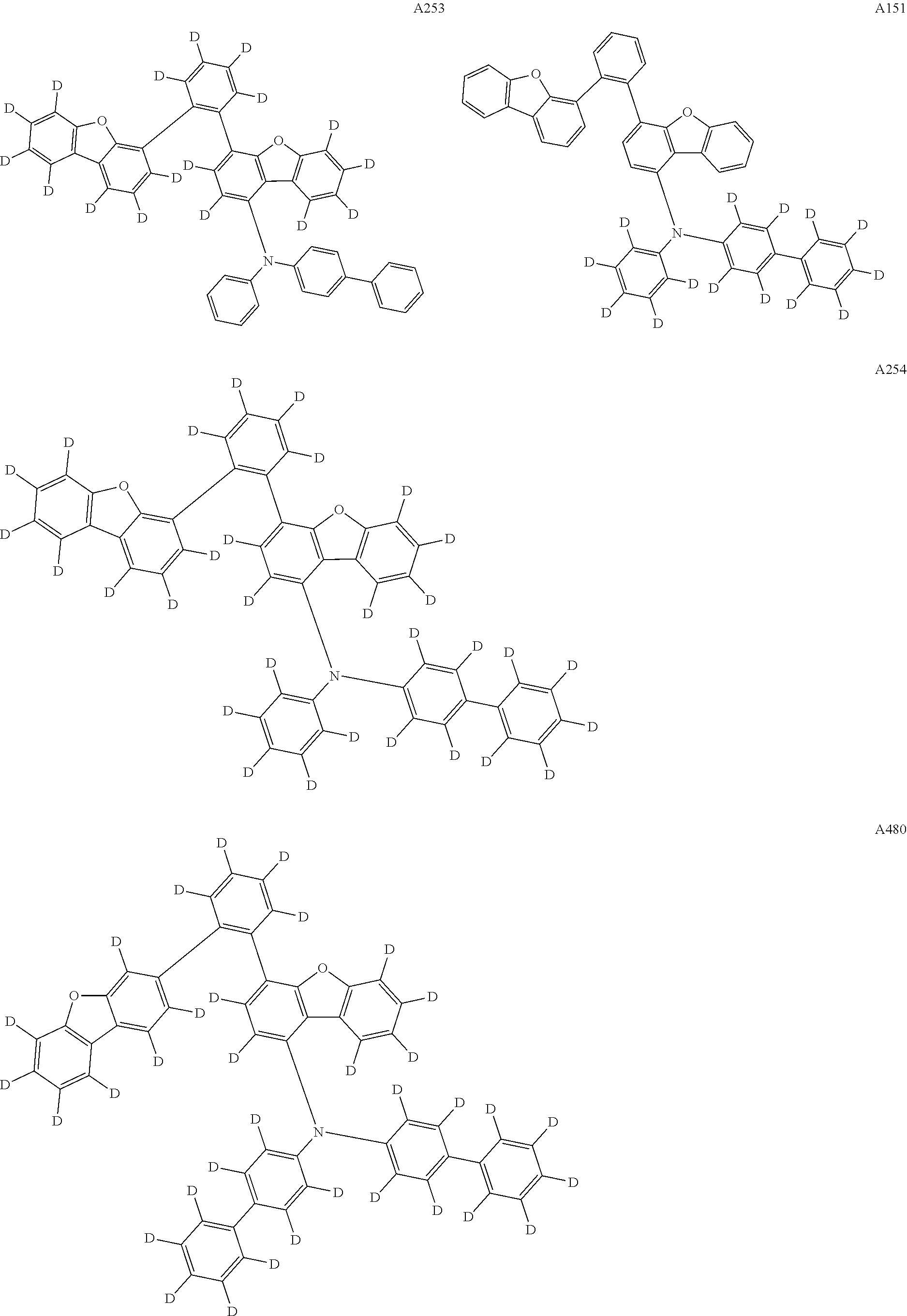

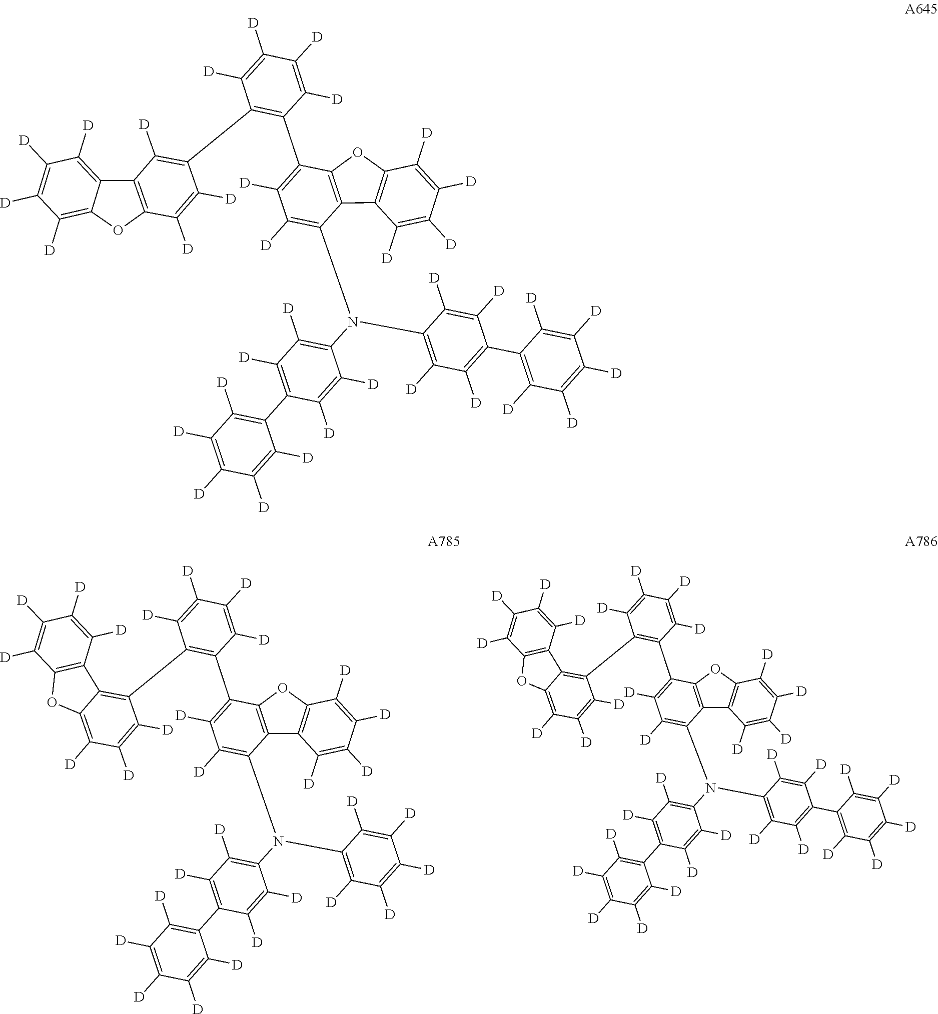

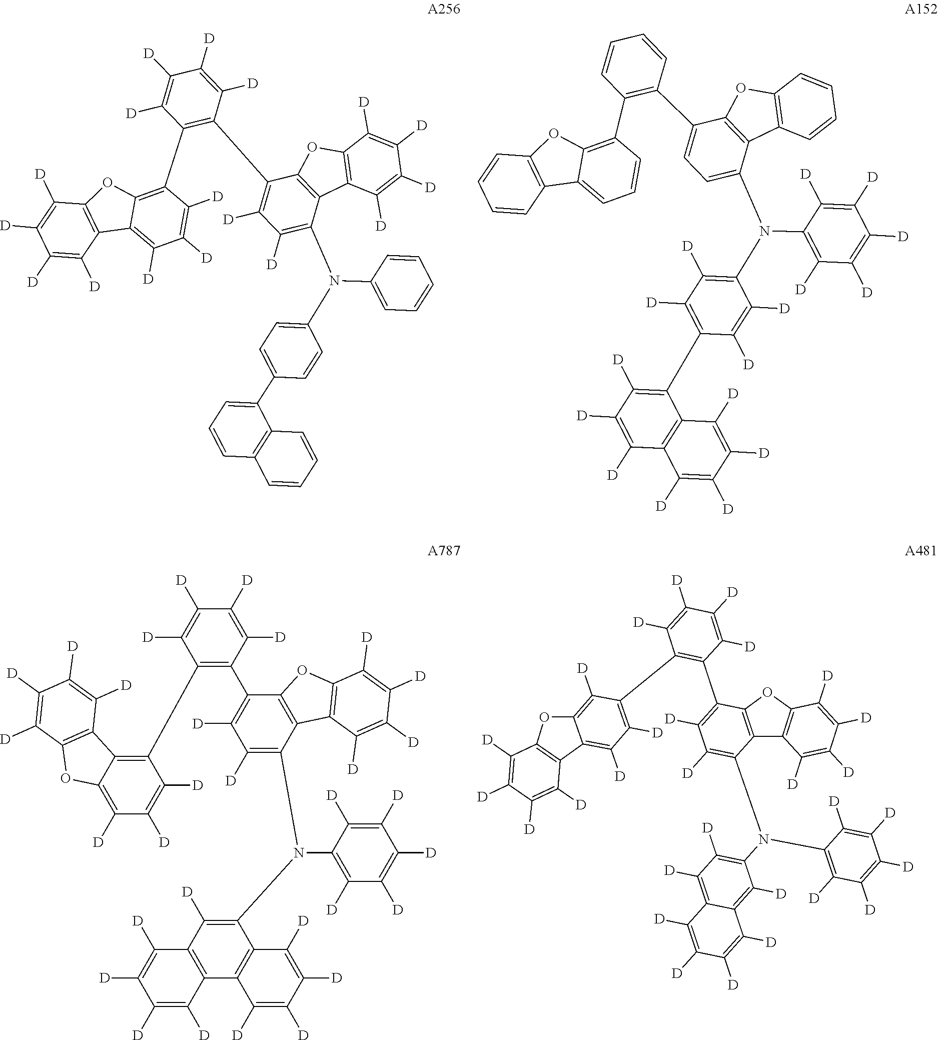

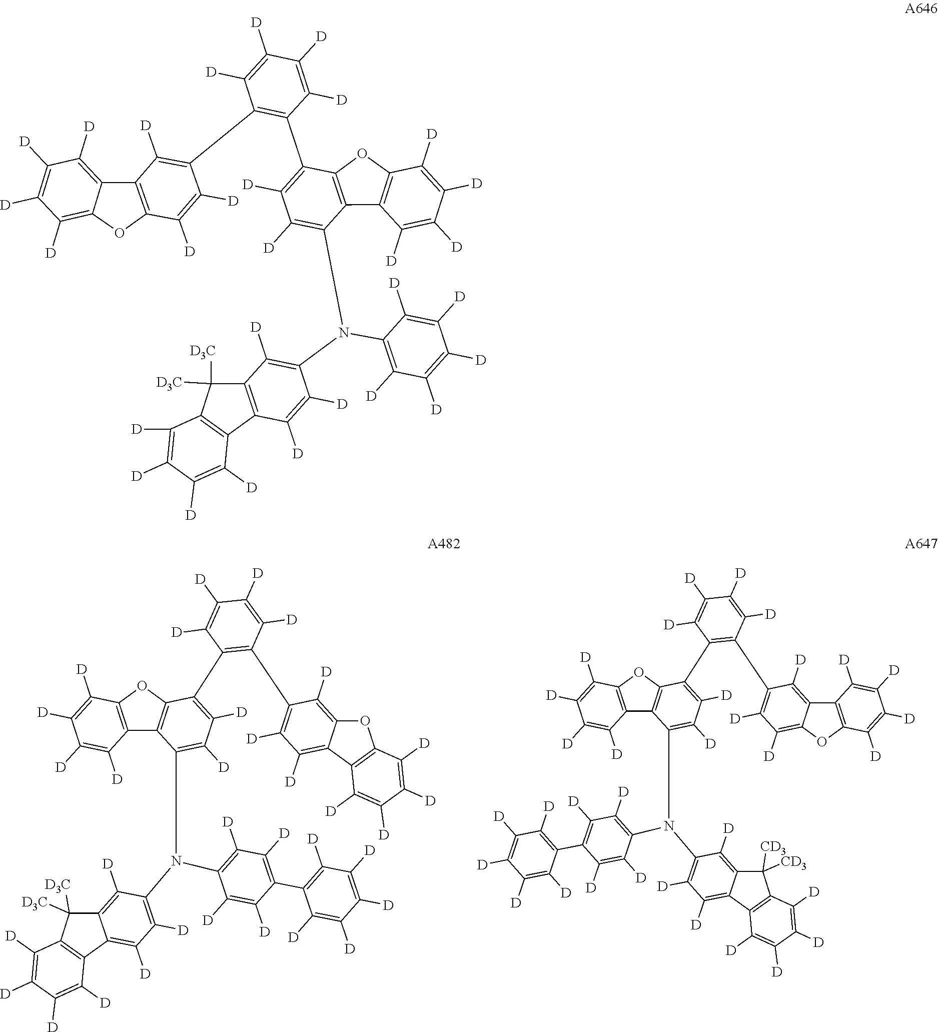

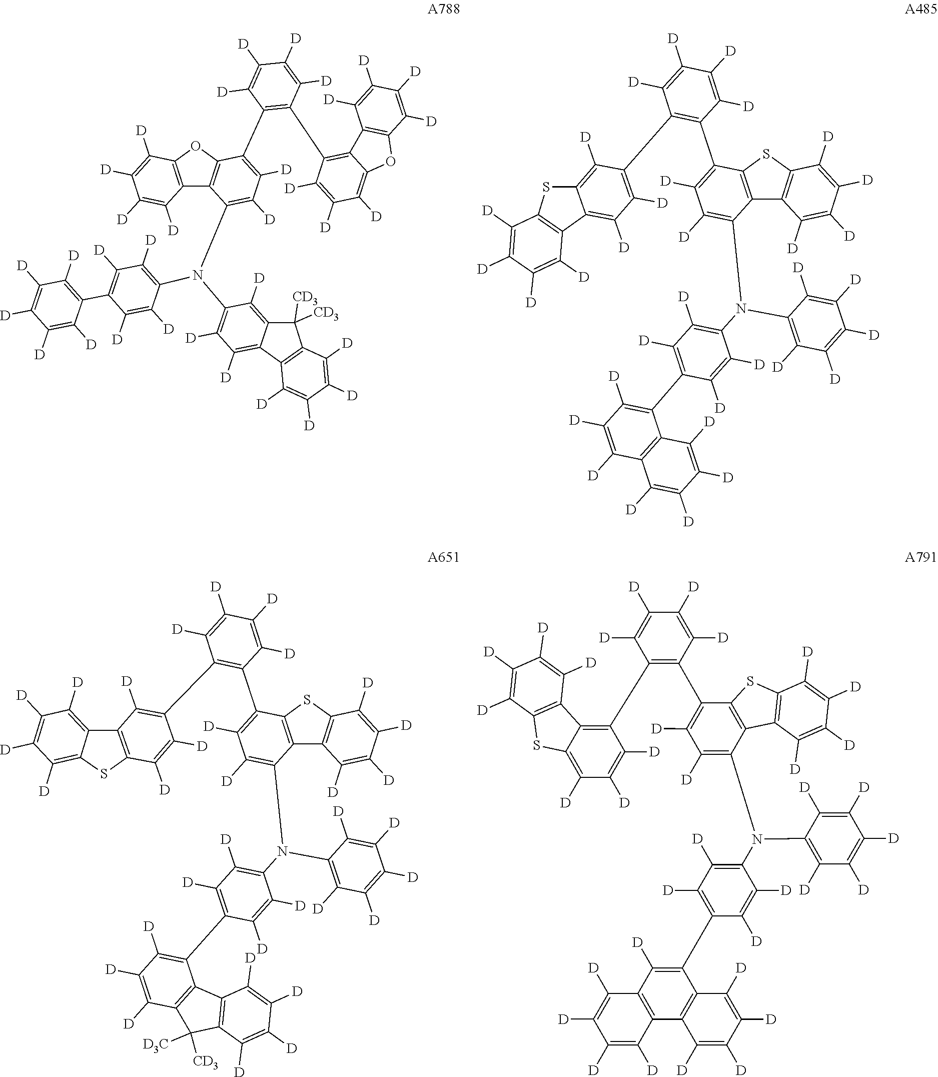

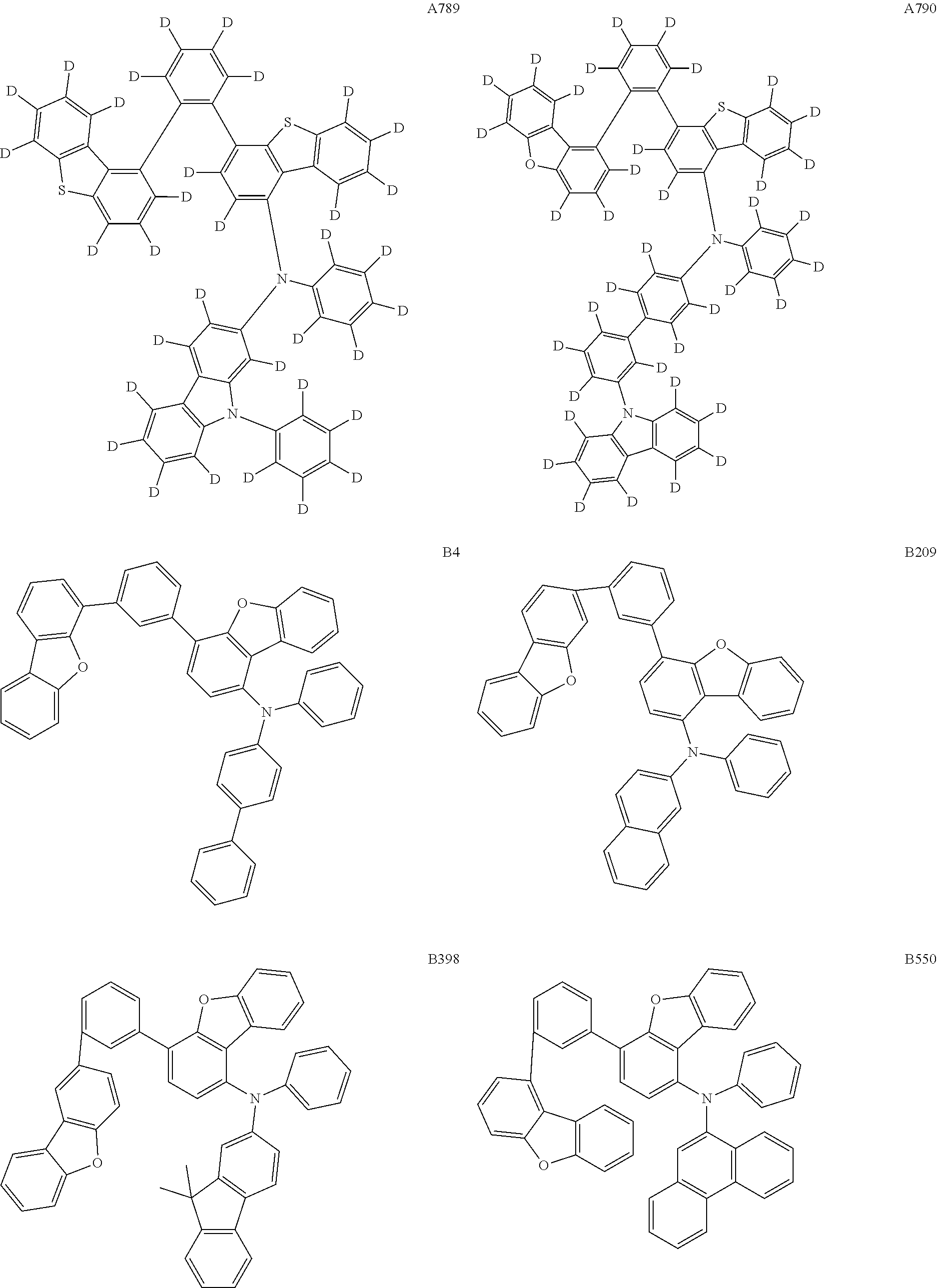

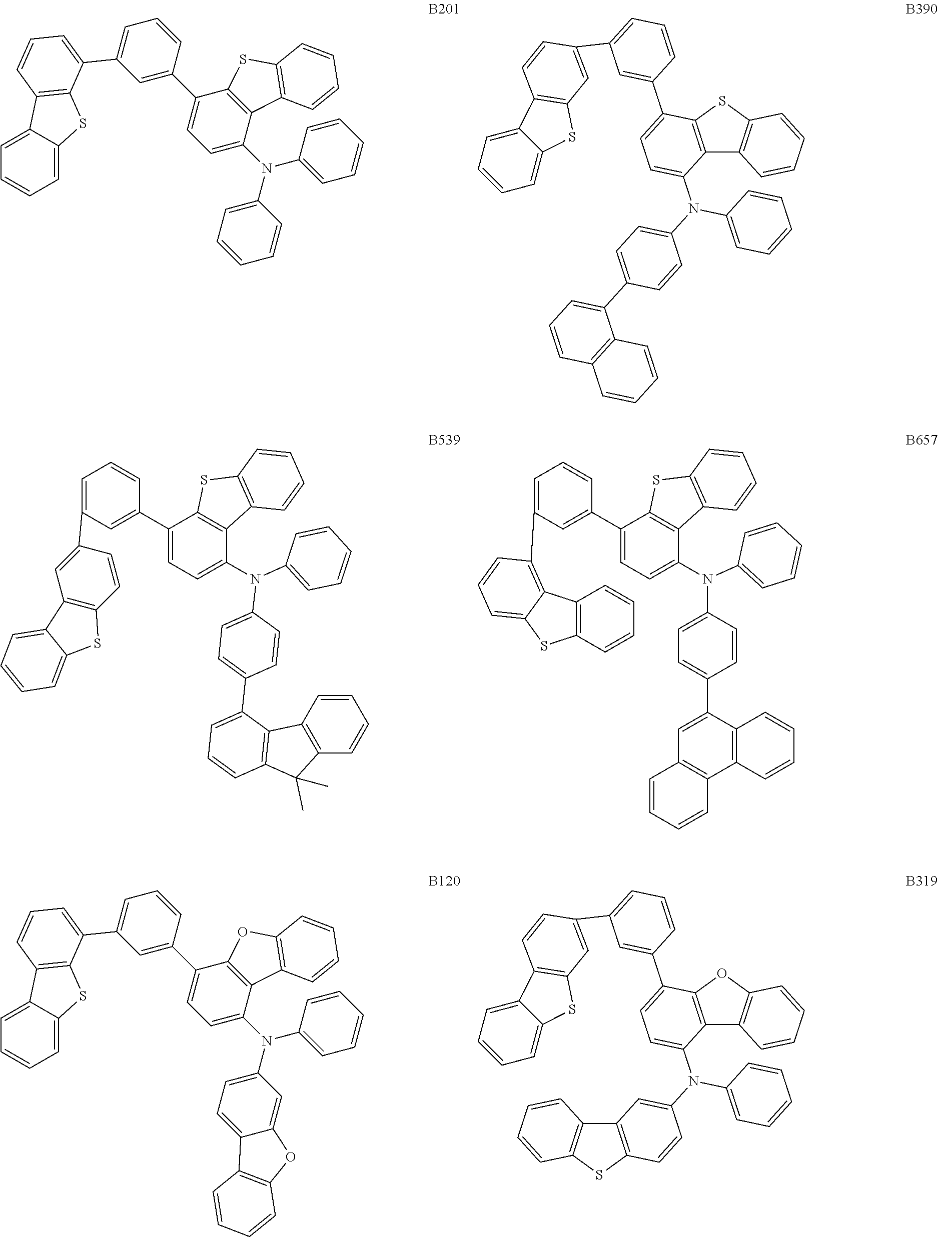

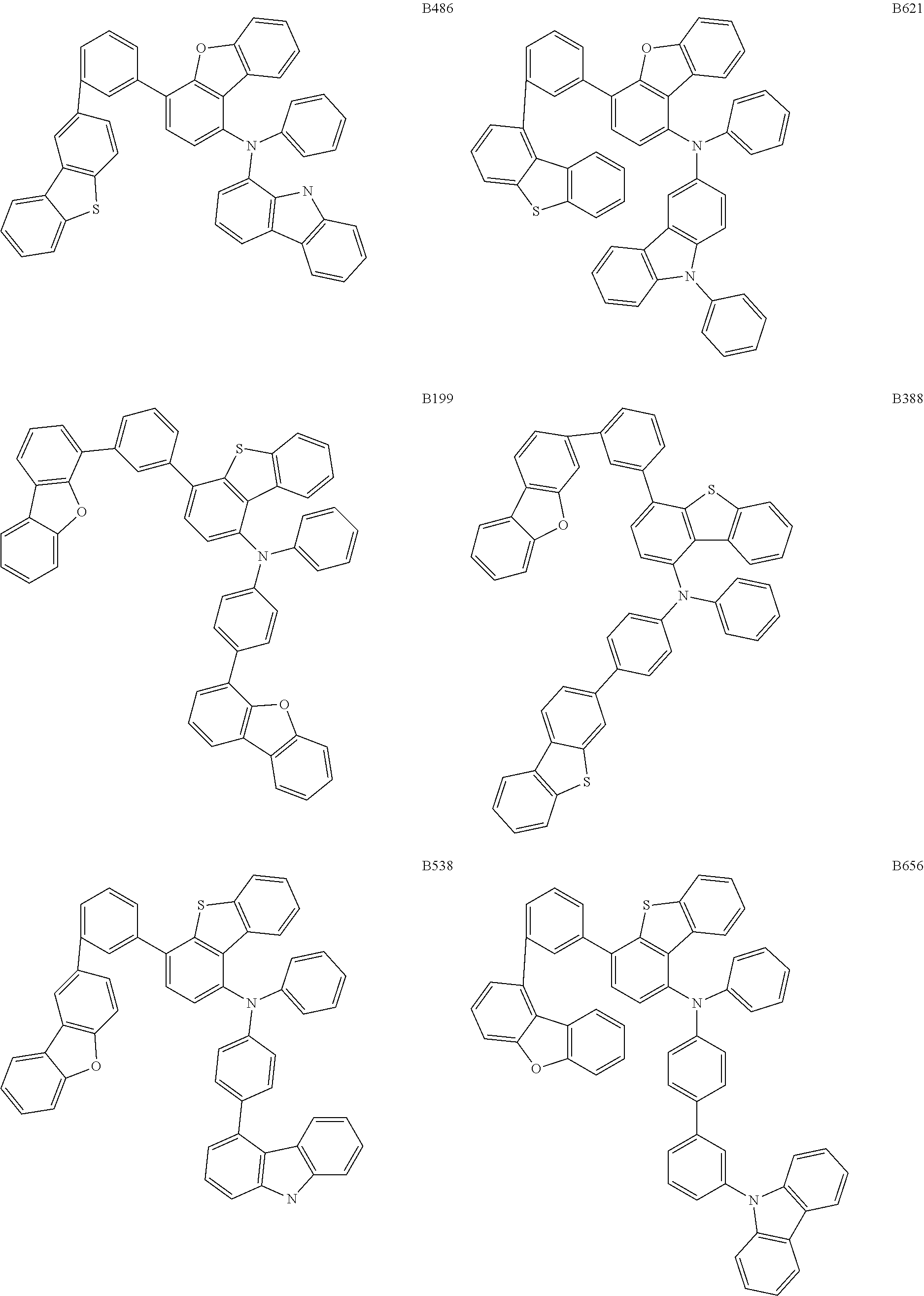

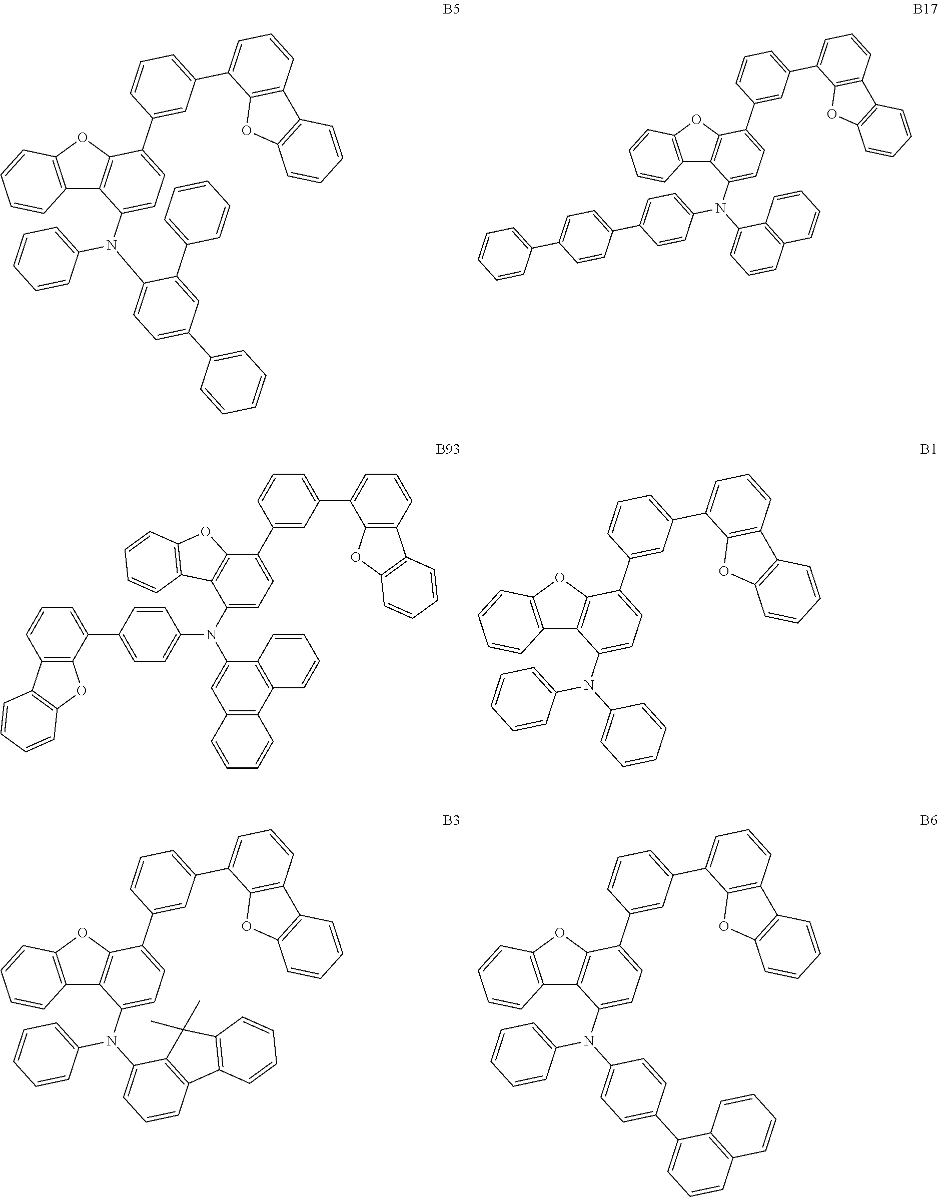

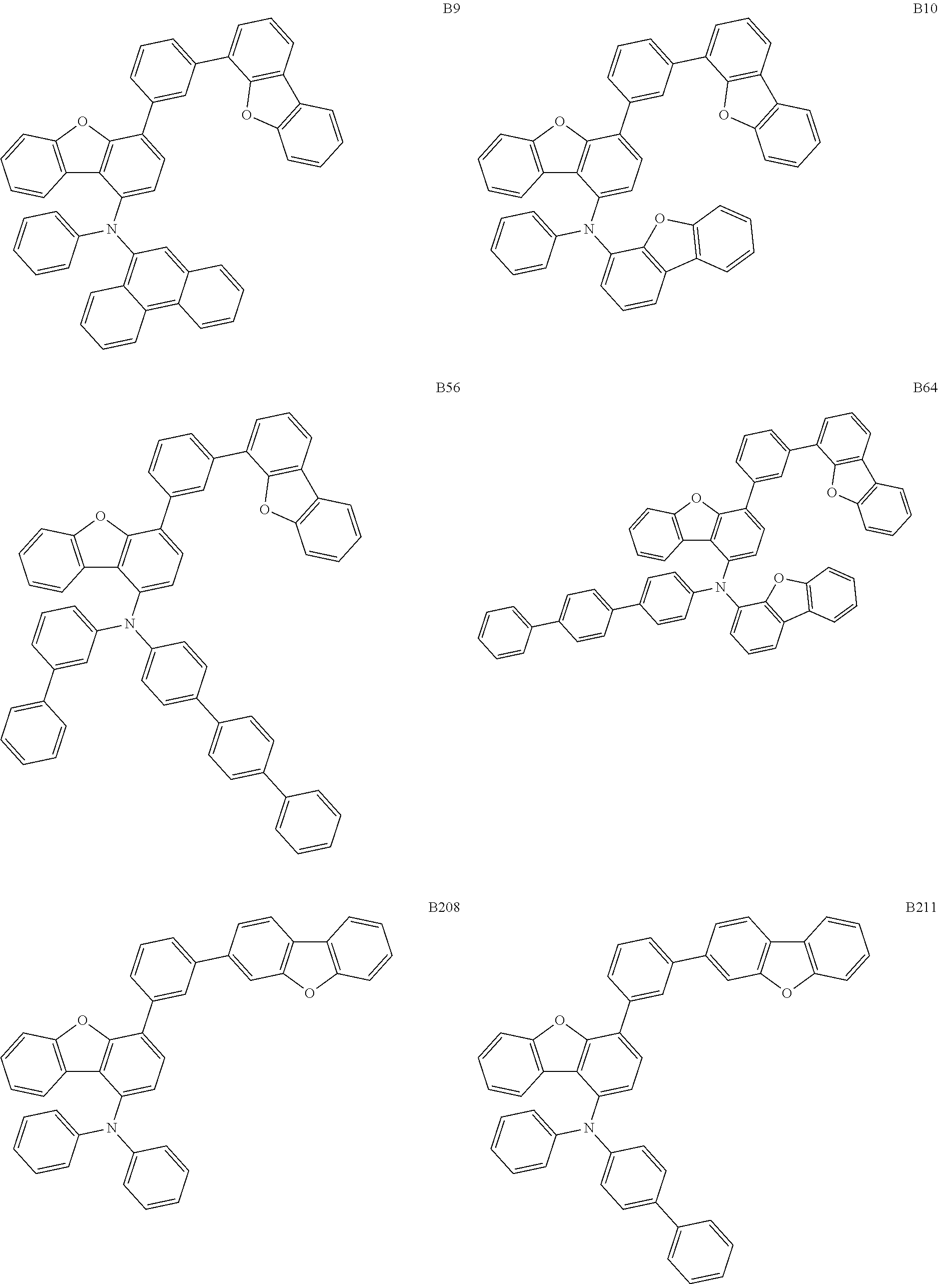

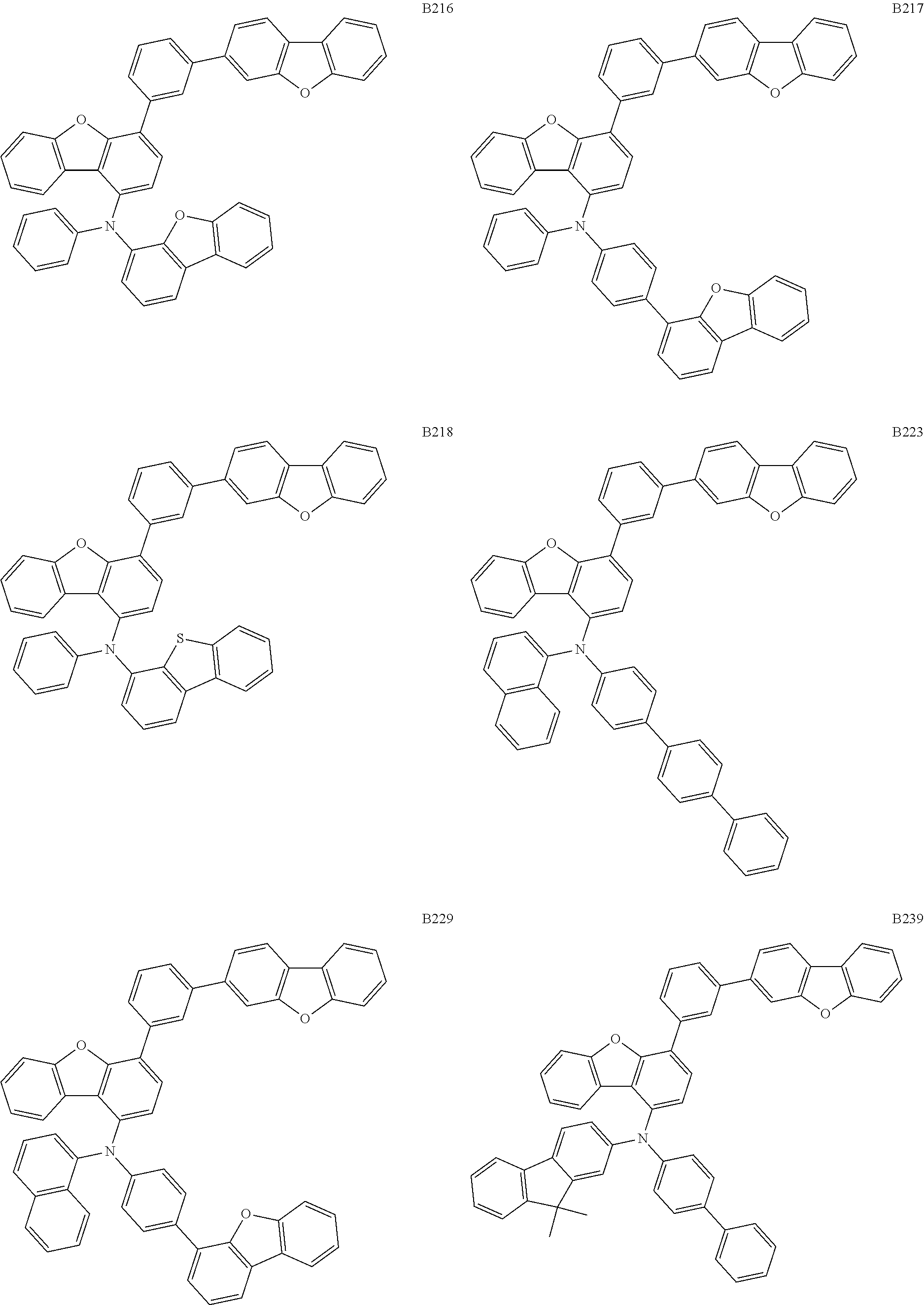

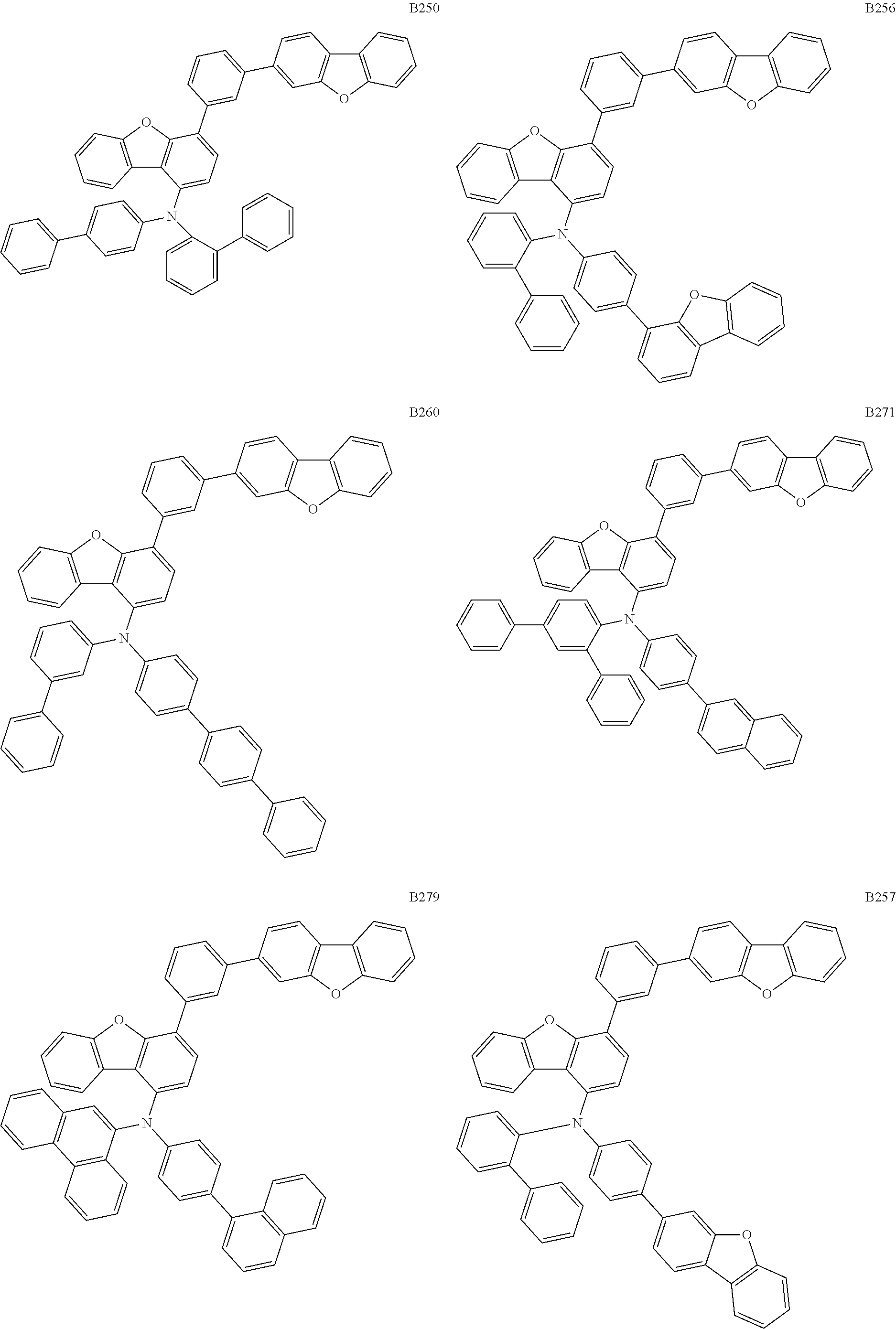

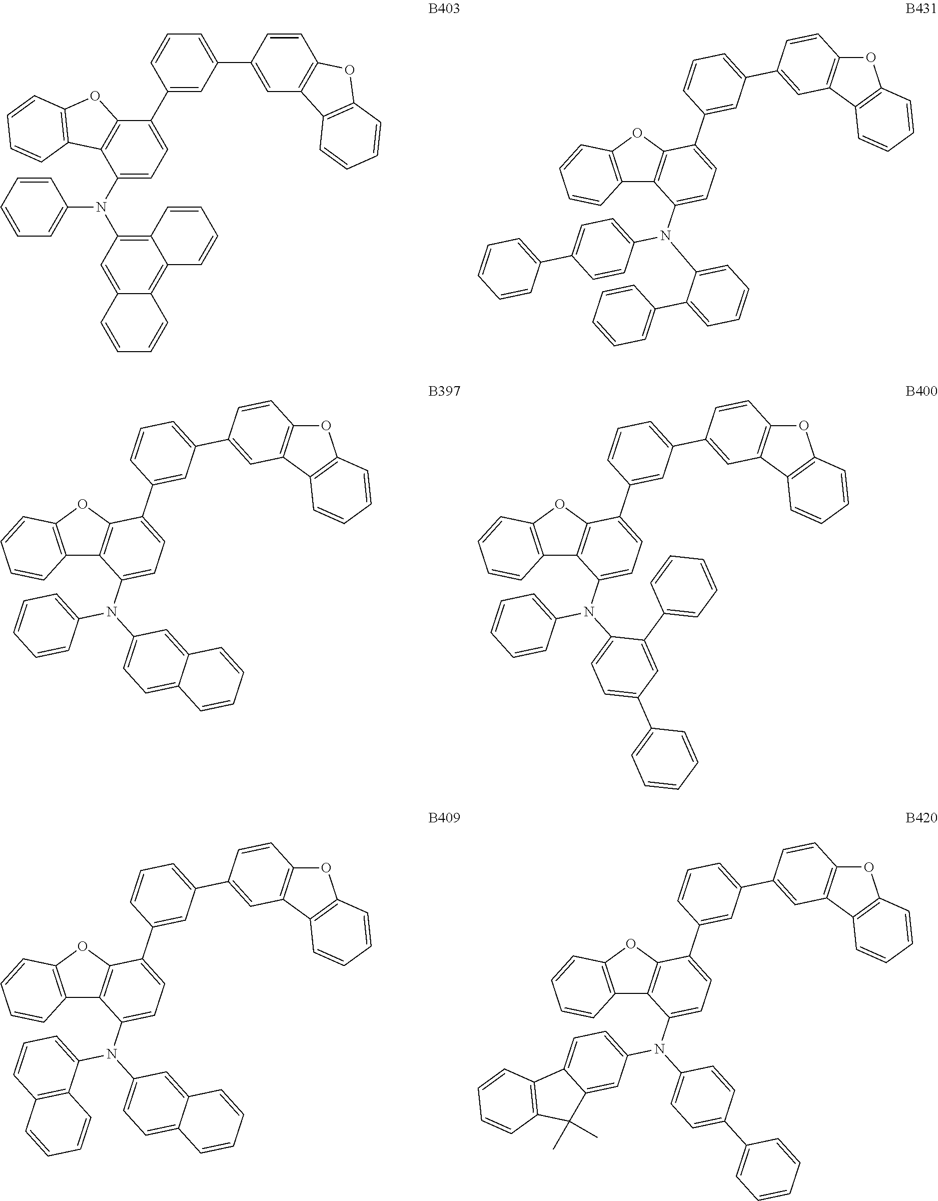

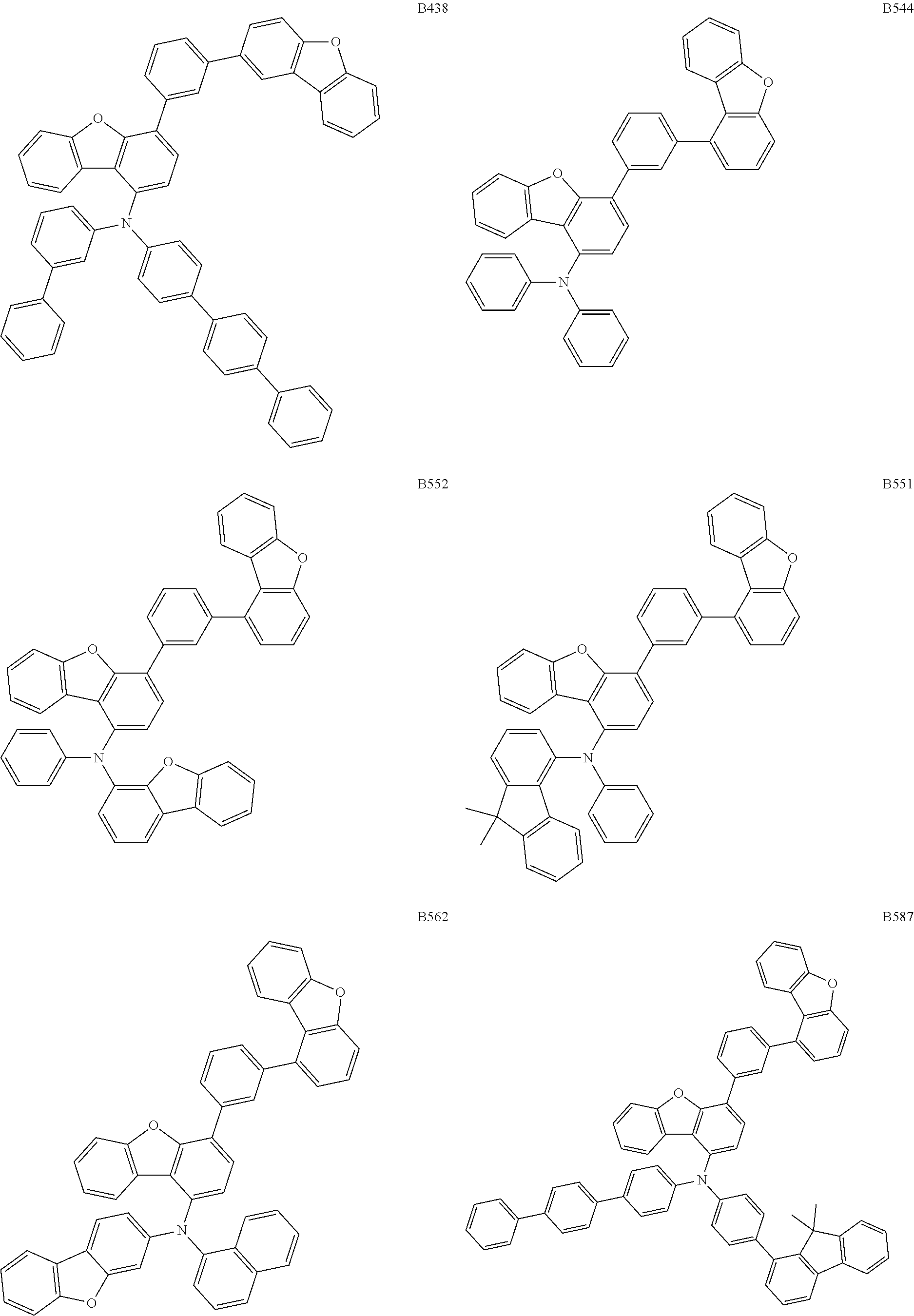

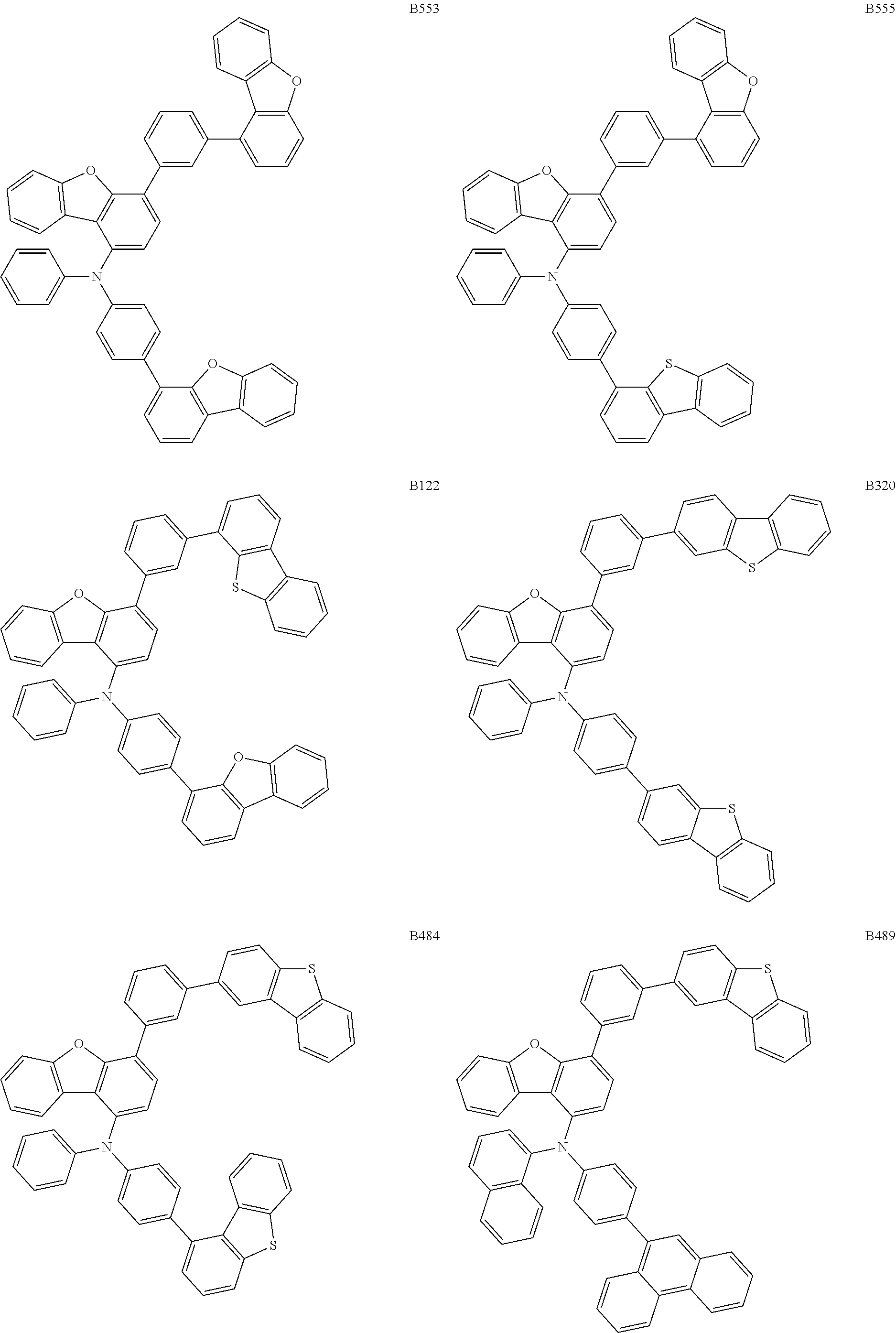

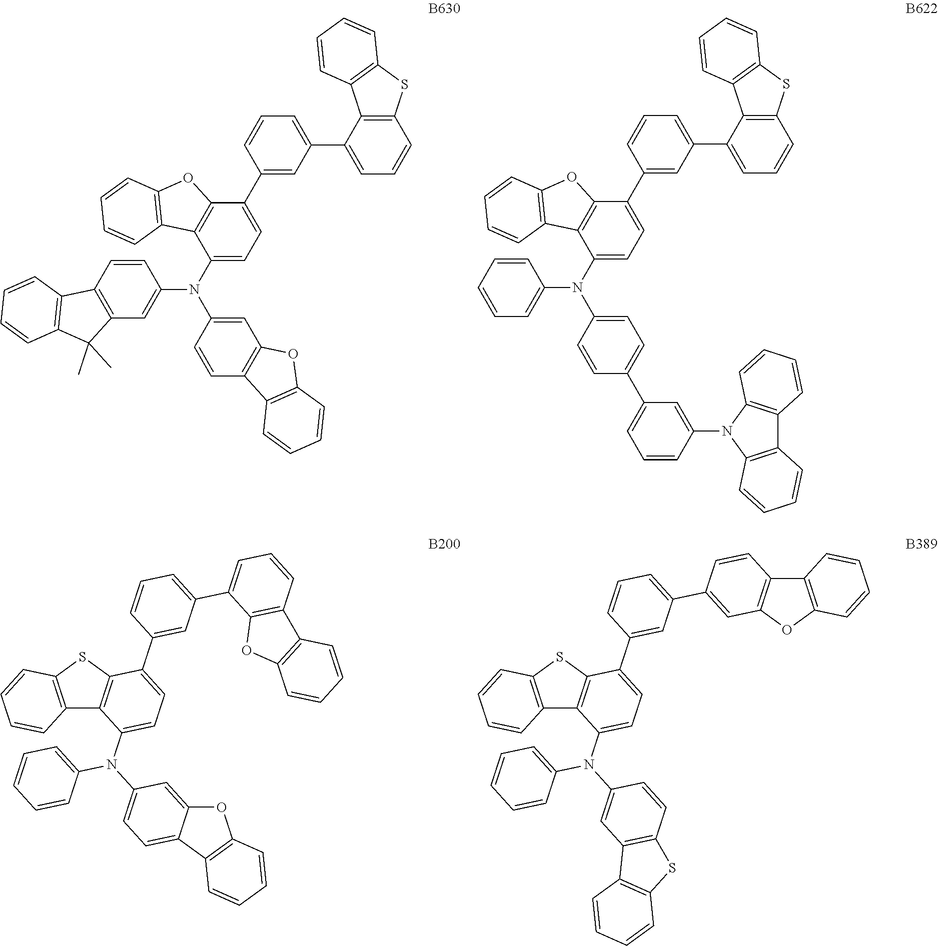

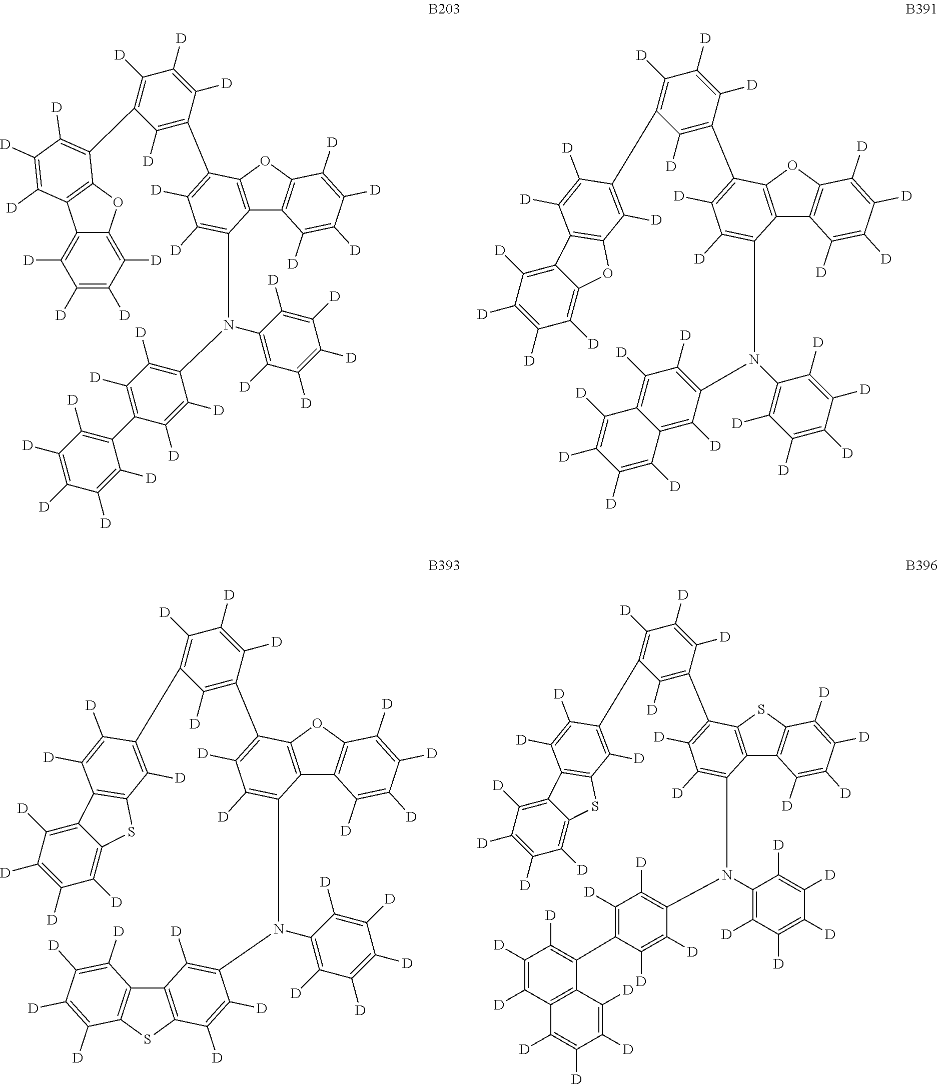

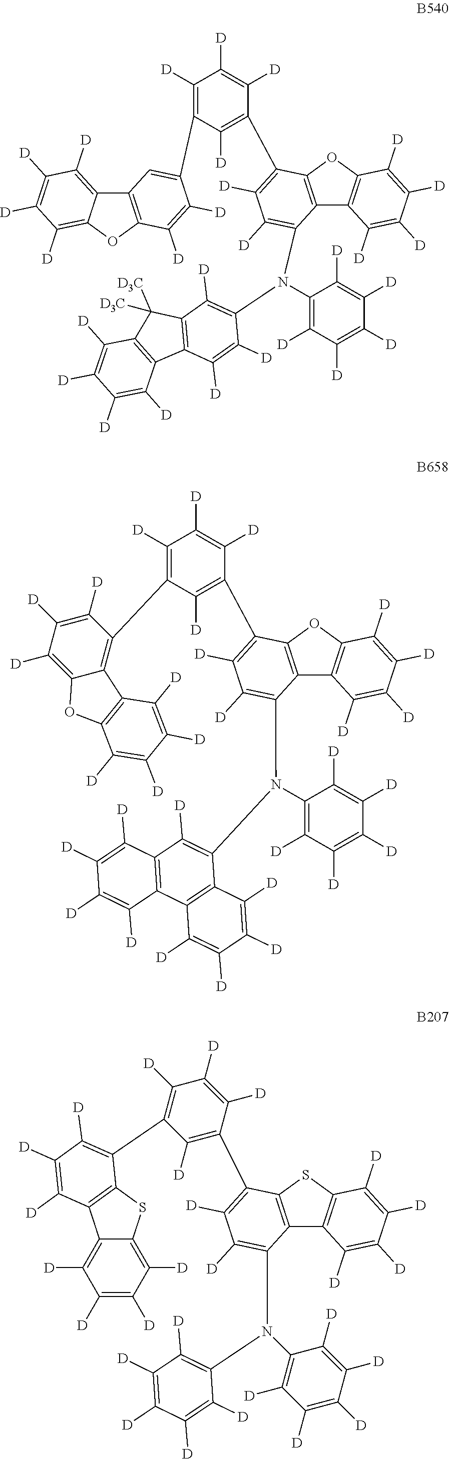

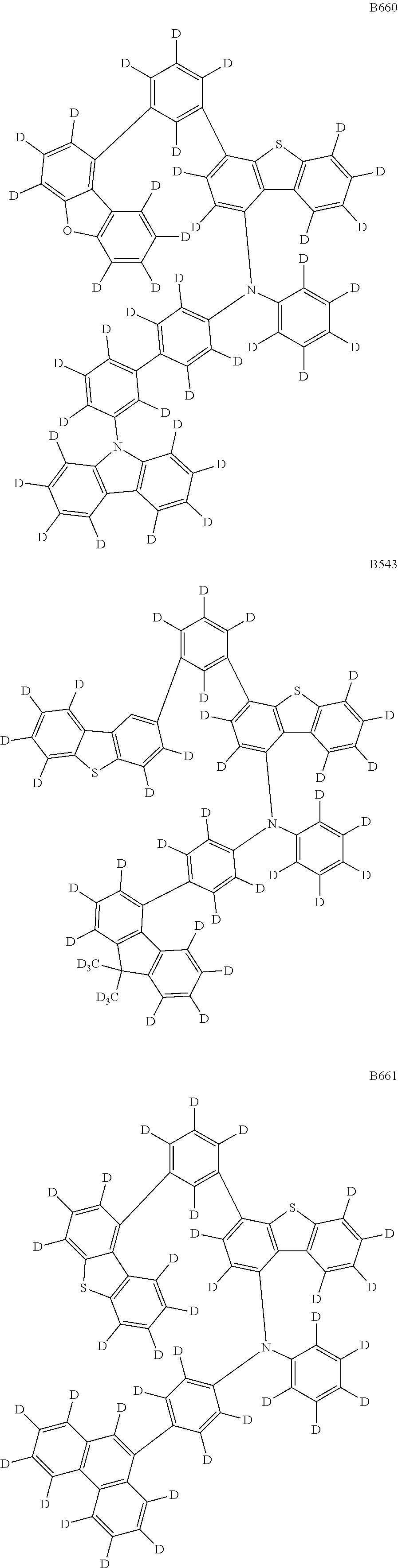

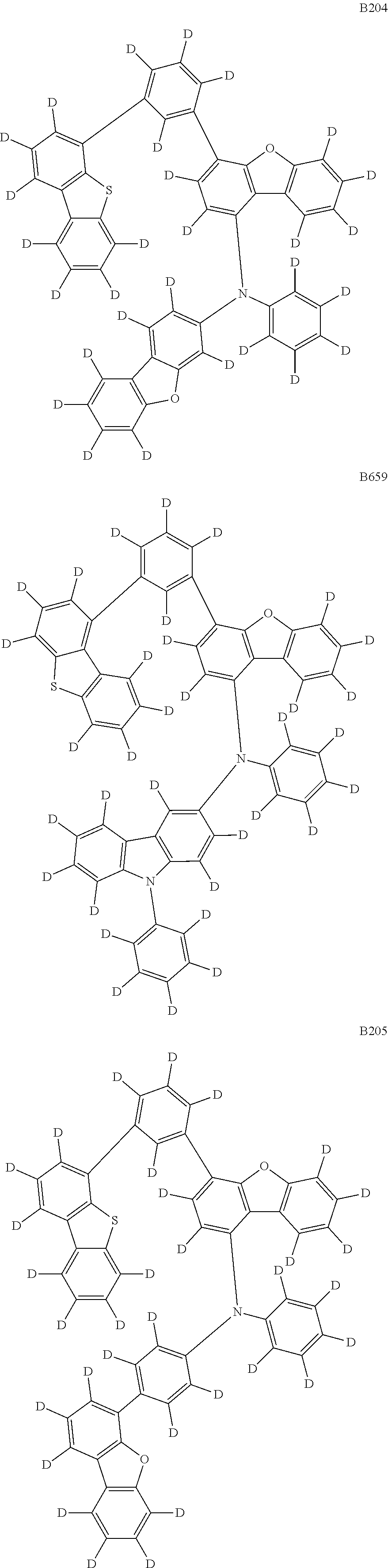

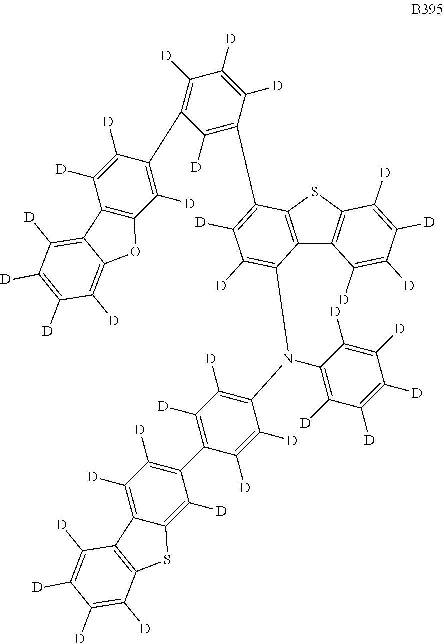 [SMILES: [2H]c1c([2H])c([2H])c(N(c2c([2H])c([2H])c(-c3c([2H])c([2H])c4c(sc5c([2H])c([2H])c([2H])c([2H])c54)c3[2H])c([2H])c2[2H])c2c([2H])c([2H])c(-c3c([2H])c([2H])c([2H])c(-c4c([2H])c([2H])c5c(oc6c([2H])c([2H])c([2H])c([2H])c65)c4[2H])c3[2H])c3sc4c([2H])c([2H])c([2H])c([2H])c4c23)c([2H])c1[2H]]

According to one example of the present disclosure, the Compounds represented by the Chemical Formulas 3-1 to 3-3 may be selected from the group consisting of Compounds as set forth below, but is not limited thereto.

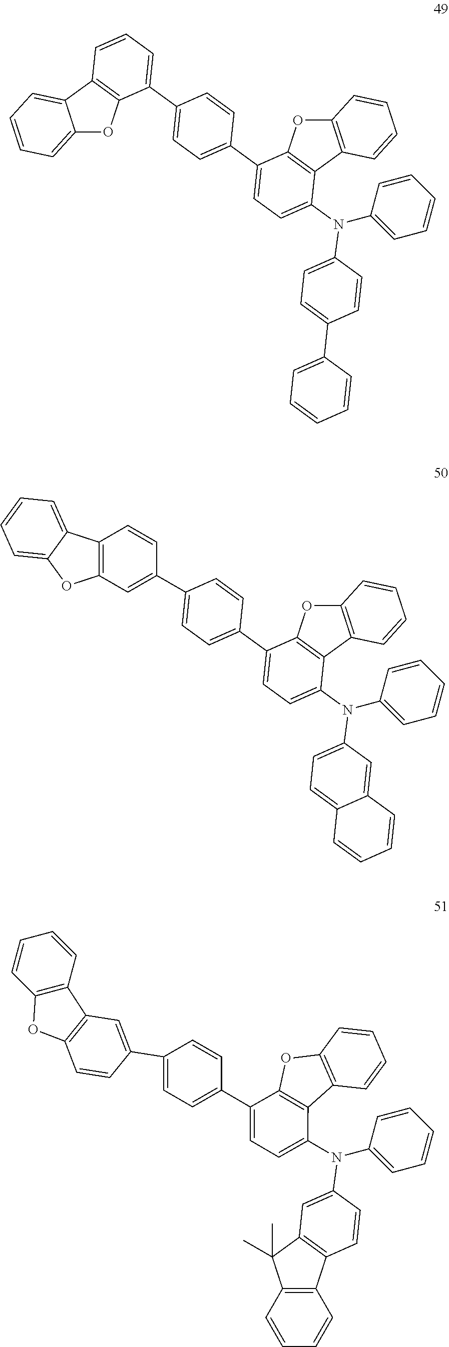 [SMILES: c1ccc(-c2ccc(N(c3ccccc3)c3ccc(-c4ccc(-c5cccc6c5oc5ccccc56)cc4)c4oc5ccccc5c34)cc2)cc1 ; c1ccc(N(c2ccc3ccccc3c2)c2ccc(-c3ccc(-c4ccc5c(c4)oc4ccccc45)cc3)c3oc4ccccc4c23)cc1 ; CC1(C)c2ccccc2-c2ccc(N(c3ccccc3)c3ccc(-c4ccc(-c5ccc6oc7ccccc7c6c5)cc4)c4oc5ccccc5c34)cc21]

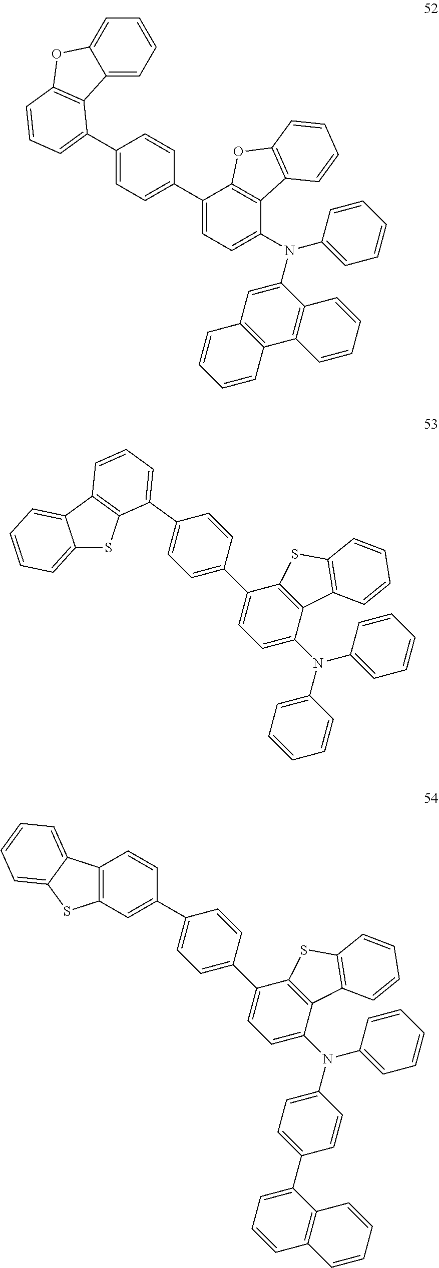 [SMILES: c1ccc(N(c2cc3ccccc3c3ccccc23)c2ccc(-c3ccc(-c4cccc5oc6ccccc6c45)cc3)c3oc4ccccc4c23)cc1 ; c1ccc(N(c2ccccc2)c2ccc(-c3ccc(-c4cccc5c4sc4ccccc45)cc3)c3sc4ccccc4c23)cc1 ; c1ccc(N(c2ccc(-c3cccc4ccccc34)cc2)c2ccc(-c3ccc(-c4ccc5c(c4)sc4ccccc45)cc3)c3sc4ccccc4c23)cc1]

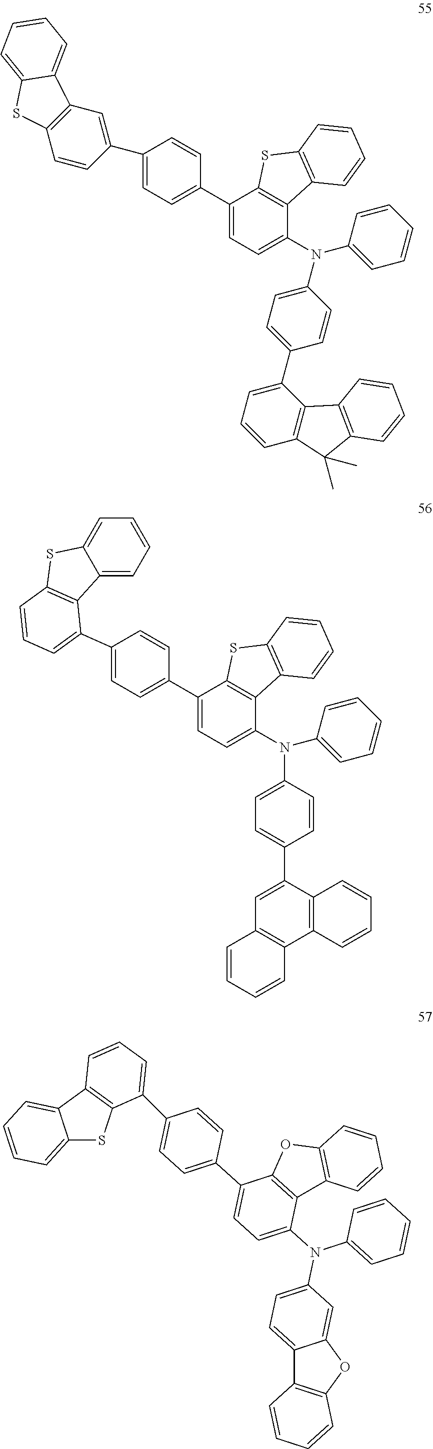 [SMILES: CC1(C)c2ccccc2-c2c(-c3ccc(N(c4ccccc4)c4ccc(-c5ccc(-c6ccc7sc8ccccc8c7c6)cc5)c5sc6ccccc6c45)cc3)cccc21 ; c1ccc(N(c2ccc(-c3cc4ccccc4c4ccccc34)cc2)c2ccc(-c3ccc(-c4cccc5sc6ccccc6c45)cc3)c3sc4ccccc4c23)cc1 ; c1ccc(N(c2ccc3c(c2)oc2ccccc23)c2ccc(-c3ccc(-c4cccc5c4sc4ccccc45)cc3)c3oc4ccccc4c23)cc1]

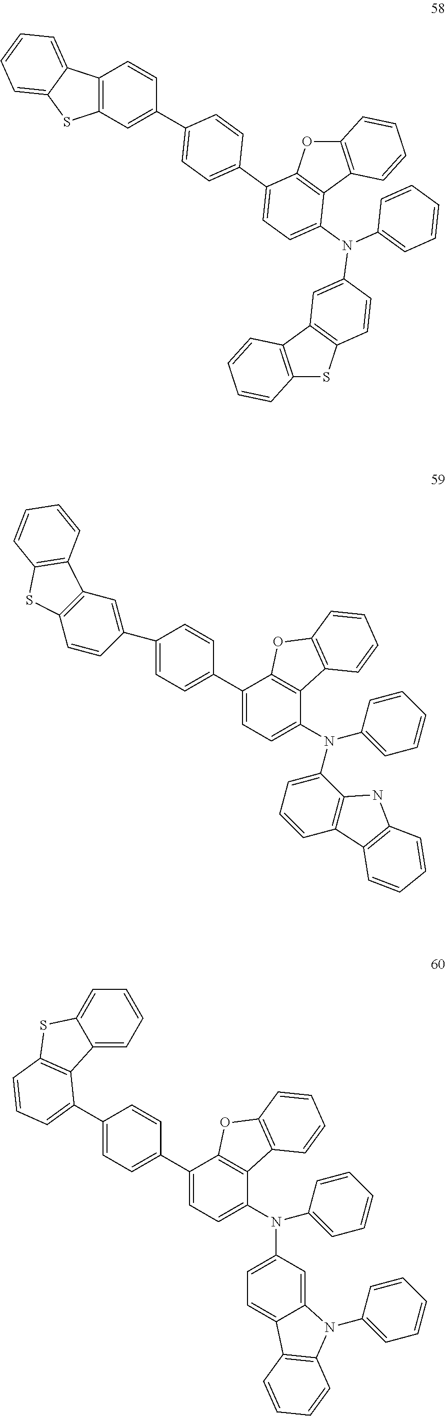 [SMILES: c1ccc(N(c2ccc3sc4ccccc4c3c2)c2ccc(-c3ccc(-c4ccc5c(c4)sc4ccccc45)cc3)c3oc4ccccc4c23)cc1 ; c1ccc(N(c2cccc3c2[nH]c2ccccc23)c2ccc(-c3ccc(-c4ccc5sc6ccccc6c5c4)cc3)c3oc4ccccc4c23)cc1 ; c1ccc(N(c2ccc3c4ccccc4n(-c4ccccc4)c3c2)c2ccc(-c3ccc(-c4cccc5sc6ccccc6c45)cc3)c3oc4ccccc4c23)cc1]

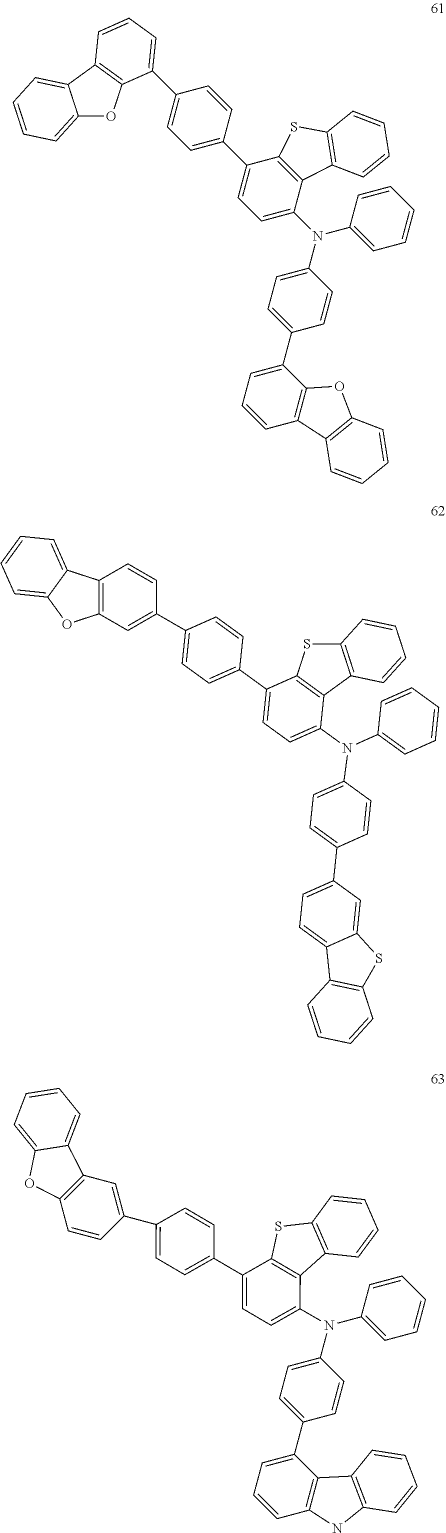 [SMILES: c1ccc(N(c2ccc(-c3cccc4c3oc3ccccc34)cc2)c2ccc(-c3ccc(-c4cccc5c4oc4ccccc45)cc3)c3sc4ccccc4c23)cc1 ; c1ccc(N(c2ccc(-c3ccc4c(c3)sc3ccccc34)cc2)c2ccc(-c3ccc(-c4ccc5c(c4)oc4ccccc45)cc3)c3sc4ccccc4c23)cc1 ; c1ccc(N(c2ccc(-c3cccc4[nH]c5ccccc5c34)cc2)c2ccc(-c3ccc(-c4ccc5oc6ccccc6c5c4)cc3)c3sc4ccccc4c23)cc1]

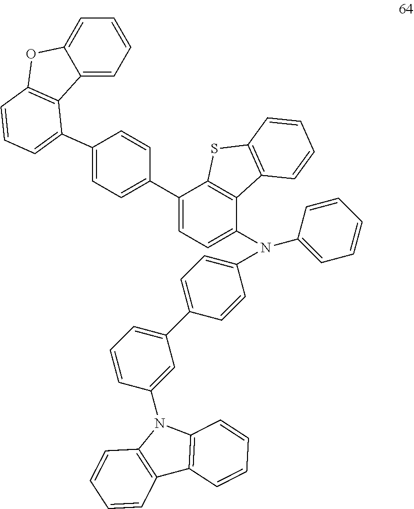 [SMILES: c1ccc(N(c2ccc(-c3cccc(-n4c5ccccc5c5ccccc54)c3)cc2)c2ccc(-c3ccc(-c4cccc5oc6ccccc6c45)cc3)c3sc4ccccc4c23)cc1]

The organic light emitting diode according to an aspect of the present disclosure may include a first electrode and, a second electrode facing the first electrode, and an organic layer between the first electrode and the second electrode.

According to one example of the present disclosure, the organic layer including the compound represented by the Chemical Formula A of the present disclosure may be one or more of a hole transport layer (HTL) and an hole transport auxiliary layer.

When the organic compound represented by the Chemical Formula A of the present disclosure is used as a material of the hole transport auxiliary layer, the organic layer may have a suitable energy level so as to act as the hole transport auxiliary layer serving to transfer holes from the hole transport layer to the light emitting layer and block electrons coming from the light emitting layer. Thus, in one example, the organic layer including the compound represented by the Chemical Formula A of the present disclosure may be the hole transport auxiliary layer.

In addition, in the organic light emitting diode of the present disclosure, even when the hole transport layer and/or the hole transport auxiliary layer including the organic compound represented by the Chemical Formula A of the present disclosure is combined with the light emitting layer emitting light of any color, the light emitting layer may emit light of a color having a target color coordinate excellently.

The organic layer may further include at least one selected from the group consisting of a Hole Injection Layer (HIL), an Emitting Layer (EML), an Electron Transport Layer (ETL) and an Electron Injection Layer (EIL), in addition to the hole transport layer and the hole transport auxiliary layer.

For example, the organic light emitting diode may have a structure in which a first electrode, a hole injection layer (HIL), a hole transport layer (HTL), an hole transport auxiliary layer, an emission layer (EML), an electron transport layer (ETL), an electron injection layer (EIL), and a second electrode are sequentially stacked.

The organic layer may further include an auxiliary electron transport layer.

The first electrode may be a positive electrode (an anode), and the first electrode may include a material such as indium tin oxide (ITO), indium zinc oxide (IZO), tin oxide (SnO₂), zinc oxide (ZnO), or the like, which is transparent and has excellent conductivity.

The second electrode may be a negative electrode (cathode), and the second electrode may include a material such as lithium (Li), aluminum (Al), aluminum-lithium (Al—Li), calcium (Ca), magnesium (Mg), magnesium-indium (Mg—In), or magnesium-silver (Mg—Ag). In addition, in the case of the top emission organic light emitting diode, a transparent second electrode through which light can transmit may be formed using indium tin oxide (ITO) or indium zinc oxide (IZO). A capping layer CPL made of a composition for forming a capping layer may be formed on a surface of the second electrode.

In addition, an encapsulation film or protecting film for protecting the organic light emitting diode or diode from moisture and oxygen or the like may be further formed on the capping layer CPL. The encapsulation film or protective film may be made of a curable adhesive composition in which an inorganic moisture absorbent is incorporated.

A hole injection layer compound or a hole transport layer compound is not specifically limited. Any compound may be used as the hole injection layer or hole transport layer compound as long as it is generally used as the hole injection layer or hole transport layer compound. Non-limiting examples of the hole injection layer or hole transport layer compound may include a phthalocyanine derivative, a porphyrin derivative, a triarylamine derivative and an indolocarbazole derivative. For example, non-limiting examples of the hole injection layer or hole transport layer compound may include 1,4,5,8,9,11-hexaazatriphenylene-hexacarbonitrile (HAT-CN), copper phthalocyanine (CuPc), 4,4′,4″-tris(3-methylphenyl)amino) triphenylamine (m-MTDATA), 4,4′,4″-tris(3-methylphenylamino) phenoxybenzene (m-MTDAPB), 4,4′,4″-tri (N-carbazolyl) triphenylamine (TCTA), 4,4′,4″-tris(N-(2-naphthyl)-N-phenylamino)-triphenylamine (2-TNATA), N4,N4,N4′,N4′-tetra([1,1′-biphenyl]-4-yl)-[1,1′-biphenyl]-4,4′-diamine, bis(N-(1-naphthyl-n-phenyl)) benzidine (α-NPD), N,N′-di(naphthalen-1-yl)-N,N′-biphenyl-benzidine (NPB) or N,N′-biphenyl-N,N′-bis(3-methylphenyl)-1,1′-biphenyl-4,4′-diamine (TPD), etc.

The compound included in the light emitting layer is not specifically limited, and any compound may be used as the compound included in the light emitting layer as long as it is generally used as the light emitting layer compound. A single light emitting compound or a light emitting host compound may be used as the light emitting layer compound.

Examples of the light emitting compound of the light emitting layer may include compounds that may cause light-emission via phosphorescence, fluorescence, thermally-activated delayed fluorescence, i.e., TADF (also referred to as E-type delayed fluorescence), triplet-triplet quenching, or a combination of these processes. However, the present disclosure is not limited thereto. The light emitting compound may be selected from a variety of materials depending on a desired color to be rendered. Non-limiting examples of the light emitting compound may include condensed cyclic derivatives such as phenanthrene, anthracene, pyrene, tetracene, pentacene, perylene, naphthopyrene, dibenzopyrene, rubrene, and chrysene, a benzoxazole derivative, a benzothiazole derivative, a benzoimidazole derivative, a benzotriazole derivative, an oxazole derivative, a oxadiazole derivative, a thiazole derivative, a imidazole derivative, a thiadiazole derivative, a triazole derivative, a pyrazoline derivative, a stilbene derivative, a thiophene derivative, a tetraphenylbutadiene derivative, a cyclopentadiene derivative, a bisstyryl derivative, a bisstyryl arylene derivative, a diazindacene derivative, a furan derivative, a benzofuran derivative, a isobenzofuran derivative, a dibenzofuran derivative, a coumarin derivative, a dicyanomethylenepyran derivative, a dicyanomethylenethiopyran derivative, a polymethine derivative, a cyanine derivative, a oxobenzoanthracene derivative, an xanthene derivative, a rhodamine derivative, a fluorescein derivative, a pyrylium derivative, a carbostyryl derivative, a acridine derivative, a oxazine derivative, a phenylene oxide derivative, a quinacridone derivative, a quinazoline derivative, a pyrrolopyridine derivative, a furopyridine derivative, a 1,2,5-thiadiazolopyrene derivative, a pyromethene derivative, a perinone derivative, a pyrrolopyrrole derivative, a squaryllium derivative, a biolanthrone derivative, a phenazine derivative, a acridone derivative, a deazaflavin derivative, a fluorene derivative, a benzofluorene derivative, an aromatic boron derivative, an aromatic nitrogen boron derivative, and a metal complex (complex in which a metal such as Ir, Pt, Au, Eu, Ru, Re, Ag, and Cu binds to a heteroaromatic ring ligand). For example, non-limiting examples of the light emitting compound may include N1,N1,N6,N6-tetrakis(4-(1-silyl)phenyl) pyrene-1,6-diamine, 2,12-di-tert-butyl-5,9-bis(4-(tert-butyl)phenyl)-7-(3,5-di-tert-butylphenyl)-5,9-dihydro-5,9-diaza-13b-boranaphtho[3,2,1-de]anthracene (t-DABNA-dtB), Platinum octaethylporphyrin (PtOEP), Ir(ppy)₃, Ir(ppy)₂(acac), Ir(mppy)₃, Ir(PPy)₂(m-bppy), BtpIr(acac), Ir(btp)₂(acac), Ir(2-phq)₃, Hex-Ir(phq)₃, Ir(fbi)₂(acac), fac-Tris(2-(3-p-xylyl)phenyl)pyridine iridium (III), Eu(dbm)₃(Phen), Ir(piq)₃, Ir(piq)₂(acac), Ir(Fliq)₂(acac), Ir(Flq)₂(acac), Ru(dtb-bpy)₃·2(PF₆), Ir(BT)₂(acac), Ir(DMP)₃, Ir(Mphq)₃IR (phq)₂tpy, fac-Ir(ppy)₂Pc, Ir(dp)PQ₂, Ir(Dpm)(Piq)₂, Hex-Ir(piq)₂(acac), Hex-Ir(piq)₃, Ir(dmpq)₃, Ir(dmpq)₂(acac), FPQIrpic, FIrpic, etc.

As a host compound of the light emitting layer, a light emitting host, a hole-transporting host, an electron-transporting host, or a combination thereof may be used. Non-limiting examples of a light emitting host compound may include condensed cyclic derivatives such as anthracene and pyrene, bisstyryl derivatives such as a bisstyryl anthracene derivative and a distyrylbenzene derivative, a tetraphenylbutadiene derivative, a cyclopentadiene derivative, a fluorene derivative, a benzofluorene derivative, a N-phenylcarbazole derivative, and a carbazonitrile derivative. Non-limiting examples of the hole-transporting host material may include a carbazole derivative, a dibenzofuran derivative, a dibenzothiophene derivative, a triarylamine derivative, an indolocarbazole derivative, and a benzoxazinophenoxazine derivative. Non-limiting examples of the electron-transporting host material may include a pyridine derivative, a triazine derivative, a phosphorus oxide derivative, a benzofuropyridine derivative, and a dibenzoxacillin derivative. For example, the non-limiting examples of the electron-transporting host material may include 9,10-bis(2-naphthyl) anthracene (ADN), tris(8-hydroxyquinolinato)aluminum (Alq₃), BAlq (8-hydroxyquinoline beryllium salt), DPVBi (4,4′-bis(2,2-biphenylethenyl)-1,1′-biphenyl), spiro-DPVBi (spiro-4,4′-bis(2,2-biphenylethenyl)-1,1′-biphenyl), LiPBO (2-(2-benzooxazolyl)-phenol lithium salt), bis(biphenylvinyl)benzene, an aluminum-quinoline metal complex, and metal complexes of imidazole, thiazole and oxazole, etc.

The electron injection layer or electron transport layer compound is not specifically limited, and any compound may be used as the electron injection layer or electron transport layer compound as long as it is generally used as the electron injection layer or electron transport layer compound. Non-limiting examples of the electron injection layer or electron transport layer compounds may include a pyridine derivative, a naphthalene derivative, a anthracene derivative, a phenanthroline derivative, a perinone derivative, a coumarin derivative, a naphthalimide derivative, a anthraquinone derivative, a diphenoquinone derivative, a diphenylquinone derivative, a perylene derivative, a oxadiazole derivative, a thiophene derivative, a triazole derivative, a thiadiazole derivative, a metal complex of an oxine derivative, a quinolinol-based metal complexe, a quinoxaline derivative, a polymer of the quinoxaline derivative, a benzazole compound, a gallium complex, a pyrazole derivative, a perfluorinated phenylene derivative, a triazine derivative, a pyrazine derivative, a benzoquinoline derivative, a imidazopyridine derivative, a borane derivative, a benzoimidazole derivative, a benzoxazole derivative, a benzothiazole derivative, a quinoline derivative, an oligopyridine derivative such as terpyridine, a bipyridine derivative, a terpyridine derivative, a naphthyridine derivative, a aldazine derivative, a carbazole derivative, an indole derivative, a phosphorus oxide derivative, a bisstyryl derivative, a quinolinol-based metal complex, a hydroxyazole-based metal complex, an azomethine-based metal complex, a tropolone-based metal complex, a flavonol-based metal complex, a benzoquinoline-based metal complex, metal salts, etc. The materials as described above may be used singly, or may also be used as mixtures with other materials. For example, non-limiting examples of the electron injection layer or electron transport layer compounds may include 2-(4-(9,10-di(naphthalen-2-yl) anthracen-2-yl)phenyl)-1-phenyl-1H-benzo[d]imidazole, tris(8-hydroxyquinolinato)aluminum (Alq₃), LiF, Liq, Li₂O, BaO, NaCl, and CsF.

The auxiliary electron transport layer may be formed between the electron transport layer and the light emitting layer. An auxiliary electron transport layer compound is not particularly limited. Any compound may be used as the auxiliary electron transport layer compound as long as it is commonly used as the auxiliary electron transport layer compound. For example, the auxiliary electron transport layer may include pyrimidine derivatives, etc.

The organic light emitting diode according to one embodiment of the present disclosure may be embodied as a top emission or bottom emission type light emitting diode.

The organic light emitting diode according to one embodiment of the present disclosure may be used as a light emitting element in a display device.

The organic light emitting diode according to one embodiment of the present disclosure may be applied, as a light emitting element, to a transparent display diode, a mobile display diode, a flexible display diode, etc. However, the present disclosure is not limited thereto.

Hereinafter, methods for synthesizing and preparing the above compounds and Experimental Examples thereon will be described based on representative Examples. However, the method of synthesis of the compounds of the present disclosure is not limited to the following examples. Further, the present disclosure is not limited to examples as set forth below.

### Synthesis Example

A final product of the present disclosure may be synthesized as shown in Reaction Formula 1 as set forth below. However, the present disclosure is not limited thereto.

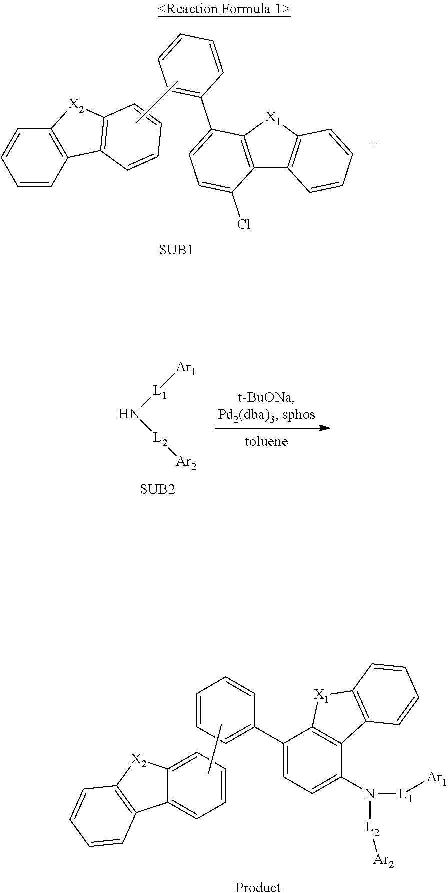

SUB 1 (53.95 mmol), SUB 2 (51.38 mmol), t-BuONa (102.76 mmol), Pd₂(dba)₃ (1.03 mmol), Sphos (2.06 mmol) and toluene as the reactants were added to a 500 mL flask under nitrogen flow and reacted with each other under stirring and refluxing. After completion of the reaction, an organic layer was extracted using toluene and water. The extracted solution was treated with MgSO₄ to remove remaining moisture therefrom, concentrated under a reduced pressure, purified using column chromatography, and then recrystallized to obtain a product.

According to the Reaction Formula 1, the Compounds as shown in Table 19 as set forth below were prepared, and the Compounds used in Table 19 refer to the same structures as those shown in Product.

TABLE 19

| Com- | | | | Yield | MS |
|---|---|---|---|---|---|
| pound | SUB 1 | SUB 2 | Product | (%) | [M + H]⁺ |
| A5 | 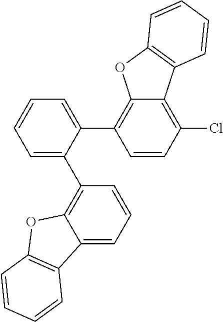 [SMILES: Clc1ccc(-c2ccccc2-c2cccc3c2oc2ccccc23)c2oc3ccccc3c12] | 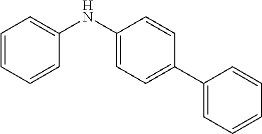 [SMILES: c1ccc(Nc2ccc(-c3ccccc3)cc2)cc1] | 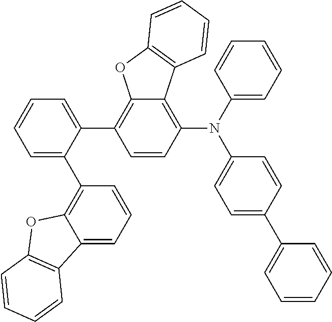 [SMILES: c1ccc(-c2ccc(N(c3ccccc3)c3ccc(-c4ccccc4-c4cccc5c4oc4ccccc45)c4oc5ccccc5c34)cc2)cc1] | 21.8 g (65%) | 653.78 |
| A266 | 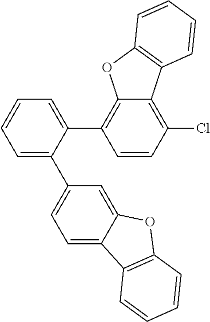 [SMILES: Clc1ccc(-c2ccccc2-c2ccc3c(c2)oc2ccccc23)c2oc3ccccc3c12] | 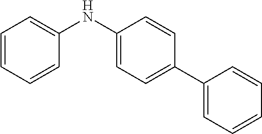 [SMILES: c1ccc(Nc2ccc(-c3ccccc3)cc2)cc1] | 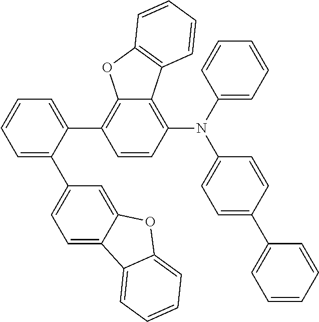 [SMILES: c1ccc(-c2ccc(N(c3ccccc3)c3ccc(-c4ccccc4-c4ccc5c(c4)oc4ccccc45)c4oc5ccccc5c34)cc2)cc1] | 20.2 g (60%) | 653.78 |
| A486 | 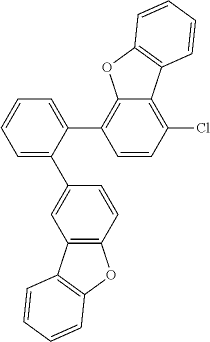 [SMILES: Clc1ccc(-c2ccccc2-c2ccc3oc4ccccc4c3c2)c2oc3ccccc3c12] | 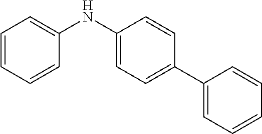 [SMILES: c1ccc(Nc2ccc(-c3ccccc3)cc2)cc1] | 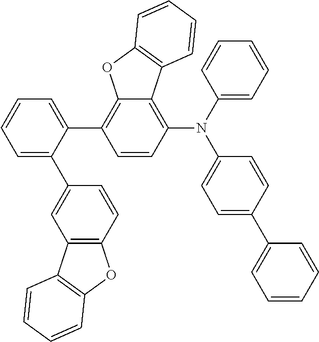 [SMILES: c1ccc(-c2ccc(N(c3ccccc3)c3ccc(-c4ccccc4-c4ccc5oc6ccccc6c5c4)c4oc5ccccc5c34)cc2)cc1] | 19.5 g (58%) | 653.78 |
| A652 | 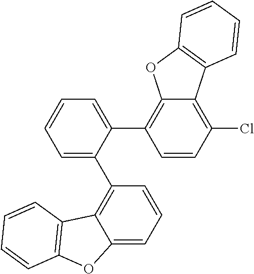 [SMILES: Clc1ccc(-c2ccccc2-c2cccc3oc4ccccc4c23)c2oc3ccccc3c12] | 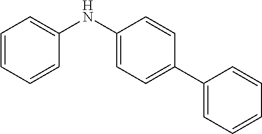 [SMILES: c1ccc(Nc2ccc(-c3ccccc3)cc2)cc1] | 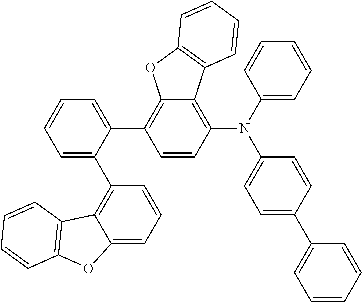 [SMILES: c1ccc(-c2ccc(N(c3ccccc3)c3ccc(-c4ccccc4-c4cccc5oc6ccccc6c45)c4oc5ccccc5c34)cc2)cc1] | 17.5 g (52%) | 653.78 |
| A60 | 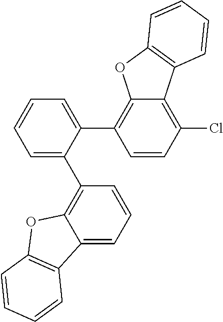 [SMILES: Clc1ccc(-c2ccccc2-c2cccc3c2oc2ccccc23)c2oc3ccccc3c12] | 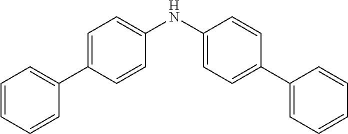 [SMILES: c1ccc(-c2ccc(Nc3ccc(-c4ccccc4)cc3)cc2)cc1] | 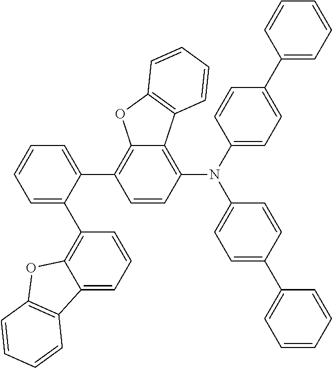 [SMILES: c1ccc(-c2ccc(N(c3ccc(-c4ccccc4)cc3)c3ccc(-c4ccccc4-c4cccc5c4oc4ccccc45)c4oc5ccccc5c34)cc2)cc1] | 25.5 g (68%) | 729.88 |
| A313 | 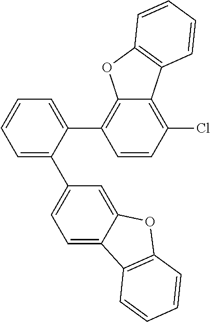 [SMILES: Clc1ccc(-c2ccccc2-c2ccc3c(c2)oc2ccccc23)c2oc3ccccc3c12] | 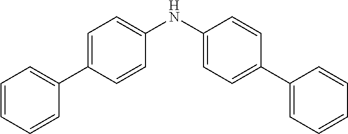 [SMILES: c1ccc(-c2ccc(Nc3ccc(-c4ccccc4)cc3)cc2)cc1] | 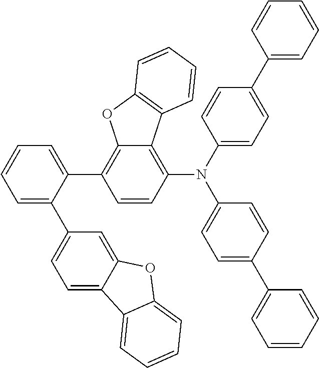 [SMILES: c1ccc(-c2ccc(N(c3ccc(-c4ccccc4)cc3)c3ccc(-c4ccccc4-c4ccc5c(c4)oc4ccccc45)c4oc5ccccc5c34)cc2)cc1] | 24.4 g (65%) | 729.88 |
| A521 | 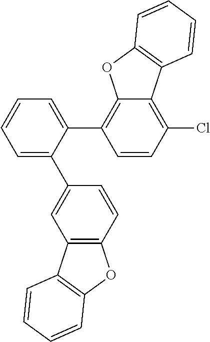 [SMILES: Clc1ccc(-c2ccccc2-c2ccc3oc4ccccc4c3c2)c2oc3ccccc3c12] | 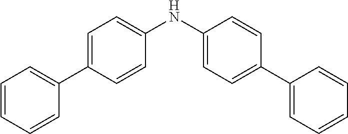 [SMILES: c1ccc(-c2ccc(Nc3ccc(-c4ccccc4)cc3)cc2)cc1] | 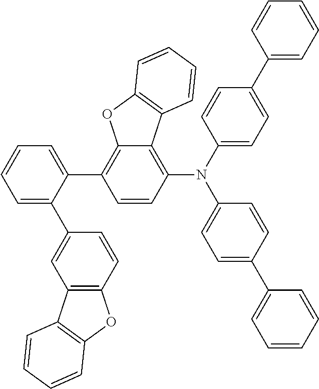 [SMILES: c1ccc(-c2ccc(N(c3ccc(-c4ccccc4)cc3)c3ccc(-c4ccccc4-c4ccc5oc6ccccc6c5c4)c4oc5ccccc5c34)cc2)cc1] | 22.5 g (60%) | 729.88 |
| A688 | 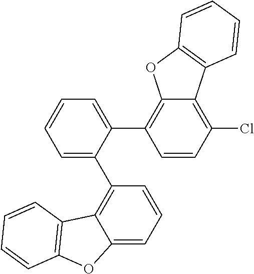 [SMILES: Clc1ccc(-c2ccccc2-c2cccc3oc4ccccc4c23)c2oc3ccccc3c12] | 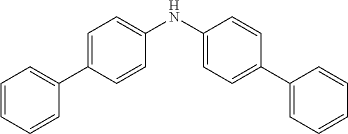 [SMILES: c1ccc(-c2ccc(Nc3ccc(-c4ccccc4)cc3)cc2)cc1] | 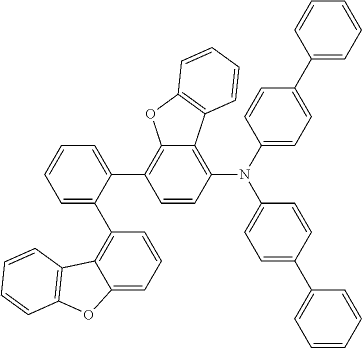 [SMILES: c1ccc(-c2ccc(N(c3ccc(-c4ccccc4)cc3)c3ccc(-c4ccccc4-c4cccc5oc6ccccc6c45)c4oc5ccccc5c34)cc2)cc1] | 20.6 g (55%) | 729.88 |
| A252 | 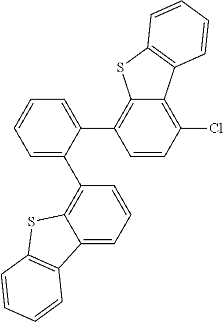 [SMILES: Clc1ccc(-c2ccccc2-c2cccc3c2sc2ccccc23)c2sc3ccccc3c12] | 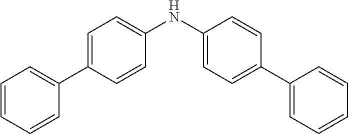 [SMILES: c1ccc(-c2ccc(Nc3ccc(-c4ccccc4)cc3)cc2)cc1] | 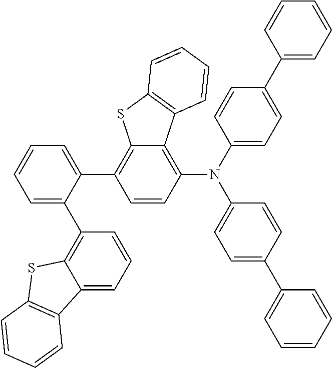 [SMILES: c1ccc(-c2ccc(N(c3ccc(-c4ccccc4)cc3)c3ccc(-c4ccccc4-c4cccc5c4sc4ccccc45)c4sc5ccccc5c34)cc2)cc1] | 25.8 g (66%) | 762.00 |
| A192 | 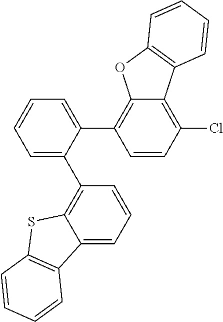 [SMILES: Clc1ccc(-c2ccccc2-c2cccc3c2sc2ccccc23)c2oc3ccccc3c12] | 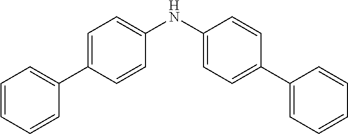 [SMILES: c1ccc(-c2ccc(Nc3ccc(-c4ccccc4)cc3)cc2)cc1] | 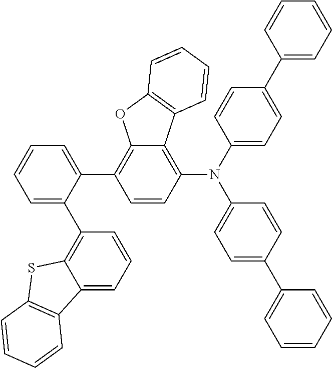 [SMILES: c1ccc(-c2ccc(N(c3ccc(-c4ccccc4)cc3)c3ccc(-c4ccccc4-c4cccc5c4sc4ccccc45)c4oc5ccccc5c34)cc2)cc1] | 23.8 g (62%) | 745.94 |
| A250 | 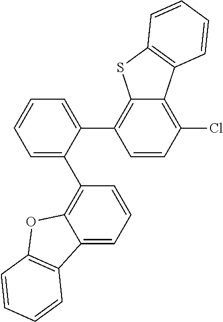 [SMILES: Clc1ccc(-c2ccccc2-c2cccc3c2oc2ccccc23)c2sc3ccccc3c12] | 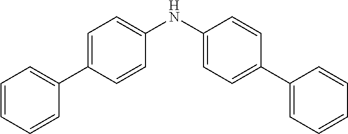 [SMILES: c1ccc(-c2ccc(Nc3ccc(-c4ccccc4)cc3)cc2)cc1] | 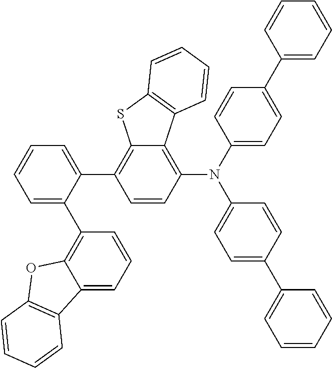 [SMILES: c1ccc(-c2ccc(N(c3ccc(-c4ccccc4)cc3)c3ccc(-c4ccccc4-c4cccc5c4oc4ccccc45)c4sc5ccccc5c34)cc2)cc1] | 23.0 g (60%) | 745.94 |
| A63 | 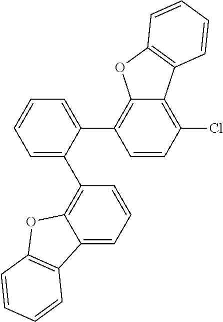 [SMILES: Clc1ccc(-c2ccccc2-c2cccc3c2oc2ccccc23)c2oc3ccccc3c12] | 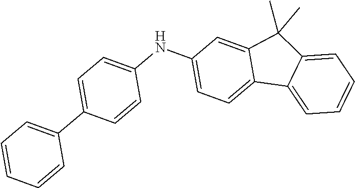 [SMILES: CC1(C)c2ccccc2-c2ccc(Nc3ccc(-c4ccccc4)cc3)cc21] | 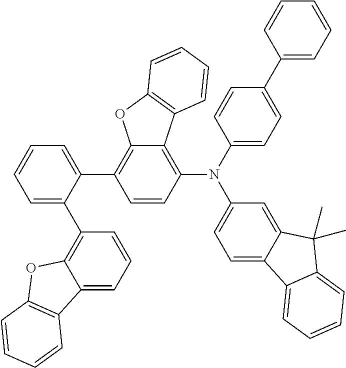 [SMILES: CC1(C)c2ccccc2-c2ccc(N(c3ccc(-c4ccccc4)cc3)c3ccc(-c4ccccc4-c4cccc5c4oc4ccccc45)c4oc5ccccc5c34)cc21] | 25.7 g (65%) | 769.94 |
| A304 | 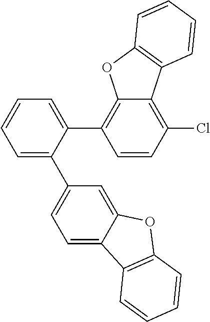 [SMILES: Clc1ccc(-c2ccccc2-c2ccc3c(c2)oc2ccccc23)c2oc3ccccc3c12] | 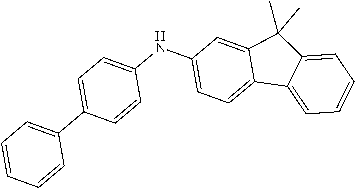 [SMILES: CC1(C)c2ccccc2-c2ccc(Nc3ccc(-c4ccccc4)cc3)cc21] | 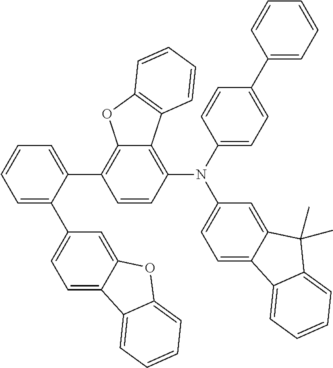 [SMILES: CC1(C)c2ccccc2-c2ccc(N(c3ccc(-c4ccccc4)cc3)c3ccc(-c4ccccc4-c4ccc5c(c4)oc4ccccc45)c4oc5ccccc5c34)cc21] | 24.5 g (62%) | 769.94 |
| A514 | 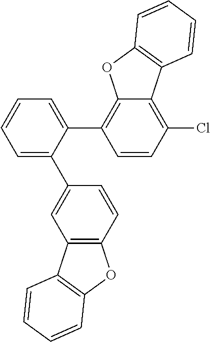 [SMILES: Clc1ccc(-c2ccccc2-c2ccc3oc4ccccc4c3c2)c2oc3ccccc3c12] | 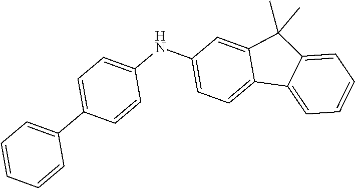 [SMILES: CC1(C)c2ccccc2-c2ccc(Nc3ccc(-c4ccccc4)cc3)cc21] | 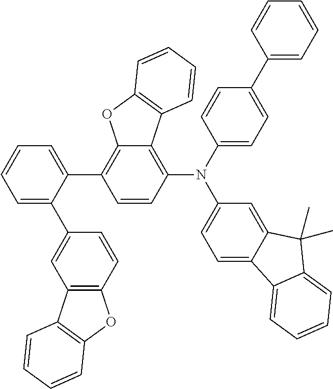 [SMILES: CC1(C)c2ccccc2-c2ccc(N(c3ccc(-c4ccccc4)cc3)c3ccc(-c4ccccc4-c4ccc5oc6ccccc6c5c4)c4oc5ccccc5c34)cc21] | 23.7 g (60%) | 769.94 |
| A681 | 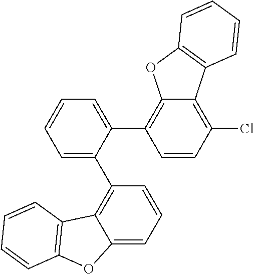 [SMILES: Clc1ccc(-c2ccccc2-c2cccc3oc4ccccc4c23)c2oc3ccccc3c12] | 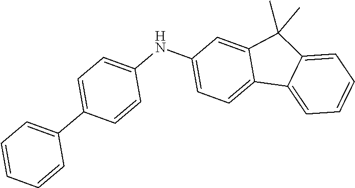 [SMILES: CC1(C)c2ccccc2-c2ccc(Nc3ccc(-c4ccccc4)cc3)cc21] | 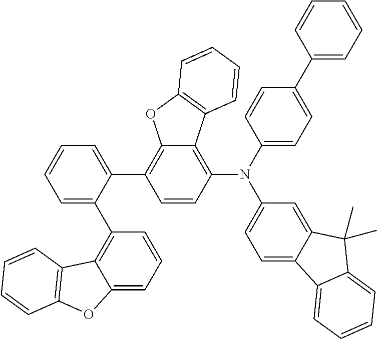 [SMILES: CC1(C)c2ccccc2-c2ccc(N(c3ccc(-c4ccccc4)cc3)c3ccc(-c4ccccc4-c4cccc5oc6ccccc6c45)c4oc5ccccc5c34)cc21] | 22.6 g (57%) | 769.94 |
| A8 | 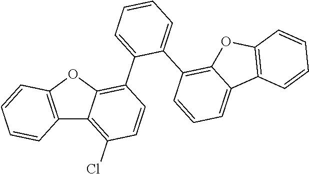 [SMILES: Clc1ccc(-c2ccccc2-c2cccc3c2oc2ccccc23)c2oc3ccccc3c12] | 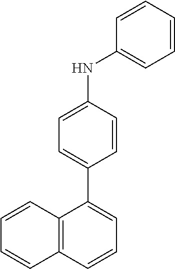 [SMILES: c1ccc(Nc2ccc(-c3cccc4ccccc34)cc2)cc1] | 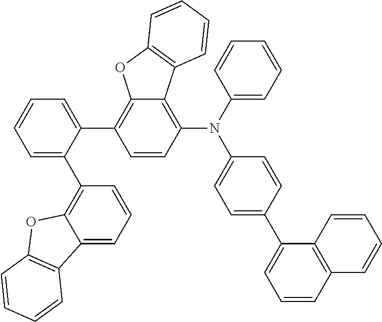 [SMILES: c1ccc(N(c2ccc(-c3cccc4ccccc34)cc2)c2ccc(-c3ccccc3-c3cccc4c3oc3ccccc34)c3oc4ccccc4c23)cc1] | 24.1 g (61%) | 703.84 |
| A39 | 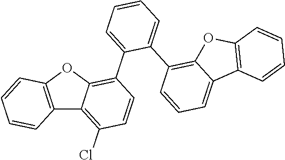 [SMILES: Clc1ccc(-c2ccccc2-c2cccc3c2oc2ccccc23)c2oc3ccccc3c12] | 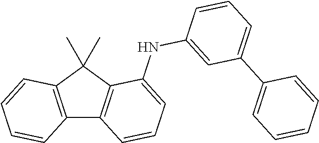 [SMILES: CC1(C)c2ccccc2-c2cccc(Nc3cccc(-c4ccccc4)c3)c21] | 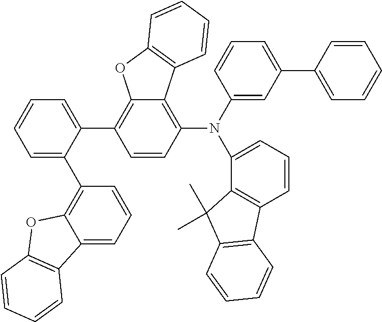 [SMILES: CC1(C)c2ccccc2-c2cccc(N(c3cccc(-c4ccccc4)c3)c3ccc(-c4ccccc4-c4cccc5c4oc4ccccc45)c4oc5ccccc5c34)c21] | 21.4 g (54%) | 769.94 |
| A65 | 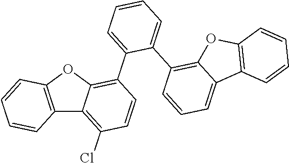 [SMILES: Clc1ccc(-c2ccccc2-c2cccc3c2oc2ccccc23)c2oc3ccccc3c12] | 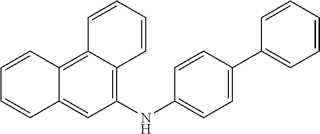 [SMILES: c1ccc(-c2ccc(Nc3cc4ccccc4c4ccccc34)cc2)cc1] | 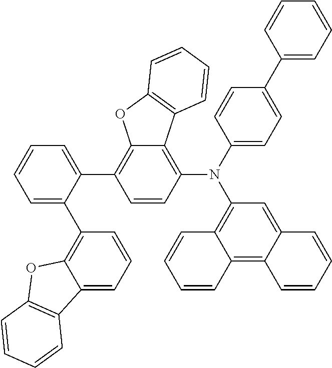 [SMILES: c1ccc(-c2ccc(N(c3cc4ccccc4c4ccccc34)c3ccc(-c4ccccc4-c4cccc5c4oc4ccccc45)c4oc5ccccc5c34)cc2)cc1] | 22.9 g (59%) | 753.90 |
| A79 | 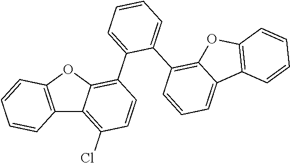 [SMILES: Clc1ccc(-c2ccccc2-c2cccc3c2oc2ccccc23)c2oc3ccccc3c12] | 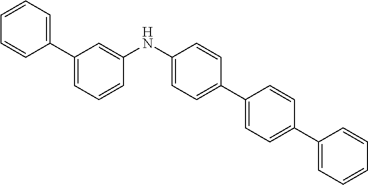 [SMILES: c1ccc(-c2ccc(-c3ccc(Nc4cccc(-c5ccccc5)c4)cc3)cc2)cc1] | 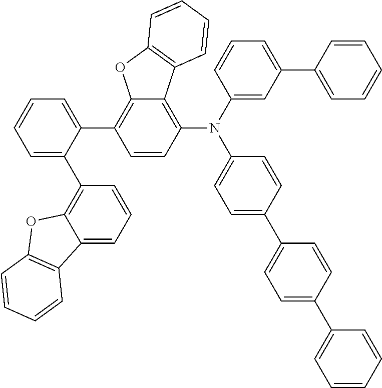 [SMILES: c1ccc(-c2ccc(-c3ccc(N(c4cccc(-c5ccccc5)c4)c4ccc(-c5ccccc5-c5cccc6c5oc5ccccc56)c5oc6ccccc6c45)cc3)cc2)cc1] | 21.5 g (52%) | 805.98 |
| A268 | 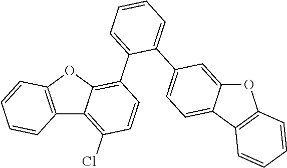 [SMILES: Clc1ccc(-c2ccccc2-c2ccc3c(c2)oc2ccccc23)c2oc3ccccc3c12] | 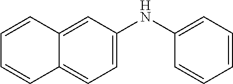 [SMILES: c1ccc(Nc2ccc3ccccc3c2)cc1] | 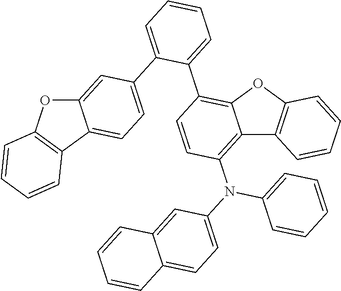 [SMILES: c1ccc(N(c2ccc3ccccc3c2)c2ccc(-c3ccccc3-c3ccc4c(c3)oc3ccccc34)c3oc4ccccc4c23)cc1] | 21.3 g (66%) | 627.74 |
| A325 | 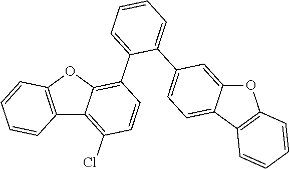 [SMILES: Clc1ccc(-c2ccccc2-c2ccc3c(c2)oc2ccccc23)c2oc3ccccc3c12] | 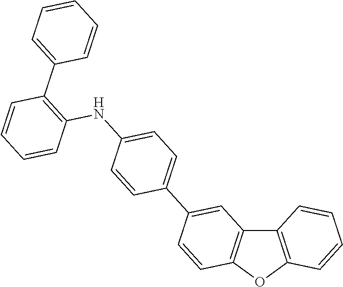 [SMILES: c1ccc(-c2ccccc2Nc2ccc(-c3ccc4oc5ccccc5c4c3)cc2)cc1] | 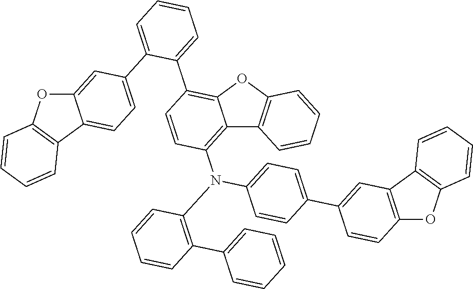 [SMILES: c1ccc(-c2ccccc2N(c2ccc(-c3ccc4oc5ccccc5c4c3)cc2)c2ccc(-c3ccccc3-c3ccc4c(c3)oc3ccccc34)c3oc4ccccc4c23)cc1] | 21.5 g (51%) | 819.96 |
| A488 | 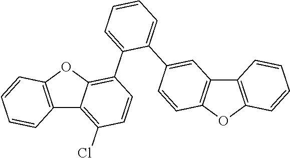 [SMILES: Clc1ccc(-c2ccccc2-c2ccc3oc4ccccc4c3c2)c2oc3ccccc3c12] | 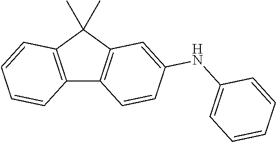 [SMILES: CC1(C)c2ccccc2-c2ccc(Nc3ccccc3)cc21] | 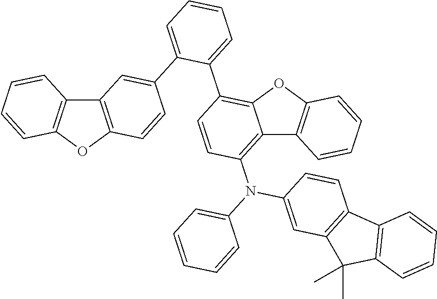 [SMILES: CC1(C)c2ccccc2-c2ccc(N(c3ccccc3)c3ccc(-c4ccccc4-c4ccc5oc6ccccc6c5c4)c4oc5ccccc5c34)cc21] | 19.7 g (56%) | 693.85 |
| A664 | 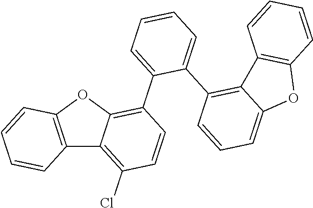 [SMILES: Clc1ccc(-c2ccccc2-c2cccc3oc4ccccc4c23)c2oc3ccccc3c12] | 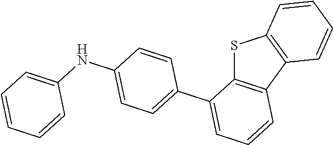 [SMILES: c1ccc(Nc2ccc(-c3cccc4c3sc3ccccc34)cc2)cc1] | 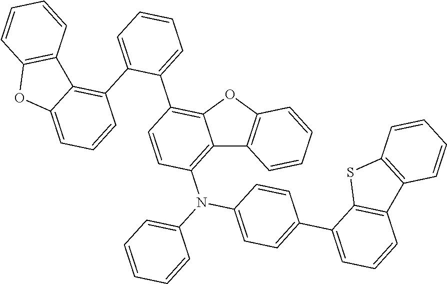 [SMILES: c1ccc(N(c2ccc(-c3cccc4c3sc3ccccc34)cc2)c2ccc(-c3ccccc3-c3cccc4oc5ccccc5c34)c3oc4ccccc4c23)cc1] | 21.5 g (55%) | 759.92 |
| B5 | 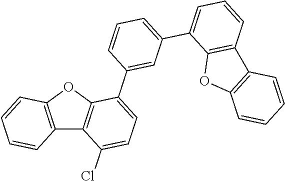 [SMILES: Clc1ccc(-c2cccc(-c3cccc4c3oc3ccccc34)c2)c2oc3ccccc3c12] | 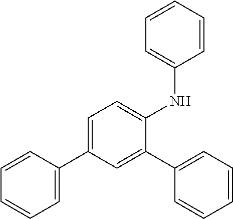 [SMILES: c1ccc(Nc2ccc(-c3ccccc3)cc2-c2ccccc2)cc1] | 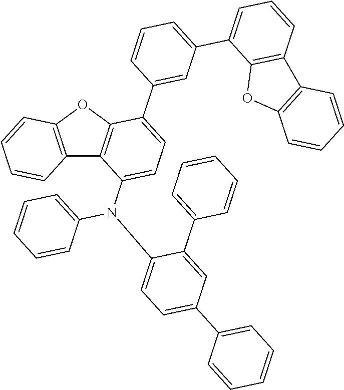 [SMILES: c1ccc(-c2ccc(N(c3ccccc3)c3ccc(-c4cccc(-c5cccc6c5oc5ccccc56)c4)c4oc5ccccc5c34)c(-c3ccccc3)c2)cc1] | 22.5 g (60%) | 729.88 |
| B17 | 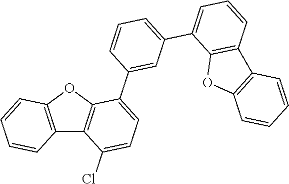 [SMILES: Clc1ccc(-c2cccc(-c3cccc4c3oc3ccccc34)c2)c2oc3ccccc3c12] | 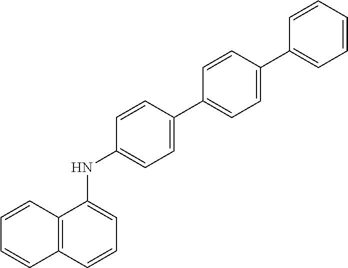 [SMILES: c1ccc(-c2ccc(-c3ccc(Nc4cccc5ccccc45)cc3)cc2)cc1] | 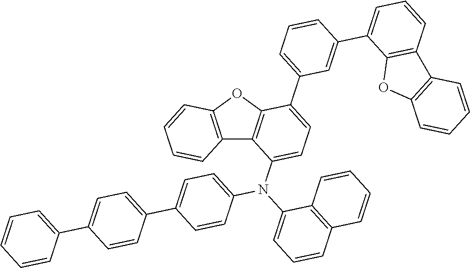 [SMILES: c1ccc(-c2ccc(-c3ccc(N(c4cccc5ccccc45)c4ccc(-c5cccc(-c6cccc7c6oc6ccccc67)c5)c5oc6ccccc6c45)cc3)cc2)cc1] | 23.2 g (58%) | 779.94 |
| B93 | 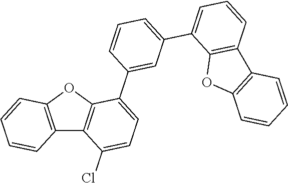 [SMILES: Clc1ccc(-c2cccc(-c3cccc4c3oc3ccccc34)c2)c2oc3ccccc3c12] | 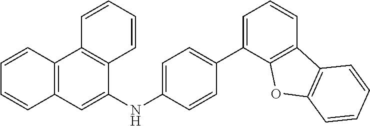 [SMILES: c1ccc2c(c1)cc(Nc1ccc(-c3cccc4c3oc3ccccc34)cc1)c1ccccc12] | 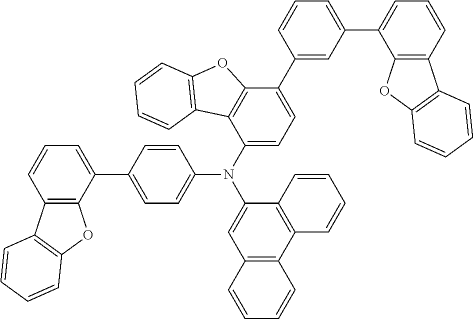 [SMILES: c1cc(-c2cccc3c2oc2ccccc23)cc(-c2ccc(N(c3ccc(-c4cccc5c4oc4ccccc45)cc3)c3cc4ccccc4c4ccccc34)c3c2oc2ccccc23)c1] | 26.9 g (62%) | 843.98 |
| B209 | 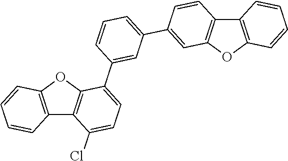 [SMILES: Clc1ccc(-c2cccc(-c3ccc4c(c3)oc3ccccc34)c2)c2oc3ccccc3c12] | 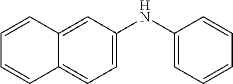 [SMILES: c1ccc(Nc2ccc3ccccc3c2)cc1] | 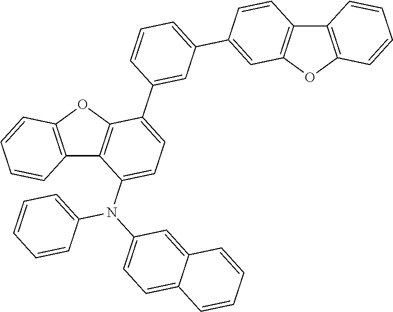 [SMILES: c1ccc(N(c2ccc3ccccc3c2)c2ccc(-c3cccc(-c4ccc5c(c4)oc4ccccc45)c3)c3oc4ccccc4c23)cc1] | 21.6 g (67%) | 627.74 |
| B211 | 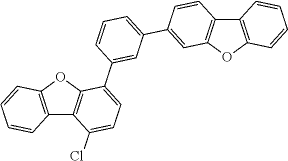 [SMILES: Clc1ccc(-c2cccc(-c3ccc4c(c3)oc3ccccc34)c2)c2oc3ccccc3c12] | 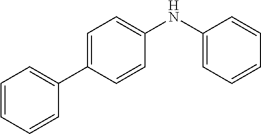 [SMILES: c1ccc(Nc2ccc(-c3ccccc3)cc2)cc1] | 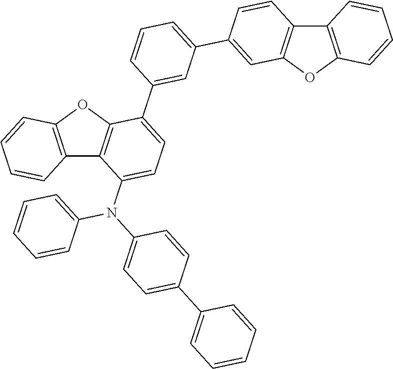 [SMILES: c1ccc(-c2ccc(N(c3ccccc3)c3ccc(-c4cccc(-c5ccc6c(c5)oc5ccccc56)c4)c4oc5ccccc5c34)cc2)cc1] | 21.2 g (63%) | 653.78 |
| B257 | 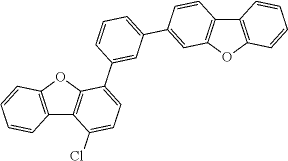 [SMILES: Clc1ccc(-c2cccc(-c3ccc4c(c3)oc3ccccc34)c2)c2oc3ccccc3c12] | 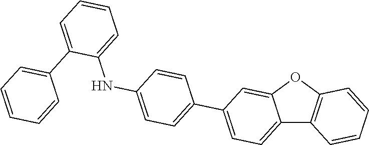 [SMILES: c1ccc(-c2ccccc2Nc2ccc(-c3ccc4c(c3)oc3ccccc34)cc2)cc1] | 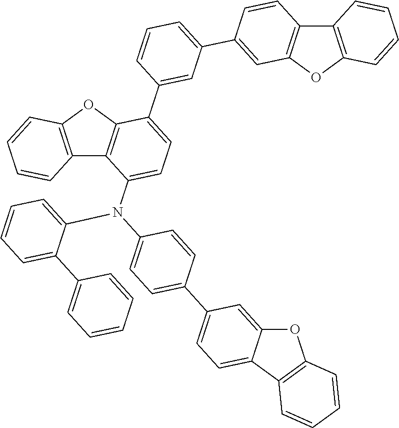 [SMILES: c1ccc(-c2ccccc2N(c2ccc(-c3ccc4c(c3)oc3ccccc34)cc2)c2ccc(-c3cccc(-c4ccc5c(c4)oc4ccccc45)c3)c3oc4ccccc4c23)cc1] | 22.7 g (54%) | 819.96 |
| B229 | 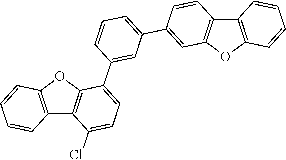 [SMILES: Clc1ccc(-c2cccc(-c3ccc4c(c3)oc3ccccc34)c2)c2oc3ccccc3c12] | 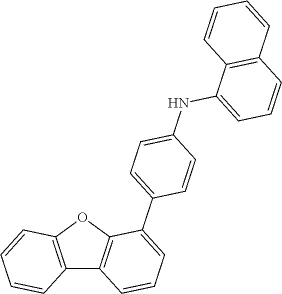 [SMILES: c1ccc2c(Nc3ccc(-c4cccc5c4oc4ccccc45)cc3)cccc2c1] | 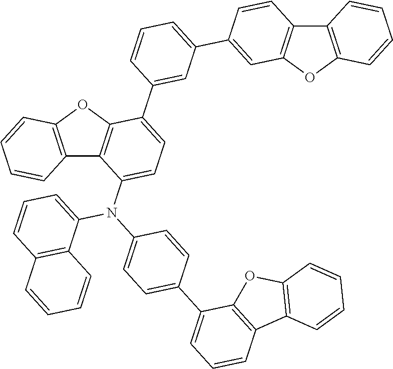 [SMILES: c1cc(-c2ccc3c(c2)oc2ccccc23)cc(-c2ccc(N(c3ccc(-c4cccc5c4oc4ccccc45)cc3)c3cccc4ccccc34)c3c2oc2ccccc23)c1] | 26.1 g (64%) | 793.92 |
| B398 | 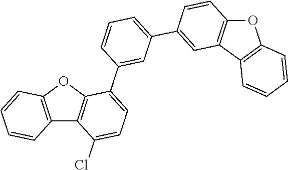 [SMILES: Clc1ccc(-c2cccc(-c3ccc4oc5ccccc5c4c3)c2)c2oc3ccccc3c12] | 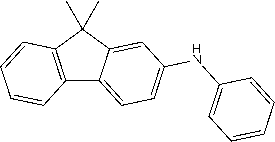 [SMILES: CC1(C)c2ccccc2-c2ccc(Nc3ccccc3)cc21] | 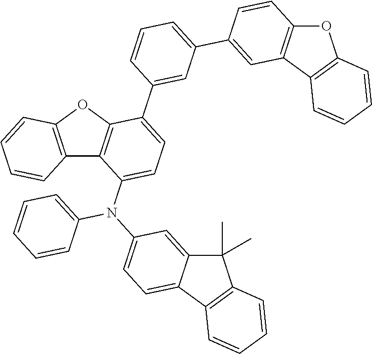 [SMILES: CC1(C)c2ccccc2-c2ccc(N(c3ccccc3)c3ccc(-c4cccc(-c5ccc6oc7ccccc7c6c5)c4)c4oc5ccccc5c34)cc21] | 25.0 g (70%) | 693.85 |
| B403 | 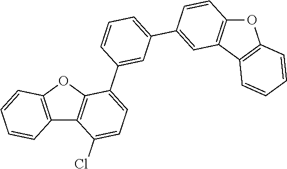 [SMILES: Clc1ccc(-c2cccc(-c3ccc4oc5ccccc5c4c3)c2)c2oc3ccccc3c12] | 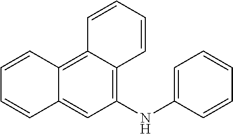 [SMILES: c1ccc(Nc2cc3ccccc3c3ccccc23)cc1] | 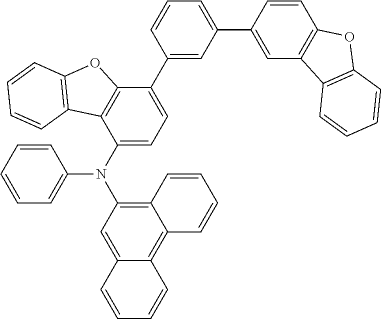 [SMILES: c1ccc(N(c2cc3ccccc3c3ccccc23)c2ccc(-c3cccc(-c4ccc5oc6ccccc6c5c4)c3)c3oc4ccccc4c23)cc1] | 22.6 g (65%) | 677.8 |
| B431 | 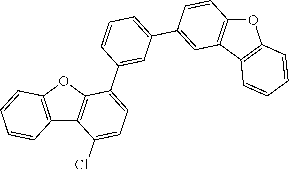 [SMILES: Clc1ccc(-c2cccc(-c3ccc4oc5ccccc5c4c3)c2)c2oc3ccccc3c12] | 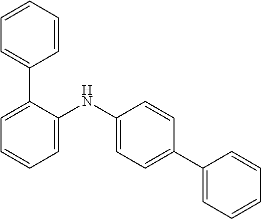 [SMILES: c1ccc(-c2ccc(Nc3ccccc3-c3ccccc3)cc2)cc1] | 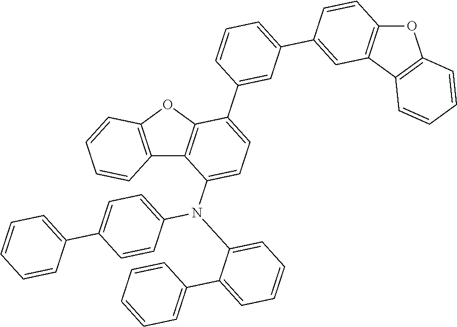 [SMILES: c1ccc(-c2ccc(N(c3ccccc3-c3ccccc3)c3ccc(-c4cccc(-c5ccc6oc7ccccc7c6c5)c4)c4oc5ccccc5c34)cc2)cc1] | 22.5 g (60%) | 729.88 |
| B550 | 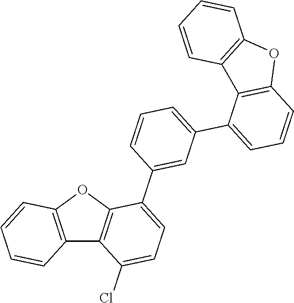 [SMILES: Clc1ccc(-c2cccc(-c3cccc4oc5ccccc5c34)c2)c2oc3ccccc3c12] | 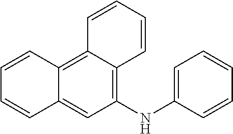 [SMILES: c1ccc(Nc2cc3ccccc3c3ccccc23)cc1] | 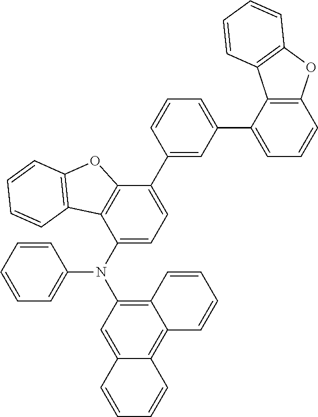 [SMILES: c1ccc(N(c2cc3ccccc3c3ccccc23)c2ccc(-c3cccc(-c4cccc5oc6ccccc6c45)c3)c3oc4ccccc4c23)cc1] | 18.1 g (52%) | 677.80 |
| B562 | 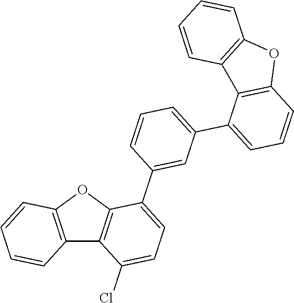 [SMILES: Clc1ccc(-c2cccc(-c3cccc4oc5ccccc5c34)c2)c2oc3ccccc3c12] | 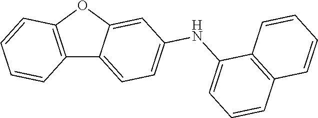 [SMILES: c1ccc2c(Nc3ccc4c(c3)oc3ccccc34)cccc2c1] | 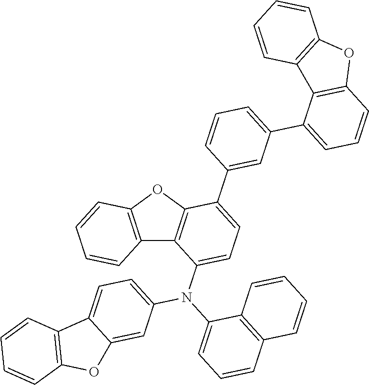 [SMILES: c1cc(-c2ccc(N(c3ccc4c(c3)oc3ccccc34)c3cccc4ccccc34)c3c2oc2ccccc23)cc(-c2cccc3oc4ccccc4c23)c1] | 25.4 g (69%) | 717.82 |
| B587 | 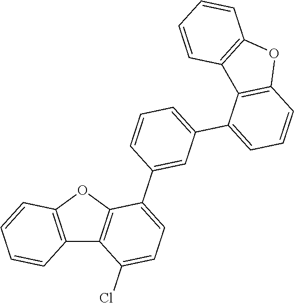 [SMILES: Clc1ccc(-c2cccc(-c3cccc4oc5ccccc5c34)c2)c2oc3ccccc3c12] | 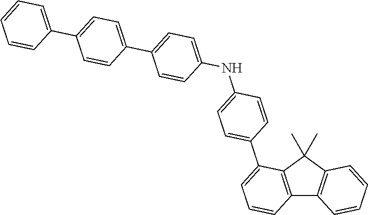 [SMILES: CC1(C)c2ccccc2-c2cccc(-c3ccc(Nc4ccc(-c5ccc(-c6ccccc6)cc5)cc4)cc3)c21] | 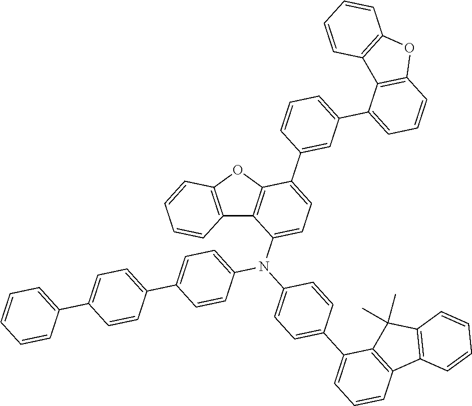 [SMILES: CC1(C)c2ccccc2-c2cccc(-c3ccc(N(c4ccc(-c5ccc(-c6ccccc6)cc5)cc4)c4ccc(-c5cccc(-c6cccc7oc8ccccc8c67)c5)c5oc6ccccc6c45)cc3)c21] | 27.0 g (57%) | 922.14 |
| A248 | 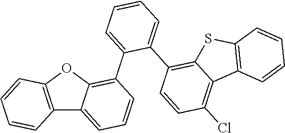 [SMILES: Clc1ccc(-c2ccccc2-c2cccc3c2oc2ccccc23)c2sc3ccccc3c12] | 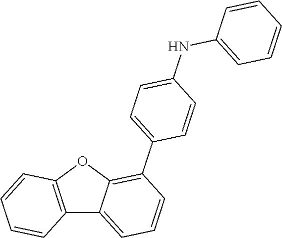 [SMILES: c1ccc(Nc2ccc(-c3cccc4c3oc3ccccc34)cc2)cc1] | 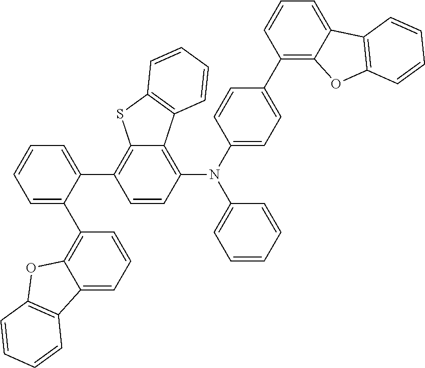 [SMILES: c1ccc(N(c2ccc(-c3cccc4c3oc3ccccc34)cc2)c2ccc(-c3ccccc3-c3cccc4c3oc3ccccc34)c3sc4ccccc4c23)cc1] | 23.0 g (59%) | 759.92 |
| A642 | 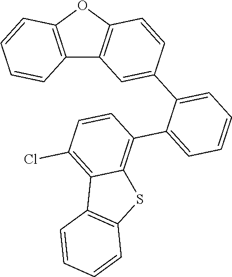 [SMILES: Clc1ccc(-c2ccccc2-c2ccc3oc4ccccc4c3c2)c2sc3ccccc3c12] | 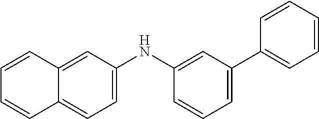 [SMILES: c1ccc(-c2cccc(Nc3ccc4ccccc4c3)c2)cc1] | 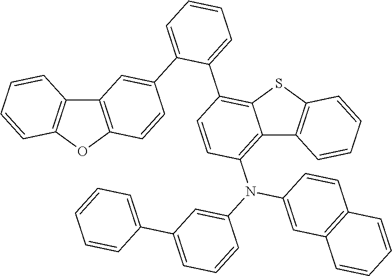 [SMILES: c1ccc(-c2cccc(N(c3ccc4ccccc4c3)c3ccc(-c4ccccc4-c4ccc5oc6ccccc6c5c4)c4sc5ccccc5c34)c2)cc1] | 25.0 g (64%) | 719.90 |
| B199 | 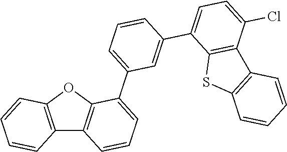 [SMILES: Clc1ccc(-c2cccc(-c3cccc4c3oc3ccccc34)c2)c2sc3ccccc3c12] | 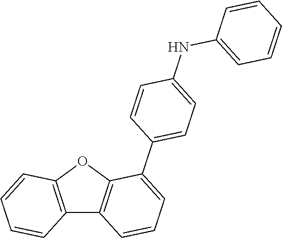 [SMILES: c1ccc(Nc2ccc(-c3cccc4c3oc3ccccc34)cc2)cc1] | 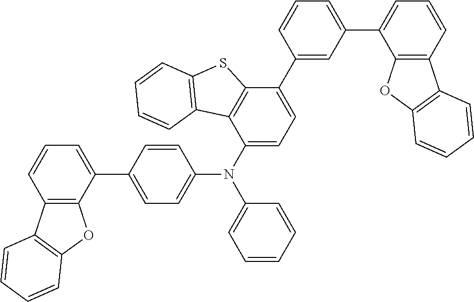 [SMILES: c1ccc(N(c2ccc(-c3cccc4c3oc3ccccc34)cc2)c2ccc(-c3cccc(-c4cccc5c4oc4ccccc45)c3)c3sc4ccccc4c23)cc1] | 22.6 g (58%) | 759.92 |
| B388 | 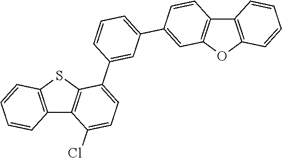 [SMILES: Clc1ccc(-c2cccc(-c3ccc4c(c3)oc3ccccc34)c2)c2sc3ccccc3c12] | 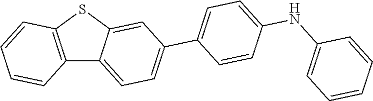 [SMILES: c1ccc(Nc2ccc(-c3ccc4c(c3)sc3ccccc34)cc2)cc1] | 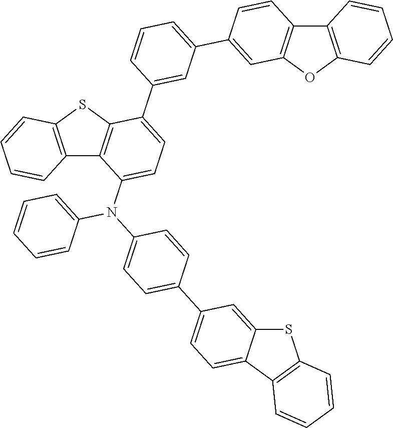 [SMILES: c1ccc(N(c2ccc(-c3ccc4c(c3)sc3ccccc34)cc2)c2ccc(-c3cccc(-c4ccc5c(c4)oc4ccccc45)c3)c3sc4ccccc4c23)cc1] | 25.1 g (64%) | 775.98 |
| A162 | 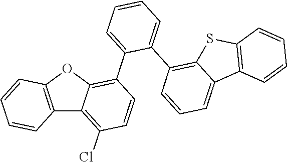 [SMILES: Clc1ccc(-c2ccccc2-c2cccc3c2sc2ccccc23)c2oc3ccccc3c12] | 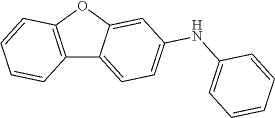 [SMILES: c1ccc(Nc2ccc3c(c2)oc2ccccc23)cc1] | 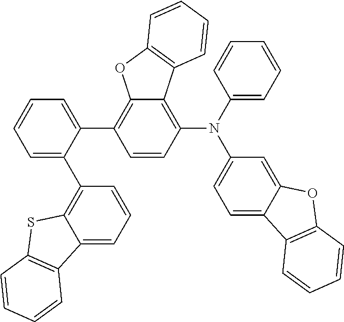 [SMILES: c1ccc(N(c2ccc3c(c2)oc2ccccc23)c2ccc(-c3ccccc3-c3cccc4c3sc3ccccc34)c3oc4ccccc4c23)cc1] | 21.1 g (60%) | 683.83 |
| B120 | 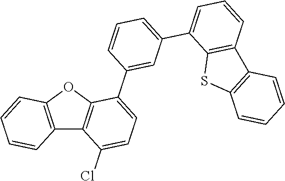 [SMILES: Clc1ccc(-c2cccc(-c3cccc4c3sc3ccccc34)c2)c2oc3ccccc3c12] | 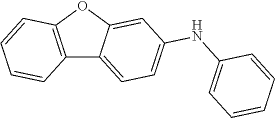 [SMILES: c1ccc(Nc2ccc3c(c2)oc2ccccc23)cc1] | 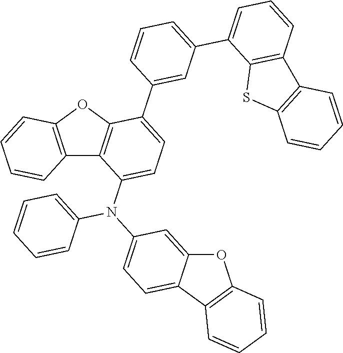 [SMILES: c1ccc(N(c2ccc3c(c2)oc2ccccc23)c2ccc(-c3cccc(-c4cccc5c4sc4ccccc45)c3)c3oc4ccccc4c23)cc1] | 22.1 g (63%) | 683.83 |
| B390 | 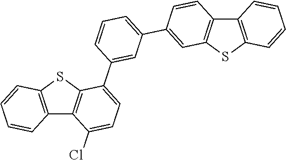 [SMILES: Clc1ccc(-c2cccc(-c3ccc4c(c3)sc3ccccc34)c2)c2sc3ccccc3c12] | 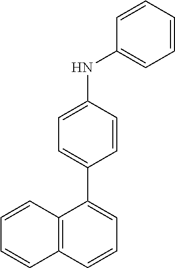 [SMILES: c1ccc(Nc2ccc(-c3cccc4ccccc34)cc2)cc1] | 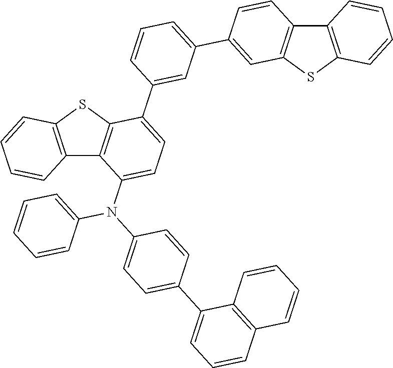 [SMILES: c1ccc(N(c2ccc(-c3cccc4ccccc34)cc2)c2ccc(-c3cccc(-c4ccc5c(c4)sc4ccccc45)c3)c3sc4ccccc4c23)cc1] | 22.7 g (60%) | 735.96 |
| A255 | 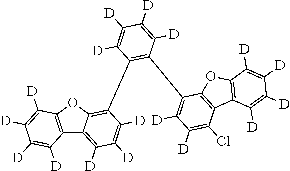 [SMILES: [2H]c1c([2H])c([2H])c(-c2c([2H])c([2H])c(Cl)c3c2oc2c([2H])c([2H])c([2H])c([2H])c23)c(-c2c([2H])c([2H])c([2H])c3c2oc2c([2H])c([2H])c([2H])c([2H])c23)c1[2H]] | 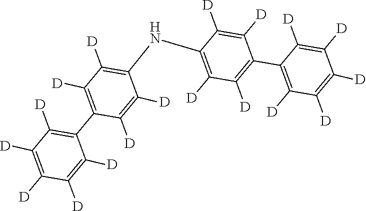 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | 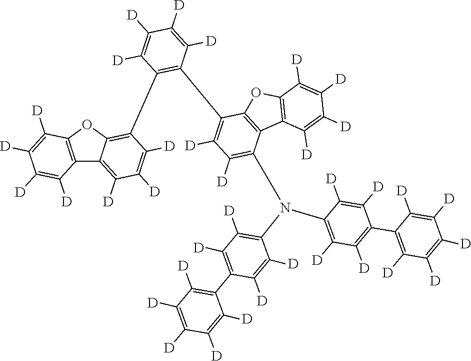 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c(-c4c([2H])c([2H])c([2H])c([2H])c4-c4c([2H])c([2H])c([2H])c5c4oc4c([2H])c([2H])c([2H])c([2H])c45)c4oc5c([2H])c([2H])c([2H])c([2H])c5c34)c([2H])c2[2H])c([2H])c1[2H]] | 25.9 g (66%) | 765.09 |
| A258 | 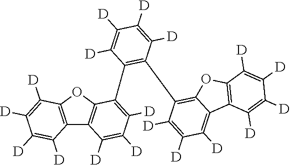 [SMILES: [2H]c1c([2H])c([2H])c(-c2c([2H])c([2H])c([2H])c3c2oc2c([2H])c([2H])c([2H])c([2H])c23)c(-c2c([2H])c([2H])c([2H])c3c2oc2c([2H])c([2H])c([2H])c([2H])c23)c1[2H]] | 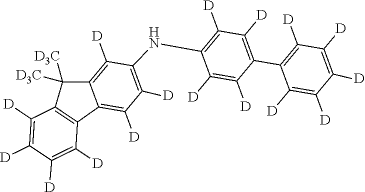 [SMILES: [2H]c1c([2H])c([2H])c(-c2c([2H])c([2H])c(Nc3c([2H])c([2H])c4c(c3[2H])C(C([2H])([2H])[2H])(C([2H])([2H])[2H])c3c([2H])c([2H])c([2H])c([2H])c3-4)c([2H])c2[2H])c([2H])c1[2H]] | 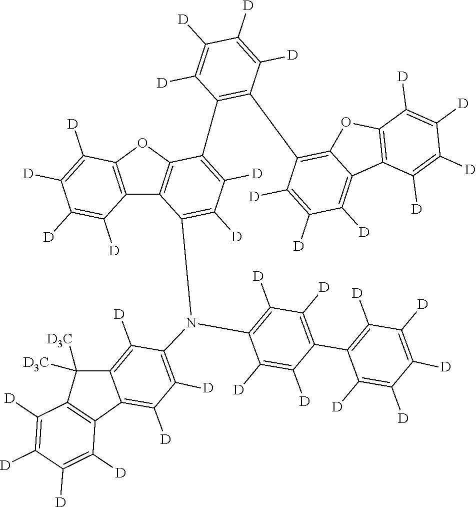 [SMILES: [2H]c1c([2H])c([2H])c(-c2c([2H])c([2H])c(N(c3c([2H])c([2H])c4c(c3[2H])C(C([2H])([2H])[2H])(C([2H])([2H])[2H])c3c([2H])c([2H])c([2H])c([2H])c3-4)c3c([2H])c([2H])c(-c4c([2H])c([2H])c([2H])c([2H])c4-c4c([2H])c([2H])c([2H])c5c4oc4c([2H])c([2H])c([2H])c([2H])c45)c4oc5c([2H])c([2H])c([2H])c([2H])c5c34)c([2H])c2[2H])c([2H])c1[2H]] | 27.9 g (67%) | 809.18 |
| B203 | 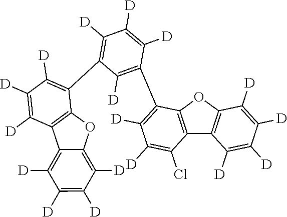 [SMILES: [2H]c1c([2H])c(-c2c([2H])c([2H])c([2H])c3c2oc2c([2H])c([2H])c([2H])c([2H])c23)c([2H])c(-c2c([2H])c([2H])c(Cl)c3c2oc2c([2H])c([2H])c([2H])c([2H])c23)c1[2H]] | 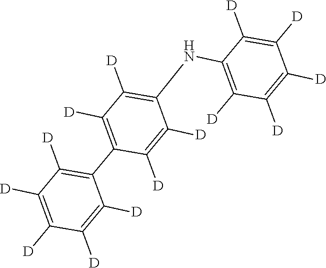 [SMILES: [2H]c1c([2H])c([2H])c(Nc2c([2H])c([2H])c(-c3c([2H])c([2H])c([2H])c([2H])c3[2H])c([2H])c2[2H])c([2H])c1[2H]] | 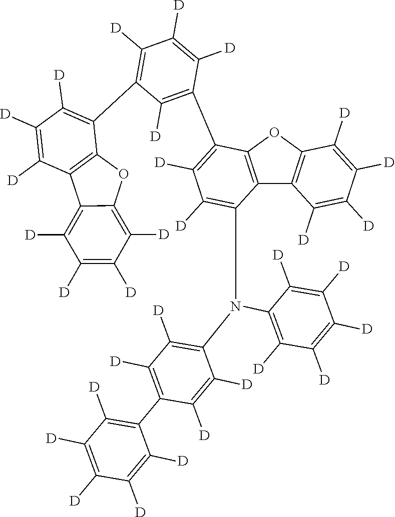 [SMILES: [2H]c1c([2H])c([2H])c(-c2c([2H])c([2H])c(N(c3c([2H])c([2H])c([2H])c([2H])c3[2H])c3c([2H])c([2H])c(-c4c([2H])c([2H])c([2H])c(-c5c([2H])c([2H])c([2H])c6c5oc5c([2H])c([2H])c([2H])c([2H])c56)c4[2H])c4oc5c([2H])c([2H])c([2H])c([2H])c5c34)c([2H])c2[2H])c([2H])c1[2H]] | 23.6 g (67%) | 684.97 |
| B391 | 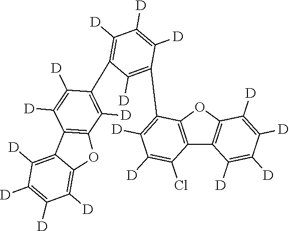 [SMILES: [2H]c1c([2H])c(-c2c([2H])c([2H])c3c(oc4c([2H])c([2H])c([2H])c([2H])c43)c2[2H])c([2H])c(-c2c([2H])c([2H])c(Cl)c3c2oc2c([2H])c([2H])c([2H])c([2H])c23)c1[2H]] | 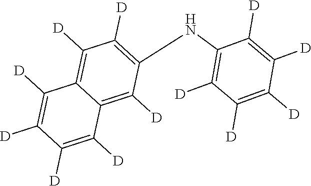 [SMILES: [2H]c1c([2H])c([2H])c(Nc2c([2H])c([2H])c3c([2H])c([2H])c([2H])c([2H])c3c2[2H])c([2H])c1[2H]] | 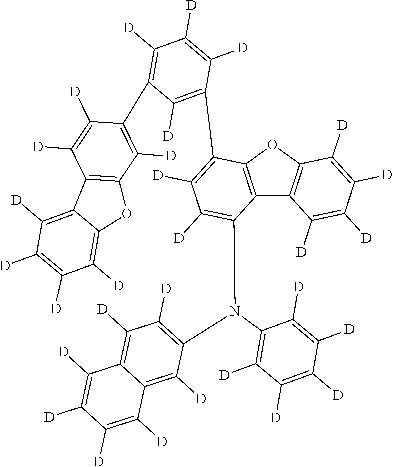 [SMILES: [2H]c1c([2H])c([2H])c(N(c2c([2H])c([2H])c3c([2H])c([2H])c([2H])c([2H])c3c2[2H])c2c([2H])c([2H])c(-c3c([2H])c([2H])c([2H])c(-c4c([2H])c([2H])c5c(oc6c([2H])c([2H])c([2H])c([2H])c65)c4[2H])c3[2H])c3oc4c([2H])c([2H])c([2H])c([2H])c4c23)c([2H])c1[2H]] | 19.9 g (59%) | 656.92 |
| A259 | 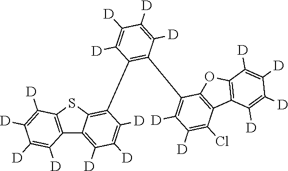 [SMILES: [2H]c1c([2H])c([2H])c(-c2c([2H])c([2H])c([2H])c3c2sc2c([2H])c([2H])c([2H])c([2H])c23)c(-c2c([2H])c([2H])c(Cl)c3c2oc2c([2H])c([2H])c([2H])c([2H])c23)c1[2H]] | 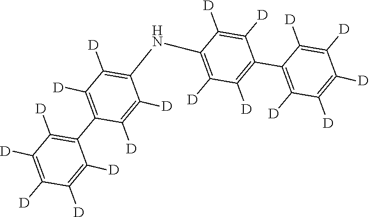 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | 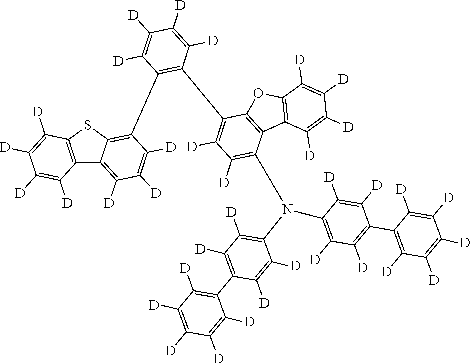 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c(-c4c([2H])c([2H])c([2H])c([2H])c4-c4c([2H])c([2H])c([2H])c5c4sc4c([2H])c([2H])c([2H])c([2H])c45)c4oc5c([2H])c([2H])c([2H])c([2H])c5c34)c([2H])c2[2H])c([2H])c1[2H]] | 28.1 g (70%) | 781.15 |
| A261 | 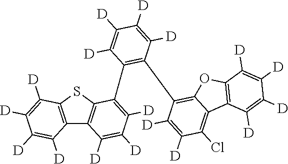 [SMILES: [2H]c1c([2H])c([2H])c(-c2c([2H])c([2H])c([2H])c3c2sc2c([2H])c([2H])c([2H])c([2H])c23)c(-c2c([2H])c([2H])c(Cl)c3c2oc2c([2H])c([2H])c([2H])c([2H])c23)c1[2H]] | 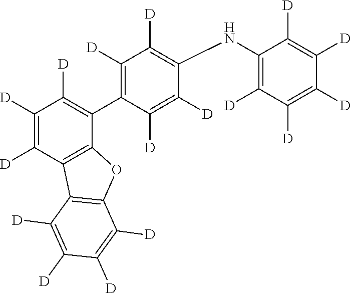 [SMILES: [2H]c1c([2H])c([2H])c(Nc2c([2H])c([2H])c(-c3c([2H])c([2H])c([2H])c4c3oc3c([2H])c([2H])c([2H])c([2H])c34)c([2H])c2[2H])c([2H])c1[2H]] | 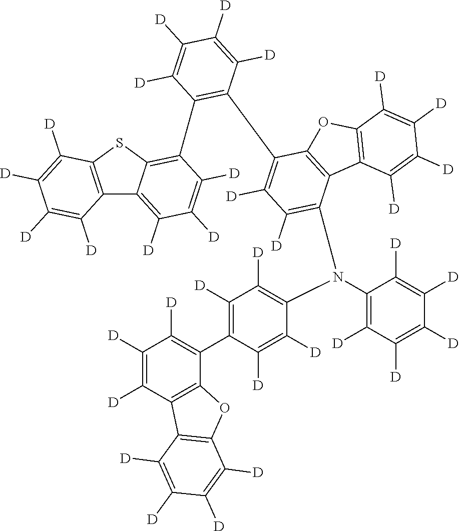 [SMILES: [2H]c1c([2H])c([2H])c(N(c2c([2H])c([2H])c(-c3c([2H])c([2H])c([2H])c4c3oc3c([2H])c([2H])c([2H])c([2H])c34)c([2H])c2[2H])c2c([2H])c([2H])c(-c3c([2H])c([2H])c([2H])c([2H])c3-c3c([2H])c([2H])c([2H])c4c3sc3c([2H])c([2H])c([2H])c([2H])c34)c3oc4c([2H])c([2H])c([2H])c([2H])c4c23)c([2H])c1[2H]] | 26.1 g (64%) | 793.12 |

### [Experimental Example 1] Measurement of HOMO and LUMO

The hole transport auxiliary layer plays a role in reducing accumulation of holes at an interface between the hole transport layer and the light emitting layer due to a difference between a HOMO level of the hole transport layer and a HOMO level of the light emitting layer. To this end, a difference between the HOMO level of the light emitting layer and a HOMO level of the hole transport auxiliary layer should be smaller than a difference between the HOMO level of the hole injection layer and the HOMO level of the hole transport auxiliary layer. Furthermore, the hole transport auxiliary layer should have a higher LUMO energy level than a LUMO energy level of the light emitting layer to minimize electrons leaking from the light emitting layer to the hole transport layer.

In order to check whether the compound represented by the Chemical Formula A in accordance with the present disclosure is suitable as a material of the hole transport auxiliary layer, the HOMO energy level (eV) and the LUMO energy level (eV) of the hole transport auxiliary layer containing the compound represented by the Chemical Formula A in accordance with the present disclosure were calculated using Spartan software (B3LYP DFT 6-31G* by spartan'16) and the calculation results are shown in Table 20 as set forth below.

TABLE 20

| Compound | HOMO (calculation) | LUMO (calculation) |
|---|---|---|
| A5 | 4.96 | 1.10 |
| A313 | 4.97 | 1.09 |
| A521 | 4.99 | 1.06 |
| A652 | 4.98 | 1.05 |
| A60 | 4.95 | 1.12 |
| A688 | 4.98 | 1.07 |
| A479 | 4.96 | 1.09 |
| A201 | 4.96 | 1.12 |
| A642 | 4.95 | 1.11 |
| A63 | 4.84 | 1.13 |
| A304 | 4.84 | 1.11 |
| A514 | 4.85 | 1.16 |
| A681 | 4.85 | 1.08 |

### [Present Example 1] Manufacturing of Organic Light Emitting Diode (Blue Light Emitting Layer)

A substrate on which ITO (100 nm) as a first electrode (a positive electrode) of an organic light emitting diode was deposited was patterned in a distinguishing manner of a first electrode (positive electrode) area, a second electrode (negative electrode) area, and an insulating layer area from each other in an exposure (Photo-Lithography) process. Then, for the purpose of increasing a work-function of the first electrode (positive electrode), that is, ITO and cleaning, a surface-treatment was performed thereon using UV-ozone and 02: N2 plasma.

Next, NDP-9 (2-(7-Dicyanomethylene-1,3,4,5,6,8,9,10-octafluoro-7H-pyren-2-ylidene)-malononitrile) and N4,N4,N4′,N4′-Tetra([1,1′-biphenyl]-4-yl)-[1,1′-biphenyl]-4,4′-diamine were mixed with each other in a ratio of 3:97 to produce a mixture which in turn was deposited on the positive electrode to form the hole injection layer (HIL) of a thickness of 10 nm.

Then, on the hole injection layer, N4,N4,N4′,N4′-tetra([1,1′-biphenyl]-4-yl)-[1,1′-biphenyl]-4,4′-diamine was vacuum-deposited to form the hole transport layer of a thickness of 100 nm. Then, the Compound A5 was deposited on the hole transport layer (HTL) to form the hole transport auxiliary layer of a thickness of 15 nm.

On the hole transport auxiliary layer, a blue light emitting layer of 25 nm was deposited using 9,10-bis(2-naphthyl) anthracene (ADN) as a host and 2,12-di-tert-butyl-5,9-bis(4-(tert-butyl)phenyl)-7-(3,5-di-tert-butylphenyl)-5,9-dihydro5,9-diaza-13b-boranaphtho[3,2,1-de]anthracene (t-DABNA-dtB) as a dopant, wherein a mixing ratio of host:dopant (by weight) was 97:3.

On the blue light emitting layer, the electron transport layer (ETL) of a thickness of 25 nm was deposited using a mixture of 2-(4-(9,10-di(naphthalene-2-yl) anthracene-2-yl)phenyl)-1-phenyl-1H-benzo[d]imidazole and Liq at a weight ratio of 1:1.

On the electron transport layer (ETL), the electron injection layer (EIL) of a thickness of 1 nm was deposited using Liq. Then, the negative electrode was deposited on the electron injection layer (EIL) so as to have a thickness of 16 nm using a mixture of magnesium and silver at a weight ratio of 1:4. Then, a capping layer made of N4,N4′-bis[4-[bis(3-methylphenyl)amino]phenyl]-N4,N4′-diphenyl-[1,1′-biphenyl]-4,4′-diamine (DNTPD) was deposited so as to have a thickness of 60 nm on the negative electrode. A seal cap containing a moisture absorbent was bonded to the capping layer using a UV curable adhesive to form a protective film (encapsulation layer or protecting layer) to protect the organic light emitting diode from atmospheric oxygen or moisture. In this way, the light emitting diode was manufactured.

### Comparative Examples 1 to 4

The organic light emitting diode of each of Comparative Examples 1 to 4 was manufactured in the same manner as in Present Example 1, except that the Compound A5 used as the hole transport auxiliary layer material in Present Example 1 was replaced with each of Compounds A to D as set forth below. The structures of Compounds A to D which are used as the hole transport auxiliary layer materials respectively used in Comparative Examples 1 to 4 are as follows:

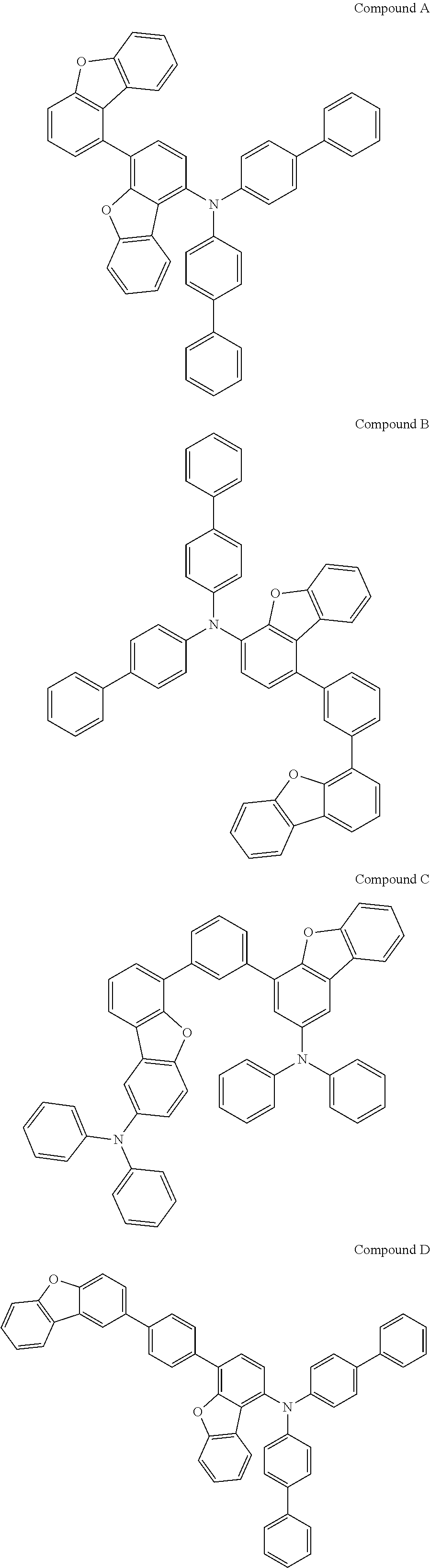

### Present Examples 2 to 171

The organic light emitting diode of each of Present Examples 2 to 171 was manufactured in the same manner as in Present Example 1, except that the Compound A5 used as the hole transport auxiliary layer material in Present Example 1 was replaced with what is shown in Table 21 as set forth below.

### [Experimental Example 2] Organic Light Emitting Diode Performance Evaluation (Blue Organic Light Emitting Diode)

A current of 10 mA/cm² was applied to each of the organic light emitting diodes of Present Examples 1 to 171 and Comparative Examples 1 to 4 using a CS-2000 from KONICA MINOLTA. Then, the operation voltage (V) and external quantum efficiency (EQE) (%) were measured. Furthermore, the lifetime (LT95) (hrs) was measured based on a time duration for which luminance decreases from initial luminance to 95% thereof under application of a constant current of 10 mA/cm² using M6000 from McScience. The measurement results are shown in the Table 21 as set forth below.

TABLE 21

| | Hole transport | Operation | | Lifetime |
|---|---|---|---|---|
| | auxiliary | voltage | EQE | (LT95) |
| Present Example | layer compound | (V) | (%) | (hrs) |
| Present Example 1 | A5 | 3.49 | 19.9 | 460 |
| Present Example 2 | A1 | 3.48 | 20.1 | 459 |
| Present Example 3 | A6 | 3.49 | 19.8 | 460 |
| Present Example 4 | A59 | 3.48 | 20.0 | 462 |
| Present Example 5 | A76 | 3.50 | 20.0 | 462 |
| Present Example 6 | A79 | 3.49 | 19.7 | 460 |
| Present Example 7 | A2 | 3.51 | 20.1 | 458 |
| Present Example 8 | A266 | 3.54 | 18.2 | 421 |
| Present Example 9 | A486 | 3.68 | 16.8 | 371 |
| Present Example 10 | A652 | 3.68 | 16.7 | 370 |
| Present Example 11 | A60 | 3.50 | 20.0 | 459 |
| Present Example 12 | A313 | 3.55 | 18.3 | 420 |
| Present Example 13 | A521 | 3.68 | 16.8 | 371 |
| Present Example 14 | A688 | 3.68 | 16.6 | 373 |
| Present Example 15 | A4 | 3.51 | 19.9 | 455 |
| Present Example 16 | A9 | 3.52 | 20.1 | 456 |
| Present Example 17 | A63 | 3.50 | 19.8 | 456 |
| Present Example 18 | A304 | 3.54 | 18.4 | 413 |
| Present Example 19 | A514 | 3.69 | 16.9 | 372 |
| Present Example 20 | A681 | 3.69 | 16.8 | 372 |
| Present Example 21 | A38 | 3.52 | 20.0 | 458 |
| Present Example 22 | A64 | 3.52 | 19.7 | 455 |
| Present Example 23 | A39 | 3.52 | 20.1 | 457 |
| Present Example 24 | A88 | 3.48 | 19.2 | 455 |
| Present Example 25 | A89 | 3.51 | 19.5 | 453 |
| Present Example 26 | A8 | 3.52 | 19.5 | 458 |
| Present Example 27 | A21 | 3.52 | 19.4 | 458 |
| Present Example 28 | A22 | 3.50 | 19.9 | 456 |
| Present Example 29 | A20 | 3.51 | 19.3 | 456 |
| Present Example 30 | A23 | 3.52 | 20.3 | 453 |
| Present Example 31 | A33 | 3.51 | 19.5 | 454 |
| Present Example 32 | A34 | 3.49 | 19.9 | 455 |
| Present Example 33 | A10 | 3.49 | 20.0 | 451 |
| Present Example 34 | A65 | 3.53 | 19.3 | 452 |
| Present Example 35 | A25 | 3.52 | 19.5 | 450 |
| Present Example 36 | A78 | 3.48 | 20.1 | 456 |
| Present Example 37 | A12 | 3.54 | 19.3 | 449 |
| Present Example 38 | A13 | 3.48 | 19.5 | 450 |
| Present Example 39 | A27 | 3.54 | 19.3 | 448 |
| Present Example 40 | A265 | 3.55 | 18.0 | 419 |
| Present Example 41 | A267 | 3.55 | 17.8 | 418 |
| Present Example 42 | A268 | 3.54 | 17.8 | 419 |
| Present Example 43 | A269 | 3.56 | 18.1 | 411 |
| Present Example 44 | A274 | 3.56 | 18.0 | 416 |
| Present Example 45 | A275 | 3.58 | 18.2 | 419 |
| Present Example 46 | A270 | 3.54 | 18.3 | 419 |
| Present Example 47 | A272 | 3.56 | 17.6 | 418 |
| Present Example 48 | A273 | 3.58 | 17.9 | 415 |
| Present Example 49 | A276 | 3.59 | 17.6 | 425 |
| Present Example 50 | A284 | 3.57 | 17.5 | 412 |
| Present Example 51 | A283 | 3.54 | 17.8 | 417 |
| Present Example 52 | A285 | 3.58 | 17.6 | 416 |
| Present Example 53 | A286 | 3.55 | 17.9 | 413 |
| Present Example 54 | A287 | 3.56 | 18.0 | 418 |
| Present Example 55 | A289 | 3.55 | 17.8 | 418 |
| Present Example 56 | A295 | 3.54 | 18.0 | 413 |
| Present Example 57 | A296 | 3.57 | 17.9 | 417 |
| Present Example 58 | A317 | 3.56 | 18.0 | 415 |
| Present Example 59 | A332 | 3.54 | 18.1 | 416 |
| Present Example 60 | A315 | 3.55 | 18.5 | 422 |
| Present Example 61 | A318 | 3.58 | 18.2 | 413 |
| Present Example 62 | A316 | 3.57 | 17.9 | 417 |
| Present Example 63 | A328 | 3.56 | 18.5 | 423 |
| Present Example 64 | A330 | 3.58 | 18.2 | 418 |
| Present Example 65 | A325 | 3.57 | 18.2 | 420 |
| Present Example 66 | A337 | 3.58 | 17.9 | 413 |
| Present Example 67 | A338 | 3.57 | 17.6 | 417 |
| Present Example 68 | A488 | 3.69 | 16.9 | 372 |
| Present Example 69 | A664 | 3.70 | 16.5 | 375 |
| Present Example 70 | A162 | 3.62 | 17.1 | 395 |
| Present Example 71 | A161 | 3.63 | 16.9 | 399 |
| Present Example 72 | A153 | 3.61 | 16.8 | 395 |
| Present Example 73 | A156 | 3.61 | 16.9 | 397 |
| Present Example 74 | A160 | 3.60 | 17.1 | 398 |
| Present Example 75 | A163 | 3.65 | 17.0 | 398 |
| Present Example 76 | A167 | 3.61 | 17.1 | 395 |
| Present Example 77 | A192 | 3.60 | 16.8 | 396 |
| Present Example 78 | A201 | 3.60 | 17.2 | 398 |
| Present Example 79 | A395 | 3.63 | 16.7 | 381 |
| Present Example 80 | A397 | 3.64 | 16.6 | 380 |
| Present Example 81 | A408 | 3.65 | 16.5 | 381 |
| Present Example 82 | A425 | 3.63 | 16.8 | 383 |
| Present Example 83 | A432 | 3.63 | 16.6 | 381 |
| Present Example 84 | A433 | 3.64 | 16.9 | 384 |
| Present Example 85 | A437 | 3.63 | 17.0 | 385 |
| Present Example 86 | A590 | 3.69 | 17.0 | 370 |
| Present Example 87 | A607 | 3.68 | 16.8 | 379 |
| Present Example 88 | A733 | 3.69 | 16.9 | 377 |
| Present Example 89 | A248 | 3.74 | 16.5 | 344 |
| Present Example 90 | A249 | 3.75 | 16.5 | 343 |
| Present Example 91 | A477 | 3.75 | 16.8 | 347 |
| Present Example 92 | A250 | 3.74 | 16.7 | 348 |
| Present Example 93 | A642 | 3.75 | 17.0 | 340 |
| Present Example 94 | A782 | 3.74 | 16.5 | 335 |
| Present Example 95 | A251 | 3.70 | 16.7 | 345 |
| Present Example 96 | A252 | 3.72 | 16.7 | 346 |
| Present Example 97 | A479 | 3.73 | 16.6 | 347 |
| Present Example 98 | A644 | 3.75 | 16.8 | 339 |
| Present Example 99 | A784 | 3.74 | 16.8 | 338 |
| Present Example 100 | A255 | 3.50 | 20.0 | 507 |
| Present Example 101 | A257 | 3.52 | 19.5 | 505 |
| Present Example 102 | A258 | 3.50 | 19.8 | 505 |
| Present Example 103 | A260 | 3.63 | 17.2 | 430 |
| Present Example 104 | A259 | 3.60 | 17.1 | 433 |
| Present Example 105 | A261 | 3.61 | 17.0 | 432 |
| Present Example 106 | A264 | 3.71 | 16.8 | 380 |
| Present Example 107 | B5 | 3.45 | 20.0 | 438 |
| Present Example 108 | B17 | 3.42 | 20.5 | 442 |
| Present Example 109 | B93 | 3.44 | 20.5 | 441 |
| Present Example 110 | B1 | 3.36 | 21.0 | 443 |
| Present Example 111 | B3 | 3.36 | 20.7 | 441 |
| Present Example 112 | B6 | 3.39 | 20.9 | 439 |
| Present Example 113 | B9 | 3.42 | 21.0 | 440 |
| Present Example 114 | B10 | 3.44 | 21.2 | 435 |
| Present Example 115 | B16 | 3.35 | 21.5 | 446 |
| Present Example 116 | B44 | 3.37 | 21.4 | 445 |
| Present Example 117 | B46 | 3.38 | 21.6 | 448 |
| Present Example 118 | B47 | 3.67 | 21.8 | 447 |
| Present Example 119 | B56 | 3.65 | 21.6 | 445 |
| Present Example 120 | B64 | 3.42 | 21.2 | 440 |
| Present Example 121 | B209 | 3.51 | 19.0 | 405 |
| Present Example 122 | B208 | 3.50 | 18.8 | 407 |
| Present Example 123 | B211 | 3.49 | 18.9 | 408 |
| Present Example 124 | B216 | 3.55 | 18.0 | 410 |
| Present Example 125 | B217 | 3.54 | 18.1 | 409 |
| Present Example 126 | B218 | 3.55 | 17.9 | 405 |
| Present Example 127 | B223 | 3.52 | 18.7 | 405 |
| Present Example 128 | B229 | 3.55 | 17.6 | 407 |
| Present Example 129 | B239 | 3.48 | 19.0 | 406 |
| Present Example 130 | B250 | 3.48 | 19.3 | 410 |
| Present Example 131 | B257 | 3.53 | 18.0 | 410 |
| Present Example 132 | B260 | 3.50 | 19.0 | 408 |
| Present Example 133 | B271 | 3.53 | 18.6 | 402 |
| Present Example 134 | B279 | 3.52 | 18.5 | 403 |
| Present Example 135 | B257 | 3.53 | 17.7 | 408 |
| Present Example 136 | B398 | 3.65 | 17.0 | 366 |
| Present Example 137 | B403 | 3.63 | 17.1 | 367 |
| Present Example 138 | B431 | 3.63 | 17.3 | 370 |
| Present Example 139 | B397 | 3.64 | 17.0 | 366 |
| Present Example 140 | B400 | 3.64 | 16.8 | 365 |
| Present Example 141 | B409 | 3.65 | 16.8 | 365 |
| Present Example 142 | B420 | 3.63 | 16.9 | 367 |
| Present Example 143 | B438 | 3.63 | 17.0 | 369 |
| Present Example 144 | B544 | 3.64 | 17.1 | 365 |
| Present Example 145 | B550 | 3.65 | 17.2 | 367 |
| Present Example 146 | B552 | 3.66 | 16.8 | 368 |
| Present Example 147 | B551 | 3.65 | 16.7 | 367 |
| Present Example 148 | B562 | 3.66 | 17.0 | 368 |
| Present Example 149 | B587 | 3.64 | 17.3 | 366 |
| Present Example 150 | B553 | 3.65 | 16.8 | 369 |
| Present Example 151 | B555 | 3.66 | 16.8 | 367 |
| Present Example 152 | B120 | 3.60 | 17.3 | 388 |
| Present Example 153 | B122 | 3.59 | 17.4 | 387 |
| Present Example 154 | B320 | 3.62 | 17.2 | 377 |
| Present Example 155 | B484 | 3.65 | 17.0 | 363 |
| Present Example 156 | B489 | 3.63 | 17.2 | 365 |
| Present Example 157 | B630 | 3.65 | 17.0 | 364 |
| Present Example 158 | B622 | 3.66 | 16.8 | 361 |
| Present Example 159 | B199 | 3.72 | 16.8 | 345 |
| Present Example 160 | B388 | 3.72 | 16.9 | 344 |
| Present Example 161 | B200 | 3.70 | 17.0 | 342 |
| Present Example 162 | B389 | 3.70 | 17.0 | 342 |
| Present Example 163 | B201 | 3.68 | 17.2 | 340 |
| Present Example 164 | B390 | 3.68 | 17.3 | 341 |
| Present Example 165 | B539 | 3.68 | 17.3 | 333 |
| Present Example 166 | B657 | 3.69 | 17.2 | 335 |
| Present Example 167 | B203 | 3.43 | 22.0 | 455 |
| Present Example 168 | B391 | 3.70 | 19.2 | 420 |
| Present Example 169 | B393 | 3.62 | 17.3 | 415 |
| Present Example 170 | B205 | 3.60 | 17.4 | 427 |
| Present Example 171 | B396 | 3.61 | 17.3 | 420 |
| Comparative | Compound A | 4.05 | 16.5 | 235 |
| Example 1 | | | | |
| Comparative | Compound B | 3.95 | 16.2 | 240 |
| Example 2 | | | | |
| Comparative | Compound C | 4.00 | 16.0 | 230 |
| Example 3 | | | | |
| Comparative | Compound D | 3.90 | 16.1 | 223 |
| Example 4 | | | | |

### [Present Example 172] Manufacturing of Organic Light Emitting Diode (Green Light Emitting Layer)

A substrate on which ITO (100 nm) as a first electrode (positive electrode) of an organic light emitting diode was deposited was patterned in a distinguishing manner of a first electrode (positive electrode) area, a second electrode (negative electrode) area, and an insulating layer area from each other in an exposure (Photo-Lithography) process. Then, for the purpose of increasing a work-function of the first electrode (positive electrode), that is, ITO and cleaning, a surface-treatment was performed thereon using UV-ozone and 02: N2 plasma.

Next, NDP-9 (2-(7-Dicyanomethylene-1,3,4,5,6,8,9,10-octafluoro-7H-pyren-2-ylidene)-malononitrile) and N4,N4,N4′,N4′-Tetra([1,1′-biphenyl]-4-yl)-[1,1′-biphenyl]-4,4′-diamine were mixed with each other in a ratio of 3:97 to produce a mixture which in turn was deposited on the positive electrode to form the hole injection layer (HIL) of a thickness of 10 nm.

Then, on the hole injection layer, N4,N4,N4′,N4′-tetra([1,1′-biphenyl]-4-yl)-[1,1′-biphenyl]-4,4′-diamine was vacuum-deposited to form the hole transport layer of a thickness of 100 nm. Then, the Compound A5 was deposited on the hole transport layer (HTL) to form the hole transport auxiliary layer of a thickness of 15 nm.

On the hole transport auxiliary layer, a green light emitting layer of 35 nm was deposited using 4,4′-N,N′-dicarbazole-biphenyl (CBP) as a host and Ir(ppy)₃ [tris(2-phenylpyridine)-iridium] as a dopant, wherein a mixing ratio of host:dopant (by weight) was 95:5.

On the green light emitting layer, the electron transport layer (ETL) of a thickness of 25 nm was deposited using a mixture of 2-(4-(9,10-di(naphthalene-2-yl) anthracene-2-yl)phenyl)-1-phenyl-1H-benzo[d]imidazole and Liq at a weight ratio of 1:1.

On the electron transport layer (ETL), the electron injection layer (EIL) of a thickness of 1 nm was deposited using Liq. Then, the negative electrode was deposited on the electron injection layer (EIL) so as to have a thickness of 16 nm using a mixture of magnesium and silver at a weight ratio of 1:4. Then, a capping layer made of N4,N4′-bis[4-[bis(3-methylphenyl)amino]phenyl]-N4,N4′-diphenyl-[1,1′-biphenyl]-4,4′-diamine (DNTPD) was deposited so as to have a thickness of 60 nm on the negative electrode. A seal cap containing a moisture absorbent was bonded to the capping layer using a UV curable adhesive to form a protective film (encapsulation layer or protecting layer) to protect the organic light emitting diode from atmospheric oxygen or moisture. In this way, the light emitting diode was manufactured.

### Comparative Examples 5 to 8

The organic light emitting diode of each of Comparative Examples 5 to 8 was manufactured in the same manner as in Present Example 172, except that the Compound A5 used as the hole transport auxiliary layer material in Present Example 172 was replaced with what is shown in Table 22 as set forth below. The structures of Compounds A to D which are used as the hole transport auxiliary layer materials respectively used in Comparative Examples 5 to 8 are the same as those used in Comparative Examples 1 to 4, respectively.

### Present Examples 173 to 221

The organic light emitting diode of each of Present Examples 173 to 221 was manufactured in the same manner as in Present Example 172, except that the Compound A5 used as the hole transport auxiliary layer material in Present Example 172 was replaced with what is shown in Table 22 as set forth below.

### [Experimental Example 3] Organic Light Emitting Diode Performance Evaluation (Green Organic Light Emitting Diode)

A current of 10 mA/cm² was applied to each of the organic light emitting diodes of Present Examples 172 to 221 and Comparative Examples 5 to 8 using a CS-2000 from KONICA MINOLTA. Then, the operation voltage and external quantum efficiency (EQE) (%) were measured. Furthermore, the lifetime (LT95) was measured based on a time duration for which luminance decreases from initial luminance to 95% thereof under application of a constant current of 10 mA/cm² using M6000 from McScience. The measurement results are shown in Table 22 as set forth below.

TABLE 22

| | Hole transport | Operation | | Lifetime |
|---|---|---|---|---|
| | auxiliary | voltage | EQE | (LT95) |
| Present Example | layer compound | (V) | (%) | (hrs) |
| Present Example | A5 | 3.49 | 41.3 | 581 |
| 172 | | | | |
| Present Example | A266 | 3.52 | 37.3 | 574 |
| 173 | | | | |
| Present Example | A486 | 3.55 | 38.4 | 556 |
| 174 | | | | |
| Present Example | A652 | 3.56 | 38.4 | 555 |
| 175 | | | | |
| Present Example | A60 | 3.51 | 40.5 | 582 |
| 176 | | | | |
| Present Example | A313 | 3.53 | 37.4 | 575 |
| 177 | | | | |
| Present Example | A521 | 3.55 | 38.1 | 555 |
| 178 | | | | |
| Present Example | A688 | 3.55 | 38.2 | 556 |
| 179 | | | | |
| Present Example | A63 | 3.50 | 40.9 | 580 |
| 180 | | | | |
| Present Example | A304 | 3.52 | 37.5 | 573 |
| 181 | | | | |
| Present Example | A514 | 3.55 | 38.3 | 567 |
| 182 | | | | |
| Present Example | A681 | 3.56 | 38.1 | 556 |
| 183 | | | | |
| Present Example | A8 | 3.49 | 41.0 | 588 |
| 184 | | | | |
| Present Example | A39 | 3.51 | 39.5 | 585 |
| 185 | | | | |
| Present Example | A65 | 3.50 | 39.8 | 589 |
| 186 | | | | |
| Present Example | A79 | 3.51 | 39.2 | 590 |
| 187 | | | | |
| Present Example | A268 | 3.52 | 37.4 | 571 |
| 188 | | | | |
| Present Example | A325 | 3.53 | 37.2 | 575 |
| 189 | | | | |
| Present Example | A488 | 3.55 | 38.3 | 568 |
| 190 | | | | |
| Present Example | A664 | 3.57 | 38.1 | 569 |
| 191 | | | | |
| Present Example | A192 | 3.54 | 37.1 | 564 |
| 192 | | | | |
| Present Example | A250 | 3.58 | 36.5 | 552 |
| 193 | | | | |
| Present Example | A252 | 3.58 | 36.5 | 551 |
| 194 | | | | |
| Present Example | A255 | 3.51 | 40.5 | 645 |
| 195 | | | | |
| Present Example | A258 | 3.50 | 40.9 | 640 |
| 196 | | | | |
| Present Example | A260 | 3.54 | 37.2 | 629 |
| 197 | | | | |
| Present Example | A259 | 3.53 | 37.2 | 623 |
| 198 | | | | |
| Present Example | A261 | 3.54 | 37.0 | 625 |
| 199 | | | | |
| Present Example | A264 | 3.57 | 36.7 | 607 |
| 200 | | | | |
| Present Example | B5 | 3.56 | 38.0 | 575 |
| 201 | | | | |
| Present Example | B17 | 3.57 | 38.3 | 578 |
| 202 | | | | |
| Present Example | B93 | 3.56 | 37.9 | 580 |
| 203 | | | | |
| Present Example | B209 | 3.56 | 37.8 | 566 |
| 204 | | | | |
| Present Example | B211 | 3.57 | 37.6 | 567 |
| 205 | | | | |
| Present Example | B257 | 3.56 | 37.6 | 569 |
| 206 | | | | |
| Present Example | B229 | 3.57 | 37.7 | 567 |
| 207 | | | | |
| Present Example | B398 | 3.53 | 37.2 | 552 |
| 208 | | | | |
| Present Example | B403 | 3.54 | 37.0 | 554 |
| 209 | | | | |
| Present Example | B431 | 3.53 | 37.1 | 555 |
| 210 | | | | |
| Present Example | B550 | 3.55 | 37.1 | 553 |
| 211 | | | | |
| Present Example | B562 | 3.55 | 37.2 | 550 |
| 212 | | | | |
| Present Example | B587 | 3.53 | 37.0 | 553 |
| 213 | | | | |
| Present Example | A248 | 3.58 | 36.5 | 554 |
| 214 | | | | |
| Present Example | A642 | 3.57 | 36.7 | 552 |
| 215 | | | | |
| Present Example | B388 | 3.57 | 36.8 | 549 |
| 216 | | | | |
| Present Example | A163 | 3.53 | 37.1 | 563 |
| 217 | | | | |
| Present Example | B122 | 3.53 | 37.3 | 563 |
| 218 | | | | |
| Present Example | B390 | 3.55 | 37.0 | 548 |
| 219 | | | | |
| Present Example | B203 | 3.56 | 38.0 | 638 |
| 220 | | | | |
| Present Example | B391 | 3.56 | 37.8 | 625 |
| 221 | | | | |
| Comparative | Compound A | 4.10 | 32.1 | 359 |
| Example 5 | | | | |
| Comparative | Compound B | 4.00 | 34.3 | 359 |
| Example 6 | | | | |
| Comparative | Compound C | 4.20 | 33.5 | 382 |
| Example 7 | | | | |
| Comparative | Compound D | 3.97 | 34.2 | 385 |
| Example 8 | | | | |

The compound of the present disclosure is characterized in that an arylamine group substitutes a #1 position of one side benzene of the heteroaryl including X₁, and the linker is present at a #4 position thereof such that a heteroaryl including X₂ is substituted with ortho or meta. On the other hand, in Comparative Compound A, there is no linker between both heteroaryls. Further, in Comparative Compound B, an arylamine group substitutes a #4 position (opposite to the position in the Compound of the present disclosure) of the heteroaryl including X₁. In Comparative Compound C, two arylamine groups are present as substituents. In Comparative Compound D, both heteroaryls are linked to each other via para.

Thus, according to Table 21 and Table 22, it is identified that the organic light emitting diode to which the Compound of the present disclosure is applied has much better diode performance than that of the organic light emitting diode to which each of the Comparative Compounds A to D is applied. As identified under the diode evaluation results of Table 21 and Table 22, even similar Compounds have the energy levels (e.g., HOMO, LUMO) varying depending on the presence or absence of the linker, a difference in a substitution position, the number of substituents, and the like. For this reason, it may be identified that the presence or absence of the linker, the difference in a substitution position, the number of substituents, and the like act as a major factor in performance improvement during diode deposition, such that significantly different results are derived.

Although embodiments of the present disclosure have been described with reference to the accompanying drawings, the present disclosure is not limited to the above embodiments, but may be implemented in various different forms. A person skilled in the art may appreciate that the present disclosure may be practiced in other concrete forms without changing the technical spirit or essential characteristics of the present disclosure. Therefore, it should be appreciated that the embodiments as described above is not restrictive but illustrative in all respects.

What is claimed is:

1. An organic compound represented by a following Chemical Formula A: 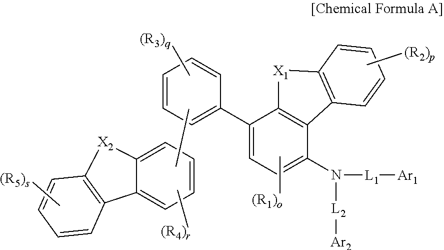
wherein in the Chemical Formula A,
o is an integer of 0 to 2, each of p, q, and s is an integer of 0 to 4, and r is an integer of 0 to 3,
X₁ and X₂ are identical with or different from each other, and each thereof is independently oxygen (O) or sulfur(S),
L₁ and L₂ are identical with or different from each other, and each thereof is independently selected from a single bond and a substituted or unsubstituted arylene group having 6 to 30 carbon atoms,
R₁ to R₅ are identical with or different from each other, and each thereof is independently selected from hydrogen and deuterium,
Ar₁ and Ar₂ are identical with or different from each other, and each thereof is independently selected from a substituted or unsubstituted aryl group having 6 to 30 carbon atoms and a substituted or unsubstituted heteroaryl group having 5 to 60 carbon atoms,
when L₁, L₂, Ar₁, and Ar₂ are respectively substituted with substituents, the substituents are identical with or different from each other, wherein each of substituents independently includes at least one selected from deuterium, a cyano group, a trifluoromethyl group, a nitro group, a halogen group, a hydroxy group, a trimethylsilyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms, wherein when each of substituents includes a plurality of substituents, the plurality of substituents are identical with or different from each other.

2. The organic compound of claim 1, wherein the Chemical Formula A is represented by one of following Chemical Formulas 1 and 2: 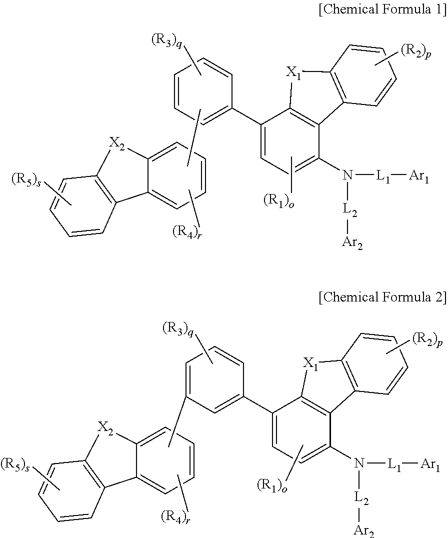
wherein in the Chemical Formula 1 or 2, each of o, p, q, r, s, X₁, X₂, L₁, L₂, R₁ to R₅, Ar₁ and Ar₂ is the same as defined in the Chemical Formula A.

3. The organic compound of claim 2, wherein the Chemical Formula 1 is represented by one of following Chemical Formulas 1-1 to 1-4: 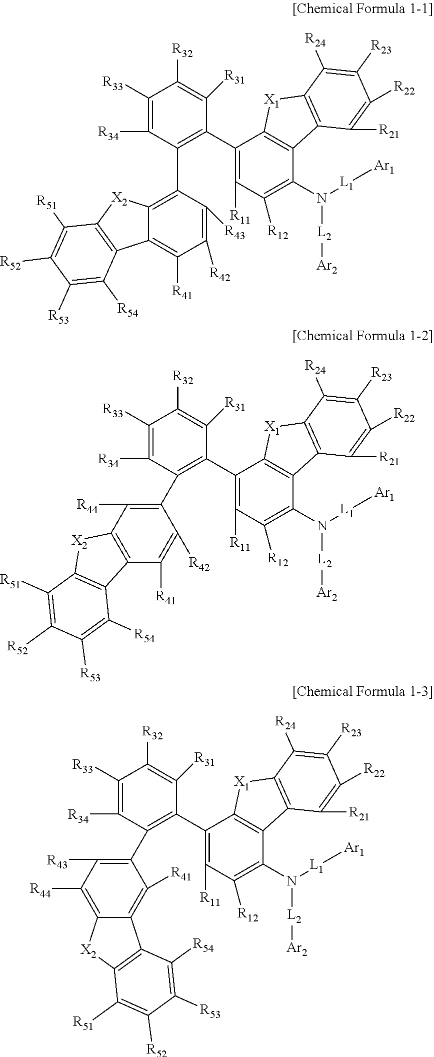 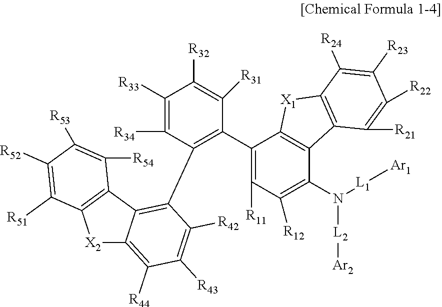
wherein the Chemical Formulas 1-1 to 1-4,
each of X₁, X₂, L₁, L₂, Ar₁ and Ar₂ is the same as defined in the Chemical Formula A,
R₁₁ to R₁₂, R₂₁ to R₂₄, R₃₁ to R₃₄, R₄₁ to R₄₄ and R₅₁ to R₅₄ are identical with or different from each other, and each thereof is independently selected from hydrogen and deuterium.

4. The organic compound of claim 2, wherein the Chemical Formula 2 is represented by one of following Chemical Formulas 2-1 to 2-4: 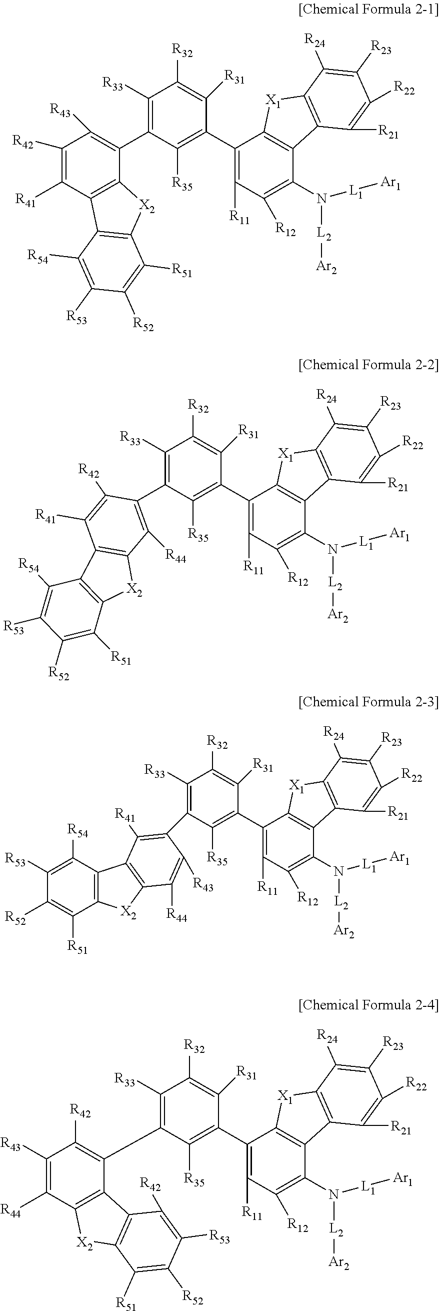
wherein in the Chemical Formulas 2-1 to 2-4,
each of X₁, X₂, L₁, L₂, Ar₁ and Ar₂ is the same as defined in the Chemical Formula A,
R₁₁ to R₁₂, R₂₁ to R₂₄, R₃₁ to R₃₃, R₃₅, R₄₁ to R₄₄ and R₅₁ to R₅₄ are identical with or different from each other, and each thereof is independently selected from hydrogen and deuterium.

5. The organic compound of claim 1, wherein each of L₁ and L₂ is selected from a single bond, and following structures F1 and F2: 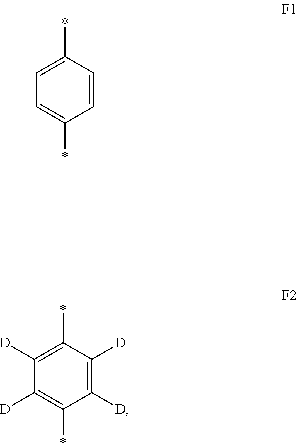
wherein in F1 and F2, “*” denotes a binding site.

6. The organic compound of claim 1, wherein each of Ar₁ and Ar₂ is selected from following structures M1 to M43: 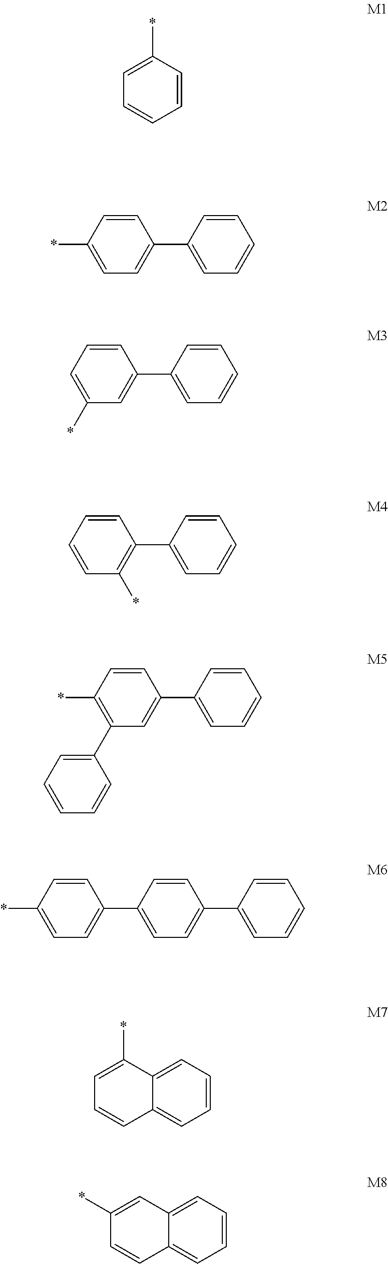 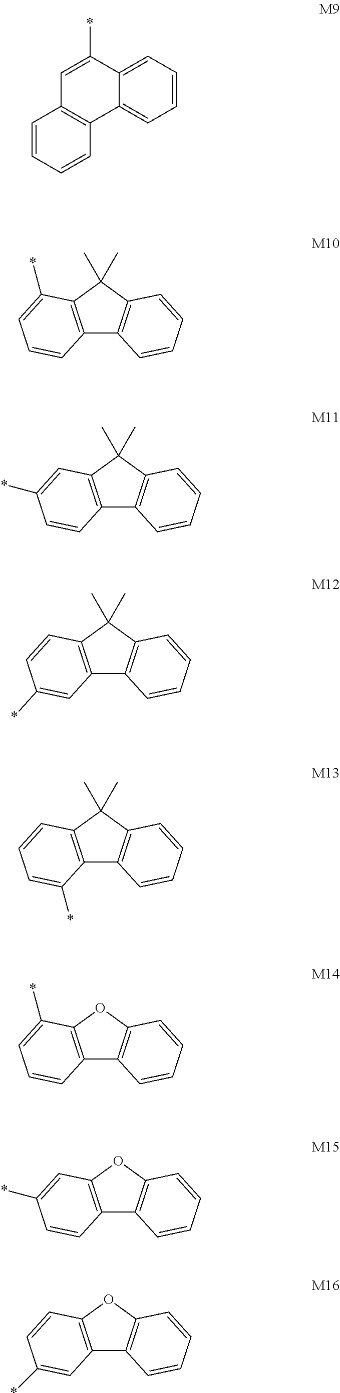 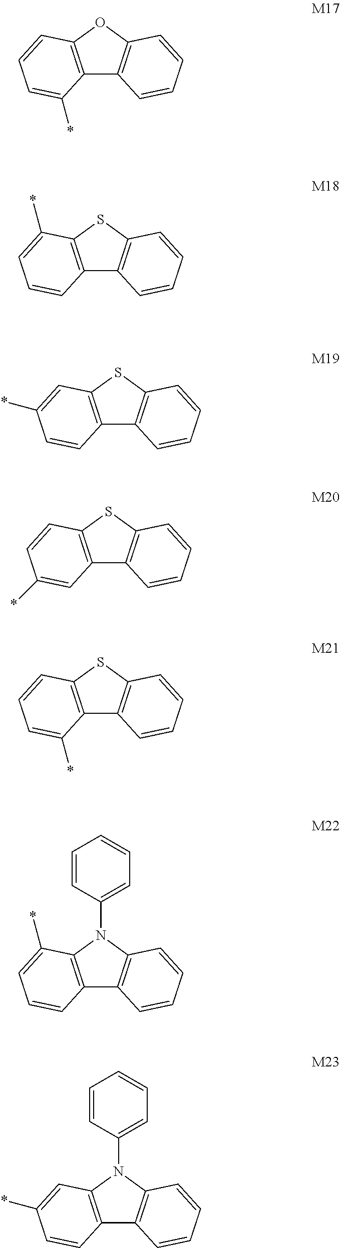 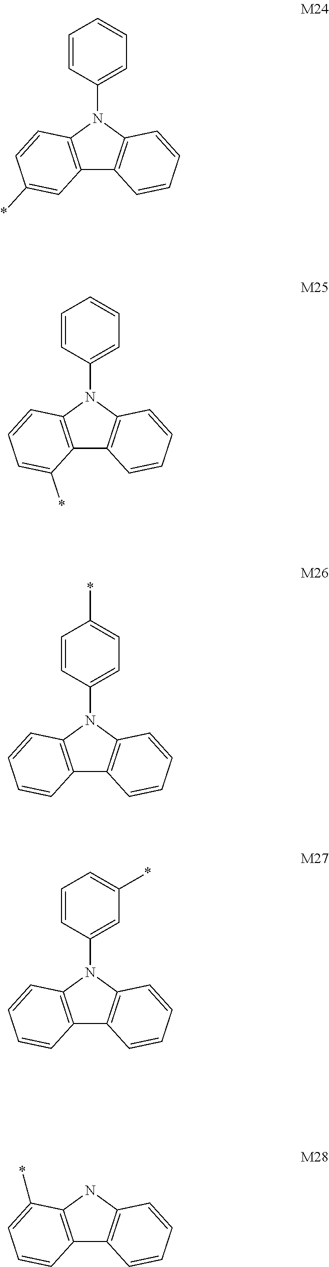 [SMILES: *c1ccc2c(c1)c1ccccc1n2-c1ccccc1 ; *c1cccc2c1c1ccccc1n2-c1ccccc1 ; *c1ccc(-n2c3ccccc3c3ccccc32)cc1 ; *c1cccc(-n2c3ccccc3c3ccccc32)c1 ; *c1cccc2c1[nH]c1ccccc12] 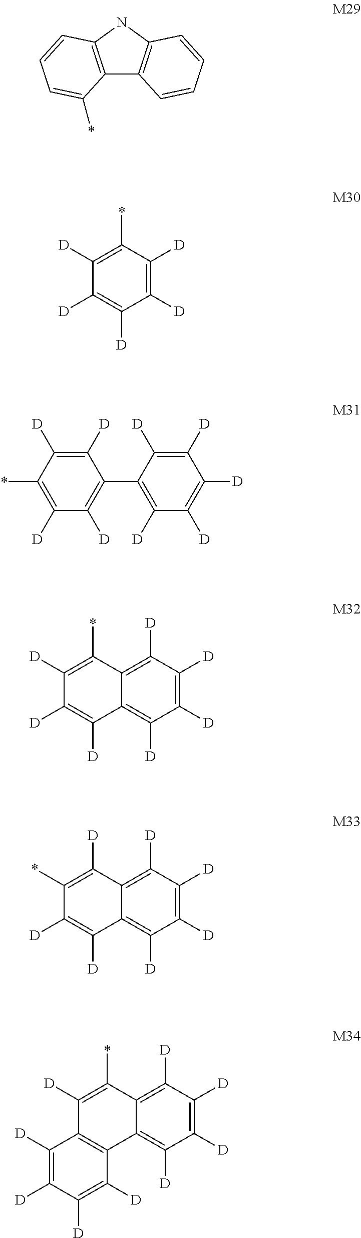 [SMILES: *c1cccc2[nH]c3ccccc3c12 ; *c1c([2H])c([2H])c([2H])c([2H])c1[2H] ; *c1c([2H])c([2H])c(-c2c([2H])c([2H])c([2H])c([2H])c2[2H])c([2H])c1[2H] ; *c1c([2H])c([2H])c([2H])c2c([2H])c([2H])c([2H])c([2H])c12 ; *c1c([2H])c([2H])c2c([2H])c([2H])c([2H])c([2H])c2c1[2H] ; *c1c([2H])c2c([2H])c([2H])c([2H])c([2H])c2c2c([2H])c([2H])c([2H])c([2H])c12] 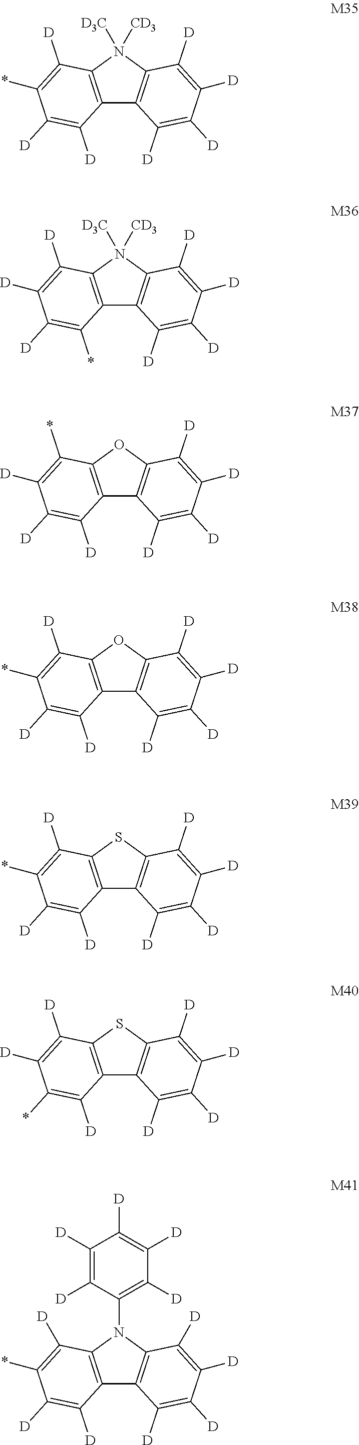 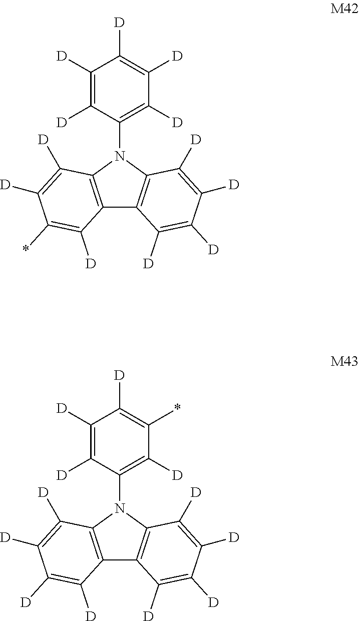 [SMILES: *c1c([2H])c([2H])c2c(c1[2H])c1c([2H])c([2H])c([2H])c([2H])c1n2-c1c([2H])c([2H])c([2H])c([2H])c1[2H] ; *c1c([2H])c([2H])c([2H])c(-n2c3c([2H])c([2H])c([2H])c([2H])c3c3c([2H])c([2H])c([2H])c([2H])c32)c1[2H]]
wherein in M1 to M43, “*” denotes a binding site,
wherein in M28 and M29, N represents NH.

7. An organic light emitting diode comprising:
a first electrode;
a second electrode facing the first electrode; and
at least one organic layer disposed between the first electrode and the second electrode,
wherein the organic layer includes a stack of a hole injection layer, a hole transport layer, an hole transport auxiliary layer, a light emitting layer, an electron transport layer, and an electron injection layer stacked between the first electrode to the second electrode and arranged in this order in a direction from the first electrode to the second electrode,
wherein at least one of the hole transport layer and the hole transport auxiliary layer includes the compound represented by the Chemical Formula A of claim 1.

8. The organic light emitting diode of claim 7, wherein the organic layer including the compound represented by the Chemical Formula A is the hole transport layer.

9. The organic light emitting diode of claim 7, wherein the organic layer including the compound represented by the Chemical Formula A is the hole transport auxiliary layer.

10. The organic light emitting diode of claim 7, wherein the light emitting layer is a blue light emitting layer or a green-light emitting layer.
